(19) United States
(12) Patent Application Publication
(10) Pub. No.: US 2025/0388583 A1
(43) Pub. Date: Dec. 25, 2025

(54) COMPOUNDS AND METHOD FOR PKMYT1 INHIBITION

(71) Applicant: Engine Biosciences Pte. Ltd., Singapore (SG)

(72) Inventors: Toni Kline, Palo Alto, CA (US); Dimitri Khrakovsky, Oakland, CA (US); Ojas Namjoshi, Cambpell, CA (US); Sameer Phadke, Redwood City, CA (US); Stephen Harrison, Bedfordshire (GB); Christine Taylor Brew, Montara, CA (US); Matthew Alexander James Duncton, San Bruno, CA (US)

(21) Appl. No.: 18/877,190

(22) PCT Filed: Jun. 23, 2023
(86) PCT No.: PCT/SG2023/050444

§ 371 Date: Dec. 19, 2024

Related U.S. Application Data
(60) Provisional application No. 63/366,911, filed on Jun. 23, 2022.
(60) Provisional application No. 63/494,522, filed on Apr. 6, 2023.

(30) Foreign Application Priority Data
Jun. 21, 2023 (TW) 112123541

(51) Int. Cl. C07D 487/04; A61K 31/437; A61K 31/444; A61K 31/4985; A61K 31/519; A61K 31/695; C07D 471/04; C07F 7/08

(52) U.S. Cl. CPC C07D 487/04; A61K 31/437; A61K 31/444; A61K 31/4985; A61K 31/519; A61K 31/695; C07D 471/04; C07F 7/0812

(57) ABSTRACT

The disclosure provides for compounds and methods for inhibiting protein kinase, membrane associate tyrosine/threonine 1 (PKMYT1).

### CROSS REFERENCE TO RELATED APPLICATIONS

This application claims the benefit of U.S. Application No. 63/366,911, filed Jun. 23, 2022, and U.S. Application No. 63/494,522, filed Apr. 6, 2023, both of which are hereby incorporated by reference in their entirety.

### FIELD OF THE DISCLOSURE

The disclosure relates to compounds which function as inhibitors of protein kinase, membrane associated tyrosine/threonine 1 (PKMYT1).

### BACKGROUND OF THE DISCLOSURE

PKMYT1 (or Myt1) is a member of the Wee family and was first reported as a kinase capable of phosphorylating Cdc2 efficiently on both threonine-14 and tyrosine-15 in a Xenopus frog. PKMYT1 inhibits cell cycle progression by inhibiting the activities of cell cycle-associated proteins, such as Cyclin A, CDK1, and CDK2. PKMYT1 also drives the progression of a variety of tumors.

The inhibitory phosphorylation of cdc2 is important for the timing of entry into mitosis. Entry into mitosis is initiated by the M phase-promoting factor (MPF), a complex containing the cdc2 protein kinase and cyclin B. Proper regulation of MPF ensures that mitosis occurs only after earlier phases of the cell cycle are complete, Phosphorylation of cdc2 at Tyr-15 and Thr-14 suppresses this activity during interphase (G1, S, and G2). At G2-M transition, cdc2 is dephosphorylated at Tyr-15 and Thr-14 allowing MPF to phosphorylate its mitotic substrates.

Studies have shown that premature activation of cdc2 leads to mitotic catastrophe and cell death. Inhibition of Myt1 is predicted to cause premature activation of cdc1 and thus would kill rapidly proliferating cells. In addition, Myt1 inhibition is predicted to reduce resistance to conventional DNA-damaging chemotherapeutics, because the mechanisms by which cells avoid death involve arrest in the G2 phase of the cell cycle, and repair or DNA damage prior to division. That arrest should be prevented by blocking: Myt1 inhibitory phosphorylation of cdc2, thus forcing the cell to enter mitosis prematurely Myt1 kinase is an important cell cycle regulator, particularly at the G2/M phase. This is due to cell cycle regulation and subsequent repair of damage to DNA or mitotic apparatus, the targets for most effective chemotherapeutic agents. Myt1 kinase offers a point of intervention downstream from these mechanisms by which tumor cells develop resistance. Inhibition of Myt1 could in and of itself have therapeutic benefit in reducing tumor proliferation and in addition, could be used in conjunction with conventional chemotherapies to overcome drug resistance.

Based on the foregoing, there is a need to identify a potent PKMYT1 (Myt1) kinase inhibitor for the treatment of cancer.

### SUMMARY OF THE DISCLOSURE

The present disclosure addresses the above need and provides additional advantages as well.

In some aspects, the present disclosure provides for compounds that function as inhibitors of protein kinase, membrane associated tyrosine/threonine 1 (PKMYT1).

In an aspect, presented herein is a compound of Formula (V), or a pharmaceutically acceptable salt thereof:

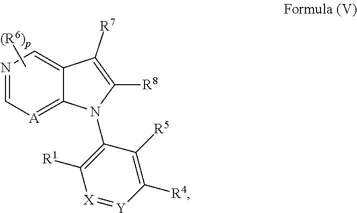

wherein;
A is N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; or
two R⁶ on adjacent atoms join together to form a 6-membered carbocyclic or heterocyclic ring each of which is optionally substituted with one or two substituents independently selected from R^{c};
R⁷ and R⁸ are each independently C₁-C₃ haloalkyl, —N(R^{b})₂, —C(═O)R^{a}, —C(═O)OR^{b}, or —C(═O)N(R^{b})₂;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —Si(CH₃)₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1, 2, or 3.

In some embodiments, provided herein is a compound of Formula (VI), or a pharmaceutically acceptable salt thereof:

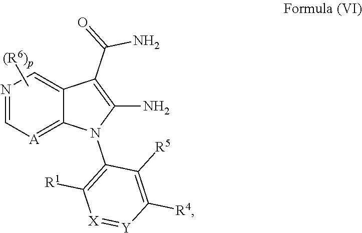

wherein;
A is N or CR⁶;
X is N or CR²;
Y is N or CR³;
R¹ and R⁵ are each independently halogen, C₁-C₆ alkyl, or C₁-C₆ haloalkyl;
R², R³, and R⁴ are each independently hydrogen, halogen, —OH, or —OR^{a}; or
R³ and R⁴ or R⁵ and R⁴ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

In some embodiments, the compound of Formula (VI) has the structure of Formula (VIa), or a pharmaceutically acceptable salt thereof:

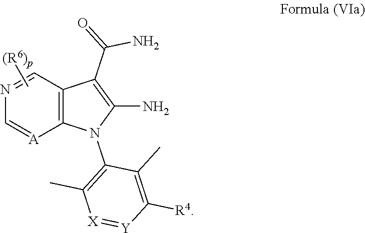

In some embodiments, provided herein is a compound of Formula (VIIa), or a pharmaceutically acceptable salt thereof:

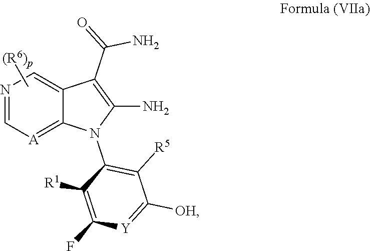

wherein;
A is N or CR⁶;
Y is N or CH;
R¹ and R⁵ are each independently halogen, C₁-C₆ alkyl, or C₁-C₆ haloalkyl;
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

In some embodiments, provided herein is a compound of Formula (VIIb), or a pharmaceutically acceptable salt thereof.

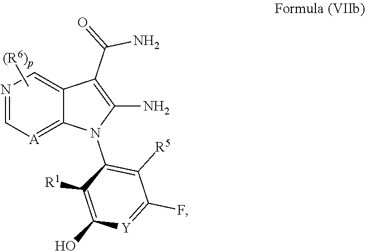

wherein;
A is N or CR⁶;
Y is N or CH;
R¹ and R⁵ are each independently halogen, C₁-C₆ alkyl, or C₁-C₆ haloalkyl;
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

In another aspect, provided herein is a compound of Formula (I), or a pharmaceutically acceptable salt thereof.

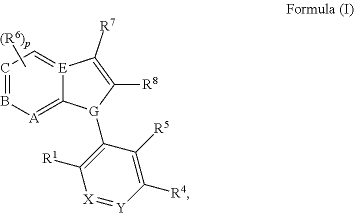

wherein;
A, B, and C are each independently N or CR⁶;
E and F are each independently N or C, wherein at least one of A, B, C, or E is N;
G is N or C;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c};
or R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
R⁷ and R⁸ are each independently —C₁-C₃ haloalkyl, —N(R^{b})₂, —C(═O)R^{a}, —C(═O)OR^{b}, or —C(═O)N(R^{b})₂;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1-4.

In some embodiments, the compound of Formula (I) has the structure of Formula (Ia), or a pharmaceutically acceptable salt thereof:

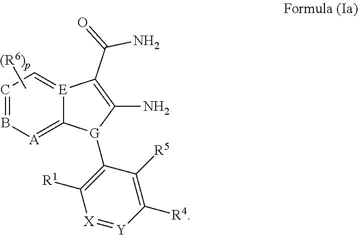

In some embodiments, provided herein is a compound of Formula (II), or a pharmaceutically acceptable salt thereof:

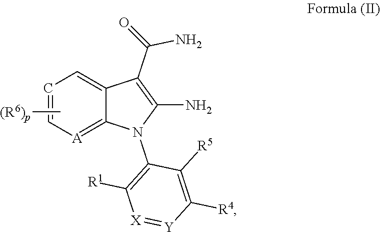

wherein;
A and C are each independently N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c};
or R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-4.

In some embodiments, the compound of Formula (II) has the structure of Formula (IIa) or a pharmaceutically acceptable salt thereof:

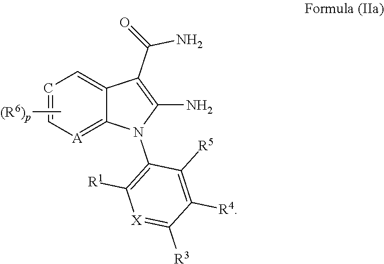

In some embodiments, provided herein is a compound of Formula (III), or a pharmaceutically acceptable salt thereof:

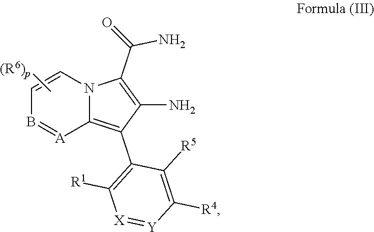

wherein;
A and B are each independently N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c};
or R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-4.

In some embodiments, the compound of Formula (III) has the structure of Formula (IIIa), or a pharmaceutically acceptable salt thereof:

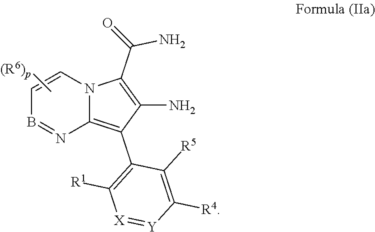

In another aspect, provided herein is a compound of Formula (VIII), or a pharmaceutically acceptable salt thereof:

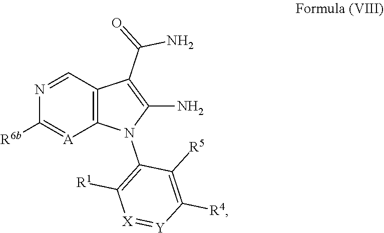

wherein;
A is N or CR^{6a};
X is N or CR²;
Y is N or CH;
R¹ and R⁵ are each independently halogen, C₁-C₆ alkyl, or C₁-C₆ haloalkyl;
R², R³, and R⁴ are each independently hydrogen, halogen, —OH, or —OR^{a};
R^{6a} and R^{6b} are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c}; and
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl.

In another aspects, provided herein is a pharmaceutical composition comprising a compound of Formula (I), (Ia), (II), (IIa), (III), (IIIa), (V), (VI), (VIa), (VIIa), (VIIb), or (VIII), or a pharmaceutically acceptable salt thereof; and a pharmaceutically acceptable excipient.

In another aspect, provided herein is a method of treating a cancer in a patient in need thereof, comprising administering to the patient a compound of Formula (I), (Ia), (II), (IIa), (III), (IIIa), (V), (VI), (VIa), (VIIa), (VIIb), or (VIII), or a pharmaceutically acceptable salt thereof.

In some embodiments, the cancer is leukemia, acute myeloid leukemia (AML), chronic myeloid leukemia, acute lymphoblastic leukemia (ALL), non-Hodgkin lymphoma (NHL), Hodgkin lymphoma (HL), or multiple myeloma (MM). In some embodiments, the cancer is AML. In some embodiments, he cancer is a solid cancer. In some embodiments, the cancer is a skin cancer, ocular cancer, gastrointestinal cancer, thyroid cancer, breast cancer, ovarian cancer, central nervous system cancer, laryngeal cancer, cervical cancer, lymphatic system cancer, genitourinary tract cancer, bone cancer, biliary tract cancer, endometrial cancer, liver cancer, lung cancer, prostate cancer, or colon cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### A. Definitions

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁-6alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀alkyl. In some embodiments, the alkyl is a C₁₋₆alkyl. In some embodiments, the alkyl is a C₁-C₅alkyl. In some embodiments, the alkyl is a C₁-C₄alkyl. In some embodiments, the alkyl is a C₁-C₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —C(═O)OH, —C(═O)OMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —C(═O)OH, C(═O)OMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —C(═O)OH, C(═O)OMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —C(═O)OH, C(═O)OMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —C(═O)OH, C(═O)OMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carbocycle” refers to a saturated, unsaturated, or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —C(═O)OH, C(═O)OMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —C(═O)OH, C(═O)OMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —C(═O)OH, C(═O)OMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### B. Compounds of the Disclosure

In some aspects, the present disclosure provides for compounds that function as modulators of PKMYT1. In some embodiments, the present disclosure provides for compounds that function as inhibitors of PKMYT1.

In an aspect, provided herein is a compound of Formula (A), or a pharmaceutically acceptable salt, solvate, stereoisomer, or atropisomer thereof:

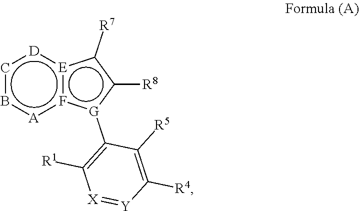

wherein;
A, B, C, and D are each independently N or CR⁶;
E and F are each independently N or C, wherein at least one of A, B, C, D, E, or F is N;
G is N or C;
X is N or CR²;
Y is CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
R⁷ and R⁸ are each independently —C₁-C₃ haloalkyl, —N(R^{b})₂, —C(═O)R^{a}, —C(═O)OR^{b}, or —C(═O)N(R^{b})₂;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with 1 to 4 substituents independently selected from R^{c}; and
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl.

In some embodiments of Formula (A), at least one of A, B, C, D, or E is N.

In some embodiments of Formula (A), at least one of A, B, C, D, or F is N.

In some embodiments of Formula (A), at least one of A, B, C, or D is N. In some embodiments of Formula (A), at least one of A, B, C, or D is N.

In another aspect, provided herein is a compound of Formula (I), or a pharmaceutically acceptable salt, solvate, stereoisomer, or atropisomer thereof:

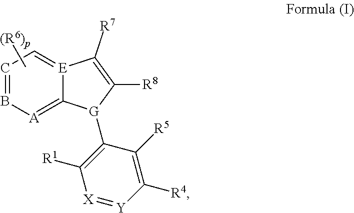

wherein;
A, B, and C are each independently N or CR⁶;
E and F are each independently N or C, wherein at least one of A, B, C, or E is N;
G is N or C;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
R⁷ and R⁸ are each independently —C₁-C₃ haloalkyl, —N(R^{b})₂, —C(═O)R^{a}, —C(═O)OR^{b}, or —C(═O)N(R^{b})₂;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c}; and
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1-4.

In some embodiments of Formula (A) or (I), R⁷ is —C(═O)R^{a}, —C(═O)OR^{b}, or —C(═O)N(R^{b})₂. In some embodiments, R⁷ —N(R^{b})₂. In some embodiments, R⁷ is —C(═O)N(R^{b})₂. In some embodiments, R⁷ is —C(═O)NH₂. In some embodiments of Formula (A) or (I), R⁷ is C₁-C₃ haloalkyl. In some embodiments of Formula (A) or (I), R⁷ is —CF₃ or —CHF₂.

In some embodiments of Formula (A) or (I), R⁸ is —C(═O)R^{a}, —C(═O)OR^{b}, or —C(═O)N(R^{b})₂. In some embodiments, R⁸ is —N(R^{b})₂. In some embodiments, R⁸ is —NH₂. In some embodiments, R⁸ is C₁-C₃ haloalkyl. In some embodiments, R⁸ is —CF₃ or —CHF₂.

In some embodiments of Formula (A) or (I), A is N. In some embodiments, A is CR⁶. In some embodiments, B is N. In some embodiments, B is CR⁶. In some embodiments, C is N. In some embodiments, C is CR⁶. In some embodiments, D is N. In some embodiments, D is CR⁶.

In some embodiments of Formula (A) or (I), at least one of A, B, C, or D is N.

In some embodiments of Formula (A) or (I), one of A, B, C, or D is N. In some embodiments, two of A, B, C, or D is N. In some embodiments, three of A, B, C, or D is N.

In some embodiments of Formula (A) or (I), F is N. In some embodiments, F is C. In some embodiments, G is N. In some embodiments, G is C.

In some embodiments, the compound of Formula (A) has the structure of Formula (I*), or a pharmaceutically acceptable salt, solvate, stereoisomer, or atropisomer thereof:

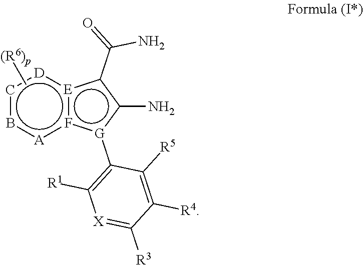

In some embodiments, the compound of Formula (I) has the structure of Formula (Ia), or a pharmaceutically acceptable salt, solvate, stereoisomer, or atropisomer thereof:

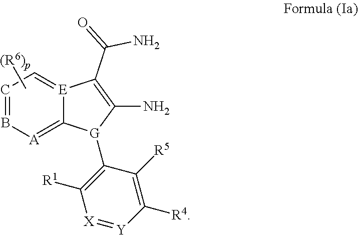

In some embodiments of Formula (A), (I*), (I) or (Ia), E is C; and G is N.

In some embodiments of Formula (A), (I*), (I), or (Ia), B is CR⁶.

In some embodiments of Formula (A), (I*), (I), or (Ia), E is N; and G is C.

In some embodiments of Formula (A), (I*), (I), or (Ia), C is CR⁶.

In some embodiments, provided herein is a compound of Formula (II), or a pharmaceutically acceptable salt, solvate, stereoisomer, or atropisomer thereof:

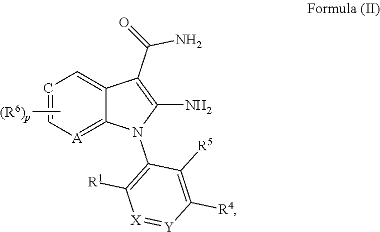

wherein;
A and C are each independently N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c};
or R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-4.

In some embodiments of Formula (II), A is N and C is N or CR⁶.

In some embodiments of Formula (II), A is CR⁶ and C is N.

In some embodiments of Formula (II), A is N and C is CR⁶.

In some embodiments, the compound of Formula (II) has the structure of Formula (IIa), or a pharmaceutically acceptable salt, solvate, stereoisomer, or atropisomer thereof:

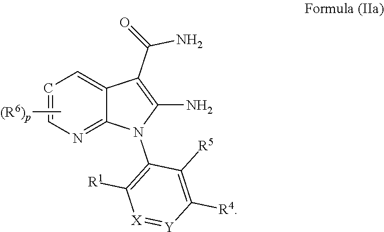

In some embodiments, the compound of Formula (II) has the structure of Formula (IIb), or a pharmaceutically acceptable salt, solvate, stereoisomer, or atropisomer thereof:

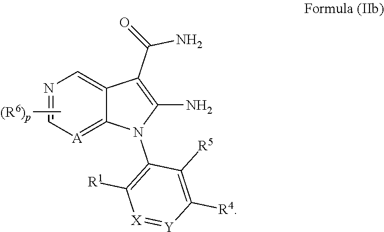

In some embodiments of Formula (II), (IIa), or (IIb), if X is CH and R⁴ is OH; then R⁵ is not halogen or C₁-C₆ alkyl.

In some embodiments of Formula (II), (IIa), or (IIb), if X is CH and R⁴ is OH; then R⁵ is —NH₂.

In some embodiments of Formula (II), (IIa), or (IIb), R⁵ is hydrogen, —CN, —OH, —OR^{a}, —NO₂, —N(R^{b})₂, C₁-C₆ haloalkyl, C₁-C₆ hydroxyalkyl, C₁-C₆ heteroalkyl, C₃-C₆ cycloalkyl, or 4 to 6 membered heterocycloalkyl. In some embodiments, R⁵ is —N(R^{b})₂.

In some embodiments of Formula (II), (IIa), or (IIb), R⁴ is hydrogen, halogen, —CN, —OH, —OR^{a}, —NO₂, —N(R^{b})₂, C₁-C₆ alkyl, C₁-C₆haloalkyl, C₁-C₆hydroxyalkyl, or C₁-C₆heteroalkyl. In some embodiments, R⁴ is —OH.

In some embodiments, the compound of Formula (I*) has the structure of Formula (III*), or a pharmaceutically acceptable salt, solvate, stereoisomer, or atropisomer thereof:

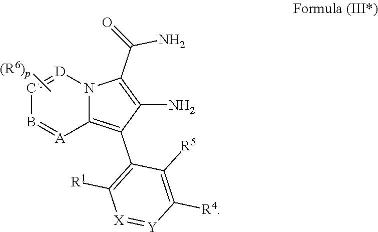

In some embodiments, provided herein is a compound of Formula (III), or a pharmaceutically acceptable salt, solvate, stereoisomer, or atropisomer thereof:

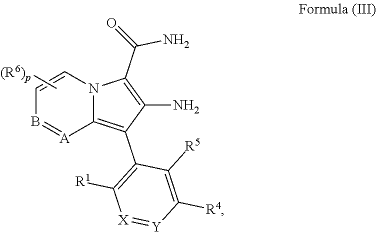

wherein;
A and B are each independently N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c};
or R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-4.

In some embodiments of the compound of Formula (III*) or (III), A is N.

In some embodiments, the compound of Formula (III) has the structure of Formula (IIIa), or a pharmaceutically acceptable salt, solvate, stereoisomer, or atropisomer thereof:

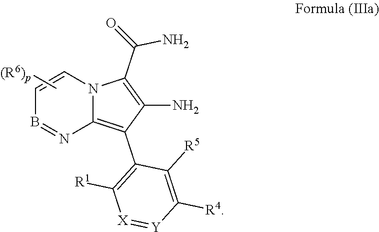

In some embodiments of the compound of Formula (III*), (III), or (IIIa), B is N. In some embodiments, B is CR⁶.

In another aspect, provided herein is a compound of Formula (IV), or a pharmaceutically acceptable salt, solvate, stereoisomer, or atropisomer thereof:

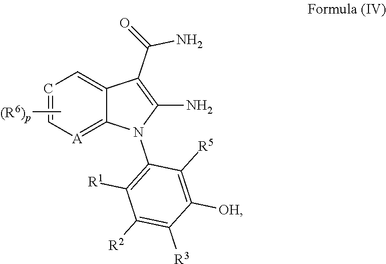

wherein;
A and C are each independently N or CR⁶; wherein one of A or C is N;
R¹, R², R³ and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁- C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
or two R⁶ on adjacent atoms join together to form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-3.

In some embodiments of Formula (IV), A is N; and C is N.

In some embodiments of Formula (IV), A is N and C is CR⁶.

In some embodiments, provided herein is a compound of Formula (IVa), or a pharmaceutically acceptable salt, solvate, stereoisomer, or atropisomer thereof:

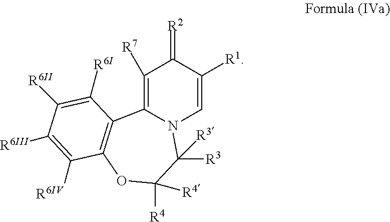

wherein;
R¹, R², R³ and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁- C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c};
R^{6b} and R^{6c} are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R_{c};
or R^{6a} and R^{6b} together with the atoms to which they are attached form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl.

In some embodiments of Formula (IVa), R^{6b} is halogen, —CN, —OR^{a}, —N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl, each of which is optionally substituted with one, two, three, or four substituents independently selected from R^{c}; and R^{6c} is hydrogen. In some embodiments, R^{6b} is halogen, —CN, —OR^{a}, —N(R^{b})₂, —NHC(═O)R^{a}, C₁-C₆ alkyl, C₂-C₆ alkynyl, C₁-C₆ haloalkyl C₁-C₆ hydroxyalkyl, C₃-C₆ cycloalkyl, or 4 to 6 membered heterocycloalkyl, each of which is optionally substituted with one, two, three, or four halogen, —OH, cycloalkyl, heterocycloalkyl, or phenyl; and R^{6c} is hydrogen.

In some embodiments of Formula (IVa), R^{6b} is hydrogen; and R^{6c} is halogen, —CN, —OR^{a}, —N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl, each of which is optionally substituted with one, two, three, or four substituents independently selected from R^{c}. In some embodiments, R^{6b} is hydrogen; and R^{6c} is halogen, —CN, —OR^{a}, —N(R^{b})₂, —NHC(═O)R^{a}, C₁-C₆ alkyl, C₂-C₆ alkynyl, C₁-C₆ hydroxyalkyl, C₃-C₆ cycloalkyl, or 4 to 6 membered heterocycloalkyl, each of which is optionally substituted with one, two, three, or four halogen, —OH, cycloalkyl, heterocycloalkyl, or phenyl.

In some embodiments of Formula (IV), R^{6a} and R^{6b} together with the atoms to which they are attached form a 6-membered carbocyclic or heterocyclic ring.

In some embodiments of Formula (IV) or (IVa), R⁵ is C₁-C₆ alkyl, C₁-C₆ haloalkyl, or C₁-C₆ hydroxyalkyl; and R² and R³ are each independently hydrogen. In some embodiments of Formula (IV) or (IVa), R¹ is C₁-C₆ alkyl. In some embodiments of Formula (IV) or (IVa), R⁵ methyl or ethyl. In some embodiments of Formula (IV) or (IVa), R⁵ methyl. In some embodiments of Formula (IV) or (IVa), R⁵ is ethyl.

In some embodiments of Formula (IV) or (IVa), R⁵ is —N(R^{b})₂. In some embodiments of Formula (IV) or (IVa), R⁵ is —NH₂.

In some embodiments of Formula (IV) or (IVa), R¹ is C₁-C₆ alkyl, C₁-C₆ haloalkyl, or C₁-C₆ hydroxyalkyl; and R² and R³ are each independently hydrogen. In some embodiments of Formula (IV) or (IVa), R¹ is C₁-C₆ alkyl. In some embodiments of Formula (IV) or (IVa), R¹ is methyl or ethyl. In some embodiments of Formula (IV) or (IVa), R¹ is methyl. In some embodiments of Formula (IV) or (IVa), R¹ is ethyl.

In another aspect, provided herein is a compound of Formula (V), or a pharmaceutically acceptable salt, solvate, stereoisomer, or atropisomer thereof:

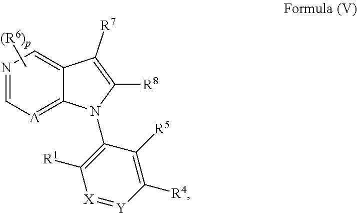

wherein;
A is N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; or
two R⁶ on adjacent atoms join together to form a 6-membered carbocyclic or heterocyclic ring;
R⁷ and R⁸ are each independently C₁-C₃ haloalkyl, —N(R^{b})₂, —C(═O)R^{a}, —C(═O)OR^{b}, or —C(═O)N(R^{b})₂;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1, 2, or 3.

In some embodiments of Formula (V), R⁷ is —C(═O)NH₂; and R⁸ is —NH₂.

In some embodiments, provided herein is a compound is of Formula (VI), or a pharmaceutically acceptable salt, solvate, stereoisomer, or atropisomer thereof:

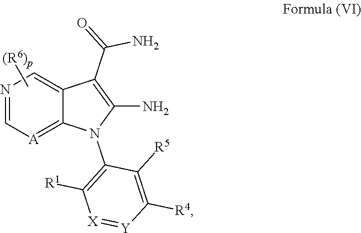

wherein;
A is N or CR⁶;
X is N or CR²;
Y is N or CR³;
R¹ and R⁵ are each independently halogen, C₁-C₆ alkyl, or C₁-C₆ haloalkyl;
R², R³, and R⁴ are each independently hydrogen, halogen, —OH, or —OR^{a}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

In some embodiments of Formula (VI), R¹ is methyl and R⁵ is methyl.

In some embodiments, provided herein is a compound of Formula (VIa), or a pharmaceutically acceptable salt, solvate, stereoisomer, or atropisomer thereof:

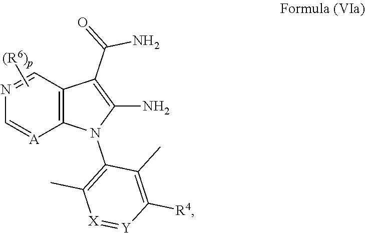

wherein;
A is N or CR⁶;
X is N or CR²;
Y is N or CR³;
R², R³, and R⁴ are each independently hydrogen, halogen, —OH, or —OR^{a}; or
R³ and R⁴ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

In some embodiments of Formula (V), (VI), or (VIa), R⁴ or R² is —OH.

In some embodiments of Formula (V), (VI), or (VIa), R⁴ is OH; R² is hydrogen or halogen; and R³ is hydrogen or halogen. In some embodiments, R² is halogen and R³ is hydrogen. In some embodiments, R² is hydrogen and R³ is halogen. In some embodiments, R² is hydrogen and R³ is hydrogen.

In some embodiments of Formula (V), (VI), or (VIa), R² is —OH; R³ is hydrogen or halogen; and R⁴ is hydrogen or halogen. In some embodiments, R³ is hydrogen and R⁴ is halogen. In some embodiments, R₃ is halogen and R⁴ is hydrogen. In some embodiments, R³ is hydrogen and R⁴ is hydrogen.

In some embodiments of Formula (V) or (VI), when R⁴ is —OH, then R² is halogen. In some embodiments, when R⁴ is —OH, then R² is —F.

In some embodiments of Formula (V) or (VI), when R² is —OH, then R⁴ is halogen. In some embodiments, when R² is —OH, then R⁴ is —F.

In some embodiments of Formula (V) or (VI), when R⁴ is —OH, then R¹ is methyl and R⁵ is methyl or ethyl.

In some embodiments of Formula (V) or (VI), when R² is —OH, then R⁵ is methyl and R¹ is methyl or ethyl.

In some embodiments, the compound has the structure of Formula (VIIa), or a pharmaceutically acceptable salt, solvate, stereoisomer, or atropisomer thereof:

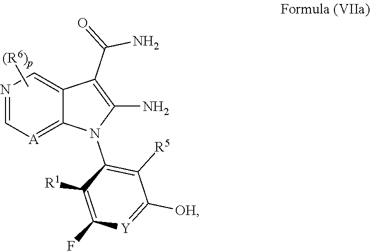

wherein;
A is N or CR⁶;
Y is N or CH;
R¹ and R⁵ are each independently halogen, C₁-C₆ alkyl, or C₁-C₆ haloalkyl;
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

In some embodiments, the compound has the structure of Formula (VIIb), or a pharmaceutically acceptable salt, solvate, stereoisomer, or atropisomer thereof:

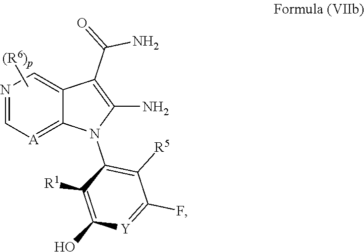

wherein;
A is N or CR⁶;
Y is N or CH;
R¹ and R⁵ are each independently halogen, C₁-C₆ alkyl, or C₁-C₆ haloalkyl;
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

In some embodiments of Formula (VIIa) or (VIIb), when R¹ is methyl, then R⁵ is methyl or ethyl. In some embodiments, when R¹ is methyl, then R⁵ is ethyl. In some embodiments, when R¹ is methyl, then R⁵ is methyl.

In some embodiments of Formula (VIIa) or (VIIb), when R⁵ is methyl, then R¹ is methyl or ethyl. In some embodiments, when R⁵ is methyl, then R¹ is ethyl. In some embodiments, when R⁵ is methyl, then R¹ is methyl.

In some embodiments of Formula (V), (VI), (VIa), (VIIa), or (VIIb), Y is CH.

In another aspect, provided herein in is a compound of Formula (VIII), or a pharmaceutically acceptable salt, solvate, stereoisomer, or atropisomer thereof:

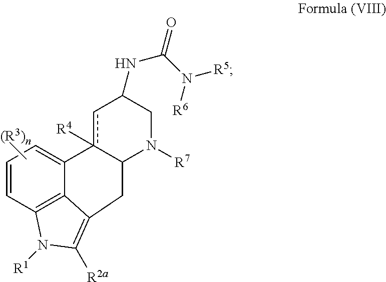

wherein;
A is N or CR^{6a};
X is N or CR²;
Y is N or CH;
R¹ and R⁵ are each independently halogen, C₁-C₆ alkyl, or C₁-C₆ haloalkyl;
R², R³, and R⁴ are each independently hydrogen, halogen, —OH, or —OR^{a};
R^{6a} and R^{6b} are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c}; and
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl.

In some embodiments of Formula (VIII), A is N. In some embodiments, A is CR^{6a}.

In some embodiments of Formula (VIII), R^{6b} is halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁- C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; and R^{6a} is hydrogen, halogen, —CN, C₁-C₆ alkyl, C₂-C₆ alkenyl, or C₂-C₆ alkynyl. In some embodiments, Re is halogen, —CN, —OR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; and R^{6a} is hydrogen, halogen, —CN, or C₂-C₆ alkynyl.

In some embodiments of Formula (V), (VI), (VIIa), (VIIb), or (VIII), R¹ and R⁵ are each independently halogen. R¹ and R⁵ are each independently C₁-C₆ alkyl. R¹ and R⁵ are each independently C₁-C₆ haloalkyl.

In some embodiments of Formula (V), (VI), (VIIa), (VIIb), or (VIII), R¹ is C₁-C₆ alkyl and R⁵ is C₁-C₆ alkyl. In some embodiments, R¹ is methyl and R⁴ is ethyl; or R⁴ is ethyl and R¹ is methyl. In some embodiments, R¹ is methyl and R⁴ is ethyl. In some embodiments, R⁴ is ethyl and R¹ is methyl. In some embodiments, R¹ is methyl and R⁵ is methyl.

In some embodiments of Formula (A), (I*), (I), (Ia), (Ib), (II), (IIa), (IIb), (III*), (III), (IIIa), (V), (VI), (VIa), (VIIa), or (VIIb), A is N. In some embodiments, A is CR⁶.

In some embodiments of Formula (A), (I*), (I), (Ia), (Ib), (II), (IIa), (IIb), (III*), (III), (IIIa), (V), (VI), (VIa), (VIIa), (VIIb), or (VIII), X is CR². In some embodiments, X is N.

In some embodiments of Formula (A), (I*), (I), (Ia), (Ib), (II), (IIa), (IIb), (III*), (III), (IIIa), (V), (VI), (VIa), (VIIa), (VIIb), or (VIII), Y is CR³. In some embodiments, Y is N.

In some embodiments of Formula (A), (I*), (I), (Ia), (II), (IIa), (IIb), (III*), (III), (IIIa), (IV), (IVa), or (V), R¹ is hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆alkyl, C₁-C₆haloalkyl, C₁-C₆hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}. In some embodiments, R¹ is hydrogen, halogen, —N(R^{b})₂, or C₁-C₆ alkyl. In some embodiments, R¹ is halogen or C₁-C₆ alkyl. In some embodiments, R¹ is C₁-C₆ alkyl. In some embodiments, R¹ is methyl or ethyl. In some embodiments, R¹ is methyl. In some embodiments, R¹ is ethyl.

In some embodiments of Formula (A), (I*), (I), (Ia), (II), (IIa), (IIb), (III*), (III), (IIIa), (IV), (IVa), (V), (VI), (VIa), or (VIII), R² is hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}. In some embodiments, R² is hydrogen, halogen, —OH, —N(R^{b})₂, or C₁-C₆ alkyl. In some embodiments, R² is halogen or C₁-C₆ alkyl. In some embodiments, R² is halogen. In some embodiments, R² is C₁-C₆ alkyl. In some embodiments, R² is hydrogen. In some embodiments, R² is hydrogen, fluoro, chloro, —OH, or methyl.

In some embodiments of Formula (A), (I*), (I), (Ia), (II), (IIa), (IIb), (III*), (III), (IIIa), (IV), (IVa), (V), (VI), or (VIa), R³ is hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}. In some embodiments, R³ is hydrogen, halogen, —OH, —N(R^{b})₂, or C₁-C₆ alkyl. In some embodiments, R³ is halogen or C₁-C₆ alkyl. In some embodiments, R³ is halogen. In some embodiments, R³ is C₁-C₆ alkyl. In some embodiments, R³ is hydrogen. In some embodiments, R³ is hydrogen, fluoro, chloro, —OH, or methyl.

In some embodiments of Formula (A), (I*), (I), (Ia), (II), (IIa), (IIb), (III*), (III), (IIIa), (V), (VI), (VIa), or (VIII), R⁴ is hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}. In some embodiments, R⁴ is hydrogen, halogen, —OH, —N(R^{b})₂, or C₁-C₆ alkyl. In some embodiments, R⁴ is halogen or C₁-C₆ alkyl. In some embodiments, R⁴ is halogen. In some embodiments, R⁴ is C₁-C₆ alkyl. In some embodiments, R⁴ is hydrogen. In some embodiments, R⁴ is hydrogen, fluoro, chloro, —OH, or methyl.

In some embodiments of Formula (A), (I*), (I), (Ia), (II), (IIa), (IIb), (III*), (III), (IIIa), (IV), (IVa), or (V), R⁵ is hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆haloalkyl, C₁-C₆hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}. In some embodiments, R⁵ is hydrogen, halogen, —N(R^{b})₂, or C₁-C₆ alkyl. In some embodiments, R⁵ is halogen or C₁-C₆ alkyl. In some embodiments, R⁵ is C₁-C₆ alkyl. In some embodiments, R⁵ is methyl or ethyl. In some embodiments, R⁵ is methyl. In some embodiments, R¹ is ethyl.

In some embodiments of Formula (A), (I*), (I), (Ia), (II), (IIa), (IIb), (III*), (III), (IIIa), (V), or (VI), R³ and R⁴ together with the atoms to which they are attached form a 5 to 6 membered heteroaryl or 5 to 6 membered heterocycloalkyl each of which is optionally substituted with one or two substituents selected from R^{c}. In some embodiments, R³ and R⁴ together with the atoms to which they are attached form a 5 to 6 membered heteroaryl or 5 to 6 membered heterocycloalkyl. In some embodiments, R³ and R⁴ combine together with the atoms to which they are attached form a 5 to 6-membered heteroaryl comprising one, two, or three heteroatoms selected from N, O, and S. In some embodiments, R³ and R⁴ combine together with the atoms to which they are attached form a 5 to 6-membered heteroaryl comprising one or two heteroatoms selected from N and O. In some embodiments, R³ and R⁴ combine together with the atoms to which they are attached form a 5-membered heteroaryl. In some embodiments, R³ and R⁴ combine together with the atoms to which they are attached form a 6-membered heteroaryl. In some embodiments, R³ and R⁴ combine together with the atoms to which they are attached form a 5 to 6-membered heterocycloalkyl comprising one or two heteroatoms selected from N and O. In some embodiments, R³ and R⁴ combine together with the atoms to which they are attached form a 5-membered heterocycloalkyl. In some embodiments, R³ and R⁴ combine together with the atoms to which they are attached form a 6-membered heterocycloalkyl.

In some embodiments of Formula (A), (I*), (I), (Ia), (II), (IIa), (IIb), (III*), (III), (IIIa), (V), or (VI), R⁴ and R⁵ together with the atoms to which they are attached form a 5 to 6 membered heteroaryl or 5 to 6 membered heterocycloalkyl each of which is optionally substituted with one or two substituents selected from R^{c}. In some embodiments, R⁴ and R⁵ together with the atoms to which they are attached form a 5 to 6 membered heteroaryl or 5 to 6 membered heterocycloalkyl. In some embodiments, R⁴ and R⁵ combine together with the atoms to which they are attached form a 5 to 6-membered heteroaryl comprising one, two, or three heteroatoms selected from O, N, and S. In some embodiments, R⁴ and R⁵ combine together with the atoms to which they are attached form a 5 to 6-membered heteroaryl comprising one or two heteroatoms selected from O and N. In some embodiments, R⁴ and R⁵ combine together with the atoms to which they are attached form a 5-membered heteroaryl. In some embodiments, R⁴ and R⁵ combine together with the atoms to which they are attached form a 6-membered heteroaryl. In some embodiments, R⁴ and R⁵ combine together with the atoms to which they are attached form a 5 to 6-membered heterocycloalkyl comprising one or two heteroatoms selected from N and O. In some embodiments, R⁴ and R⁵ combine together with the atoms to which they are attached form a 5-membered heterocycloalkyl. In some embodiments, R⁴ and R⁵ combine together with the atoms to which they are attached form a 6-membered heterocycloalkyl.

In some embodiments of Formula (A), (I*), (I), (Ia), (II), (IIa), (IIb), (III*), (III), (IIIa), (IV), (V), (VI), (VIa), (VIIa), or (VIIb), each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}. In some embodiments, each R⁶ is independently halogen, —CN, —OH, —OR^{a}, —N(R^{b})₂, —S(═O)₂R^{a}, —S(═O)₂N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}. In some embodiments, each R⁶ is independently halogen, —CN, —OR^{a}, —N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl.

In some embodiments of Formula (A), (I*), (I), (Ia), (II), (IIa), (IIb), (III*), (III), (IIIa), (IV), (V), (VI), (VIa), (VIIa), or (VIIb), each R⁶ is independently hydrogen, halogen, —CN, —OH, —OR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₂- C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}.

In some embodiments of Formula (A), (I*), (I), (Ia), (II), (IIa), (IIb), (III*), (III), (IIIa), (IV), (V), (VI), (VIa), (VIIa), or (VIIb), each R⁶ is independently hydrogen, halogen, —CN, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl, each of which is optionally substituted with one, two, three, or four substituents independently selected from R^{c}.

In some embodiments of Formula (A), (I*), (I), (Ia), (II), (IIa), (IIb), (III*), (III), (IIIa), (IV), (V), (VI), (VIa), (VIIa) or (VIIb), each R⁶ is independently halogen, C₁-C₆ alkyl, C₂-C₆ alkynyl, C₁-C₆ haloalkyl, or C₁-C₆ hydroxyalkyl. In some embodiments, each R⁶ is independently C₁-C₆ alkyl, C₂-C₆ alkynyl, C₁-C₆ haloalkyl, or C₁-C₆ hydroxyalkyl. In some embodiments, each R⁶ is independently methyl, ethyl, isopropyl, sec-butyl, tert-butyl, —CF₃, —CHF₂—CH₂F, —OCH₃, or —OCH₂CH₃. In some embodiments, each R⁶ is independently methyl or ethyl. In some embodiments, each R⁶ is independently halogen. In some embodiments, each R⁶ is independently —CN, chloro, fluoro, methyl, or ethyl.

In some embodiments of Formula (A), (I*), (I), (Ia), (II), (IIa), (IIb), (III*), (III), (IIIa), (IV), (V), (VI), (VI), (VIIa), or (VIIb), each R⁶ is independently C₁-C₆ alkyl, C₂-C₆ alkenyl, or C₂-C₆ alkynyl each or which are optionally substituted with halogen, —OH, —NH₂, —CF₃, C₃-C₆ cycloalkyl, phenyl, or 5 to 6-membered heteroaryl. In some embodiments, each R⁶ is independently

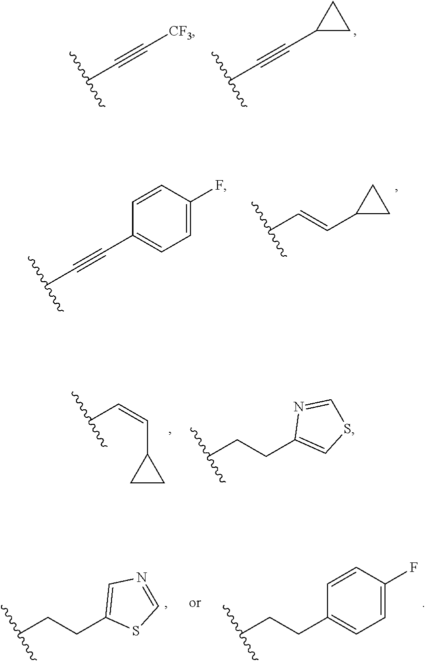

In some embodiments of Formula (A), (I*), (I), (Ia), (II), (IIa), (IIb), (III*), (III), (IIIa), (IV), (V), (VI), (VIa), (VIIa), or (VIIb), each R⁶ is independently C₃-C₈ cycloalkyl or 4 to 8 membered heterocycloalkyl, each of which is optionally substituted with one, two, or three R^{c}. In some embodiments, each R⁶ is independently C₃-C₆ cycloalkyl or 4 to 6 membered heterocycloalkyl. In some embodiments, each R⁶ is independently C₃-C₆ cycloalkyl. In some embodiments, each R⁶ is independently cyclopropyl, cyclobutyl, cyclopentyl or cyclohexyl. In some embodiments, each R⁶ is independently cyclopropyl. In some embodiments, each R⁶ is independently cyclobutyl. In some embodiments, each R⁶ is independently cyclopentyl. In some embodiments, each R⁶ is independently cyclohexyl. In some embodiments, each R⁶ is independently 4 to 8 membered heterocycloalkyl. In some embodiments, each R⁶ is independently a 4 membered, 5 membered, or 6 membered heterocycloalkyl. In some embodiments, each R⁶ is independently a 4 membered heterocycloalkyl. In some embodiments, each R⁶ is independently a 5 membered heterocycloalkyl. In some embodiments, each R⁶ is independently a 6 membered heterocycloalkyl.

In some embodiments of Formula (A), (I*), (I), (Ia), (II), (IIa), (IIb), (III*), (III), (IIIa), (IV), (V), (VI), (VIa), (VIIa), or (VIIb), each R⁶ is independently —N(R^{b})₂ or —NHC(═O)R^{a}. In some embodiments, each R⁶ is independently —N(R^{b})₂. In some embodiments, each R⁶ is independently —NH(CH₂)₂OH,

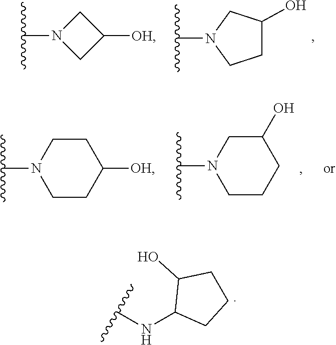

In some embodiments, each R⁶ is independently —NHC(═O)R^{a}. In some embodiments, each R⁶ is independently

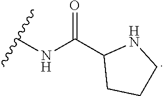 [SMILES: *NC(=O)C1CCCN1]

In some embodiments of Formula (A), (I*), (I), (Ia), (II), (IIa), (IIb), (III*), (III), (IIIa), (IV), (V), (VI), (VIa), (VIIa), or (VIIb), each R⁶ is independently —CH₃, —CH₂CH₃, —CN, —F, —Cl, —Br, —CH₂OH, —CH₂CH₂OH, —NH(CH₂)₂OH, cyclopropyl,

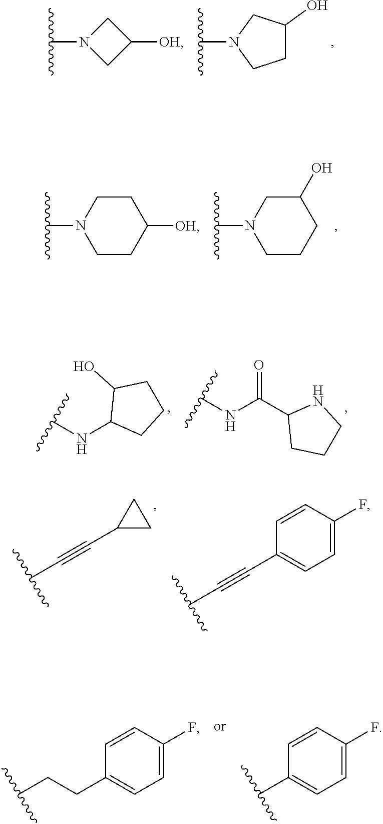

In some embodiments of Formula (A), (I*), (I), (Ia), (II), (IIa), (IIb), (III*), (III), (IIIa), (IV), (V), (VI), (VIa), (VIIa), or (VIIb), each R⁶ is independently —CH₃, —CH₂CH₃, —CN, —F, —Cl, —Br, —CH₂OH, —CH₂CH₂OH, —NH(CH₂)₂OH, cyclopropyl,

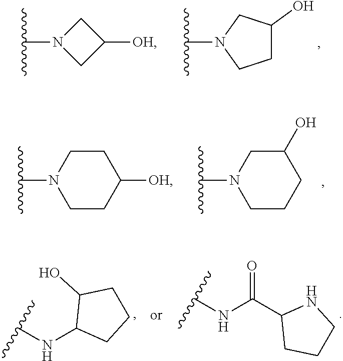 [SMILES: *N1CC(O)C1 ; *N1CCC(O)C1 ; *N1CCC(O)CC1 ; *N1CCCC(O)C1 ; *NC1CCCC1O ; *NC(=O)C1CCCN1]

In some embodiments of Formula (A), (I*), (I), (Ia), (II), (IIa), (IIb), (III*), (III), (IIIa), (IV), (V), or (VI), two R⁶ on adjacent atoms join together to form a 6-membered carbocyclic or heterocyclic ring each of which is optionally substituted with one or two substituents selected from R^{c}. In some embodiments, two R⁶ on adjacent atoms join together to form a 6-membered heterocycloalkyl comprising one to two heteroatoms selected from N and O. In some embodiments, two R⁶ on adjacent atoms join together to form a C₆ cycloalkyl.

In some embodiments of Formula (IVa) or (VIII), R^{6a} and R^{6b} are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}. In some embodiments, R^{6a} and R^{6b} are each independently halogen, —CN, —OH, —OR^{a}, —N(R^{b})₂, —S(═O)₂R^{a}, —S(═O)₂N(R^{b})₂, —NHC(═O)R^{a}, C₁-C₆ alkyl, C₂-C₆ alkenyl, C₂-C₆ alkynyl, C₁-C₆ haloalkyl, C₁-C₆hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}. In some embodiments, R^{6a} and R^{6b} are each independently halogen, —CN, —OR^{a}, —N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl.

In some embodiments of Formula (IV) or (VIII), R^{6a} and R^{6b} are each independently halogen, C₁-C₆ alkyl, C₂-C₆ alkynyl, C₁-C₆ haloalkyl, or C₁-C₆ hydroxyalkyl. In some embodiments, each R⁶ is independently C₁-C₆ alkyl, C₂-C₆ alkynyl, C₁-C₆ haloalkyl, or C₁-C₆ hydroxyalkyl. In some embodiments, each R⁶ is independently methyl, ethyl, isopropyl, sec-butyl, tert-butyl, —CF₃, —CHF₂—CH₂F, —OCH₃, or —OCH₂CH₃. In some embodiments, R^{6a} and R^{6b} are each independently methyl or ethyl. In some embodiments, R^{6a} and R^{6b} are each independently halogen. In some embodiments, R^{6a} and R^{6b} are each independently —CN, chloro, fluoro, methyl, or ethyl.

In some embodiments of Formula (IVa) and (VIII), R^{6a} and R^{6b} are each independently C₁-C₆ alkyl, C₂-C₆ alkenyl, or C₂-C₆ alkynyl each or which are optionally substituted with halogen, —OH, —NH₂, —CF₃, C₃-C₆ cycloalkyl, phenyl, or 5 to 6-membered heteroaryl. In some embodiments, R^{6a} and R^{6b} are each independently

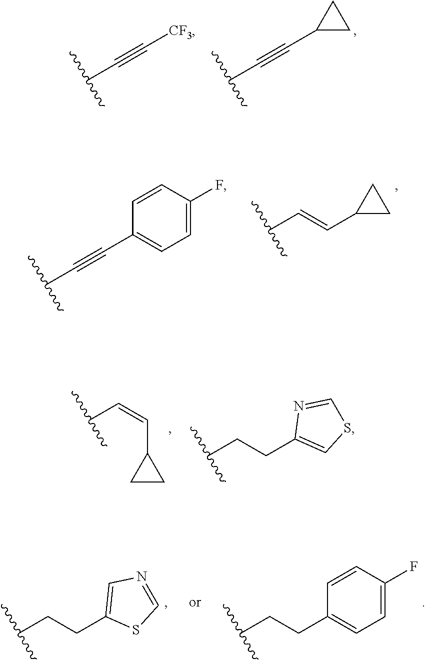

In some embodiments of Formula (IVa) or (VIII), R^{6a} and R^{6b} are each independently C₃-C₈ cycloalkyl or 4 to 8 membered heterocycloalkyl, each of which is optionally substituted with one, two, or three R^{c}. In some embodiments, R^{6a} and R^{6b} are each independently C₃-C₆ cycloalkyl or 4 to 6 membered heterocycloalkyl. In some embodiments, R^{6a} and R^{6b} are each independently C₃-C₆ cycloalkyl. In some embodiments, R^{6a} and R^{6b} are each independently cyclopropyl, cyclobutyl, cyclopentyl or cyclohexyl. In some embodiments, R^{6a} and R^{6b} are each independently cyclopropyl. In some embodiments, R^{6a} and R^{6b} are each independently cyclobutyl. In some embodiments, R^{6a} and R^{6b} are each independently cyclopentyl. In some embodiments, R^{6a} and R^{6b} are each independently cyclohexyl. In some embodiments, R^{6a} and R^{6b} are each independently 4 to 8 membered heterocycloalkyl. In some embodiments, R^{6a} and R^{6b} are each independently a 4 membered, 5 membered, or 6 membered heterocycloalkyl. In some embodiments, R^{6a} and R^{6b} are each independently a 4 membered heterocycloalkyl. In some embodiments, R^{6a} and R^{6b} are each independently a 5 membered heterocycloalkyl. In some embodiments, R^{6a} and R^{6b} are each independently a 6 membered heterocycloalkyl.

In some embodiments of Formula (IVa) or (VIII), R^{6a} and R^{6b} are each independently —N(R^{b})₂ or —NHC(═O)R^{a}. In some embodiments, R^{6a} and R^{6b} are each independently —N(R^{b})₂. In some embodiments, R⁶ and R^{6b} are each independently —NH(CH₂)₂OH,

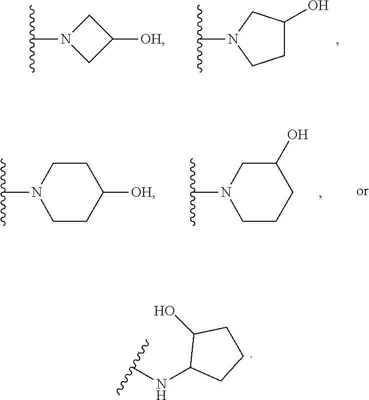

In some embodiments, R^{6a} and R^{6b} are each independently —NHC(═O)R^{a}. In some embodiments, R^{6a} and R^{6b} are each independently

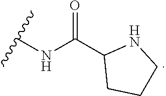 [SMILES: *NC(=O)C1CCCN1]

In some embodiments of Formula (IVa) and (VIII), R^{6a} and R^{6b} are each independently —CH₃, —CH₂CH₃, —CN, —F, —Cl, —Br, —CH₂OH, —CH₂CH₂OH, —NH(CH₂)₂OH, cyclopropyl,

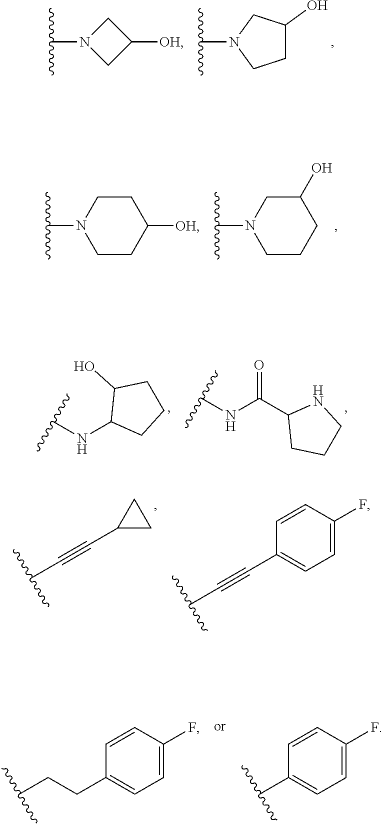

In some embodiments of Formula (IVa) or (VIII), R^{6a} and R^{6b} are each independently —CH₃, —CH₂CH₃, —CN, —F, —Cl, —Br, —CH₂OH, —CH₂CH₂OH, —NH(CH₂)₂OH, cyclopropyl,

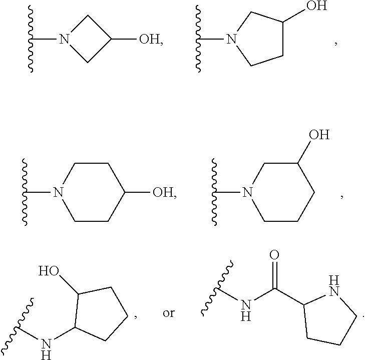

In some embodiments of Formula (A), (I*), (I), (Ia), (II), (IIa), (IIb), (III*), (III), (IIIa), (IV), (V), (VI), (VIa), (VIIa), or (VIIb), p is 1, 2 or 3. In some embodiments, p is 1 or 2. In some embodiments, p is 3. In some embodiments, p is 2. In some embodiments, p is 1.

In some embodiments of a compound disclosed herein, each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl. In some embodiments, each R^{a} is independently C₁-C₆ alkyl, C₂-C₆ alkenyl, C₂-C₆ alkynyl, or C₁-C₆ heteroalkyl. In some embodiments, each R^{a} is independently cycloalkyl or heterocycloalkyl. In some embodiments, each R^{a} is independently aryl or heteroaryl. In some embodiments, each R^{a} is independently —C₁-C₆ alkyl(aryl), —C₁-C₆ alkyl(heteroaryl), —C₁-C₆ alkyl(cycloalkyl), or —C₁-C₆ alkyl(heterocycloalkyl).

In some embodiments of a compound disclosed herein, each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is independently unsubstituted or substituted with one, two, or three halogen, —OH, C₁-C₆ alkyl, or C₁-C₆ haloalkyl. In some embodiments, each R^{b} is independently hydrogen, C₁-C₆ alkyl, C₂-C₆ alkenyl, C₂-C₆ alkynyl, or C₁-C₆heteroalkyl. In some embodiments, each R^{b} is independently cycloalkyl or heterocycloalkyl. In some embodiments, each R^{b} is independently aryl, or heteroaryl.

In some embodiments of a compound disclosed herein are taken together with the atom to which they are attached to form a heterocycloalkyl optionally substituted with one or more R^{c}.

In some embodiments of a compound disclosed herein, each R^{c} is independently halogen, —CN, —OH, —NH₂, —NHCH₃, —N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, C₃-C₆cycloalkyl, C₆-C₁₀aryl, or 5 to 10 membered heteroaryl comprising one or more heteroatoms selected from O, N, and S; or two R^{c} on the same atom form an oxo. In some embodiments of a compound disclosed herein, each R^{c} is independently halogen, —CN, —OH, —NH₂, —NHCH₃, —N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R^{c} on the same atom form an oxo. In some embodiments of a compound disclosed herein, each R^{c} is independently halogen, —CN, —OH, C₁-C₆alkyl, C₁-C₆alkoxy, or C₁-C₆haloalkyl; or two R^{c} on the same atom form an oxo. In some embodiments of a compound disclosed herein, each R^{c} is independently halogen, —CN, —OH, or C₁-C₆alkyl; or two R^{c} on the same atom form an oxo. In some embodiments of a compound disclosed herein, each R^{c} is independently halogen, —OH, C₁-C₆alkyl, C₃-C₆ cycloalkyl, 4 to 6 membered heteroalkyl, phenyl, or 5-membered heteroaryl. In some embodiments of a compound disclosed herein, each R^{c} is independently halogen, —OH, or C₁-C₆alkyl. In some embodiments of a compound disclosed herein, each R^{c} is independently halogen or C₁-C₆alkyl. In some embodiments of a compound disclosed herein, each R^{c} is independently halogen. In some embodiments of a compound disclosed herein, each R^{c} is independently C₃-C₆ cycloalkyl. In some embodiments of a compound disclosed herein, each R^{c} is independently 4 to 6 membered heteroalkyl. In some embodiments of a compound disclosed herein, each R^{c} is independently phenyl or 5-membered heteroaryl.

In some embodiments, the compounds made in the examples below are made from racemic starting materials (and/or intermediates) and separated into the individual enantiomers by chiral chromatography as final products or intermediates. Unless otherwise stated, it is understood that the absolute configuration of the separated intermediates and final compounds as drawn is arbitrarily assigned and was not determined. In some embodiments, the compounds are a racemate. In some embodiments, the compounds are resolved into R and S configuration. In some embodiments, the compounds exist as atropisomers.

Non-limiting examples of compounds described herein, are compounds presented in Table 1 and pharmaceutically acceptable salts, solvates, stereoisomers, or atropisomers thereof.

TABLE 1
Representative compounds of the disclosure

| Com- | | |
|---|---|---|
| pound ID | Structure | Analytical Data |
| 542 | 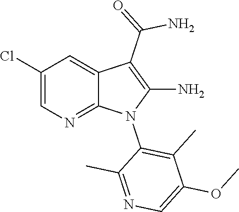 [SMILES: COc1cnc(C)c(-n2c(N)c(C(N)=O)c3cc(Cl)cnc32)c1C] | ¹H NMR (400 MHz, DMSO-d₆) δ 8.35 (s, 1H), 8.17 (d, J = 2.0 Hz, 1H), 7.76 (d, J = 2.4 Hz, 1H), 7.38 (br s, 2H), 6.90 (br s, 2H), 3.96 (s, 3H), 1.99 (s, 3H), 1.75 (s, 3H). LCMS R_{T} = 1.361 min, m/z = 346.3 [M + H]⁺. |
| 543 | 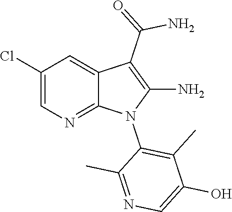 [SMILES: Cc1ncc(O)c(C)c1-n1c(N)c(C(N)=O)c2cc(Cl)cnc21] | ¹H NMR (400 MHz, DMSO-d₆) δ 12.03 (brs, 1H), 8.38 (s, 1H), 8.22 (s, 1H), 7.81 (d, J = 2.4 Hz, 1H), 7.51 (brs, 2H), 6.98 (brs, 2H), 2.24 (s, 3H), 1.90 (s, 3H). LCMS R_{T} = 1.091 min, m/z = 331.9 [M + H]⁺. |
| 544 | 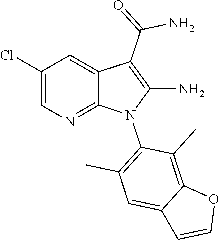 [SMILES: Cc1cc2ccoc2c(C)c1-n1c(N)c(C(N)=O)c2cc(Cl)cnc21] | ¹H NMR (400 MHz, DMSO-d₆) δ 8.43 (s, 1 H of formic acid), 8.16 (d, J = 2.4 Hz, 1H), 8.11 (d, J = 2.0 Hz, 1H), 7.73 (d, J = 2.4 Hz, 1H), 7.54 (s, 1H), 7.24 (br s, 2H), 7.02 (d, J = 2.4 Hz, 1H), 6.87 (br s, 2H), 2.04 (s, 3H), 1.91 (s, 3H). LCMS R_{T} = 2.087 min, m/z = 355.3 [M + H]. |
| 546 | 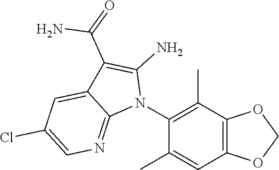 [SMILES: Cc1cc2c(c(C)c1-n1c(N)c(C(N)=O)c3cc(Cl)cnc31)OCO2] | ¹H NMR (400 MHz, CD₃OD) δ 8.02 (d, J = 2.0 Hz, 1H), 7.76 (d, J = 2.0 Hz, 1H), 6.78 (s, 1H), 6.04 (s, 2H), 1.86 (s, 3H), 1.80 (s, 3H). LCMS R_{T} = 0.508 min, m/z = 358.9 [M + H]⁺. |
| 547 | 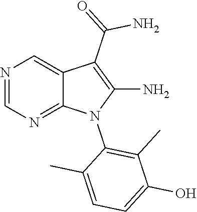 [SMILES: Cc1ccc(O)c(C)c1-n1c(N)c(C(N)=O)c2cncnc21] | ¹H NMR (400 MHz, DMSO-d₆): δ 9.67 (bs, 1H), 8.99 (s, 1H), 8.40 (s, 1H), 7.20 (bs, 2H), 7.07 (d, J = 7.6 Hz, 1H), 6.96-6.92 (m, 3H), 1.74 (s, 3H), 1.66 (s, 3H). LCMS RT = 1.08 min, m/z = 298.16 [M + H]⁺ |
| 548 | 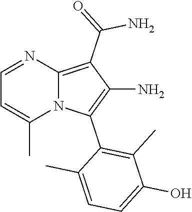 [SMILES: Cc1ccc(O)c(C)c1-c1c(N)c(C(N)=O)c2nccc(C)n12] | ¹H NMR (400 MHz, CD₃OD) δ 7.52 (d, J = 6.8 Hz, 1H), 7.05 (d, J = 8.0 Hz, 1H), 6.84 (d, J = 8.0 Hz, 1H), 6.65 (d, J = 6.8 Hz, 1H), 2.55 (s, 3H), 1.92 (s, 3H), 1.87 (s, 3H). LCMS R_{T} = 1.135 min, m/z = 311 [M + H]⁺ |
| 550 | 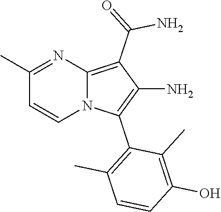 [SMILES: Cc1ccn2c(-c3c(C)ccc(O)c3C)c(N)c(C(N)=O)c2n1] | ¹H NMR (400 MHz, CD₃OD) δ 8.03 (d, J = 4.4 Hz, 1H), 6.98 (d, J = 8.4 Hz, 1H), 6.84 (d, J = 8.4 Hz, 1H), 6.56 (d, J = 4.4 Hz, 1H), 2.02 (s, 3H), 1.91 (s, 3H), 1.89 (s, 3H). LCMS R_{T} = 1.016 min, m/z = 311 [M + H]⁺ |
| 552 | 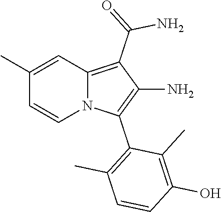 [SMILES: Cc1ccn2c(-c3c(C)ccc(O)c3C)c(N)c(C(N)=O)c2c1] | ¹H NMR (400 MHz, DMSO-d₆): δ 9.46 (bs, 1H), 7.76 (s, 1H), 7.10 (d, J = 6.4 Hz, 1H), 7.01 (d, J = 8.4 Hz, 1H), 6.87 (d, J = 8.0 Hz, 1H), 6.81 (s, 2H), 6.45 (d, J = 6.8 Hz, 1H), 4.81 (bs, 2H), 2.29 (s, 3H), 1.82 (s, 3H), 1.74 (s, 3H). LCMS RT = 2.27 min, m/z = 310.3 [M + H]⁺ |
| 553 | 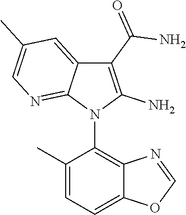 [SMILES: Cc1cnc2c(c1)c(C(N)=O)c(N)n2-c1c(C)ccc2ocnc12] | ¹H NMR (400 MHz, DMSO-d₆): δ 9.30-8.84 (bs, 2H), 7.91 (s, 1H), 7.65 (s, 1H), 7.40 (s, 1H), 7.14 (q, J = 8.4 Hz, 1H), 7.02 (t, J = 7.6 Hz, 2H), 6.75 (bs, 1H), 2.30 (s, 3H), 1.71 (d, J = 12 Hz, 3H). LCMS RT = 1.672 min, m/z = 322.16 [M + H]⁺ |
| 555 | 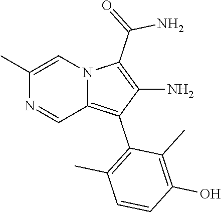 [SMILES: Cc1cn2c(C(N)=O)c(N)c(-c3c(C)ccc(O)c3C)c2cn1] | ¹H NMR (400 MHz, CD₃OD) δ 9.03 (s, 1H), 8.03 (s, 1H), 7.03 (d, J = 8.0 Hz, 1H), 6.81 (d, J = 8.4 Hz, 1H), 2.43 (s, 3H), 1.94 (s, 3H), 1.90 (s, 3H). LCMS R_{T} = 0.669 min, m/z = 311 [M + H]⁺ |
| 556 | 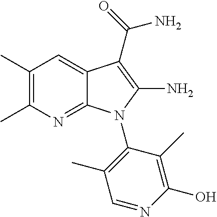 | ¹H NMR (400 MHz, DMSO-d₆) δ 9.31 (s, 1H), 7.86 (s, 1H), 7.59 (s, 1H), 6.80-6.67 (m, 5H), 6.44 (d, J = 8.0 Hz, 1H), 4.01 (s, 2H), 2.31 (s, 3H), 1.64 (s, 3H). LCMS R_{T} = 1.320 min, m/z = 312.2 [M + H]⁺ |
| 559 | 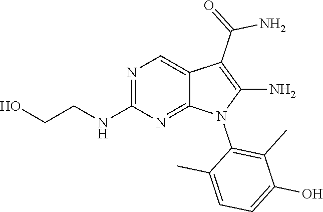 [SMILES: Cc1ccc(O)c(C)c1-n1c(N)c(C(N)=O)c2cnc(NCCO)nc21] | ¹H NMR: (DMSO-d₆, 400 MHz) δ 9.50 (s, 1H), 8.53 (s, 1H), 7.04 (d, J = 8 Hz, 1H), 6.89 (d, J = 8.4 Hz, 1H), 6.70 (bs, 2H), 6.62 (bs, 2H), 6.40 (t, J = 5.6 Hz, 1H), 4.61 (bs, 1H), 3.44 (d, J = 4.8 Hz, 2H), 3.22 (q, J = 6.0 Hz, 12 Hz, 2H), 1.79 (s, 3H), 1.71 (s, 3H). LCMS RT = 1.083 min, m/z = 357.32 [M + H]⁺ |
| 560 | 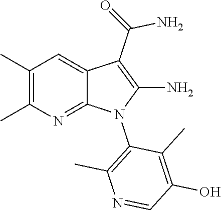 [SMILES: Cc1cc2c(C(N)=O)c(N)n(-c3c(C)ncc(O)c3C)c2nc1C] | ¹H NMR (400 MHz, DMSO-d₆) δ 12.03 (bs s, 1H), 8.35 (s, 1H), 7.97 (s, 1H), 7.21 (br s, 2H), 6.81 (br s, 2H), 2.28 (s, 6H), 2.24 (s, 3H), 1.90 (s, 3H). LCMS R_{T} = 0.759 min, m/z = 326.4 [M + H]⁺. |
| 561 | 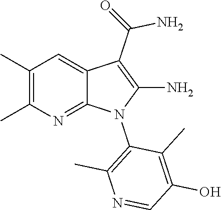 | ¹H NMR (400 MHz, DMSO-d₆) δ 12.21 (br s, 1H), 8.41 (d, J = 1.6 Hz, 1H), 8.02 (s, 1H), 7.67 (s, 1H), 7.38 (br s, 2H), 6.69 (br s, 2H), 2.33 (s, 3H), 2.26 (s, 3H), 1.90 (s, 3H). LCMS R_{T} = 0.985 min, m/z = 312.2 [M + H]⁺ |
| 565 | 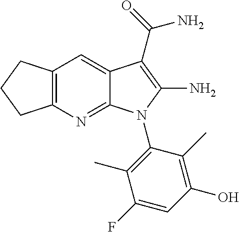 | ¹H NMR (400 MHz, DMSO-d₆) δ 10.05 (s, 1H), 8.15 (s, 1H), 7.76 (s, 1H), 7.26 (bs, 2H), 6.88 (bs, 2H), 6.80 (d, J = 11.2 Hz, 1H), 1.65 (s, 3H), 1.61 (s, 3H). LCMS RT = 1.879 min, m/z = 349.2 [M + H]⁺ |
| 566 | 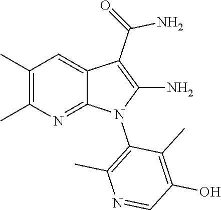 [SMILES: Cc1cc2c(C(N)=O)c(N)n(-c3c(C)ncc(O)c3C)c2nc1C] | ¹H NMR (400 MHz, DMSO-d₆): δ 9.57 (s, 1H), 7.07 (d, J = 8.4 Hz, 1H), 6.92 (d, J = 8.0 Hz, 1H), 6.88 (bs, 2H), 6.51 (bs, 2H), 2.71 (s, 3H), 2.39 (s, 3H), 1.75 (s, 3H), 1.65 (s, 3H). LCMS RT = 4.434 min, m/z = 325 [M + H]⁺ |
| 567 | 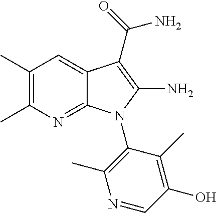 | ¹H NMR (300 MHz, dmso) δ 9.58 (s, 1H), 8.86 (s, 1H), 7.06 (d, J = 8.7 Hz, 3H), 6.92 (d, J = 8.2 Hz, 1H), 6.83 (s, 2H), 2.42 (s, 3H), 1.73 (s, 3H), 1.65 (s, 3H). LCMS: RT = 0.49 min, m/z 312.2 [M + H]⁺ |
| 571 | 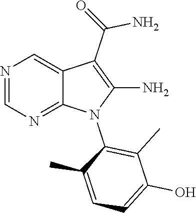 | ¹H NMR (400 MHz, DMSO-d₆) δ 9.67 (bs, 1H), 8.99 (s, 1H), 8.40 (s, 1H), 7.20 (bs, 2H), 7.07 (d, J = 7.6 Hz, 1H), 6.94 (m, 3H), 1.74 (s, 3H), 1.66 (s, 3H). LCMS RT = 1.072 min, m/z = 297 [M + H]⁺ |
| 572 | 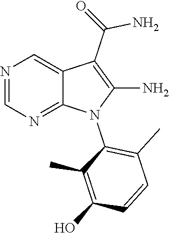 | ¹H NMR (400 MHz, DMSO-d₆) δ 9.67 (bs, 1H), 8.99 (s, 1H), 8.40 (s, 1H), 7.20 (bs, 2H), 7.07 (d, J = 7.6 Hz, 1H), 6.94 (m, 3H), 1.74 (s, 3H), 1.66 (s, 3H). LCMS RT = 1.045 min, m/z = 297 [M + H]⁺ |
| 573 | 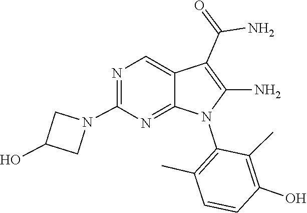 [SMILES: Cc1ccc(O)c(C)c1-n1c(N)c(C(N)=O)c2cnc(N3CC(O)C3)nc21] | ¹H NMR (400 MHz, DMSO-d₆) δ 9.55 (brs, 1H), 8.60 (s, 1H), 7.06 (d, J = 8.4 Hz, 1H), 6.90 (d, J = 8.0 Hz, 1H), 6.72 (brs, 2H), 6.68 (brs, 2H), 5.49 (d, J = 6.8 Hz, 1H), 4.42-4.34 (m, 1H), 4.02 (t, J = 9.2 Hz, 2H), 3.58 (t, J = 4.8 Hz, 2H), 1.79 (s, 3H), 1.70 (s, 3H). LCMS R_{T} = 0.770 min, m/z = 368.9 [M + H]⁺. |
| 574 | 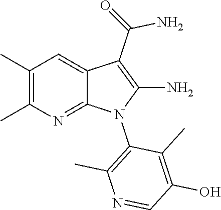 | ¹H NMR (400 MHz, DMSO-d₆) δ 11.52 (brs, 1H), 9.11 (s, 1H), 8.68 (d, J = 8.4 Hz, 1H), 8.38 (s, 1H), 7.82 (brs, 2H), 7.39 (brs, 2H), 2.21 (s, 3H), 1.92 (s, 3H). LCMS R_{T} = 0.283 min, m/z = 316.3 [M + H]⁺. |
| 575 | 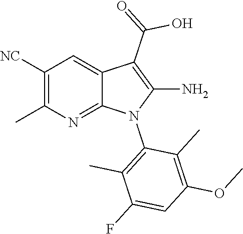 | ¹H NMR (400 MHz, DMSO-d₆) δ 10.05 (s, 1H), 8.15 (s, 1H), 7.76 (s, 1H), 7.26 (bs, 2H), 6.89 (bs, 2H), 6.81 (d, J = 11.2 Hz, 1H), 1.65 (s, 3H), 1.61 (s, 3H). LCMS RT = 1.912 min, m/z = 349.2 [M + H]⁺ |
| 576 | 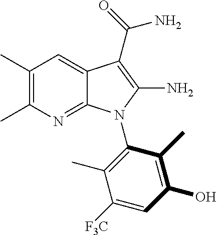 | ¹H NMR (400 MHz, DMSO-d₆) δ 10.06 (s, 1H), 8.16 (s, 1H), 7.76 (s, 1H), 7.26 (bs, 2H), 6.89 (bs, 2H), 6.81 (d, J = 11.2 Hz, 1H), 1.65 (s, 3H), 1.61 (s, 3H). LCMS RT = 1.916 min, m/z = 349.2 [M + H]⁺ |
| 577 | 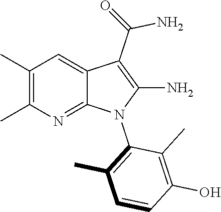 | ¹H NMR (400 MHz, DMSO-d₆) δ 9.59 (brs, 1H), 8.88 (s, 1H), 8.14 (s, 1H of formic acid), 7.08 (d, J = 8.4 Hz, 1H), 7.05 (brs, 2H), 6.93 (d, J = 8.4 Hz, 1H), 6.84 (brs, 2H), 2.43 (s, 3H), 1.75 (s, 3H), 1.67 (s, 3H). LCMS R_{T} = 1.302 min, m/z = 312.2 [M + H]⁺. |
| 578 | 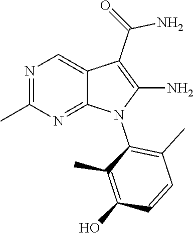 | ¹H NMR (400 MHz, DMSO-d₆) δ 9.59 (brs, 1H), 8.88 (s, 1H), 8.14 (s, 1H of formic acid), 7.08 (d, J = 8.4 Hz, 1H), 7.05 (brs, 2H), 6.93 (d, J = 8.4 Hz, 1H), 6.84 (brs, 2H), 2.43 (s, 3H), 1.75 (s, 3H), 1.67 (s, 3H). LCMS R_{T} = 1.050 min, m/z = 312.2 [M + H]⁺. |
| 579 | 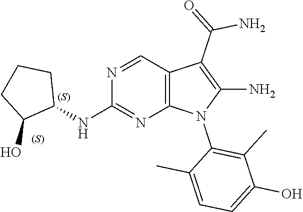 | ¹H NMR (400 MHz, DMSO-d₆) δ 9.51 (brs, 1H), 8.53 (s, 1H), 7.04 (d, J = 8.0 Hz, 1H), 6.88 (d, J = 8.4 Hz, 1H), 6.71 (brs, 2H), 6.63 (brs, 2H), 6.55 (d, J = 5.6 Hz, 1H), 4.96 (brs, 1H), 3.83-3.75 (m, 1H), 3.74-3.64 (m, 1H), 1.98-1.91 (m, 1H), 1.83-1.75 (m, 4H), 1.71 |
| 580 | 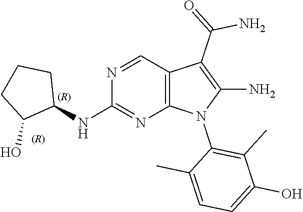 | ¹H NMR (400 MHz, DMSO-d₆) δ 9.51 (brs, 1H), 8.54 (s, 1H), 7.03 (d, J = 7.6 Hz, 1H), 6.88 (d, J = 8.4 Hz, 1H), 6.71 (brs, 2H), 6.63 (brs, 2H), 6.55 (d, J = 5.6 Hz, 1H), 4.95 (brs, 1H), 3.83-3.75 (m, 1H), 3.74-3.65 (m, 1H), 1.99-1.91 (m, 1H), 1.83-1.74 (m, 4H), 1.71 (s, 3H), 1.60-1.51 (m, 2H), 1.45-1.35 (m, 2H). LCMS R_{T} = 0.955 min, m/z = 397.1 [M + H]⁺. |
| 581 | 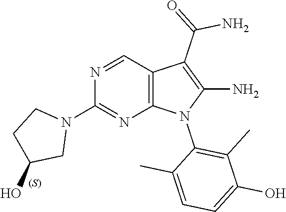 | ¹H NMR (400 MHz, DMSO-d₆) δ 9.51 (brs, 1H), 8.58 (s, 1H), 7.05 (d, J = 8.4 Hz, 1H), 6.89 (d, J = 8.4 Hz, 1H), 6.66 (brs, 2H), 6.62 (brs, 2H), 4.80 (brs, 1H), 4.29-4.22 (m, 1H), 3.29-3.18 (m, 4H), 1.99-1.93 (m, 1H), 1.91 (s, 3H), 1.90-1.84 (m, 1H), 1.72 (s, 3H). LCMS R_{T} = 0.900 min, m/z = 383.4 [M + H]⁺. |
| 582 | 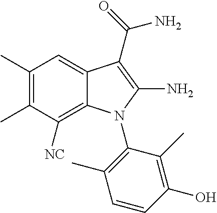 | ¹H NMR (400 MHz, DMSO-d₆) δ 9.60 (brs, 1H), 7.82 (s, 1H), 7.11-7.00 (m, 3H), 6.91 (d, J = 8.4 Hz, 1H), 6.17 (brs, 2H), 2.70 (s, 3H), 1.80 (s, 3H), 1.71 (s, 1H). LCMS R_{T} = 0.767 min, m/z = 328.9 [M + H]⁺. |
| 583 | 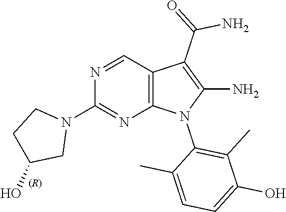 | ¹H NMR (400 MHz, DMSO-d₆) δ 9.53 (brs, 1H), 8.58 (s, 1H), 8.13 (s, 1H), 7.05 (d, J = 8.4 Hz, 1H), 6.89 (d, J = 8.0 Hz, 1H), 6.66 (brs, 2H), 6.61 (brs, 2H), 4.83 (brs, 1H), 4.30-4.23 (m, 1H), 3.28-3.20 (m, 4H), 1.90 (td, J = 4.0, 12.4 Hz, 1H), 1.81 (s, 3H), 1.80-1.74 (m, 1H), 1.72 (s, 3H). LCMS R_{T} = 1.101 min, m/z = 383.4 [M + H]⁺. |
| 584 | 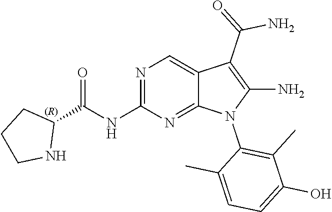 | ¹H NMR (400 MHz, MeOD) δ 8.76 (s, 1H), 7.15 (d, J = 8.4 Hz, 1H), 6.97 (d, J = 8.4 Hz, 1H), 4.49 (bs, 1H), 3.50-3.41 (m, 2H), 2.50 (bs, 2H), 2.11 (bs, 2H), 1.93 (s, 3H), 1.86 (s, 3H) LCMS RT = 1.77 min, m/z = 410.5 [M + H]⁺ |
| 585 | 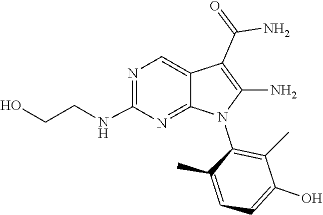 | ¹H NMR (400 MHz, DMSO-d₆) δ 9.57 (s, 1H), 8.51 (s, 1H), 7.05 (d, J = 8 Hz, 1H), 6.90 (d, J = 8.4 Hz, 1H), 6.73 (bs, 2H), 3.44 (t, J = 4.8 Hz, 2H), 3.22 (q, J = 6.0 Hz, 12 Hz, 2H), 1.80 (s, 3H), 1.72 (s, 3H). (—OH and —NH proton not observed in NMR) LCMS RT = 1.098 min, m/z = 357.32 [M + H]⁺ |
| 586 | 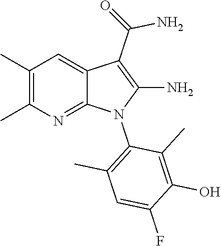 | ¹H NMR (400 MHz, DMSO-d₆) δ 10.08 (brs, 1H), 8.65 (s, 1H), 8.16 (s, 1H), 7.03 (brs, 2H), 6.88 (brs, 2H), 2.34 (d, J = 4.0 Hz, 3H), 1.99 (s, 3H), 1.78 (s, 3H). LCMS R_{T} = 1.089 min, m/z = 330.3 [M + H]⁺. |
| 587 | 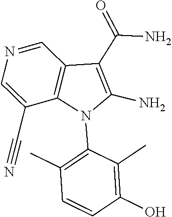 [SMILES: Cc1ccc(O)c(C)c1-n1c(N)c(C(N)=O)c2cncc(C#N)c21] | ¹H NMR (400 MHz, DMSO-d₆) δ 9.71 (bs, 1H), 9.13 (s, 1H), 8.37 (s, 1H), 7.16 (s, 2H), 7.09 (d, J = 8 Hz, 1H), 7.05 (s, 2H), 6.98 (d, J = 8 Hz, 1H), 1.77 (s, 3H), 1.68 (s, 3H). LCMS RT = 1.201 min, m/z = 322 [M + H]⁺ |
| 588 | 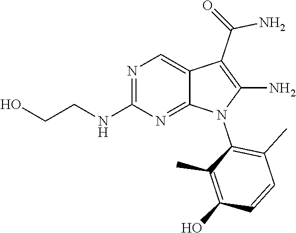 | ¹H NMR (400 MHz, DMSO-d₆) δ 9.55 (bs, 1H), 8.53 (s, 1H), 7.04 (d, J = 8.4 Hz, 1H), 6.89 (d, J = 8.4 Hz, 1H), 6.71 (bs, 2H), 6.64 (bs, 2H), 6.42 (t, J = 5.6 Hz, 1H), 4.62 (bs, 1H), 3.44-1.43 (m, 2H), 3.22 (q, J = 6.0 Hz, 11.6 Hz, 2H), 1.80 (s, 3H), 1.71 (s, 3H). LCMS RT = 1.153 min, m/z = 357.37 [M + H]⁺ |
| 591 | 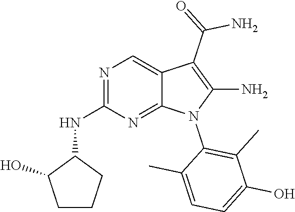 | ¹H NMR (400 MHz, CD₃OD) δ 8.43 (s, 1H), 7.09 (d, J = 8.4 Hz, 1H), 6.91 (d, J = 8.4 Hz, 1H), 4.12 (d, J = 2.4 Hz, 1H), 3.97 (d, J = 4.8 Hz, 1H), 1.92-1.83 (m, 8H), 1.49-1.71 (m, 4H). LCMS R_{T} = 1.636 min, m/z = 397.2 [M + H]⁺. |
| 592 | 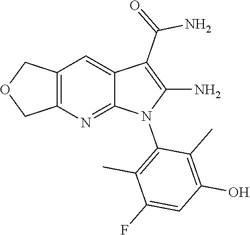 | ¹H NMR (400 MHz, DMSO-d₆) δ 9.61 (s, 1H), 8.95 (s, 1H), 7.12 (s, 2H), 7.07 (d, J = 8.4 Hz, 1H), 6.98 (d, J = 8.4 Hz, 1H), 6.91 (s, 2H), 5.07 (t, J = 6 Hz, 1H), 4.40 (d, J = 5.6 Hz, 2H), 1.74 (s, 3H), 1.65 (s, 3H). LCMS RT = 1.053 min, m/z = 328 [M + H]⁺ |
| 593 | 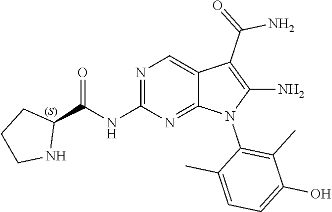 | ¹H NMR (400 MHz, DMSO-d₆) δ 11.54 (bs, 1H), 10.07 (s, 1H), 9.80 (s, 1H), 8.88 (s, 1H), 8.66 (s, 1H), 7.35 (s, 2H), 7.09 (d, J = 8.4 Hz, 2H), 7.00 (d, J = 8.4 Hz, 2H), 4.41 (bs, 1H), 3.232-3.177 (m, 2H), 2.24 (bs, 2H), 1.84 (d, J = 9.2 Hz, 2H), 1.80 (d, J = 3.6 Hz, 3H), 1.72 (d, J = 4.8 Hz, 3H). LCMS RT = 1.77 min, m/z = 410.5 [M + H]⁺ |
| 594 | 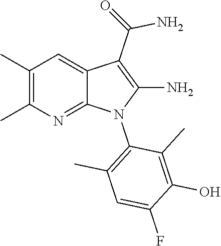 | ¹H NMR (400 MHz, DMSO-d₆) δ 9.31 (d, J = 1.2 Hz, 1H), 8.98 (s, 1H), 7.94 (d, J = 2.0 Hz, 1H), 6.97 (brs, 2H), 6.91 (d, J = 8.4 Hz, 1H), 6.74 (d, J = 8.0 Hz, 1H), 4.84 (brs, 2H), 2.23 (s, 3H), 1.86 (s, 3H), 1.80 (s, 3H). LCMS R_{T} = 0.481 min, m/z = 311.1 [M + H]⁺. |
| 595 | 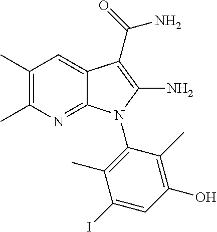 | ¹H NMR (400 MHz, CD₃OD) δ 8.81 (s, 1H), 6.80 (d, J = 11.2 Hz, 1H), 2.59 (s, 3H), 1.79 (s, 6H). ¹⁹F NMR (400 MHz, DMSO-d₆) δ −119.510. LCMS R_{T} = 0.781 min, m/z = 330 [M + H]⁺. |
| 596 | 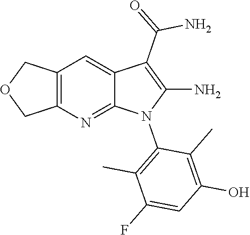 | ¹H NMR (400 MHz, DMSO-d₆) δ 9.55 (brs, 1H), 8.53 (s, 1H), 7.04 (d, J = 8.0 Hz, 1H), 6.89 (d, J = 8.0 Hz, 1H), 6.73 (brs, 2H), 6.64 (brs, 2H), 6.59 (t, J = 6.0 Hz, 3H), 3.97-3.88 (m, 1H), 3.26-3.15 (m, 2H), 2.42-2.30 (m, 1H), 2.20-2.07 (m, 1H), 1.78 (s, 3H), 1.71 (s, 3H). LCMS R_{T} = 0.746 min, m/z = 439.4 [M + H]⁺. |
| 597 | 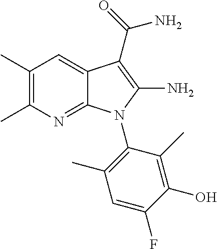 | ¹H NMR (400 MHz, DMSO-d₆) δ 9.63 (brs, 1H), 8.90 (s, 1H), 8.01 (s, 1H), 7.28 (brs, 2H), 6.67 (d, J = 11.2 Hz, 1H), 4.82 (brs, 2H), 2.35 (s, 3H), 1.78 (d, J = 2.0 Hz, 3H), 1.77 (s, 3H). ¹⁹F NMR (400 MHz, DMSO-d₆) δ −118.296. LCMS R_{T} = 0.722 min, m/z = 328.9 [M + H]⁺. |
| 599 | 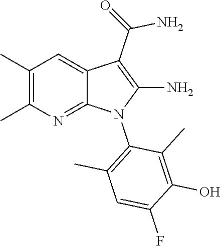 | ¹H NMR (400 MHz, DMSO-d₆) δ 10.16 (brs, 1H), 8.80 (d, J = 1.6 Hz, 1H), 8.14 (s, 1H), 7.98 (d, J = 2.8 Hz, 1H), 7.06 (brs, 2H), 6.95 (brs, 2H), 6.82 (d, J = 10.8 Hz, 1H), 1.71 (s, 3H), 1.67 (s, 3H). ¹⁹F NMR (400 MHz, DMSO-d₆) δ −118.126, −154.838. LCMS R_{T} = 0.864 min, m/z = 333.3 [M + H]⁺. |
| 600 | 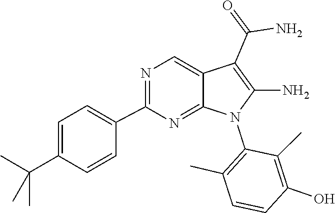 [SMILES: Cc1ccc(O)c(C)c1-n1c(N)c(C(N)=O)c2cnc(-c3ccc(C(C)(C)C)cc3)nc21] | ¹H NMR (400 MHz, DMSO-d₆) δ 9.54 (s, 1H), 8.98 (s, 1H), 7.97 (d, J = 8.3 Hz, 2H), 7.35 (d, J = 8.1 Hz, 2H), 7.13 (s, 2H), 7.05 (d, J = 8.3 Hz, 1H), 6.90 (d, J = 8.3 Hz, 1H), 6.86 (s, 2H), 1.73 (s, 3H), 1.65 (s, 3H), 1.20 (s, 9H). LCMS: RT = 1.016 min, m/z 430.3 [M + H]⁺. |
| 602 | 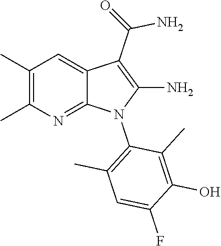 | ¹H NMR (400 MHz, DMSO-d₆) δ 9.66 (brs, 1H), 8.88 (s, 1H), 7.21-7.08 (m, 3H), 6.84 (brs, 2H), 2.44 (s, 3H), 1.77 (s, 3H), 1.71 (s, 3H). ¹⁹F NMR (400 MHz, DMSO-d₆) δ −133.712. LCMS R_{T} = 1.392 min, m/z = 330.4 [M + H]⁺. |
| 603 | 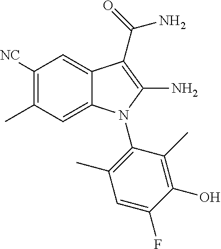 | ¹H NMR (400 MHz, DMSO-d₆) δ 9.62 (brs, 1H), 8.14 (d, J = 2.0 Hz, 1H), 7.75 (d, J = 2.0 Hz, 1H), 7.21 (brs, 2H), 7.11 (d, J = 11.6 Hz, 1H), 6.87 (brs, 2H), 1.75 (s, 3H), 1.70 (s, 3H). ¹⁹F NMR (400 MHz, DMSO-d₆) δ −133.930. LCMS R_{T} = 0.634 min, m/z = 349 [M + H]⁺. |
| 604 | 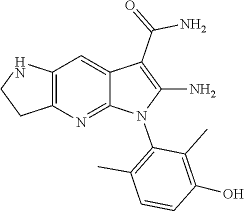 | ¹H NMR (400 MHz, DMSO-d₆) δ 9.59 (brs, 1H), 8.67 (s, 1H), 8.13 (s, 1H of formic acid), 7.06 (d, J = 8.4 Hz, 1H), 6.92 (d, J = 8.4 Hz, 1H), 6.85 (brs, 4H), 2.76-2.60 (m, 2H), 1.80 (s, 3H), 1.72 (s, 3H), 1.16 (t, J = 7.2 Hz, 3H). ¹⁹F NMR (400 MHz, DMSO-d₆) δ −155.099. LCMS R_{T} = 1.527 min, m/z = 343.1 [M + H]⁺. |
| 605 | 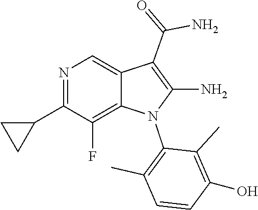 [SMILES: Cc1ccc(O)c(C)c1-n1c(N)c(C(N)=O)c2cnc(C3CC3)c(F)c21] | ¹H NMR (400 MHz, DMSO-d₆) δ 9.59 (brs, 1H), 8.58 (s, 1H), 7.06 (d, J = 8.0 Hz, 1H), 6.92 (d, J = 8.0 Hz, 1H), 6.82 (brs, 4H), 2.17- 2.06 (m, 1H), 1.81 (s, 3H), 1.73 (s, 3H), 0.92- 0.84 (m, 4H). ¹⁹F NMR (400 MHz, DMSO-d₆) δ −158.784. LCMS R_{T} = 1.519 min, m/z = 355.3 [M + H]⁺. |
| 606 | 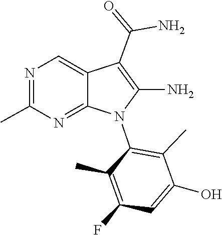 | ¹H NMR (400 MHz, DMSO-d₆) δ 10.11 (brs, 1H), 8.89 (s, 1H), 7.17 (brs, 2H), 8.86 (brs, 2H), 6.82 (d, J = 11.2 Hz, 1H), 2.44 (s, 3H), 1.66 (d, J = 1.6 Hz, 3H), 1.62 (s, 3H). ¹⁹F NMR (400 MHz, DMSO-d₆) δ −118.198. LCMS R_{T} = 0.428 min, m/z = 330.3 [M + H]⁺. |
| 607 | 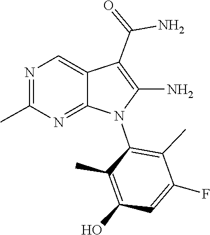 | ¹H NMR (400 MHz, DMSO-d₆) δ 10.12 (brs, 1H), 8.89 (s, 1H), 7.17 (brs, 2H), 8.86 (brs, 2H), 6.82 (d, J = 11.2 Hz, 1H), 2.44 (s, 3H), 1.66 (s, 3H), 1.62 (s, 3H). ¹⁹F NMR (400 MHz, DMSO-d₆ δ −118.179. LCMS RT = 0.350 min, m/z = 330.4 [M + H]⁺. |
| 608 | 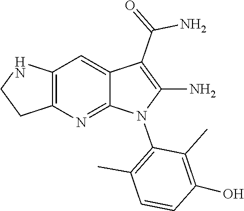 | 1H NMR (500 MHz, DMSO) δ 9.69 (s, 1H), 9.13 (s, 1H), 7.50 (s, 2H), 7.13 (d, J = 8.5 Hz, 3H), 6.98 (d, J = 8.4 Hz, 1H), 2.55 (s, 4H), 1.76 (s, 3H), 1.68 (s, 3H). LCMS: RT = 0.871 min, m/z 366.2 [M + H]⁺ |
| 609 | 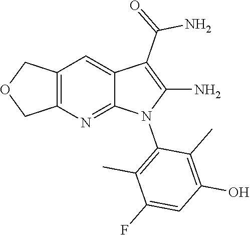 | 1H NMR (500 MHz, DMSO) δ 9.48 (s, 1H), 8.58 (s, 1H), 7.06 (d, J = 8.2 Hz, 1H), 6.89 (d, J = 8.3 Hz, 1H), 6.64 (d, J = 31.5 Hz, 4H), 3.41 (qt, J = 9.3, 4.7 Hz, 4H), 1.81 (s, 3H), 1.73 (s, 3H), 1.00 (t, J = 7.0 Hz, 6H). LCMS: RT = 0.671 min, m/z 369.3 [M + H]⁺. |
| 616 | 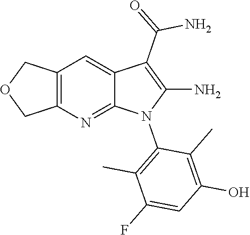 | ¹H NMR (400 MHz, DMSO-d₆) δ 9.72 (br s, 1H), 8.81 (s, 1H), 7.30 (br s, 2H), 7.10 (d, J = 8.4 Hz, 1H), 7.02 (br s, 2H), 6.97 (d, J = 8.4 Hz, 1H), 4.24 (q, J = 6.8 Hz, 2H), 1.80 (s, 3H), 1.71 (s, 3H), 1.25 (t, J = 7.2 Hz, 3H). LCMS R_{T} = 1.040 min, m/z = 342.2 [M + H]⁺. |
| 617 | 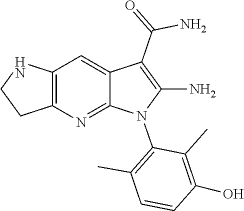 | ¹H NMR (400 MHz, DMSO-d₆) δ 13.32 (br s, 1H), 8.92 (s, 1H), 7.68 (d, J = 8.8 Hz, 1H), 7.54 (s, 1H), 7.45 (d, J = 8.4 Hz, 1H), 7.12 (br s, 2H), 6.88 (br s, 2H), 2.38 (s, 3H), 2.03 (s, 3H). LCMS R_{T} = 1.028 min, m/z = 321.1 [M + H]⁺. |
| 618 | 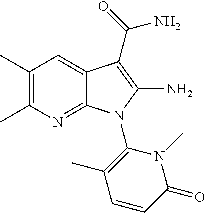 [SMILES: Cc1cc2c(C(N)=O)c(N)n(-c3c(C)ccc(=O)n3C)c2nc1C] | ¹H NMR (500 MHz, DMSO) δ 9.67 (s, 1H), 8.90 (s, 1H), 7.32 (s, 2H), 7.11 (d, J = 8.3 Hz, 1H), 7.04-6.94 (m, 2H), 1.77 (s, 3H), 1.69 (s, 2H). LCMS: RT = 0.766 min, m/z 332.2 [M + H]⁺. |
| 619 | 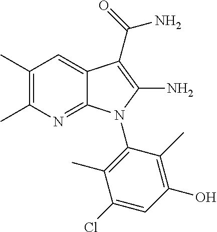 | ¹H NMR (500 MHz, DMSO) δ 10.11 (s, 1H), 8.89 (s, 1H), 7.18 (s, 2H), 7.08 (s, 1H), 6.86 (s, 2H), 2.44 (s, 3H), 1.77 (s, 3H), 1.64 (s, 3H). LCMS RT = 0.59 min, m/z = 346.2 [M + H]⁺ |
| 620 | 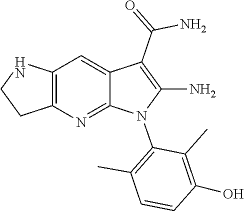 [SMILES: Cc1ccc(O)c(C)c1-n1c(N)c(C(N)=O)c2cc3c(nc21)CCN3] | ¹H NMR (400 MHz, DMSO-d₆) δ 9.59 (br s, 1H), 8.82 (s, 1H), 7.08 (d, J = 8.4 Hz, 1H), 7.06 (br s, 2H), 6.93 (d, J = 8.4 Hz, 1H), 6.84 (br s, 2H), 6.93 (d, J = 8.4 Hz, 1H), 6.84 (br s, 2H), 2.05-1.90 (m, 1H), 1.76 (s, 3H), 1.67 (s, 3H), 0.90-0.76 (m, 4H). LCMS R_{T} = 1.658 min, m/z = 338.1 [M + H]⁺. |
| 621 | 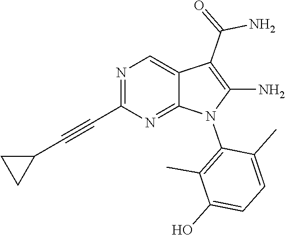 [SMILES: Cc1ccc(O)c(C)c1-n1c(N)c(C(N)=O)c2cnc(C#CC3CC3)nc21] | ¹H NMR (400 MHz, DMSO-d₆) δ 9.65 (br s, 1H), 8.90 (s, 1H), 7.28 (s, 2H), 7.09 (d, J = 8.0 Hz, 1H), 6.95 (d, J = 8.0 Hz, 1H), 6.94 (br s, 2H), 1.73 (s, 3H), 1.65 (s, 3H), 1.55-1.45 (m, 1H), 0.90-0.81 (m, 2H), 0.78-0.70 (m, 2H). LCMS R_{T} = 1.148 min, m/z = 362.1 [M + H]⁺. |
| 622 | 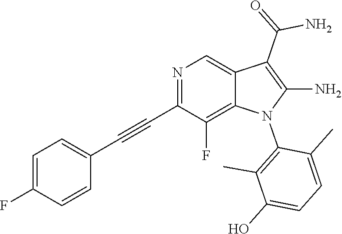 [SMILES: Cc1ccc(O)c(C)c1-n1c(N)c(C(N)=O)c2cnc(C#Cc3ccc(F)cc3)c(F)c21] | ¹H NMR (400 MHz, DMSO-d₆) δ 9.66 (br s, 1H), 8.80 (d, J = 1.6 Hz, 1H), 7.59 (dd, J = 8.8, 5.6 Hz, 2H), 7.26 (t, J = 8.8 Hz, 2H), 7.12 (br s, 2H), 7.09 (d, J = 8.4 Hz, 1H), 7.04 (br s, 2H), 6.94 (d, J = 8.0 Hz, 1H), 1.82 (s, 3H), 1.74 (s, 3H). ¹⁹F NMR (400 MHz, DMSO-d₆) δ −110.082, −146.126. LCMS R_{T} = 1.943 min, m/z = 433.1 [M + H]⁺. |
| 623 | 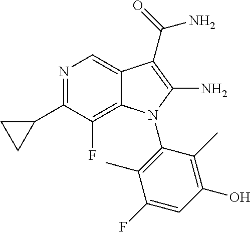 [SMILES: Cc1c(O)cc(F)c(C)c1-n1c(N)c(C(N)=O)c2cnc(C3CC3)c(F)c21] | ¹H NMR (400 MHz, DMSO-d₆) δ 10.10 (br s, 1H), 8.59 (s, 1H), 6.95 (br s, 2H), 6.85 (br s, 2H), 6.81 (d, J = 11.2 Hz, 2H), 2.17-2.05 (m, 1H), 1.73 (s, 3H), 1.69 (s, 3H), 0.95-0.82 (m, 4H). ¹⁹F NMR (400 MHz, DMSO-d₆) δ −118.264, −118.269, −158.904. LCMS R_{T} = 1.884 min, m/z = 373.2 [M + H]⁺. |
| 624 | 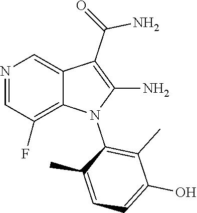 | ¹H NMR (400 MHz, DMSO-d₆) δ 9.94-9.77 (m, 1H), 9.12-9.04 (m, 1H), 8.60 (d, J = 5.2 Hz, 1H), 7.52 (br s, 2H), 7.32 (br s, 2H), 7.12 (d, J = 8.4 Hz, 1 H), 7.06-6.97 (m, 1H), 1.82 (s, 3H), 1.74 (s, 3H). ¹⁹F NMR (400 MHz, DMSO-d₆) δ −148.517. LCMS R_{T} = 1.500 min, m/z = 315.1 [M + H]⁺. |
| 625 | 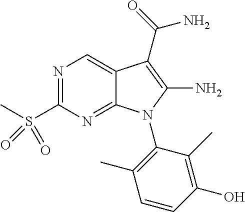 [SMILES: Cc1ccc(O)c(C)c1-n1c(N)c(C(N)=O)c2cnc(S(C)(=O)=O)nc21] | ¹H NMR (500 MHz, DMSO) δ 9.70 (s, 1H), 9.10 (s, 1H), 7.56 (s, 2H), 7.16-7.10 (m, 3H), 6.98 (d, J = 8.3 Hz, 1H), 3.24 (s, 3H), 1.77 (s, 3H), 1.68 (s, 3H). LCMS R_{T} = 0.596 min, m/z = 376.2 [M + H]⁺ |
| 626 | 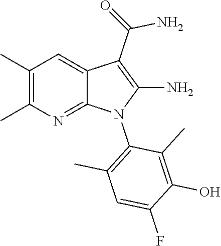 | ¹H NMR (400 MHz, DMSO-d₆) δ 9.60 (brs, 1H), 8.63 (d, J = 1.6 Hz, 1H), 7.05 (d, J = 8.4 Hz, 1H), 6.91 (d, J = 8.0 Hz, 1H), 6.85 (brs, 2H), 6.84 (brs, 2H), 2.33 (d, J = 3.2 Hz, 3H), 1.80 (s, 3H), 1.71 (s, 3H). ¹⁹F NMR (400 MHz, DMSO-d₆) δ −152.996. LCMS R_{T} = 1.640 min, m/z = 329.1 [M + H]⁺. |
| 627 | 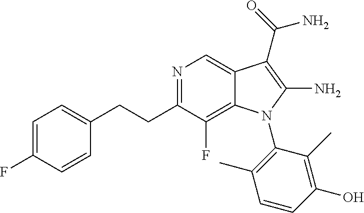 [SMILES: Cc1ccc(O)c(C)c1-n1c(N)c(C(N)=O)c2cnc(CCc3ccc(F)cc3)c(F)c21] | ¹H NMR (400 MHz, DMSO-d₆) δ 9.58 (brs, 1H), 8.70 (s, 1H), 7.09 (dd, J = 5.6, 3.2 Hz, 2H), 7.01 (dd, J = 18.0, 8.8 Hz, 3H), 6.89 (d, J = 8.4 Hz, 1H), 6.88 (brs, 2H), 6.84 (brs, 2H), 2.92 (s, 4H), 1.74 (s, 3H), 1.66 (s, 3H). ¹⁹F NMR (400 MHz, DMSO-d₆) δ −117.652, −154.419. LCMS R_{T} = 1.91 min, m/z = 437.4 [M + H]⁺. |
| 629 | 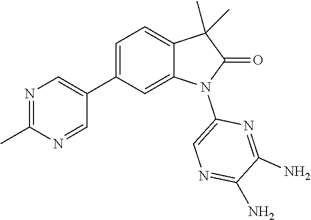 | ¹H NMR (400 MHz, DMSO-d₆) δ 9.62 (brs, 1H), 9.08 (s, 1H), 8.16 (dd, J = 8.8, 5.6 Hz, 2H), 7.29-7.15 (m, 4H), 7.12 (d, J = 8.4 Hz, 1H), 6.97 (d, J = 8.4 Hz, 1H), 6.96 (brs, 2H), 1.81 (s, 3H), 1.72 (s, 3H). ¹⁹F NMR (400 MHz, DMSO-d₆) δ −113.132. LCMS R_{T} = 1.111 min, m/z = 392.1 [M + H]⁺. |
| 630 | 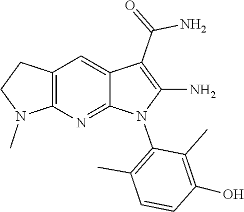 | ¹H NMR (400 MHz, DMSO-d₆) δ 9.56 (br s, 1H), 8.96 (s, 1H), 7.19 (br s, 2H), 7.06 (d, J = 8.4 Hz, 1H), 6.92 (d, J = 8.4 Hz, 1H), 6.98 (br s, 2H), 6.14 (d, J = 11.6 Hz, 1H), 5.10 (t, J = 11.2 Hz, 1H), 3.19-3.07 (m, 1H), 1.77 (s, 3H), 1.68 (s, 3H), 0.75-0.64 (m, 2H), 0.44-0.35 (m, 2H). LCMS R_{T} = 1.054 min, m/z = 364.1 [M + H]⁺. |
| 631 | 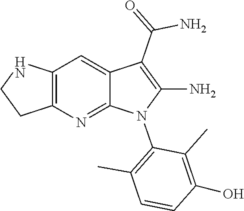 | ¹H NMR (400 MHz, DMSO-d₆) δ 10.09 (br s, 1H), 8.82 (s, 1H), 7.13 (br s, 2H), 6.83 (br s, 2H), 6.82 (d, J = 10.8 Hz, 1H), 2.02-1.83 (m, 1H), 1.67 (s, 3H), 1.63 (s, 3H), 0.93-0.76 (m, 4H). ¹⁹F NMR (400 MHz, DMSO-d₆) δ −118.190. LCMS R_{T} = 1.364 min, m/z = 356.1 [M + H]⁺. |
| 632 | 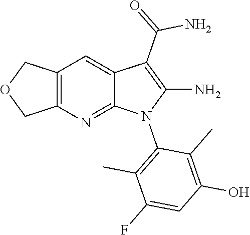 | ¹H NMR (400 MHz, CD₃OD) δ 8.23 (s, 1H), 7.07 (d, J = 8.4 Hz, 1H), 6.90 (d, J = 8.4 Hz, 1H), 3.30 (q, J = 8.4 Hz, 4H), 1.93 (s, 3H), 1.87 (s, 3H), 1.06 (t, J = 6.8 Hz, 6H). ¹⁹F NMR (400 MHz, DMSO-d₆) δ −159.445. LCMS R_{T} = 1.445 min, m/z = 386.2 [M + H]⁺. |
| 633 | 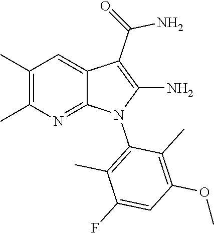 | ¹H NMR (400 MHz, DMSO-d₆) δ 9.58 (br s, 1H), 8.89 (s, 1H), 7.08 (d, J = 8.4 Hz, 1H), 7.04 (br s, 2H), 6.93 (d, J = 8.4 Hz, 1H), 6.84 (br s, 2H), 2.98-2.85 (m, 1H), 1.76 (s, 3H), 1.67 (s, 3H), 1.15 (d, J = 6.8 Hz, 6H). LCMS R_{T} = 0.839 min, m/z = 340.2 [M + H]⁺. |
| 634 | 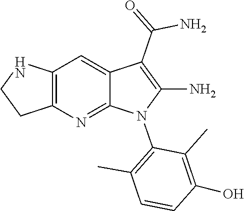 | ¹H NMR (400 MHz, DMSO-d₆) δ 9.68 (br s, 1H), 8.57 (s, 1H), 7.08 (d, J = 8.4 Hz, 1H), 7.07 (br s, 2H), 6.98 (br s, 2H), 6.94 (d, J = 8.4 Hz, 1H), 1.80 (s, 3H), 1.72 (s, 3H). ¹⁹F NMR (400 MHz, DMSO-d₆) δ −147.839. LCMS R_{T} = 2.538 min, m/z = 349.0 [M + H]⁺. |
| 635 | 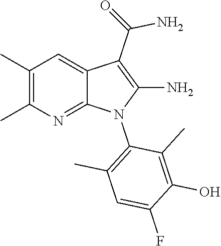 | ¹H NMR (400 MHz, DMSO-d₆) δ 9.61 (br s, 1H), 8.68 (d, J = 1.2 Hz, 1H), 8.15 (s, 1H), 7.06 (d, J = 8.4 Hz, 1H), 6.92 (d, J = 8.4 Hz, 1H), 6.85 (br s, 2H), 6.83 (br s, 2H), 3.31-3.12 (m, 1H), 1.81 (s, 3H), 1.72 (s, 3H), 1.18 (d, J = 6.8 Hz, 6H). ¹⁹F NMR (400 MHz, DMSO-d₆) δ −156.113. LCMS R_{T} = 1.977 min, m/z = 357.2 [M + H]⁺. |
| 638 | 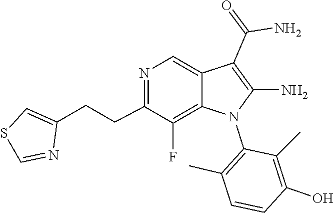 [SMILES: Cc1ccc(O)c(C)c1-n1c(N)c(C(N)=O)c2cnc(CCc3cscn3)c(F)c21] | ¹H NMR (400 MHz, DMSO-d₆) δ 9.60 (br s, 1H), 8.97 (d, J = 2.0 Hz, 1H), 8.70 (s, 1H), 8.13 (s, 1H), 7.23 (d, J = 1.6 Hz, 1H), 7.05 (d, J = 8.0 Hz, 1H), 6.91 (d, J = 8.4 Hz, 1H), 6.88 (br s, 2H), 6.85 (br s, 2H), 3.13-3.01 (m, 4H), 1.78 (s, 3H), 1.69 (s, 3H). ¹⁹F NMR (400 MHz, DMSO-d₆) δ −154.437. LCMS R_{T} = 1.179 min, m/z = 426.2 [M + H]⁺. |
| 639 | 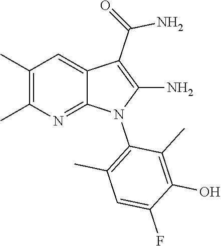 | ¹H NMR (400 MHz, DMSO-d₆) δ 9.84 (br s, 1H), 8.88 (s, 1H), 7.49 (br s, 2H), 7.32 (br s, 2H), 7.12 (d, J = 8.4 Hz, 1H), 7.00 (d, J = 8.4 Hz, 1H), 3.28-3.18 (m, 1H), 1.83 (d, J = 6.8 Hz, 3H), 1.75 (d, J = 6.8 Hz, 5H), 1.35 (d, J = 7.2 Hz, 3H), 0.74 (t, J = 7.2 Hz, 3H). ¹⁹F NMR (400 MHz, DMSO-d₆) δ −149.856. LCMS R_{T} = 1.326 min, m/z = 371.1 [M + H]⁺. |
| 640 | 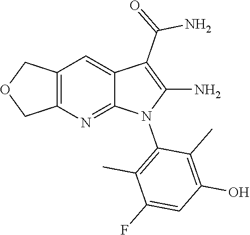 | ¹H NMR (400 MHz, DMSO-d₆) δ 9.75 (br s, 1H), 9.07 (s, 1H), 7.96-7.43 (m, 2H), 7.38- 7.15 (m, 2H), 7.13 (d, J = 8.4 Hz, 1H), 6.98 (d, J = 8.4 Hz, 1H), 3.05-2.89 (m, 1H), 1.77 (d, J = 2.8 Hz, 3H), 1.69 (d, J = 2.8 Hz, 3H), 1.66- 1.50 (m, 2H), 1.18 (d, J = 6.8 Hz, 3H), 0.72 (t, J = 7.2 Hz, 3H). LCMS R_{T} = 1.216 min, m/z = 354.1 [M + H]⁺. |
| 641 | 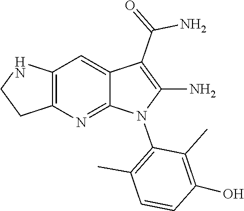 | ¹H NMR (400 MHz, DMSO-d₆) δ 9.55 (br s, 1H), 8.86 (s, 1H), 7.11 (br s, 2H), 7.02 (d, J = 8.4 Hz, 1H), 6.87 (d, J = 8.4 Hz, 2H), 6.85 (br s, 2H), 1.69 (s, 3H), 1.61 (s, 3H), 1.26 (t, J = 2.9 Hz, 2H), 1.29-1.23 (m, 2H), 1.22-1.14 (m, 2H). ¹⁹F NMR (400 MHz, DMSO-d₆) δ −65.365. LCMS R_{T} = 1.109 min, m/z = 406.1 [M + H]⁺. |
| 642 | 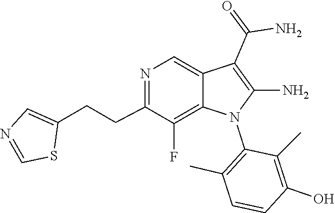 [SMILES: Cc1ccc(O)c(C)c1-n1c(N)c(C(N)=O)c2cnc(CCc3cncs3)c(F)c21] | ¹H NMR (400 MHz, DMSO-d₆) δ 9.60 (br s, 1H), 8.82 (s, 1H), 8.72 (s, 1H), 7.56 (s, 1H), 7.04 (d, J = 8.4 Hz, 1H), 6.94-6.78 (m, 5H), 3.24 (t, J = 7.2 Hz, 2H), 2.99 (t, J = 6.8 Hz, 2H), 1.77 (s, 3H), 1.68 (s, 3H). ¹⁹F NMR (400 MHz, DMSO-d₆) δ −154.235. LCMS R_{T} = 1.009 min, m/z = 426.1 [M + H]⁺. |
| 647 | 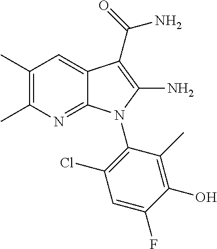 | ¹H NMR (400 MHz, DMSO-d₆) δ 10.21 (br s, 1H), 9.14 (s, 1H), 7.62 (br s, 2H), 7.14 (br s, 2H), 6.86 (d, J = 11.2 Hz, 1H), 1.67 (s, 3H), 1.63 (s, 3H). ¹⁹F NMR (400 MHz, DMSO-d₆) δ −66.664, −117.733. LCMS R_{T} = 1.825 min, m/z = 384.1 [M + H]⁺. |
| 648 | 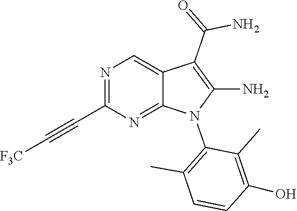 [SMILES: Cc1ccc(O)c(C)c1-n1c(N)c(C(N)=O)c2cnc(C#CC(F)(F)F)nc21] | ¹H NMR (400 MHz, CD₃OD) δ 8.93 (s, 1H), 7.14 (d, J = 8.4 Hz, 1H), 6.97 (d, J = 8.0 Hz, 1H), 1.86 (s, 3H), 1.81 (s, 3H). ¹⁹F NMR (400 MHz, DMSO-d₆) δ −51.963. LCMS R_{T} = 1.216 min, m/z = 389.9 [M + H]⁺. |
| 649 | 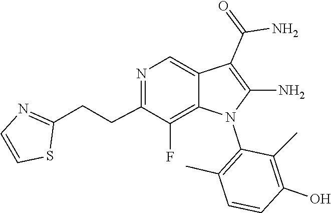 [SMILES: Cc1ccc(O)c(C)c1-n1c(N)c(C(N)=O)c2cnc(CCc3nccs3)c(F)c21] | ¹H NMR (400 MHz, DMSO-d₆) δ 9.59 (br s, 1H), 8.71 (d, J = 1.6 Hz, 1H), 7.62 (d, J = 3.2 Hz, 1H), 7.49 (d, J = 3.2 Hz, 1H), 7.05 (d, J = 8.0 Hz, 1H), 6.91 (d, J = 8.4 Hz, 1H), 6.88 (br s, 2H), 6.86 (br s, 2H), 3.44-3.34 (m, 2H), 3.14-3.06 (m, 2H), 1.78 (s, 3H), 1.69 (s, 3H). ¹⁹F NMR (400 MHz, DMSO-d₆) δ −154.189. LCMS R_{T} = 1.213 min, m/z = 426.1 [M + H]⁺. |
| 650 | 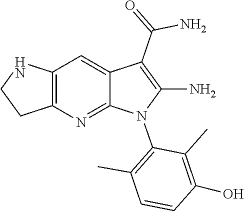 | ¹H NMR (400 MHz, DMSO-d₆) δ 9.55 (br s, 1H), 8.82 (s, 1H), 7.07 (d, J = 8.4 Hz, 1H), 6.97 (br s, 2H), 6.92 (d, J = 8.4 Hz, 1H), 6.78 (br s, 2H), 2.27 (s, 2H), 1.75 (s, 3H), 1.67 (s, 3H), −0.10 (s, 9H). LCMS R_{T} = 1.509 min, m/z = 384.2 [M + H]⁺. |
| 651 | 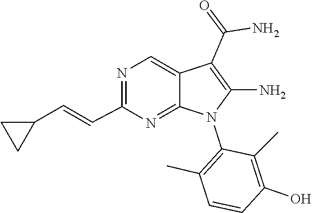 [SMILES: Cc1ccc(O)c(C)c1-n1c(N)c(C(N)=O)c2cnc(/C=C/C3CC3)nc21] | ¹H NMR (400 MHz, DMSO-d₆) δ 9.60 (br s, 1H), 8.88 (s, 1H), 7.09 (br s, 2H), 7.08 (d, J = 7.6 Hz, 1H), 6.93 (d, J = 8.0 Hz, 1H), 6.88 (br s, 2H), 6.44-6.22 (m, 2H), 1.76 (s, 3H), 1.67 (s, 3H), 1.62-1.50 (m, 1H), 0.83-0.76 (m, 2H), 0.56-0.49 (m, 2H). LCMS R_{T} = 1.091 min, m/z = 364.0 [M + H]⁺ |
| 652 | 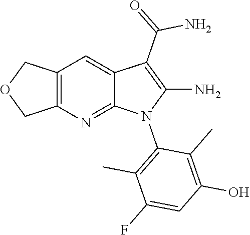 | ¹H NMR (400 MHz, CD₃OD) δ 8.72 (s, 1H), 6.77 (d, J = 10.8 Hz, 1H), 2.40 (s, 2H), 1.77 (s, 6H), −0.05 (s, 9H). ¹⁹F NMR (400 MHz, CD₃OD) δ −119.717. LCMS R_{T} = 0.459 min, m/z = 402.2 [M + H]⁺. |
| + | | |
| 653 | 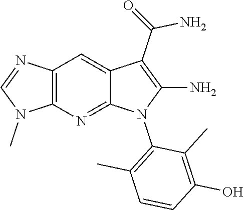 | ¹H NMR (400 MHz, DMSO-d₆) δ 10.08 (d, J = 6.8 Hz, 1H), 8.87 (s, 1H), 7.16 (br s, 2H), 6.84 (br s, 2H), 6.82 (d, J = 11.2 Hz, 1H), 1.97 (dd, J = 8.0, 5.6 Hz, 1H), 1.66 (d, J = 11.2 Hz, 3H), 1.62 (d, J = 9.6 Hz, 3H), 1.14 (s, 3H), 1.13-1.05 (m, 1H), 0.91 (d, J = 1.6 Hz, 3H), 0.76 (d, J = 8.4, 3.2 Hz, 1H). ¹⁹F NMR (400 MHz, DMSO-d₆) δ −118.257. LCMS R_{T} = 2.298 & 2.321 min, m/z = 384.1 [M + H]⁺. |
| 655 | 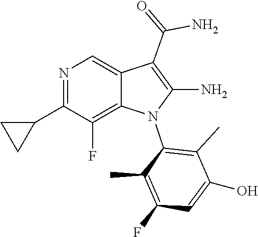 | ¹H NMR (400 MHz, DMSO-d₆) δ 10.12 (br s, 1H), 8.59 (s, 1H), 6.95 (br s, 2H), 6.85 (br s, 2H), 6.81 (d, J = 11.2 Hz, 1H), 2.20-2.07 (m, 1H), 1.73 (s, 3H), 1.68 (s, 3H), 0.96-0.82 (m, 4H). ¹⁹F NMR (400 MHz, DMSO-d₆) δ −118.262, −158.904. LCMS R_{T} = 1.424 min, m/z = 373.1 [M + H]⁺. |
| 656 | 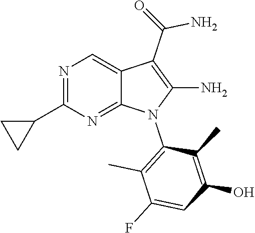 | ¹H NMR (400 MHz, DMSO-d₆) δ 10.09 (br s, 1H), 8.82 (s, 1H), 7.13 (br s, 2H), 6.83 (br s, 2H), 6.82 (d, J = 11.2 Hz, 1H), 2.03-1.91 (m, 1H), 1.67 (d, J = 1.2 Hz, 3H), 1.63 (s, 3H), 0.89-0.75 (m, 4H). ¹⁹F NMR (400 MHz, DMSO-d₆) δ −118.198. LCMS R_{T} = 1.318 min, m/z = 356.1 [M + H]⁺. |
| 659 | 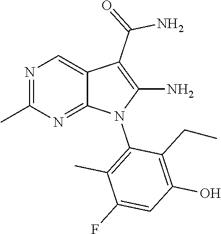 [SMILES: CCc1c(O)cc(F)c(C)c1-n1c(N)c(C(N)=O)c2cnc(C)nc21] | ¹H NMR (400 MHz, DMSO-d₆) δ 10.07 (br s, 1H), 8.88 (s, 1H), 7.16 (br s, 2H), 6.85 (br s, 2H), 6.83 (d, J = 10.8 Hz, 1H), 2.44 (s, 3H), 2.16-1.98 (m, 2H), 1.62 (d, J = 1.2 Hz, 3H), 0.82 (t, J = 7.2 Hz, 3H). ¹⁹F NMR (400 MHz, DMSO-d₆) δ −117.795. LCMS R_{T} = 1.378 min, m/z = 344.1 [M + H]⁺. |
| 660 | 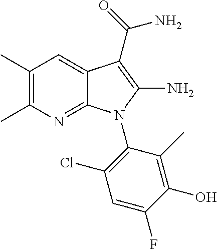 | ¹H NMR (400 MHz, DMSO-d₆) δ 10.21 (br s, 1H), 9.15 (s, 1H), 8.39 (s, 1H), 7.26 (br s, 2H), 7.05 (br s, 2H), 6.86 (d, J = 11.2 Hz, 1H), 1.69 (s, 3H), 1.64 (s, 3H). ¹⁹F NMR (400 MHz, DMSO-d₆) δ −117.873. LCMS R_{T} = 0.892 min, m/z = 340.1 [M + H]⁺. |
| 661 | 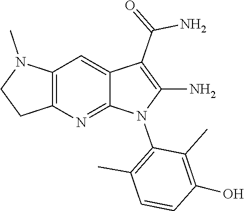 | ¹H NMR (400 MHz, DMSO-d₆) δ 9.64 (br s, 1H), 9.13 (s, 1H), 8.37 (s, 1H), 7.14 (br s, 2H), 7.10 (d, J = 8.4 Hz, 1H), 7.03 (br s, 2H), 6.97 (d, J = 8.0 Hz, 1H), 1.77 (s, 3H), 1.69 (s, 3H). LCMS R_{T} = 1.468 min, m/z = 322.0 [M + H]⁺. |
| 662 | 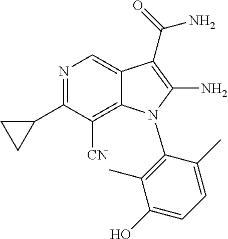 [SMILES: Cc1ccc(O)c(C)c1-n1c(N)c(C(N)=O)c2cnc(C3CC3)c(C#N)c21] | ¹H NMR (400 MHz, DMSO-d₆) δ 9.63 (br s, 1H), 8.93 (s, 1H), 7.09 (d, J = 8.4 Hz, 1H), 7.06-6.88 (m 5H), 2.32-2.18 (m, 1H), 1.78 (s, 3H), 1.70 (s, 3H), 1.04-0.92 (m, 4H). LCMS R_{T} = 1.794 min, m/z = 362.1 [M + H]⁺. |
| 663 | 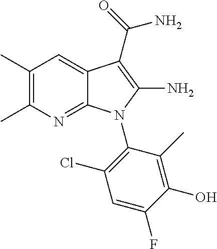 [SMILES: Cc1cc2c(C(N)=O)c(N)n(-c3c(Cl)cc(F)c(O)c3C)c2nc1C] | ¹H NMR (400 MHz, DMSO-d₆) δ 10.15 (br s, 1H), 8.94 (s, 1H), 7.09 (br s, 2H), 6.95 (br s, 2H), 6.85 (d, J = 11.2 Hz, 1H), 2.32-2.19 (m, 1H), 1.70 (d, J = 1.2 Hz, 3H), 1.65 (s, 3H), 1.00 (d, J = 6.4 Hz, 4H). ¹⁹F NMR (400 MHz, DMSO-d₆) δ −118.002. LCMS R_{T} = 2.964 min, m/z = 380.1 [M + H]⁺. |
| 663-B | 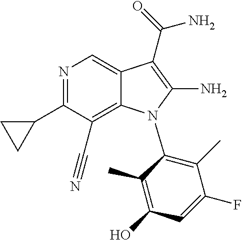 | ¹H NMR (400 MHz, DMSO-d₆) δ 10.11 (br s, 1H), 8.95 (s, 1H), 7.09 (br s, 2H), 6.95 (br s, 2H), 6.85 (d, J = 11.2 Hz, 1H), 2.33-2.20 (m, 1H), 1.70 (s, 3H), 1.65 (s, 3H), 1.00 (d, J = 6.4 Hz, 4H). ¹⁹F NMR (400 MHz, DMSO-d₆) δ −118.025. LCMS R_{T} = 1.965 min, m/z = 380.2 [M + H]⁺. |
| 667 | 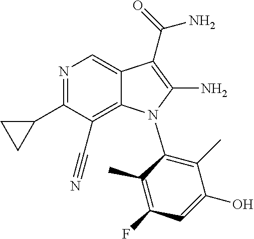 | ¹H NMR (400 MHz, DMSO-d₆) δ 10.11 (br s, 1H), 8.95 (s, 1H), 7.09 (br s, 2H), 6.95 (br s, 2H), 6.85 (d, J = 11.2 Hz, 1H), 2.33-2.20 (m, 1H), 1.70 (s, 3H), 1.65 (s, 3H), 1.00 (d, J = 6.0 Hz, 4H). ¹⁹F NMR (400 MHz, DMSO-d₆) δ −118.025. LCMS R_{T} = 1.987 min, m/z = 380.2 [M + H]⁺. |
| 673 | 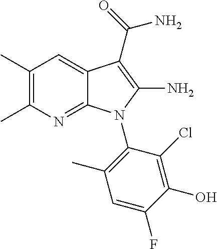 | ¹H NMR (400 MHz, DMSO-d₆) δ 10.21 (br s, 1H), 8.94 (s, 1H), 7.10 (br s, 2H), 6.95 (br s, 2H), 6.88 (d, J = 11.2 Hz, 1H), 2.28-2.22 (m, 1H), 2.16-2.07 (m, 2H), 1.67 (s, 3H), 1.00 (d, J = 6.4 Hz, 4H), 0.85 (t, J = 7.2 Hz, 3H). ¹⁹F NMR (400 MHz, DMSO-d₆) δ −117.631. LCMS R_{T} = 0.972 min, m/z = 394.2 [M + H]⁺. |
| 673-B | 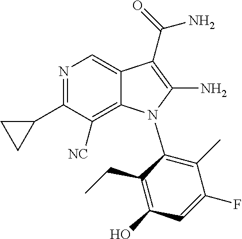 | ¹H NMR (400 MHz, CD₃OD) δ 8.81 (s, 1H), 6.82 (d, J = 10.8 Hz, 1H), 2.42-2.33 (m, 1H), 2.29-2.19 (m, 2H), 1.67 (s, 3H), 1.12-1.06 (m, 2H), 1.06-1.00 (m, 2H), 0.97 (t, J = 7.6 Hz, 3H). ¹⁹F NMR (400 MHz, DMSO-d₆) δ 117.581. LCMS R_{T} = 0.953 min, m/z = 394.0 [M + H]⁺. |
| 675 | 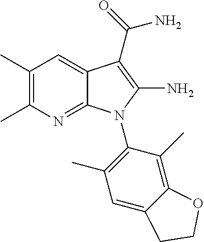 [SMILES: Cc1cc2c(C(N)=O)c(N)n(-c3c(C)cc4c(c3C)OCC4)c2nc1C] | ¹H NMR (400 MHz, DMSO-d₆) δ 8.91 (s, 1H), 8.58 (s, 1H), 7.48 (s, 1H), 7.22 (br s, 2H), 6.88 (br s, 2H), 4.46-4.30 (m, 2H), 2.43 (s, 3H), 2.19 (s, 3H), 1.94 (s, 3H). LCMS R_{T} = 1.167 min, m/z = 351.0 [M + H]⁺. |
| 678 | 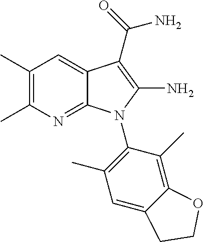 [SMILES: Cc1cc2c(C(N)=O)c(N)n(-c3c(C)cc4c(c3C)OCC4)c2nc1C] | ¹H NMR (400 MHz, DMSO-d₆) δ 10.89 (br s, 1H), 10.82 (br s, 1H), 8.91 (s, 1H), 7.24 (br s, 2H), 6.92 (br s, 2H), 6.85 (s, 1H), 2.47 (s, 3H), 1.82 (d, J = 2.8 Hz, 6H). LCMS R_{T} = 0.821 min, m/z = 352.1 [M + H]⁺. |
| 680 | 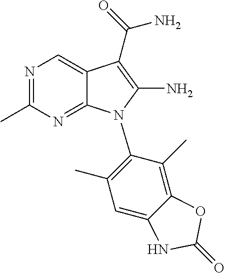 [SMILES: Cc1ncc2c(C(N)=O)c(N)n(-c3c(C)cc4[nH]c(=O)oc4c3C)c2n1] | ¹H NMR (400 MHz, DMSO-d₆) δ 11.86 (br s, 1H), 8.89 (s, 1H), 7.16 (br s, 2H), 7.04 (s, 1H), 6.85 (br s, 2H), 2.43 (s, 3H), 1.86 (s, 6H). LCMS R_{T} = 1.760 min, m/z = 353.1 [M + H]⁺. |
| 681 | 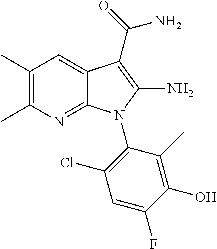 | ¹H NMR (400 MHz, DMSO-d₆) δ 10.07 (br s, 1H), 8.72 (s, 1H), 6.86 (br s, 2H), 6.83 (br s, 2H), 6.83 (d, J = 10.8 Hz, 1H), 2.47-2.39 (m, 1H), 1.67 (s, 3H), 1.62 (s, 3H), 0.95-0.82 (m, 4H). ¹⁹F NMR (400 MHz, DMSO-d₆) δ −118.644. LCMS R_{T} = 1.949 min, m/z = 435.1 (bromo isotope) [M + H]⁺. |
| 684 | 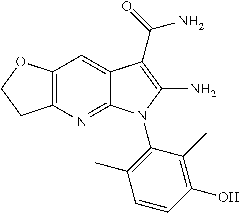 | ¹H NMR (400 MHz, DMSO-d₆) δ 9.69 (br s, 1H), 8.83 (s, 1H), 7.97-7.56 (m, 3H), 6.96- 6.70 (m, 3H), 2.36 (s, 3H), 2.19 (s, 3H). LCMS R_{T} = 1.518 min, m/z = 355.1 [M + H]⁺. |
| 685 | 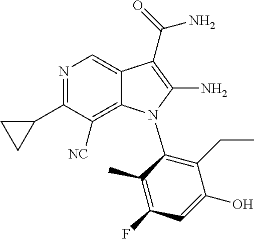 | ¹H NMR (400 MHz, CD₃OD) δ 8.81 (s, 1H), 6.82 (d, J = 11.2 Hz, 1H), 2.40-2.32 (m, 1H), 2.30-2.21 (m, 2H), 1.67 (s, 3H), 1.13-1.07 (m, 2H), 1.06-0.99 (m, 2H), 0.97 (t, J = 7.6 Hz, 3H). ¹⁹F NMR (400 MHz, DMSO-d₆) δ −117.581. LCMS R_{T} = 0.960 min, m/z = 394.0 [M + H]⁺. |

Included in the present disclosure are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

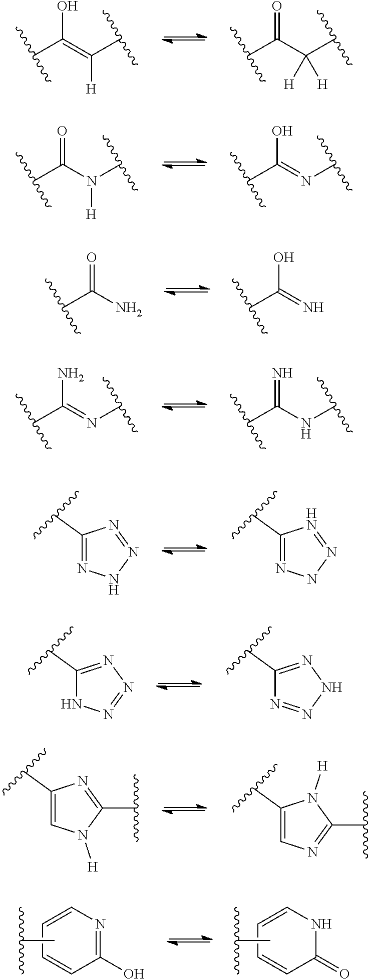

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)]2000, 110 pp; George W.; Varma, Rajender S. The Synthesis of Radiolabeled Compounds via Organometallic Intermediates, Tetrahedron, 1989, 45 (21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

In some embodiments of a compound disclosed herein, one or more of R¹, R², R³, R⁴, R⁵, R⁶, R⁷, R⁸, R^{a}, and R^{b} groups comprise deuterium at a percentage higher than the natural abundance of deuterium.

In some embodiments of a compound disclosed herein, the abundance of deuterium in each of R¹, R², R³, R⁴, R⁵, R⁶, R^{6a}, R^{6b}, R⁷, R⁸, R^{a}, and R^{b} is independently at least 1%, at least 10%, at least 20%, at least 30%, at least 40%, at least 50%, at least 60%, at least 70%, at least 80%, at least 90%, or 100% of a total number of hydrogen and deuterium.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, atropisomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269: G210-218 (1995); McLoed et al., Gastroenterol, 106: 405-413 (1994); Hochhaus et al., Biomed. Chrom., 6: 283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64: 181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### C. Pharmaceutical Compositions

Provided herein, in certain embodiments, are compositions comprising a therapeutically effective amount of any compound or salt of any one of Formulas (A), (I*), (I), (Ia) (II), (IIa) (IIb), (III*), (III), (IV), (IVa), (V), (VI), (VIa), (VIIa), (VIIb) or (VIII), (also referred to herein as “a pharmaceutical agent”).

Pharmaceutical compositions may be formulated using one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical composition comprising, for example, a pharmaceutical agent and a pharmaceutically acceptable carrier or excipient. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue; absorption through the oral mucosa, e.g., sublingually; anally, rectally or vaginally, for example, as a pessary, cream or foam; parenterally, including intramuscularly, intravenously, subcutaneously or intrathecally as, for example, a sterile solution or suspension; nasally; intraperitoneally; subcutaneously; transdermally, for example, as a patch applied to the skin; and topically, for example, as a cream, ointment or spray applied to the skin, or as an eye drop. The compound may also be formulated for inhalation. In certain embodiments, a compound may be simply dissolved or suspended in sterile water.

A pharmaceutical composition may be a sterile aqueous or non-aqueous solution, suspension or emulsion, e.g., a microemulsion. The excipients described herein are examples and are in no way limiting. An effective amount or therapeutically effective amount refers to an amount of the one or more pharmaceutical agents administered to a subject, either as a single dose or as part of a series of doses, which is effective to produce a desired therapeutic effect.

Subjects may generally be monitored for therapeutic effectiveness using assays and methods suitable for the condition being treated, which assays will be familiar to those having ordinary skill in the art and are described herein. Pharmacokinetics of a pharmaceutical agent, or one or more metabolites thereof, that is administered to a subject may be monitored by determining the level of the pharmaceutical agent or metabolite in a biological fluid, for example, in the blood, blood fraction, e.g., serum, and/or in the urine, and/or other biological sample or biological tissue from the subject. Any method practiced in the art and described herein to detect the agent may be used to measure the level of the pharmaceutical agent or metabolite during a treatment course.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

Pharmaceutical compositions comprising a pharmaceutical agent can be formulated in a manner appropriate for the delivery method by using techniques routinely practiced in the art. The composition may be in the form of a solid, e.g., tablet, capsule, semi-solid, e.g., gel, liquid, or gas, e.g., aerosol. In other embodiments, the pharmaceutical composition is administered as a bolus infusion.

Pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the pharmaceutical agent(s) of the composition upon administration. In other embodiments, the pharmaceutical agent may be encapsulated within liposomes using technology known and practiced in the art. In certain particular embodiments, a pharmaceutical agent is not formulated within liposomes for application to a stent that is used for treating highly, though not totally, occluded arteries. Pharmaceutical compositions may be formulated for any appropriate manner of administration described herein and in the art.

A pharmaceutical composition, e.g., for oral administration or for injection, infusion, subcutaneous delivery, intramuscular delivery, intraperitoneal delivery or other method, may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical agents described herein can be used alone or in combination with appropriate additives to make tablets, powders, granules or capsules, and if desired, with diluents, buffering agents, moistening agents, preservatives, coloring agents, and flavoring agents. The pharmaceutical agents may be formulated with a buffering agent to provide for protection of the compound from low pH of the gastric environment and/or an enteric coating. A pharmaceutical agent included in a pharmaceutical composition may be formulated for oral delivery with a flavoring agent, e.g., in a liquid, solid or semi-solid formulation and/or with an enteric coating.

A pharmaceutical composition comprising any one of the pharmaceutical agents described herein may be formulated for sustained or slow release, also called timed release or controlled release. Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of pharmaceutical agent contained within a sustained release formulation depends upon the site of implantation, the rate and expected duration of release, and the nature of the condition, disease or disorder to be treated or prevented.

In certain embodiments, the pharmaceutical compositions comprising a pharmaceutical agent are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated, e.g., intradermally or subcutaneously. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Pharmaceutical compositions comprising a pharmaceutical agent can be formulated as emulsions for topical application. An emulsion contains one liquid distributed in the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions, patches, bandages and articles. These systems are semisolid emulsions, micro-emulsions, or foam emulsion systems.

In some embodiments, the pharmaceutical agent described herein can be formulated as in inhalant. Inhaled methods can deliver medication directly to the airway. The pharmaceutical agent can be formulated as aerosols, microspheres, liposomes, or nanoparticles. The pharmaceutical agent can be formulated with solvents, gases, nitrates, or any combinations thereof. Compositions described herein are optionally formulated for delivery as a liquid aerosol or inhalable dry powder. Liquid aerosol formulations are optionally nebulized predominantly into particle sizes that can be delivered to the terminal and respiratory bronchioles. Liquid aerosol and inhalable dry powder formulations are preferably delivered throughout the endobronchial tree to the terminal bronchioles and eventually to the parenchymal tissue.

Aerosolized formulations described herein are optionally delivered using an aerosol forming device, such as a jet, vibrating porous plate or ultrasonic nebulizer, preferably selected to allow the formation of aerosol particles having with a mass medium average diameter predominantly between 1 to 5μ. Further, the formulation preferably has balanced osmolarity ionic strength and chloride concentration, and the smallest aerosolizable volume able to deliver effective dose of the pharmaceutical agent. Additionally, the aerosolized formulation preferably does not impair negatively the functionality of the airways and does not cause undesirable side effects.

Aerosolization devices suitable for administration of aerosol formulations described herein include, for example, jet, vibrating porous plate, ultrasonic nebulizers and energized dry powder inhalers, that are able to nebulize the formulation into aerosol particle size predominantly in the size range from 1-5μ. Predominantly in this application means that at least 70% but preferably more than 90% of all generated aerosol particles are within 1-5μ range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb™ and AeroDose™ vibrating porous plate nebulizers (AeroGen, Inc., Sunnyvale, California), Sidestream® nebulizers (Medic-Aid Ltd., West Sussex, England), Pari LC® and Pari LC Star® jet nebulizers (Pari Respiratory Equipment, Inc., Richmond, Virginia), and Aerosonic™ (DeVilbiss Medizinische Produkte (Deutschland) GmbH, Heiden, Germany) and UltraAire® (Omron Healthcare, Inc., Vernon Hills, Illinois) ultrasonic nebulizers.

In some embodiments, the pharmaceutical agent(s) can be formulated with oleaginous bases or ointments to form a semisolid composition with a desired shape. In addition to the pharmaceutical agent, these semisolid compositions can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

Controlled or sustained release transdermal or topical formulations can be achieved by the addition of time-release additives, such as polymeric structures, matrices, that are available in the art. For example, the compositions may be administered through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

An insert, transdermal patch, bandage or article can comprise a mixture or coating of polymers that provide release of the pharmaceutical agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients.

Transdermal devices (inserts, patches, bandages) may also comprise a water insoluble polymer. Rate controlling polymers may be useful for administration to sites where pH change can be used to effect release. These rate controlling polymers can be applied using a continuous coating film during the process of spraying and drying with the active compound. In one embodiment, the coating formulation is used to coat pellets comprising the active ingredients that are compressed to form a solid, biodegradable insert.

A polymer formulation can also be utilized to provide controlled or sustained release. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the pharmaceutical agent. The matrices can be formed by solvent evaporation, spray drying, solvent extraction and other methods known to those skilled in the art.

Kits with unit doses of one or more of the agents described herein, usually in oral or injectable doses, are provided. Such kits may include a container containing the unit dose, an informational package insert describing the use and attendant benefits of the drugs in treating disease, and optionally an appliance or device for delivery of the composition.

### D. Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-recloseable containers. Alternatively, multiple-dose recloseable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

In another aspect, disclosed herein is a method of modulating PKMYT1 in a subject, the method comprising administering to the subject a compound, or a pharmaceutically acceptable salt, solvate, or stereoisomer thereof, disclosed herein. Disclosed herein is a method of inhibiting PKMYT1 in a subject, the method comprising administering to the subject a compound, or a pharmaceutically acceptable salt, solvate, or stereoisomer thereof, disclosed herein. Further disclosed herein is a method of selectively inhibiting PKMYT1 in a subject (e.g., selective over WEE1 or LCK), the method comprising administering to the subject a compound, or a pharmaceutically acceptable salt, solvate, or stereoisomer thereof, disclosed herein.

In an aspect, disclosed herein is a method for treating a subject having or suspected of having a cancer, comprising administering to the subject a therapeutically effective amount of a compounds disclosed herein, or a pharmaceutically acceptable salt or solvate thereof, that modulates the expression or activity of PKMYT1.

In some embodiments, the cancer is associated with PKMYT1 activity.

In some embodiments, the cancer has an inactivating mutation in the FBXW7 gene. In some embodiments cancers which have a deficiency in FBXW7 include, e.g., breast cancer, colorectal cancer, esophageal cancer, lung cancer, and uterine cancer.

In some embodiments, the cancer is a skin cancer, ocular cancer, gastrointestinal cancer, thyroid cancer, breast cancer, ovarian cancer, central nervous system cancer, laryngeal cancer, cervical cancer, lymphatic system cancer, genitourinary tract cancer, bone cancer, biliary tract cancer, endometrial cancer, liver cancer, lung cancer, prostate cancer, or colon cancer.

In some embodiments, the cancer is leukemia, acute myeloid leukemia (AML), chronic myeloid leukemia, acute lymphoblastic leukemia (ALL), non-Hodgkin lymphoma (NHL), Hodgkin lymphoma (HL), or multiple myeloma (MM).

In some embodiments, the cancer is a solid cancer.

In some embodiments, the cancer is not a solid cancer.

In some embodiments, the cancer is a cancer metastasis.

It can be beneficial to use compounds that selectively inhibit PKMYT1 over for example WEE or LCK in methods of treatment described herein. In some embodiments, compounds disclosed herein do not inhibit LCK. In some embodiments, a compound disclosed herein has a LCK IC₅₀ value of at least about 100 nM, at least about 500 nM, at least about 1000 nM, or at least about 10,000 nM as determined by the procedure described in Example B-1.

In some embodiments, the compounds disclosed herein are about 2-fold, 3-fold, 4-fold, 5-fold, 10-fold, 25-fold, 50-fold or 100-fold more selective for inhibiting PKMYT1 over LCK. In some embodiments, the compounds disclosed herein are at least about 2-fold, 3-fold, 4-fold, 5-fold, 10-fold, 25-fold, 50-fold or 100-fold more selective for inhibiting PKMYT1 over LCK.

Comparative data for PKMYT1 and LCK inhibition can be seen in Table 2.

In some embodiments, the method comprises administering a second therapeutic agent. In some embodiments, the second therapeutic agent is a monoclonal antibody. In some embodiments, the second therapeutic agent is an immune checkpoint inhibitor. In some embodiments, the second therapeutic is chemotherapy or radiation therapy.

### E. Biomarkers

In another aspect, provided herein is a method of identifying a subject having a disease for treatment with a compound disclosed herein (e.g., a compound of Formula (A), or a pharmaceutically acceptable salt thereof), the method comprising determining the presence of a mutation in the expression level of, and/or the activity of one or more biomarkers in a diseased tissue sample obtained from the subject.

In another aspect, provided herein is a method of determining the responsiveness of a subject having a disease or disorder to a compound disclosed herein (e.g., a compound of Formula (A), or a pharmaceutically acceptable salt thereof), the method comprising determining the presence of a mutation in the expression level of, and/or the activity of one of more biomarkers in a diseased tissue sample obtained from the subject.

In some embodiments, the diseased tissue sample comprises an altered expression level and/or activity of the one or more biomarkers relative to a reference tissue sample. In some embodiments, the expression level and/or activity of the one or more biomarkers is reduced relative to a reference tissue sample.

In some embodiments, the diseased tissue sample is from a cancer. In some embodiments, the cancerous tissue is breast tissue, pancreatic tissue, uterine tissue, bladder tissue, colorectal tissue, prostate tissue, liver tissue, or ovarian tissue. In some embodiments, the cancerous tissue is liver tissue. In some embodiments, the cancerous tissue is ovarian tissue

In some embodiments, the subject has a tumor, and wherein the diseased tissue sample comprises a tumor sample, a circulating tumor DNA sample, a tumor biopsy sample, or a fixed tumor sample. In some embodiments, the tumor sample is selected from acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).

In some embodiments, the healthy control is from one or more subjects that do not exhibit the cancer or tumor (e.g., liver or ovarian cancer).

In another aspect, provided herein is a method of identifying a cancer subject to receive a compound disclosed herein (e.g., a compound of Formula (A), or a pharmaceutically acceptable salt or solvate thereof), the method comprising: (i) determining the presence of the mutation in the expression level of, and/or the activity of one or more biomarkers in the tumor sample obtained from the subject; and (ii) administering the compound disclosed herein to the subject based on the presence of a mutation in, a reduced expression level, and/or a reduced activity of the one or more biomarkers relative to a healthy control.

In some embodiments, the one or more biomarkers is selected from the group consisting of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some embodiments, the one or more biomarkers is selected from the group consisting of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, the one or more biomarkers is selected from the group consisting of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some embodiments, the one or more biomarker is selected from the group consisting of SLITRK1, ZNF521, CCNB1, CDK7, MYTIL, FZR1, SERFIA, GADD45B, ADGRL2, TTK, NRXN2, UNC13A, ZBTB7A, POLD1, PCDH19, SLC8A2, E2F4, UTS2, KCNN2, CCNH, FRG2C, PLK2, MYO18A, and DCAF12L1.

In some embodiments, the one or more biomarkers is selected from the group consisting of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBCID22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ADPM, GABRB3, CTNN3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some embodiments, the one or more biomarkers is selected form the group consisting of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, NCAPG, IGF1F, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2RC, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMTG, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNP02, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, PEC8, RECQL4, ZNF853, SRSF4, PPP2R5A, ZBTB12, MMP12, KIF2C, HSP90AA1, PPP2R2D, CDC7, NANS, MOS, RBX1, NAGED4B, KIP23, SCLM1, SPANXA2, TRIM28, SRRM5, MEGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MEGEA9, F8A3, ARL17A, CTAG1A, MAD2L1, HSFX1, BNIP3, MRGPRG, ANAPC2, and RAD2.

In some embodiments, the cancer is associated with cancerous tissue comprising a cell that displays mutations and/or deletions in genes encoding subunits of Protein Phosphatase 2 (PP2A) as compared to a healthy control. In some embodiments, the presence or absence of the mutations and/or deletions is identified by an assay of cells derived from tissue obtained from the subject. In some embodiments, the assay is a next generation sequencing-based assay.

In some embodiments, the PP2A subunit is selected from the group consisting of 65 kDa regulatory subunit A alpha (PPP2R1A), 65 kDa regulatory subunit A beta (PPP2R1B), 55 kDa regulatory subunit B alpha (PPP2R2A), 55 kDa regulatory subunit B beta (PPP2R2B), 55 kDa regulatory subunit B gamma (PPP2R2C), 55 kDa regulatory subunit B delta (PPP2R2D), 72/130 kDa regulatory subunit B (PPP2R3A), 48 kDa regulatory subunit B (PPP2R3B), regulatory subunit B″ subunit gamma (PPP2R3C), regulatory subunit B′ (PPP2R4), 56 kDa regulatory subunit alpha (PPP2R5A), 56 kDa regulatory subunit beta (PPP2R5B), 56 kDa regulatory subunit gamma (PPP2R5C), 56 kDa regulatory subunit delta (PPP2R5D), 56 kDa regulatory subunit epsilon (PPP2R5E), catalytic subunit alpha (PPP2CA), and catalytic subunit beta (PPP2CB). In some embodiments, the PP2A subunit is PPP2R2A.

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

The compounds and salts of Formulas (A), (I*), (I), (Ia), (II), (IIa), (IIb), (III*), (III), (IIIa), (IV), (IVa), (V), (VI), (VIa), (VIIa), (VIIb), and (VIII) can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in the synthesis schemes below, the steps in some cases may be performed in a different order than the order shown below. Numberings or R groups in each scheme do not necessarily correspond to that of the claims or other schemes or tables herein.

### Example 1. Synthesis of Compounds 550 and 548

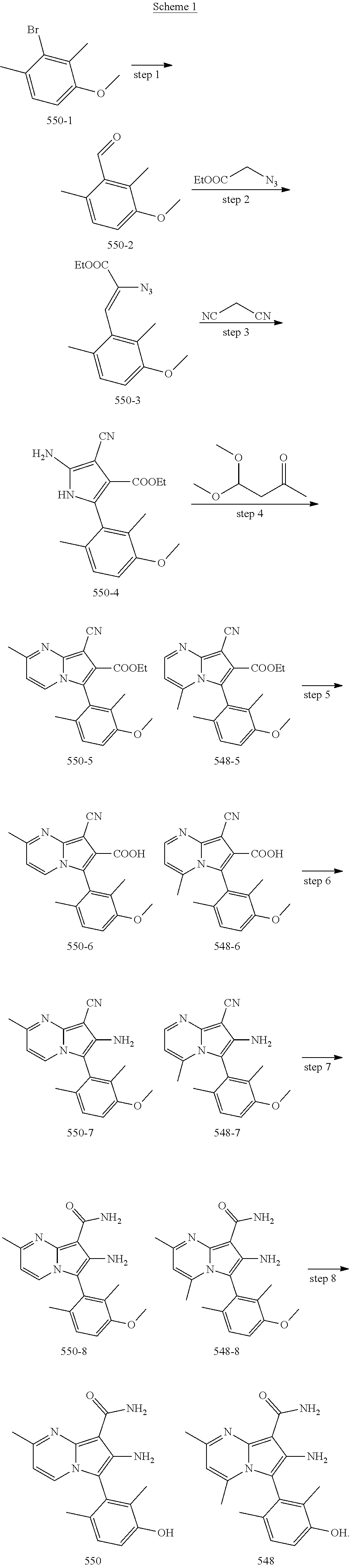

### Step 1: Synthesis of 3-methoxy-2,6-dimethyl-benzaldehyde

To a solution of 3-bromo-1-methoxy-2,4-dimethyl-benzene (5.0 g, 23.25 mmol) in tetrahydrofuran (50 mL) was added n-butyllithium (3.0 g, 46.49 mmol) dropwise at −78° C. The mixture was stirred at −78° C. for 0.5 hour under nitrogen atmosphere. The mixture was added N,N-dimethylformamide (5.8 g, 79.04 mmol) and stirred at −78° C. for 1 hour. The mixture was then allowed to warm to 20° C. and stirred for 1.5 hours. The mixture was poured into water (60 mL) and extracted with ethyl acetate (50 mL×3), washed with brine (30 mL×2). The organic layers were dried over anhydrous sodium sulphate and filtered. The filtrate was concentrated under reduced pressure. The residue was purified by column chromatography (silica gel, 100-200 mesh, 0-1% ethyl acetate in petroleum ether) to afford the title compound (3.2 g, 60%) as a white solid. LCMS R_{T}=0.548 min, m/z=165.0 [M+H]⁺. ¹H NMR (400 MHz, CD₃OD) δ 10.49 (s, 1H), 7.10 (s, 2H), 3.79 (s, 3H), 2.43 (s, 3H), 2.36 (s, 3H).

### Step 2: Synthesis of (Z)-2-azido-3-(3-methoxy-2,6-dimethyl-phenyl)prop-2-enoate

To a solution of sodium ethoxide (2.6 g, 38.37 mmol) in ethyl alcohol (40 mL) was added 3-methoxy-2,6-dimethyl-benzaldehyde (1.4 g, 8.53 mmol) and ethyl 2-azidoacetate (4.4 g, 34.10 mmol) in ethyl alcohol (20 mL) at −20° C. The mixture was warmed to 15° C. and stirred for 12 hours. The mixture was quenched by addition of saturated aqueous ammonium chloride (100 mL) and water (50 mL). The mixture was extracted with ethyl acetate (80 mL×2). The combined organic layers were washed with brine (80 mL), dried over anhydrous sodium sulphate and filtered. The filtrate was concentrated under reduced pressure. The residue was purified by column chromatography (silica gel, 100-200 mesh, 0-30% dichloromethane in petroleum ether) to afford the title compound (1.2 g, 36%) as yellow oil.

### Step 3: Synthesis of ethyl 5-amino-4-cyano-2-(3-methoxy-2,6-dimethyl-phenyl)-1H-pyrrole-3-carboxylate

To a solution of ethyl (Z)-2-azido-3-(3-methoxy-2,6-dimethyl-phenyl)prop-2-enoate (1.2 g, 2.92 mmol) in ethyl alcohol (0.5 mL) and water (0.5 mL) was added propanedinitrile (251 mg, 3.80 mmol). The mixture was stirred at 75° C. for 12 hours. The mixture was concentrated under reduced pressure. The residue was purified by column chromatography (silica gel, 100-200 mesh, 0-9% methanol in dichloromethane) to afford the title compound (320 mg, 35%) as a yellow solid.

### Step 4: Synthesis of ethyl 8-cyano-6-(3-methoxy-2,6-dimethyl-phenyl)-2-methyl-pyrrolo[1,2-a]pyrimidine-7-carboxylate & ethyl 8-cyano-6-(3-methoxy-2,6-dimethylphenyl)-4-methylpyrrolo[1,2-a]pyrimidine-7-carboxylate

To a solution of ethyl 5-amino-4-cyano-2-(3-methoxy-2,6-dimethyl-phenyl)-1H-pyrrole-3-carboxylate (320 mg, 1.02 mmol) and 4,4-dimethoxybutan-2-one (162 mg, 1.23 mmol) in acetic acid (3 mL). The mixture was stirred at 110° C. for 1 hour under nitrogen atmosphere. The mixture was concentrated under reduced pressure to afford the title mixture compound (360 mg, 85%) as a brown solid. LCMS R_{T}=1.767 min, m/z=364.1 [M+H]⁺.

### Step 5: Synthesis of 8-cyano-6-(3-methoxy-2,6-dimethyl-phenyl)-2-methyl-pyrrolo[1,2-a]pyrimidine-7-carboxylic acid & 8-cyano-6-(3-methoxy-2,6-dimethylphenyl)-4-methylpyrrolo[1,2-a]pyrimidine-7-carboxylic acid

To a mixture of ethyl 8-cyano-6-(3-methoxy-2,6-dimethyl-phenyl)-2-methyl-pyrrolo[1,2-a]pyrimidine-7-carboxylate and ethyl 8-cyano-6-(3-methoxy-2,6-dimethylphenyl)-4-methylpyrrolo[1,2-a]pyrimidine-7-carboxylate (360 mg, 0.99 mmol) in tetrahydrofuran (1.5 mL), water (1 mL) and methanol (0.5 mL) was added lithium hydrate (237 mg, 9.91 mmol). The mixture was stirred at 20° C. for 12 hours under nitrogen atmosphere. The mixture was extracted with ethyl acetate (60 mL), the aqueous phase was neutralized with dilute hydrochloric acid, then extracted with ethyl acetate. The organic extract was dried over anhydrous sodium sulphate and filtered. The filtrate was concentrated under reduced pressure to afford the title mixture compound (280 mg, 80%) as a red solid. LCMS R_{T}=0.499 min, m/z=336.0 [M+H]⁺

### Step 6: Synthesis of 7-amino-6-(3-methoxy-2,6-dimethyl-phenyl)-2-methyl-pyrrolo[1,2-a]pyrimidine-8-carbonitrile & 7-amino-6-(3-methoxy-2,6-dimethylphenyl)-4-methylpyrrolo[1,2-a]pyrimidine-8-carbonitrile

To a solution of 8-cyano-6-(3-methoxy-2,6-dimethyl-phenyl)-2-methyl-pyrrolo[1,2-a]pyrimidine-7-carboxylic acid and 8-cyano-6-(3-methoxy-2,6-dimethylphenyl)-4-methylpyrrolo[1,2-a]pyrimidine-7-carboxylic acid (280 mg, 0.83 mmol) in tetrahydrofuran (3 mL) was added [azido(phenoxy)phosphoryl]oxybenzene (368 mg, 1.34 mmol) and triethylamine (253 mg, 2.50 mmol). The mixture was stirred at 30° C. for 16 hours under nitrogen atmosphere. Then the mixture was added water (15 mL) and heated to 100° C. and stirred for 2 hours. The mixture was poured into water (10 mL) and extracted with dichloromethane (10 mL×3), washed with brine (10 mL×2). The organic extract was dried over anhydrous sodium sulphate and filtered. The filtrate was concentrated under reduced pressure to afford the title mixture compound (200 mg, 33%) as a brown solid. LCMS R_{T}=1.434 min, m/z=307.0 [M+H]⁺

### Step 7: Synthesis of 7-amino-6-(3-methoxy-2,6-dimethyl-phenyl)-2-methyl-pyrrolo[1,2-a]pyrimidine-8-carboxamide & 7-amino-6-(3-methoxy-2,6-dimethylphenyl)-4-methylpyrrolo[1,2-a]pyrimidine-8-carboxamide

A mixture of 7-amino-6-(3-methoxy-2,6-dimethyl-phenyl)-2-methyl-pyrrolo[1,2-a]pyrimidine-8-carbonitrile and 7-amino-6-(3-methoxy-2,6-dimethylphenyl)-4-methylpyrrolo[1,2-a]pyrimidine-8-carbonitrile (200 mg, 0.65 mmol) in sulfuric acid (1 mL) was stirred at 20° C. for 1 hour. The mixture was poured into sodium hydroxide solution (100 mL, 10%) and extracted with dichloromethane (100 mL×3). The organic extract was dried over anhydrous sodium sulphate and filtered. The filtrate was concentrated under reduced pressure. The residue was purified by prep-TLC to afford the title compound (80 mg, 26%) as a red solid. LCMS R_{T}=1.406 min, m/z=325.0 [M+H]⁺. ¹H NMR (400 MHz, DMSO-d₆) δ 8.01-7.90 (m, 2H), 7.54 (d, J=7.2 Hz, 1H), 7.56-6.95 (m, 4H), 6.67-6.60 (m, 1H), 3.81 (s, 3H), 1.90 (s, 3H), 1.80 (s, 3H), 1.23 (s, 3H).

### Step 8: Synthesis of 7-amino-6-(3-hydroxy-2,6-dimethyl-phenyl)-4-methyl-pyrrolo[1,2-a]pyrimidine-8-carboxamide and 7-amino-6-(3-hydroxy-2,6-dimethyl-phenyl)-2-methyl-pyrrolo[1,2-a]pyrimidine-8-carboxamide

To a solution of 7-amino-6-(3-methoxy-2,6-dimethyl-phenyl)-2-methyl-pyrrolo[1,2-a]pyrimidine-8-carboxamide (30 mg, 0.09 mmol) in 1,2-dichloroethane (1 mL) was added boron tribrothermide (0.277 mL, 1 M in dichloromethane) at 0° C. The mixture was stirred at 20° C. for 1 hour under nitrogen atmosphere. The mixture was quenched by addition of methanol (10 mL) and concentrated under reduced pressure. The residue was purified by RP-HPLC (12 to 52% acetonitrile in water and 10 mM ammonium bicarbonate) to afford 7-amino-6-(3-hydroxy-2,6-dimethyl-phenyl)-4-methyl-pyrrolo[1,2-a]pyrimidine-8-carboxamide (3.2 mg, 11%) and 7-amino-6-(3-hydroxy-2,6-dimethyl-phenyl)-2-methyl-pyrrolo[1,2-a]pyrimidine-8-carboxamide (5.7 mg, 19%) as yellow solid.

### 7-amino-6-(3-hydroxy-2,6-dimethyl-phenyl)-4-methyl-pyrrolo[1,2-a]pyrimidine-8-carboxamide (Compound 548)

LCMS R_{T}=1.016 min, m/z=311.0 [M+H]⁺. ¹H NMR (400 MHz, CD₃OD) δ 8.03 (d, J=4.4 Hz, 1H), 6.98 (d, J=8.4 Hz, 1H), 6.84 (d, J=8.4 Hz, 1H), 6.56 (d, J=4.4 Hz, 1H), 2.02 (s, 3H), 1.91 (s, 3H), 1.89 (s, 3H).

### 7-amino-6-(3-hydroxy-2,6-dimethyl-phenyl)-2-methyl-pyrrolo[1,2-a]pyrimidine-8-carboxamide (Compound 550)

LCMS R_{T}=1.135 min, m/z=311.0 [M+H]⁺. ¹H NMR (400 MHz, CD₃OD) δ 7.52 (d, J=6.8 Hz, 1H), 7.05 (d, J=8.0 Hz, 1H), 6.84 (d, J=8.0 Hz, 1H), 6.65 (d, J=6.8 Hz, 1H), 2.55 (s, 3H), 1.92 (s, 3H), 1.87 (s, 3H).

### Example 2. Synthesis of Compound 560

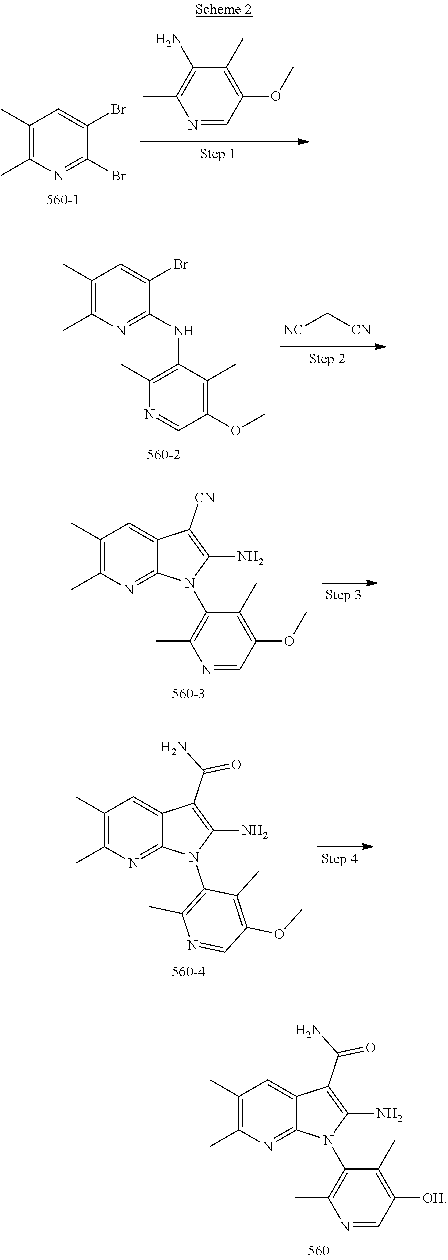

### Step 1: Synthesis of 3-bromo-N-(5-methoxy-2,4-dimethyl-3-pyridyl)-5,6-dimethyl-pyridin-2-amine

To a solution of 2,3-dibromo-5,6-dimethyl-pyridine (496 mg, 1.87 mmol) in 1,2-dimethoxyethane (12 mL) was added 5-methoxy-2,4-dimethyl-pyridin-3-amine (300 mg, 1.97 mmol), cesium carbonate (1.28 g, 3.94 mmol), Xantphos (114 mg, 0.20 mmol) and Pd₂(dba)₃ (90 mg, 0.10 mmol). The mixture was stirred at 84° C. for 12 hours. The reaction mixture was added water (80 mL) and extracted with ethyl acetate (100 mL×3). The combined organic layers were washed with brine (120 mL). The separated organic layer was dried over anhydrous sodium sulphate, filtered and concentrated under reduced pressure. The residue was purified by flash column chromatography (silica gel, 100-200 mesh, 0-43% ethyl acetate in petroleum ether) to afford the title compound (540 mg, 80%) as a white solid. LCMS R_{T}=0.475 min, m/z=335.9 [M+H]⁺.

### Step 2: Synthesis of 2-amino-1-(5-methoxy-2,4-dimethyl-3-pyridyl)-5,6-dimethyl-pyrrolo[2,3-b]pyridine-3-carbonitrile

To a solution of propanedinitrile (86 mg, 1.31 mmol) in 1,2-dimethoxyethane (4 mL) was added sodium hydride (52 mg, 1.31 mmol, 60% purity) at 0° C. The mixture was stirred at 0° C. for 0.5 hour. Then 3-bromo-N-(5-methoxy-2,4-dimethyl-3-pyridyl)-5,6-dimethyl-pyridin-2-amine (200 mg, 0.59 mmol) and 1,1′-bis(diphenylphosphino)ferrocene-palladium (II) dichloride dichloromethane complex (49 mg, 0.06 mmol) was added. The mixture was stirred at 110° C. for 2.5 hours. The reaction mixture was added water (50 mL) and extracted with ethyl acetate (60 mL×3). The combined organic layers were washed with brine (60 mL). The separated organic layer was dried over anhydrous sodium sulphate, filtered and concentrated under reduced pressure. The residue was purified by flash column chromatography (silica gel, 100-200 mesh, 0-85% ethyl acetate in petroleum ether) to afford the title compound (120 mg, 63%) as a yellow solid. LCMS R_{T}=1.060 min, m/z=322.1 [M+H]⁺.

### Step 3: Synthesis of 2-amino-1-(5-methoxy-2,4-dimethyl-3-pyridyl)-5,6-dimethyl-pyrrolo[2,3-b]pyridine-3-carboxamide

To a solution of 2-amino-1-(5-methoxy-2,4-dimethyl-3-pyridyl)-5,6-dimethyl-pyrrolo[2,3-b]pyridine-3-carbonitrile (70 mg, 0.22 mmol) was added sulfuric acid (1 mL). The mixture was stirred at 20° C. for 1 hour. After the completion of reaction, the reaction mixture was adjusted to pH=7 with saturated aqueous sodium bicarbonate solution and extracted with ethyl acetate (80 mL×3). The combined organic layers were washed with brine (60 mL). The separated organic layer was dried over anhydrous sodium sulphate, filtered, and concentrated under reduced pressure. The organic phase was concentrated to afford the crude title compound (68 mg, 92%) as a white solid which was used directly. LCMS R_{T}=0.395 min, m/z=340.2 [M+H]⁺.

### Step 4: Synthesis of 2-amino-1-(5-hydroxy-2,4-dimethylpyridin-3-yl)-5,6-dimethyl-1H-pyrrolo[2,3-b]pyridine-3-carboxamide (Compound 560)

To a solution of 2-amino-1-(5-methoxy-2,4-dimethyl-3-pyridyl)-5,6-dimethyl-pyrrolo[2,3-b]pyridine-3-carboxamide (15 mg, 0.04 mmol) in toluene (1 mL) was added aluminum chloride (59 mg, 0.44 mmol). The mixture was stirred at 100° C. for 3 hours. The mixture was quenched by addition of 2 M hydrochloric acid (1.5 mL) and concentrated to remove toluene. The residue was purified by RP-HPLC (0 to 90% acetonitrile in water and 0.05% hydrochloric acid water) to afford the title compound (3 mg, 23%) as a white solid. ¹H NMR (400 MHz, DMSO-d₆) δ 12.27-11.91 (m, 1H), 8.42-8.28 (m, 1H), 8.00-7.90 (m, 1H), 7.67-5.97 (m, 4H), 2.28 (s, 6H), 2.25-2.21 (m, 3H), 1.94-1.87 (m, 3H). LCMS R_{T}=0.760 min, m/z=326.4 [M+H]⁺.

### Example 3. Synthesis of Compound 561

2-Amino-1-(5-hydroxy-2,4-dimethyl-3-pyridyl)-5-methyl-pyrrolo[2,3-b]pyridine-3-carboxamide was made by the method used from 3-bromo-2-fluoro-5-methyl-pyridine. The final compound was purified by RP-HPLC (0 to 90% acetonitrile in water and 10% hydrochloric acid) to afford the title compound (18.2 mg, 52%) as a white solid. ¹H NMR (400 MHz, DMSO-d₆) δ 12.22 (s, 1H), 8.41 (d, J=1.6 Hz, 1H), 8.02 (s, 1H), 7.67 (s, 1H), 2.33 (s, 3H), 2.26 (s, 3H), 1.90 (s, 3H). LCMS R_{T}=0.958 min, m/z=312.3 [M+H]⁺

### Example 4. Synthesis of Compound 555

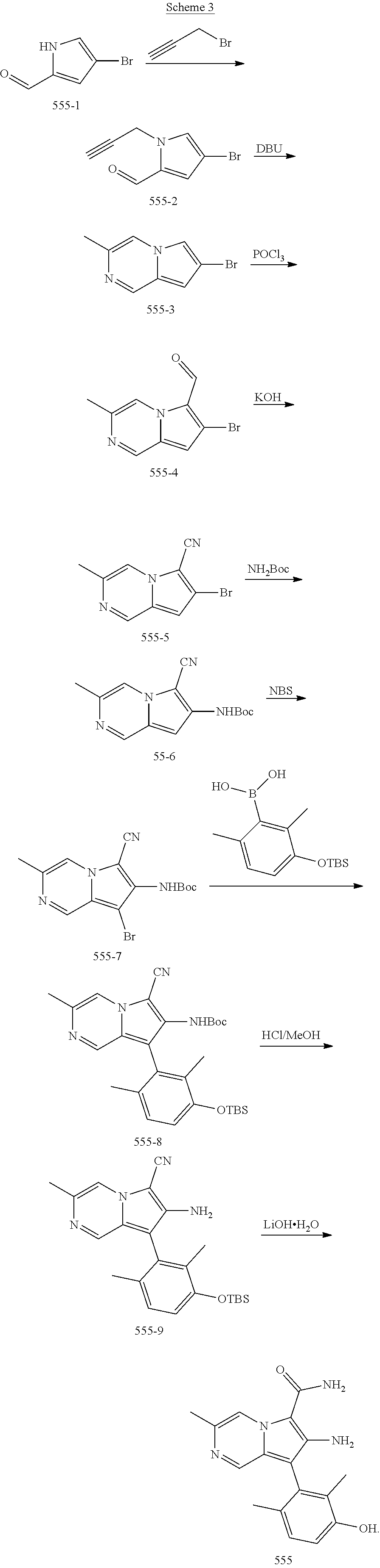

### Step 1: Synthesis of 4-bromo-1-(prop-2-yn-1-yl)-1H-pyrrole-2-carbaldehyde

To a solution of 4-bromo-1H-pyrrole-2-carbaldehyde (5.0 g, 28.74 mmol) in N,N-dimethylformamide (25 mL) was added sodium hydride (1.72 g, 43.10 mmol, 60% purity). The mixture was stirred at 0° C. for 0.5 hour under nitrogen atmosphere. Then the mixture was added 3-bromoprop-1-yne (3.76 g, 31.61 mmol) and was stirred at 0° C. for 4 hours under nitrogen atmosphere. The reaction was quenched by addition of saturated aqueous ammonium chloride (300 mL). The reaction mixture was diluted with water (10 mL) and was extracted with dichloromethane (100 mL×3). The combined organic layers were washed with brine (100 mL), dried over anhydrous sodium sulfate, filtered and concentrated under reduced pressure to give a residue. The residue was purified by flash column chromatography (silica gel, 100-200 mesh, 0-5% methanol in dichloromethane) to afford the title compound (1.5 g, 25%) as a white solid which was used directly for next step. ¹H NMR (400 MHz, DMSO-d₆) δ 9.52 (s, 1H), 7.54 (d, J=1.2 Hz, 1H), 7.18 (s, 1H), 5.15 (d, J=2.4 Hz, 2H), 3.46 (m, 1H).

### Step 2: Synthesis of 7-bromo-3-methylpyrrolo[1,2-a]pyrazine

To a solution of 4-bromo-1-prop-2-ynyl-pyrrole-2-carbaldehyde (1.5 g, 7.07 mmol) in ethanol (15 mL) was added ammonium hydroxide (3.97 g, 113.18 mmol) and cesium carbonate (2.30 g, 7.07 mmol). The mixture was stirred at 80° C. for 12 hours under nitrogen atmosphere. The reaction mixture was diluted with water (100 mL) and was extracted with ethyl acetate (50 mL×3). The combined organic layers were washed with brine (50 mL), dried over anhydrous sodium sulfate, filtered and concentrated under reduced pressure. The residue was purified by flash column chromatography (silica gel, 100-200 mesh, 0-50% ethyl acetate in petroleum ether) to afford the title compound (1.0 g, 67%) as a yellow solid which was used directly for next step. LCMS R_{T}=0.317 min, m/z=212.9 [M+H+2]⁺.

### Step 3: Synthesis of 7-bromo-3-methylpyrrolo[1,2-a]pyrazine-6-carbaldehyde

To a solution of 7-bromo-3-methyl-pyrrolo[1,2-a]pyrazine (1.0 g, 4.74 mmol) in dichloromethane (15 mL) was added N,N-dimethylformamide (416 mg, 5.69 mmol) and phosphorus oxychloride (872 mg, 5.69 mmol). The mixture was stirred at 20° C. for 3 hour under nitrogen atmosphere. The reaction was quenched by addition of water (100 mL) and extracted with ethyl acetate (50 mL×3), washed with brine (50 mL×3). The organic layers were dried over anhydrous sodium sulfate, filtered and concentrated under reduced pressure to give a residue. The residue was purified by flash column chromatography (silica gel, 100-200 mesh, 0-50% ethyl acetate in petroleum ether) to afford the title compound (360 mg, 32%) as a yellow solid which was used directly for next step. LCMS R_{T}=0.475 min, m/z=239.0 [M+H]⁺.

### Step 4: Synthesis of 7-bromo-3-methylpyrrolo[1,2-a]pyrazine-6-carbonitrile

To a solution of 7-bromo-3-methyl-pyrrolo[1,2-a]pyrazine-6-carbaldehyde (270 mg, 1.13 mmol) in water (10 mL) was added amino hydrogen sulfate (383 mg, 3.39 mmol) at 20° C. Then the mixture was stirred at 20° C. for 2.5 hour and then added potassium hydroxide (190 mg, 3.39 mmol). After stirred at 20° C. for 0.5 hours, the reaction mixture was diluted with water (10 mL) and extracted with dichloromethane (10 mL×3). The combined organic layers were washed with brine (10 mL), dried over anhydrous sodium sulfate, filtered and concentrated under reduced pressure to give a residue. The residue was purified by flash column chromatography (silica gel, 100-200 mesh, 0-3% methanol in dichloromethane) to afford the title compound (160 mg, 60%) as a white solid which was used directly for next step. LCMS R_{T}=0.813 min, m/z=235.9 [M+H]⁺.

### Step 5: Synthesis of tert-butyl (6-cyano-3-methylpyrrolo[1,2-a]pyrazin-7-yl)carbamate

To a mixture of 7-bromo-3-methyl-pyrrolo[1,2-a]pyrazine-6-carbonitrile (160 mg, 0.68 mmol) in dioxane (3 mL) was added N,N′-dimethylethane-1,2-diamine (12 mg, 0.13 mmol), copper(I) iodide (26 mg, 0.13 mmol), potassium phosphate (288 mg, 1.36 mmol) and tert-butyl carbamate (159 mg, 1.36 mmol) at 20° C. Then the mixture was stirred at 100° C. for 8 hour. The reaction mixture was added water (20 mL) and extracted with dichloromethane (20 mL×3). The combined organic layers were washed with brine (10 mL), dried over anhydrous sodium sulfate, filtered and concentrated under reduced pressure to give a residue. The residue was purified by prep-TLC (40% ethyl acetate in petroleum ether) to afford the title compound (70 mg, 38%) as a yellow solid which was used directly for next step. LCMS R_{T}=0.473 min, m/z=273.0 [M+H]⁺.

### Step 6: Synthesis of tert-butyl (8-bromo-6-cyano-3-methylpyrrolo[1,2-a]pyrazin-7-yl)carbamate

To a solution of tert-butyl N-(6-cyano-3-methyl-pyrrolo[1,2-a]pyrazin-7-yl)carbamate (70 mg, 0.26 mmol) in N,N-dimethylformamide (1 mL) was added 1-bromopyrrolidine-2,5-dione (41 mg, 0.23 mmol) at 0° C. After the mixture stirred at 20° C. for 1 hour, the reaction mixture was added water (10 mL) and extracted with ethyl acetate (5 mL×3). The combined organic layers were washed with brine (5 mL), dried over anhydrous sodium sulfate, filtered, and concentrated under reduced pressure to give a residue. The residue was purified by prep-TLC (40% ethyl acetate in petroleum ether) to afford the title compound (30 mg, 85%) as a white solid which was used directly for next step. LCMS R_{T}=1.353 min, m/z=352.9 [M+H]⁺.

### Step 7: Synthesis of tert-butyl N-[8-[3-[tert-butyl(dimethyl)silyl]oxy-2,6-dimethyl-phenyl]-6-cyano-3-methyl-pyrrolo[1,2-a]pyrazin-7-yl]carbamate

To a solution of tert-butyl N-(8-bromo-6-cyano-3-methyl-pyrrolo[1,2-a]pyrazin-7-yl)carbamate (30 mg, 0.085 mmol) in dioxane (1 mL) and water (0.1 mL) was added [3-[tert-butyl(dimethyl)silyl]oxy-2,6-dimethyl-phenyl]boronic acid (72 mg, 0.26 mmol), Pd₂(dba)₃ (8 mg, 0.009 mmol), potassium carbonate (35 mg, 0.26 mmol) and SPhos (7 mg, 0.017 mmol). The mixture was stirred at 100° C. for 1 hour under a nitrogen atmosphere. The reaction was filtered and concentrated under reduced pressure to give the residue which was purified by prep-TLC to afford the title compound (10 mg, 21%) as a yellow oil. LCMS R_{T}=0.673 min, m/z=507.3 [M+H]⁺.

### Step 8: Synthesis of 7-amino-8-(3-hydroxy-2,6-dimethyl-phenyl)-3-methyl-pyrrolo[1,2-a]pyrazine-6-carbonitrile

To a solution of tert-butyl N-[8-[3-[tert-butyl(dimethyl)silyl]oxy-2,6-dimethyl-phenyl]-6-cyano-3-methyl-pyrrolo[1,2-a]pyrazin-7-yl]carbamate (10 mg, 0.020 mmol) in hydrogen chloric acid in methanol (4 M, 2 mL) was stirred at 25° C. for 2 hour. The reaction was concentrated under reduced pressure to give the title compound (12 mg, crude) as a yellow oil which was used for next step directly.

### Step 9: Synthesis of 7-amino-8-(3-hydroxy-2,6-dimethyl-phenyl)-3-methyl-pyrrolo[1,2-a]pyrazine-6-carboxamide (Compound 555)

To a solution of 7-amino-8-(3-hydroxy-2,6-dimethyl-phenyl)-3-methyl-pyrrolo[1,2-a]pyrazine-6-carbonitrile (12 mg, 0.041 mmol) in ethanol (2 mL) and water (0.4 mL) was added hydrogen peroxide (23 mg, 0.21 mmol, 30% purity) and lithium hydroxide monohydrate (9 mg, 0.21 mmol). The mixture was stirred at 60° C. for 2 hour. The reaction mixture was extracted with dichloromethane (10 mL×3). The combined organic phase was washed with brine (20 mL), dried over anhydrous sodium sulfate, filtered, and concentrated under reduced pressure to give the residue which was purified by RP-HPLC (0 to 24% acetonitrile in water and 0.225% formic acid) to afford the title compound (1 mg, 8%) as a yellow solid. ¹H NMR (400 MHz, CD₃OD) δ 9.03 (s, 1H), 8.03 (s, 1H), 7.03 (d, J=8.0 Hz, 1H), 6.81 (d, J=8.4 Hz, 1H), 2.43 (s, 3H), 1.94 (s, 3H), 1.90 (s, 3H). LCMS R_{T}=0.669 min, m/z=311.0 [M+H]⁺.

### Example 5. Synthesis of Compound 562

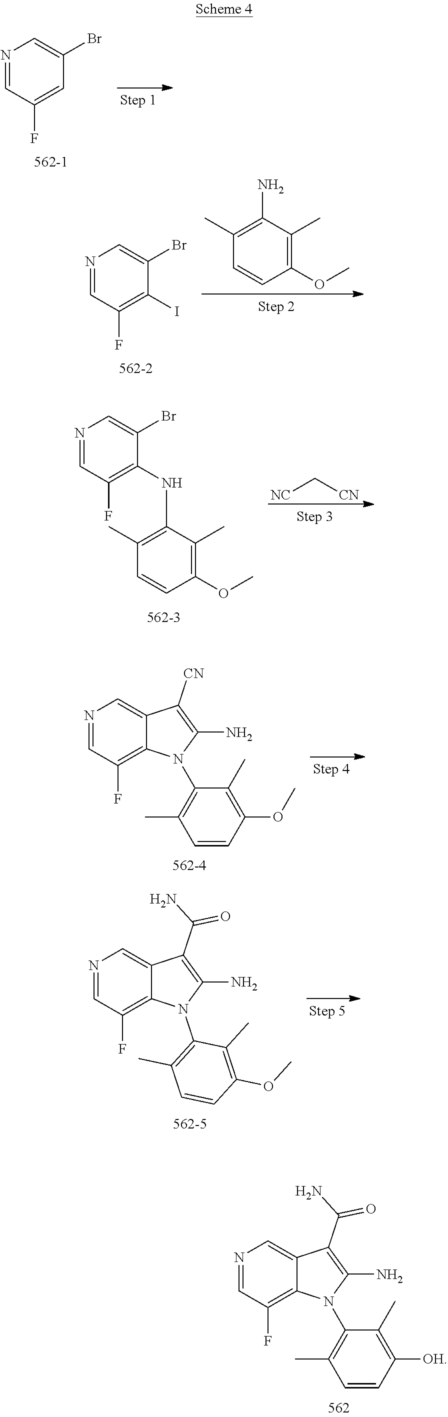

### Step 1: Synthesis of 3-bromo-5-fluoro-4-iodo-pyridine

To a solution of 3-bromo-5-fluoro-pyridine (5.0 g, 28.41 mmol) in tetrahydrofuran (15 mL) was added lithium diisopropylamide (1 M, 43 mL). The mixture was then stirred at −78° C. for 0.25 hour under nitrogen atmosphere. Then the mixture was added iodine (8.65 g, 34.09 mmol) in tetrahydrofuran (10 mL) stirred at −78° C. for 2 hours. The mixture was quenched by addition of saturated aqueous ammonium chloride, and extracted with ethyl acetate (50 mL×3). The combined organic phase was washed with brine (100 mL), dried over anhydrous sodium sulfate, filtered and concentrated under reduced pressure to give the residue which was purified by flash column chromatography (silica gel, 100-200 mesh, 0-7% ethyl acetate in petroleum ether) to afford the title compound (1.66 g, crude) as a yellow solid. ¹H NMR (400 MHz, DMSO-d₆) δ 8.63-8.59 (m, 2H), 8.57 (s, 1H), 8.44 (s, 1H), 8.23-8.17 (m, 1H).

### Step 2: Synthesis of 3-bromo-5-fluoro-N-(3-methoxy-2,6-dimethyl-phenyl)pyridin-4-amine

To a solution of 3-methoxy-2,6-dimethyl-aniline (484 mg, 3.20 mmol) and 3-bromo-5-fluoro-4-iodo-pyridine (1.61 g, 3.20 mmol, 60% purity) in 1,2-dimethoxyethane (40 mL) was added cesium carbonate (2.09 g, 6.40 mmol) and Xantphos (185 mg, 0.32 mmol), Pd₂(dba)₃ (147 mg, 0.16 mmol). The mixture was stirred at 85° C. for 13 hour under a nitrogen atmosphere. The reaction was filtered and concentrated under reduced pressure to give a residue which was purified by flash column chromatography (silica gel, 100-200 mesh, 0-5% ethyl acetate in petroleum ether) to afford the title compound (278 mg, 25%) as a yellow oil. ¹H NMR (400 MHz, DMSO-d₆) δ 8.26 (s, 1H), 8.09 (d, J=4.0 Hz, 1H), 7.86 (d, J=2.8 Hz, 1H), 7.03 (d, J=8.4 Hz, 1H), 6.83 (d, J=8.4 Hz, 1H), 3.77 (s, 3H), 2.05 (s, 3H), 1.97 (s, 3H). LCMS R_{T}=0.482 min, m/z=326.6 [M+H]⁺.

### Step 3: Synthesis of 2-amino-7-fluoro-1-(3-methoxy-2,6-dimethyl-phenyl)pyrrolo[3,2-c]pyridine-3-carbonitrile

To a solution of propanedinitrile (114 mg, 1.73 mmol) in 1,2-dimethoxyethane (3 mL) was added sodium hydrogen (59 mg, 1.48 mmol, 60% purity). The mixture was stirred at 25° C. for 0.5 hour. Then the mixture was added 3-bromo-5-fluoro-N-(3-methoxy-2,6-dimethyl-phenyl)pyridin-4-amine (268 mg, 0.82 mmol) and 1,1′-bis(diphenylphosphino)ferrocene-palladium (II) dichloride dichloromethane complex₂ (67 mg, 0.082 mmol) and stirred at 110° C. for 4 hours under nitrogen atmosphere. The reaction solution was quenched by addition of water (10 mL) and concentrated under reduced pressure to remove the organic solvent. The mixture was then extracted with ethyl acetate (50 mL×3). The combined organics were washed with brine (100 mL), dried over anhydrous sodium sulfate, filtered, and concentrated under reduced pressure to give a residue which was purified by flash column chromatography (silica gel, 100-200 mesh, 0-25% ethyl acetate in petroleum ether) to afford the title compound (101 mg, 35%) as a white solid. LCMS R_{T}=0.463 min, m/z=311.0 [M+H]⁺.

### Step 4: Synthesis of 2-amino-7-fluoro-1-(3-methoxy-2,6-dimethyl-phenyl)pyrrolo[3,2-c]pyridine-3-carboxamide

To a solution of 2-amino-7-fluoro-1-(3-methoxy-2,6-dimethyl-phenyl)pyrrolo[3,2-c]pyridine-3-carbonitrile (96 mg, 0.31 mmol) in concentrated sulfuric acid (2 mL) was stirred at 25° C. for 1 hour. The reaction solution was poured into ice water (40 mL), the mixture was quenched by addition of aqueous saturated sodium bicarbonate (30 mL) to pH=8. The mixture was then extracted with ethyl acetate (30 mL×3). The organic phase was washed with brine (50 mL), dry over anhydrous sodium sulfate, filtered, and concentrated under reduced pressure to give a residue which was purified by prep-TLC to afford the title compound (71 mg, 67%) as a white solid. ¹H NMR (400 MHz, DMSO-d₆) δ 8.80 (s, 1H), 7.96 (d, J=2.4 Hz, 1H), 7.25 (d, J=8.0 Hz, 1H), 7.10 (d, J=8.4 Hz, 1H), 6.94 (d, J=12.0 Hz, 4H), 3.84 (s, 3H), 1.84 (s, 3H), 1.75 (s, 3H). LCMS R_{T}=0.445 min, m/z=329.1 [M+H]⁺.

### Step 5: Synthesis of 2-amino-7-fluoro-1-(3-hydroxy-2,6-dimethyl-phenyl)pyrrolo[3,2-c]pyridine-3-carboxamide (Compound 562)

To a solution of 2-amino-7-fluoro-1-(3-methoxy-2,6-dimethyl-phenyl)pyrrolo[3,2-c]pyridine-3-carboxamide (30 mg, 0.091 mmol) in toluene (3 mL) was added aluminum chloride (122 mg, 0.91 mmol) and stirred at 100° C. for 2 hours. The reaction mixture was added hydrochloric acid (2 M, 5 mL), methanol (5 mL) and water (5 mL). The mixture was then filtered and concentrated under reduced pressure to give the residue which was purified by prep-HPLC (0 to 90% acetonitrile in water and 1% hydrochloric acid) to afford the title compound (14 mg, 48%) as a white solid. ¹H NMR (400 MHz, DMSO-d₆) δ 9.88 (s, 1H), 9.10 (s, 1H), 8.61 (d, J=5.2 Hz, 1H), 7.53 (s, 2H), 7.34 (s, 2H), 7.12 (d, J=8.4 Hz, 1H), 7.02 (d, J=8.4 Hz, 1H), 1.82 (s, 3H), 1.74 (s, 3H). LCMS R_{T}=0.938 min, m/z=315.3 [M+H]⁺.

### Example 6. Synthesis of Compound 546

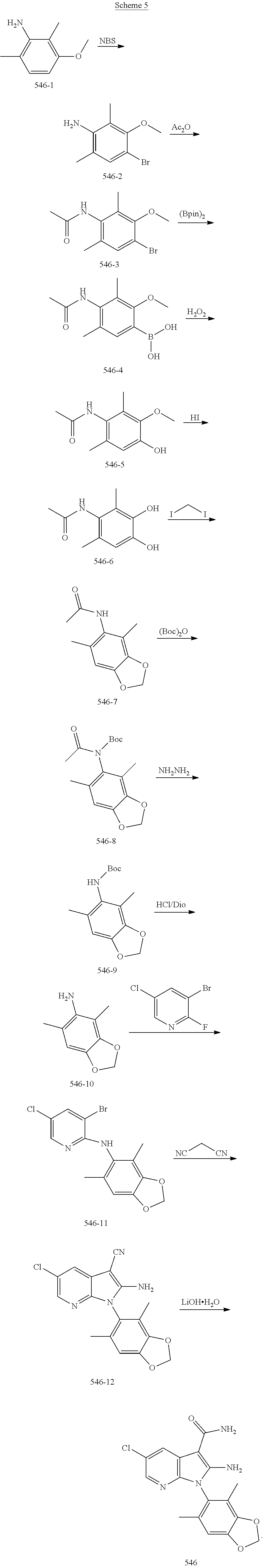

### Step 1: Synthesis of 4-bromo-3-methoxy-2,6-dimethylaniline

To a solution of 3-methoxy-2,6-dimethyl-aniline (10.0 g, 66.14 mmol) in N,N-dimethylformamide (100 mL) was added 1-bromopyrrolidine-2,5-dione (10.6 g, 59.52 mmol) at 20° C. The reaction mixture was purged with nitrogen three times and was stirred at 20° C. for 1 hours under a nitrogen atmosphere. The reaction mixture was diluted with water (1 L) and extracted with ethyl acetate (500 mL×3). The combined organic layers were washed with brine (500 mL), dried over anhydrous sodium sulfate, filtered, and concentrated under reduced pressure to give a residue. The residue was purified by flash column chromatography (silica gel, 100-200 mesh, 50% ethyl acetate in petroleum ether) to afford the title compound (15 g, 79%) as a yellow oil which was used directly for next step. LCMS R_{T}=1.617 min, m/z=231.9 [M+H]⁺.

### Step 2: Synthesis of N-(4-bromo-3-methoxy-2,6-dimethylphenyl)acetamide

To a mixture of 4-bromo-3-methoxy-2,6-dimethyl-aniline (15.0 g, 65.19 mmol) in dichloromethane (150 mL) was added acetic anhydride (6.66 g, 65.19 mmol), pyridine (5.16 g, 65.19 mmol) at 20° C. The reaction mixture was purged with nitrogen 3 times and was stirred at 20° C. for 2 hours under a nitrogen atmosphere. The reaction mixture was diluted with water (1 L) and extracted with dichloromethane (500 mL×3). The combined organic layers were washed with brine (1 L), dried over anhydrous sodium sulfate, filtered, and concentrated under reduced pressure to give a residue. The residue was purified by flash column chromatography (silica gel, 100-200 mesh, 50% ethyl acetate in petroleum ether) to afford the title compound (16 g, 90%) as a yellow solid which was used directly for next step. LCMS R_{T}=1.313 min, m/z=271.9 [M+H]⁺.

### Step 3: Synthesis of (4-acetamido-2-methoxy-3,5-dimethylphenyl)boronic acid

To a solution of N-(4-bromo-3-methoxy-2,6-dimethyl-phenyl)acetamide (14.5 g, 53.28 mmol) in dioxane (150 mL) was added 4,4,5,5-tetramethyl-2-(4,4,5,5-tetramethyl-1,3,2-dioxaborolan-2-yl)-1,3,2-dioxaborolane (27.06 g, 106.56 mmol), 1,1′-bis(diphenylphosphino)ferrocene-palladium (II) dichloride dichloromethane complex (3.90 g, 5.33 mmol), and potassium acetate (15.69 g, 159.85 mmol) at 20° C. The reaction mixture was purged with nitrogen 3 times and was stirred at 110° C. for 4 hours under a nitrogen atmosphere. The reaction mixture was diluted with water (200 mL) and extracted with ethyl acetate (100 mL×3). The combined organic layers were washed with brine (100 mL), dried over anhydrous sodium sulfate, filtered, and concentrated under reduced pressure to afford crude title compound (17 g, 90%) as a yellow oil which was used directly for next step. LCMS R_{T}=1.347 min, m/z=320.0 [M+H]⁺.

### Step 4: Synthesis of N-(4-hydroxy-3-methoxy-2,6-dimethylphenyl)acetamide

To a mixture of N-[3-methoxy-2,6-dimethyl-4-(4,4,5,5-tetramethyl-1,3,2-dioxaborolan-2-yl)phenyl]acetamide (16.0 g, 50.1 mmol) in tetrahydrofuran (40 mL) was added sodium hydroxide (1 M, 50.1 mmol) and hydrogen peroxide (8.52 g, 75.2 mmol, 30%). After the reaction was completed, sodium thiosulfate solution (1 M, 20 mL) was added and the reaction mixture was extracted with dichloromethane (10 mL×3). The organic layers were washed with 5% sodium hydrogen carbonate solution, brine, dried over sodium sulfate, filtered, and concentrated under reduced pressure to give a residue. The residue was purified by flash column chromatography (silica gel, 100-200 mesh, 50% ethyl acetate in petroleum ether) to afford the title compound (8.8 g, 84%) as a yellow oil which was used directly for next step. LCMS R_{T}=0.824 min, m/z=209.9 [M+H]⁺.

### Step 5: Synthesis of N-(3,4-dihydroxy-2,6-dimethylphenyl)acetamide

To a solution of N-(4-hydroxy-3-methoxy-2,6-dimethyl-phenyl)acetamide (8.6 g, 41.1 mmol) in dichloromethane (90 mL) was added tribromoborane (1 M in dichloromethane, 82.20 mmol) at 20° C. The reaction mixture was stirred at 20° C. for 1 hours. The reaction mixture was quenched with water (100 mL) and extracted with ethyl acetate (50 mL×3). The combined organic layers were washed with brine (50 mL), dried over anhydrous sodium sulfate, filtered, and concentrated under reduced pressure to give a residue. The residue was purified by RP-HPLC (0 to 20% acetonitrile in water and 0.225% formic acid) to afford the title compound (3.5 g, 44%) as a white solid which was used directly for next step. LCMS R_{T}=0.250 min, m/z=196.1 [M+H]⁺.

### Step 6: Synthesis of N-(4,6-dimethylbenzo[d][1,3]dioxol-5-yl)acetamide

To a solution of N-(3,4-dihydroxy-2,6-dimethyl-phenyl)acetamide (3.5 g, 17.93 mmol) in N,N-dimethylformamide (30 mL) was added diiodomethane (14.4 g, 53.79 mmol) and cesium carbonate (11.68 g, 35.86 mmol) at 20° C. The mixture was stirred at 100° C. for 2 hour under a nitrogen atmosphere. The reaction mixture was diluted with water (20 mL) and extracted with ethyl acetate (20 mL×3). The combined organic layers were washed with brine (10 mL), dried over anhydrous sodium sulfate, filtered, and concentrated under reduced pressure to give a residue. The residue was purified by flash column chromatography (silica gel, 100-200 mesh, 50% ethyl acetate in petroleum ether) to afford the title compound (3.6 g, 97%) as a white solid which was used directly for next step. LCMS R_{T}=0.429 min, m/z=207.9 [M+H]⁺.

### Step 7: Synthesis of tert-butyl acetyl(4,6-dimethylbenzo[d][1,3]dioxol-5-yl)carbamate

To a solution of N-(4,6-dimethyl-1,3-benzodioxol-5-yl)acetamide (1.8 g, 8.69 mmol) in N,N-dimethylformamide (40 mL) was added di-tert-butyldicarbonate (3.79 g, 17.37 mmol), N,N-dimethylpyridin-4-amine (1.06 g, 8.69 mmol) and N,N-diethylethanamine (2.64 g, 26.06 mmol) at 20° C. The reaction mixture was stirred at 20° C. for 1 hours. The reaction mixture was diluted with water (300 mL) and extracted with ethyl acetate (100 mL×3). The combined organic layers were washed with brine (100 mL), dried over anhydrous sodium sulfate, filtered, and concentrated under reduced pressure to give a residue. The residue was purified by flash column chromatography (silica gel, 100-200 mesh, 21% ethyl acetate in petroleum ether) to afford the title compound (3.5 g, 65%) as a white solid which was used directly for next step. LCMS R_{T}=1.478 min, m/z=252.0 [M+H−56]⁺.

### Step 8: Synthesis of tert-butyl (4,6-dimethylbenzo[d][1,3]dioxol-5-yl)carbamate

To a solution of tert-butyl N-acetyl-N-(4,6-dimethyl-1,3-benzodioxol-5-yl)carbamate (3.5 g, 11.39 mmol) in tert-butanol (20 mL) was added hydrazine hydrate (20.60 g, 411.50 mmol) at 20° C. The reaction mixture was stirred at 110° C. for 6 hours. The reaction mixture was quenched with water (200 mL) and extracted with ethyl acetate (100 mL×3). The combined organic layers were washed with brine (100 mL), dried over anhydrous sodium sulfate, filtered, and concentrated under reduced pressure to give a residue. The residue was purified by flash column chromatography (silica gel, 100-200 mesh, 30% ethyl acetate in petroleum ether) to afford the title compound (2.5 g, 83%) as a white solid which was used directly for next step. LCMS R_{T}=0.927 min, m/z=210.1 [M+H−56]⁺.

### Step 9: Synthesis of 4,6-dimethylbenzo[d][1,3]dioxol-5-amine

The mixture of tert-butyl N-(4,6-dimethyl-1,3-benzodioxol-5-yl)carbamate (2.5 g, 9.42 mmol) in hydrogen chloride and dioxane (4 M, 5 mL) was stirred at 20° C. for 1 hour. The reaction mixture was concentrated under reduced pressure to remove the solvent. The residue was purified by flash column chromatography (silica gel, 100-200 mesh, 34% ethyl acetate in petroleum ether) to afford the title compound (1.25 g, 80%) as a white solid which was used directly in the next step. ¹H NMR (400 MHz, CD₃OD) δ 6.43 (s, 1H), 5.78 (s, 2H), 2.12 (s, 3H), 2.08 (s, 3H)

### Step 10: Synthesis of 3-bromo-5-chloro-N-(4,6-dimethylbenzo[d][1,3]dioxol-5-yl)pyridin-2-amine

To a solution of 4,6-dimethyl-1,3-benzodioxol-5-amine (200 mg, 1.21 mmol) in tetrahydrofuran (4 mL) was added 3-bromo-5-chloro-2-fluoro-pyridine (255 mg, 1.21 mmol) and lithium hexamethyldisilazide (1 M, 2.42 mmol) at 0° C., then the mixture was stirred at 20° C. for 1 hour. The reaction was quenched with saturated ammonium chloride (20 mL). Then the reaction mixture was diluted with water (20 mL) and extracted with ethyl acetate (20 mL×3). The combined organic layers were washed with brine (10 mL), dried over anhydrous sodium sulfate, filtered, and concentrated under reduced pressure to give a residue. The residue was purified by flash column chromatography (silica gel, 100-200 mesh, 20% ethyl acetate in petroleum ether) to afford the title compound (400 mg, 93%) as a yellow solid which was used directly in the next step. LCMS R_{T}=0.614 min, m/z=356.8 [M+H+2]⁺.

### Step 11: Synthesis of 2-amino-5-chloro-1-(4,6-dimethylbenzo[d][1,3]dioxol-5-yl)-1H-pyrrolo[2,3-b]pyridine-3-carbonitrile

To a solution of sodium hydride (72 mg, 1.80 mmol, 60% purity) in 1,2-dimethoxyethane (2 mL) was added propanedinitrile (119 mg, 1.80 mmol) in 1,2-dimethoxyethane (2 mL) dropwise. After stirring for 0.5 h, 3-bromo-5-chloro-N-(4,6-dimethyl-1,3-benzodioxol-5-yl)pyridin-2-amine (400 mg, 0.9 mmol) in 1,2-dimethoxyethane (2 mL) and 1,1′-bis(diphenylphosphino)ferrocene-palladium (II) dichloride dichloromethane complex (110 mg, 0.14 mmol) were added. The reaction mixture was stirred at 100° C. for 2.5 hours. The reaction was quenched with water (10 mL) and was extracted with ethyl acetate (10 mL×3). The combined organic layers were washed with brine (10 mL), dried over anhydrous sodium sulfate, filtered, and concentrated under reduced pressure to give a residue. The residue was purified by flash column chromatography (silica gel, 100-200 mesh, 56% ethyl acetate in petroleum ether) to afford the title compound (200 mg, 65%) as a yellow solid which was used directly in the next step. LCMS R_{T}=0.553 min, m/z=341.2 [M+H]⁺.

### Step 12: Synthesis of 2-amino-5-chloro-1-(4,6-dimethylbenzo[d][1,3]dioxol-5-yl)-1H-pyrrolo[2,3-b]pyridine-3-carboxamide (Compound 546)

To a solution of 2-amino-5-chloro-1-(4,6-dimethyl-1,3-benzodioxol-5-yl)pyrrolo[2,3-b]pyridine-3-carbonitrile (50 mg, 0.15 mmol) was added lithium hydroxide hydrate (31 mg, 0.73 mmol) and hydrogen peroxide (92 mg, 0.73 mmol, 27% purity) in ethanol (2 mL) and water (0.4 mL) at 20° C. Then the mixture was stirred at 60° C. for 1 hour. The reaction mixture was filtered and concentrated under reduced pressure to remove the solvent. The crude product was purified by RP-HPLC (18% to 58% acetonitrile in water and 0.225% ammonium hydrogencarbonate) to give the title compound (2.53 mg, 5%). ¹H NMR (400 MHz, CD₃OD) δ 8.25 (d, J=2.0 Hz, 1H), 7.76 (d, J=2.0 Hz, 1H), 6.78 (s, 1H), 6.04 (s, 2H), 1.86 (s, 3H), 1.80 (s, 3H). LCMS R_{T}=0.508 min, m/z=358.9 [M+H]⁺.

### Example 7. Synthesis of Compound 542

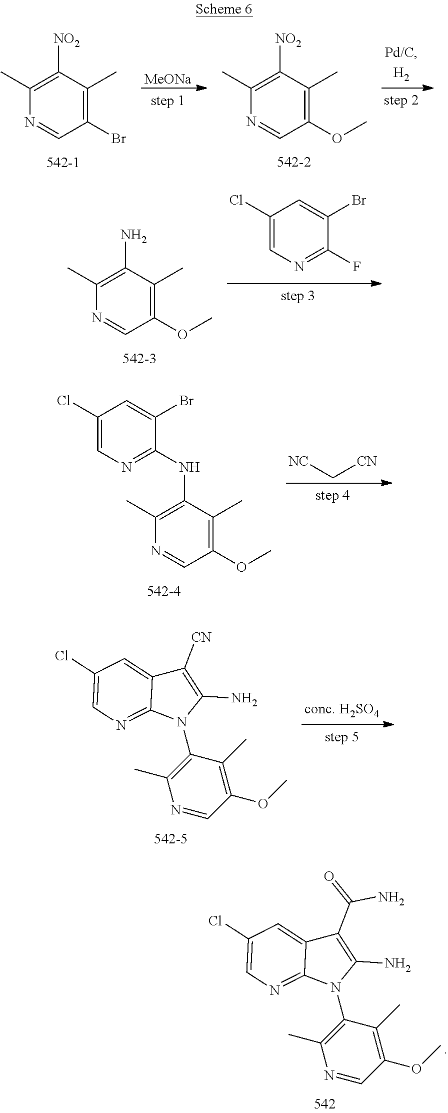

### Step 1: Synthesis of 5-methoxy-2,4-dimethyl-3-nitro-pyridine

To a solution of 5-bromo-2,4-dimethyl-3-nitro-pyridine (300 mg, 1.30 mmol) in methyl alcohol (2 mL) and N,N-dimethylformamide (2 mL) was added sodium methylate (210 mg, 3.90 mmol) and cuprous bromide (37 mg, 260 mmol). The mixture was stirred at 110° C. for 12 hours. The reaction mixture was quenched with water (10 mL) and extracted with ethyl acetate (10 mL×2). The combined organic layers were washed with brine (10 mL), dried over anhydrous sodium sulphate, filtered, and concentrated under reduced pressure. The residue was purified by flash column chromatography (silica gel, 100-200 mesh, 0-30% ethyl acetate in petroleum ether) to afford the title compound (66 mg, 28%) as a yellow solid. LCMS R_{T}=0.485 min, m/z=183.1 [M+H]⁺.

### Step 2: Synthesis of 5-methoxy-2,4-dimethyl-pyridin-3-amine

To a solution of 5-methoxy-2,4-dimethyl-3-nitro-pyridine (66 mg, 0.362 mmol) in methyl alcohol (3 mL) was added 5% palladium on carbon (39 mg). The mixture was stirred at 20° C. for 1 hour under hydrogen (15 psi). The reaction mixture was filtered and the filtrate was concentrated under reduced pressure to give a residue. The residue was purified by prep-TLC to afford the title compound (45 mg, 82%) as a yellow solid. LCMS R_{T}=0.325 min, m/z=153.1 [M+H]⁺.

### Step 3: Synthesis of 3-bromo-5-chloro-N-(5-methoxy-2,4-dimethylpyridin-3-yl)pyridin-2-amine

To a solution of 5-methoxy-2,4-dimethyl-pyridin-3-amine (100 mg, 0.66 mmol) and 3-bromo-5-chloro-2-fluoro-pyridine (140 mg, 0.67 mmol) in tetrahydrofuran (1 mL) was added sodium bis(trimethylsilyl)amide (1 M, 1.31 mL) a 0° C. The mixture was stirred at 0° C. for 1 hour. The reaction mixture was quenched with water (15 mL) and extracted with ethyl acetate (10 mL×2). The combined organic layers were washed with brine (10 mL×2), dried over sodium sulphate, filtered, and concentrated under reduced pressure to give a residue. The residue was purified by flash column chromatography (silica gel, 100-200 mesh, 90% ethyl acetate in petroleum ether) to afford the title compound (200 mg, 95%) as a yellow solid. LCMS R_{T}=0.458 min, m/z=344.0 [M+H]⁺.

### Step 4: Synthesis of 2-amino-5-chloro-1-(5-methoxy-2,4-dimethylpyridin-3-yl)-1H-pyrrolo[2,3-b]pyridine-3-carbonitrile

To a solution of propanedinitrile (289 mg, 4.38 mmol) in 1,2-dimethoxyethane (10 mL) was added sodium hydride (175 mg, 4.38 mmol, 60% purity) at 0° C. and stirred for 0.5 hour, followed by N-(3-bromo-5-chloro-2-pyridyl)-5-methoxy-2,4-dimethyl-pyridin-3-amine (500 mg, 1.46 mmol) and 1,1′-bis(diphenylphosphino)ferrocene-palladium (II) dichloride dichloromethane complex (180 mg, 0.22 mmol). The mixture was stirred at 100° C. for 2 hours under microwave. The reaction mixture was quenched with water (10 mL) and extracted with ethyl acetate (10 mL×2). The combined organic layers were washed with brine (10 mL), dried over sodium sulphate, filtered, and concentrated under reduced pressure to give a residue. The residue was purified by flash column chromatography (silica gel, 100-200 mesh, 100% ethyl acetate in petroleum ether) to afford the title compound (220 mg, 90%) as a yellow solid. LCMS R_{T}=0.478 min, m/z=327.9 [M+H]⁺.

### Step 5: Synthesis of 2-amino-5-chloro-1-(5-methoxy-2,4-dimethylpyridin-3-yl)-1H-pyrrolo[2,3-b]pyridine-3-carboxamide (Compound 542)

To a solution of 2-amino-5-chloro-1-(5-methoxy-2,4-dimethyl-3-pyridyl)pyrrolo[2,3-b]pyridine-3-carbonitrile (200 mg, 0.52 mmol) in sulfuric acid (3 mL). The mixture was stirred at 20° C. for 1 hour. The reaction solution was poured into ice water (8 mL) and adjusted to pH=7 and then extracted with ethyl acetate (10 mL×2). The organics were washed with brine (10 mL), dry over anhydrous sodium sulfate, filtered, and concentrated under reduced pressure to give a residue which was purified by RP-HPLC (45 to 75% acetonitrile in water and 0.05% hydrochloric acid) to give the title compound (70 mg, 90%) as a colorless solid. LCMS R_{T}=0.412 min, m/z=346.0 [M+H]⁺.

### Example 8. Synthesis of Compound 543

### Synthesis of 2-amino-5-chloro-1-(5-hydroxy-2,4-dimethylpyridin-3-yl)-1H-pyrrolo[2,3-b]pyridine-3-carbonitrile

To a solution of 2-amino-5-chloro-1-(5-methoxy-2,4-dimethyl-3-pyridyl)pyrrolo[2,3-b]pyridine-3-carboxamide (50 mg, 0.14 mmol) in toluene (3 mL) was added aluminum chloride (193 mg, 1.45 mmol). The mixture was stirred at 100° C. for 2 hours. The reaction mixture was quenched with the addition of hydrochloric acid (1 M, 0.5 mL) and concentrated under reduced pressure to give a residue which was purified by RP-HPLC (3 to 33% acetonitrile in water and 0.05% hydrochloric acid) to give the title compound (21.9 mg, 99.5%) as a colorless solid. ¹H NMR (400 MHz, DMSO-d₆) δ 12.06 (s, 1H), 8.38 (s, 1H), 8.23 (d, J=2.0 Hz, 1H), 7.81 (d, J=2.4 Hz, 1H), 7.57 (s, 2H), 6.97 (s, 2H), 2.24 (s, 3H), 1.90 (s, 3H). LCMS R_{T}=1.091 min, m/z=331.9 [M+H]⁺.

### Example 9. Synthesis of Compound 544

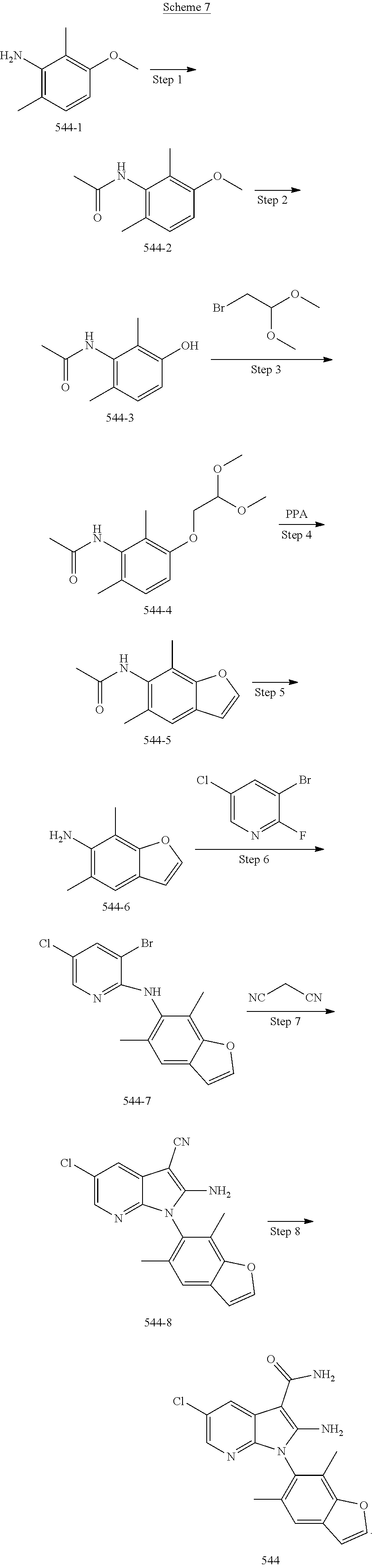

### Step 1: Synthesis of N-(3-methoxy-2,6-dimethylphenyl)acetamide

To a solution of 3-methoxy-2,6-dimethylaniline (2.0 g, 13.23 mmol) in dichloromethane (30 mL) was added acetic anhydride (2.7 g, 26.45 mmol) and pyridine (2.09 g, 26.45 mmol). The mixture was stirred at 20° C. for 1 hour. The reaction was diluted with water (50 mL) and extracted with ethyl acetate (50 mL×3). The combined organics were washed with brine (30 mL), dried over anhydrous sodium sulfate, filtered and concentrated under reduced pressure to give the crude title compound (3.3 g, 100%) as light yellow oil which was used in the next step without further purification. LCMS R_{T}=0.433 min, m/z=194.0 [M+H]⁺.

### Step 2: Synthesis of N-(3-hydroxy-2,6-dimethylphenyl)acetamide

To a solution of N-(3-methoxy-2,6-dimethylphenyl)acetamide (3.3 g, 17.08 mmol) in dichloromethane (50 mL) was added boron tribromide (68.31 mmol, 1 M in dichloromethane) at 0° C. The mixture was stirred at 0° C. for 1 hour. The reaction was quenched with the addition of methanol (10 mL) at 0° C. and then concentrated under reduced pressure to afford the crude title compound (3.0 g, 100%) as light yellow oil which was used in next step without further purification. LCMS R_{T}=0.352 min, m/z=180.0 [M+H]⁺.

### Step 3: Synthesis of N-(3-(2,2-dimethoxyethoxy)-2,6-dimethylphenyl)acetamide

To a solution of N-(3-hydroxy-2,6-dimethylphenyl)acetamide (1.00 g, 5.58 mmol) in N,N-dimethylformamide (10 mL) was added potassium carbonate (2.31 g, 16.74 mmol) and 2-bromo-1,1-dimethoxyethane (1.89 g, 11.16 mmol). The mixture was stirred at 140° C. for 12 hour. The reaction mixture was concentrated under reduced pressure to give a residue which was purified by flash column chromatography (silica gel, 100-200 mesh, 0-100% ethyl acetate in petroleum ether) to afford the title compound (1.3 g, 82%) as a white solid. LCMS R_{T}=0.438 min, m/z=268.0 [M+H]⁺.

### Step 4: Synthesis of N-(5,7-dimethylbenzofuran-6-yl)acetamide

To a solution of N-(3-(2,2-dimethoxyethoxy)-2,6-dimethylphenyl)acetamide (1.0 g, 3.74 mmol) in toluene (20 mL) was added polyphosphoric acid (2.8 g, 3.74 mmol). The mixture was stirred at 90° C. for 1 hour. The reaction was diluted with water (30 mL) and ethyl acetate (30 mL). The mixture was filtered and the filtrate was extracted with ethyl acetate (50 mL×3). The combined organics were washed with brine (30 mL), dried over anhydrous sodium sulfate, filtered, and concentrated under reduced pressure to give a residue which was purified by flash column chromatography (silica gel, 100-200 mesh, 0-100% ethyl acetate in petroleum ether) to afford the title compound (160 mg, 16%) as a yellow solid. LCMS R_{T}=0.447 min, m/z=204.0 [M+H]⁺.

### Step 5: Synthesis of 5,7-dimethylbenzofuran-6-amine

To a solution of N-(5,7-dimethylbenzofuran-6-yl)acetamide (110 mg, 0.54 mmol) in methanol (1 mL) was added hydrochloric acid (1 mL, 12 M). The mixture was stirred at 120° C. for 12 hours. The reaction was quenched with the addition of aqueous saturated sodium bicarbonate to pH=8. The mixture was extracted with ethyl acetate (20 mL×3). The combined organic phase was washed with brine (10 mL), dried over anhydrous sodium sulfate, filtered, and concentrated under reduced pressure to give the residue, which was purified by flash column chromatography (silica gel, 100-200 mesh, 0-30% ethyl acetate in petroleum ether) to afford the title compound (90 mg, 94%) as a light brown solid. LCMS R_{T}-0.400 min, m/z=162.2 [M+H]⁺.

### Step 6: Synthesis of 3-bromo-5-chloro-N-(5,7-dimethylbenzofuran-6-yl)pyridin-2-amine

To a solution of 5,7-dimethylbenzofuran-6-amine (80 mg, 0.50 mmol) and 3-bromo-5-chloro-2-fluoropyridine (104 mg, 0.50 mmol) in tetrahydrofuran (3 mL) was added lithium hexamethyldisilazide (1 M, 1.49 mL). The mixture was stirred at 0° C. for 1 hour. The reaction was quenched with the addition of methanol (1 mL) at 0° C. The mixture was concentrated under reduced pressure to give a residue which was purified by flash column chromatography (silica gel, 100-200 mesh, 0-30% ethyl acetate in petroleum ether) to afford the title compound (130 mg, 28%) as a light yellow solid. LCMS R_{T}=2.077 min, m/z=352.9 [M+H+2]⁺.

### Step 7: Synthesis of 2-amino-5-chloro-1-(5,7-dimethylbenzofuran-6-yl)-1H-pyrrolo[2,3-b]pyridine-3-carbonitrile

To a solution of propanedinitrile (62 mg, 0.94 mmol) in 1,2-dimethoxyethane (2 mL) was added sodium hydride (38 mg, 0.94 mmol, 60% purity) at 0° C. The mixture was stirred at 0° C. for 0.5 hour. Then the mixture was added to 3-bromo-5-chloro-N-(5,7-dimethylbenzofuran-6-yl)pyridin-2-amine (110 mg, 0.31 mmol) and 1,1′-bis(diphenylphosphino)ferrocene-palladium (II) dichloride dichloromethane complex (26 mg, 0.031 mmol) and was stirred at 100° C. for 12 hours under a nitrogen atmosphere. The reaction solution was quenched by addition of water (1 mL) and concentrated under reduced pressure to give a residue which was purified by flash column chromatography (silica gel, 100-200 mesh, 0-50% ethyl acetate in petroleum ether) to afford the title compound (110 mg, 32%) as a light brown solid. LCMS R_{T}=1.789 min, m/z=337.0 [M+H]⁺.

### Step 8: Synthesis of 2-amino-5-chloro-1-(5,7-dimethylbenzofuran-6-yl)-1H-pyrrolo[2,3-b]pyridine-3-carboxamide (Compound 544)

To a solution of 2-amino-5-chloro-1-(5,7-dimethylbenzofuran-6-yl)-1H-pyrrolo[2,3-b]pyridine-3-carbonitrile (90 mg, 0.27 mmol) in ethanol (20 mL) and water (0.5 mL) was added hydrogen peroxide (121 mL, 30%) and lithium hydroxide monohydrate (45 mg, 1.07 mmol). The mixture was stirred at 60° C. for 1 hour. The reaction mixture was diluted with water (10 mL) and extracted with ethyl acetate (20 mL×3). The combined organics were washed with brine (10 mL), dried over anhydrous sodium sulfate, filtered, and concentrated under reduced pressure to give the residue, which was purified by RP-HPLC (50 to 70% acetonitrile in water and 0.225% formic acid) to afford the title compound (2.6 mg, 3%) as a light gray solid. ¹H NMR (400 MHz, DMSO-d₆) δ 8.43 (s, 1H), 8.16 (m, 1H), 8.11 (m, 1H), 7.73 (m, 1H), 7.54 (m, 1H), 7.24 (s, 2H), 7.02 (m, 1H), 6.87 (s, 2H), 2.04 (s, 3H), 1.91 (s, 3H). LCMS R_{T}=2.087 min, m/z=355.3 [M+H]⁺.

### Example 10. Synthesis of Compound 547

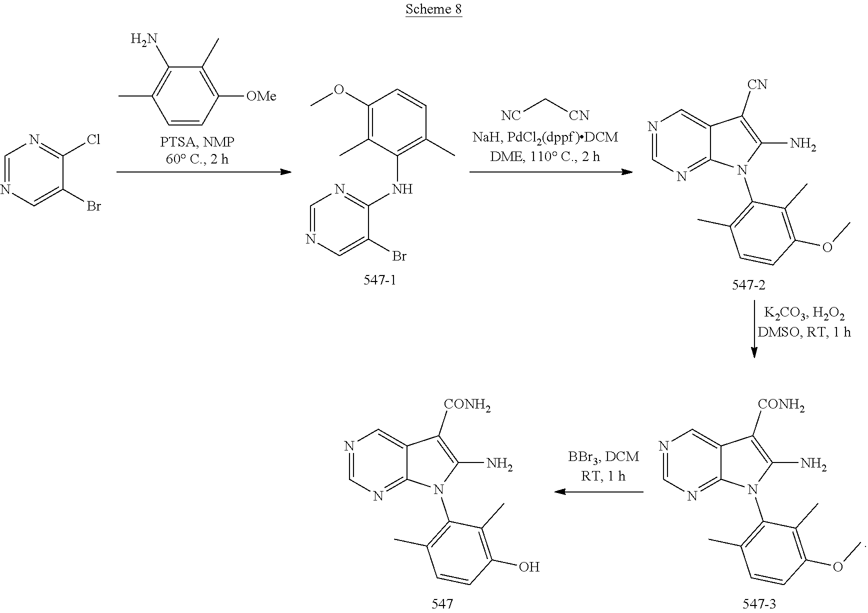

### Step 1: Synthesis of 5-bromo-N-(3-methoxy-2, 6-dimethylphenyl) pyrimidin-4-amine

To a stirred solution of 5-bromo-4-chloropyrimidine (1 g, 5.16 mmol, 1.0 eq) and 3-methoxy-2,6-dimethylaniline (0.781 g, 5.16 mmol, 1.0 eq) in NMP (10 mL). PTSA (2.95 g, 15.51 mmol, 3.0 eq) was added to the reaction and heated at 60° C. for 2 hours. After completion of the reaction as indicated by TLC, the reaction mixture was quenched with saturated NaHCO₃ (200 mL) and extracted with ethyl acetate (3×100 mL). The combined organic layer was washed with water, dried over Na₂SO₄ and concentrated under vacuum. The crude compound was purified by column chromatography (230-400 mesh silica gel, 30% EtOAc/hexane) to afford the title compound as a yellow solid (0.6 g, 7.53%). LCMS: RT=1.826 min, m/z 308 (M+H)⁺; ¹H NMR: (DMSO-d₆, 400 MHz): δ 8.69 (bs, 1H), 8.45 (s, 1H), 8.28 (s, 1H), 7.08 (d, J=8 Hz, 1H), 6.87 (d, J=8.4 Hz, 1H), 3.78 (s, 3H), 2.02 (s, 3H), 1.92 (s, 3H).

### Step 2: Synthesis of 6-amino-7-(3-methoxy-2,6-dimethylphenyl)-7H-pyrrolo[2,3-d]pyrimidine-5-carbonitrile

To a stirred solution of malononitrile (0.257 g, 3.89 mmol, 2.0 eq) in DME (6 mL). NaH (60% in mineral oil) (0.155 g, 3.89 mmol, 2.0 eq) was added in to the reaction mixture and stirred at room temperature for 10 min. 5-bromo-N-(3-methoxy-2,6-dimethylphenyl) pyrimidin-4-amine (0.6 g, 1.94 mmol, 1.0 eq) and PdCl₂(dppf)·DCM (0.079 g, 0.097 mmol, 0.05 eq) were added and the reaction was purge with argon for 5 min. The reaction was stirred at 110° C. for 2 hours. After completion of reaction as indicated by TLC, the reaction mixture was diluted with water (30 mL) and extraction was carried out using ethyl acetate (3×40 mL). The combined organic layer was washed with water, dried over Na₂SO₄ and concentrated under vacuum. The crude was purified by reverse phase column chromatography (C18 silica gel, 30% ACN in water) to afford the title compound as a pale-yellow solid (0.5 g, 87.55%). LCMS: RT=1.55 min, m/z 294.11 (M+H)⁺; ¹H NMR (DMSO-d₆, 400 MHz): δ 8.60 (bs, 1H), 8.46 (bs, 1H), 7.47 (bs, 2H), 7.26 (d, J=8.4 Hz, 1H), 7.12 (q, J=8.4 Hz, 1H), 3.84 (s, 3H), 1.79 (s, 3H), 1.70 (s, 3H).

### Step 3: Synthesis of 6-amino-7-(3-methoxy-2,6-dimethylphenyl)-7H-pyrrolo[2,3-d]pyrimidine-5-carboxamide

To a stirred solution of 6-amino-7-(3-methoxy-2,6-dimethylphenyl)-7H-pyrrolo[2,3-d]pyrimidine-5-carbonitrile (0.3 g, 1.02 mmol, 1.0 eq) in DMSO (10 mL), K₂CO₃ (0.848 mL, 6.13 mmol, 6.0 eq) was added at 0° C. and the reaction was stirred for 5 min. Then H₂O₂ (30%) (2.49 mL, 8.3 V) was added dropwise at 0° C. and the reaction was stirred at room temperature for 1 hour. After completion of the reaction as indicated by TLC, the reaction mixture was diluted with water (80 ml) and the resulting precipitate was filtered and concentrated under reduced pressure to afford the title compound as a pale-yellow solid (0.12 g, 37.69%). LCMS: RT=1.33 min, m/z 312.2 (M+H)⁺; ¹H NMR (DMSO-d₆, 400 MHz): δ 9.00 (s, 1H), 8.40 (s, 1H), 7.28-7.24 (m, 3H), 7.12 (d, J=8.4 Hz, 1H), 6.93 (bs, 2H), 3.85 (s, 3H), 1.87 (s, 3H), 1.69 (s, 3H).

### Step 4: Synthesis of 6-amino-7-(3-hydroxy-2, 6-dimethylphenyl)-7H-pyrrolo [2, 3-d]pyrimidine-5-carboxamide (Compound 547)

To a stirred solution of 6-amino-7-(3-methoxy-2, 6-dimethylphenyl)-7H-pyrrolo [2, 3-d]pyrimidine-5-carboxamide (0.120 g, 0.38 mmol, 1.0 eq) in DCM (1.5 mL), BBr3 (1M in DCM) (1.5 mL, 1.54 mmol, 4.0 eq) was added at 0° C. and the reaction mixture was stirred at room temperature for 1 hour. After completion of the reaction as indicated by TLC, the reaction mixture was quenched with MeOH (50 mL) and concentrated under vacuum. The crude product was purified by reverse phase column chromatography (C18 silica gel, 25% ACN in water) to afford the title compound as a pale-yellow solid (0.078 g, 68.07%). LCMS: RT=1.08 min, m/z 298.16 (M+H)⁺; ¹H NMR (DMSO-d₆, 400 MHz): δ 9.67 (bs, 1H), 8.99 (s, 1H), 8.40 (s, 1H), 7.20 (bs, 2H), 7.07 (d, J=7.6 Hz, 1H), 6.96-6.92 (m, 3H), 1.74 (s, 3H), 1.66 (s, 3H).

### Example 11. Synthesis of Compounds 553 and 556

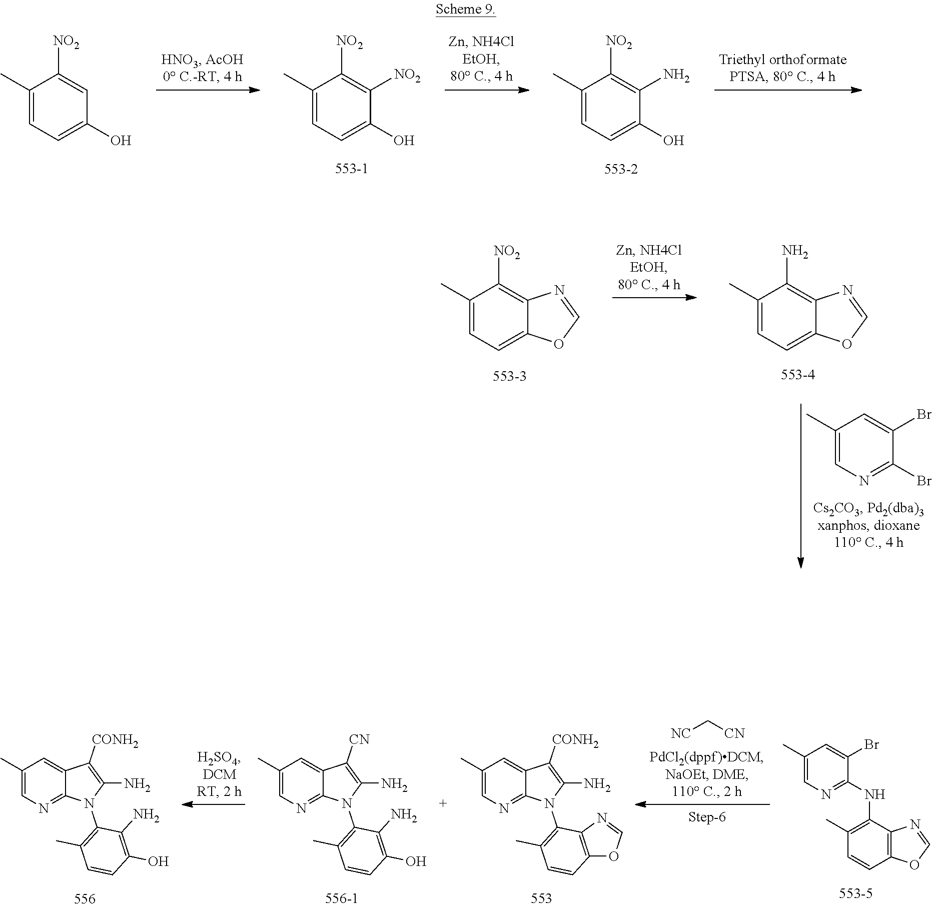

### Step 1: Synthesis of 4-methyl-2,3-dinitrophenol

To a stirred solution of 4-methyl-3-nitrophenol (10.0 g, 65.29 mmol, 1.0 eq) in AcOH (100 mL) at 10° C. HNO₃ (70%) (4.67 mL, 78.35 mmol, 1.2 eq) was added dropwise and the reaction mixture was stirred at room temperature for 4 hours. After completion of the reaction as indicated by TLC, the reaction mixture was quenched with ice water (200 mL). The resulting precipitate was collected by filtration. The crude product was purified by column chromatography (230-400 mesh silica gel, 0-20% EtOAc/hexane) to afford the title compound as a yellow solid (5.0 g, 38.65%). LCMS: RT=1.983 min, m/z 197.0 (M−H)⁺; ¹H NMR (DMSO-d6, 400 MHz): δ 11.92 (s, 1H), 7.56 (d, J=8.8 Hz, 1H), 7.30 (d, J=8.8 Hz, 1H), 2.30 (s, 3H).

### Step 2: Synthesis of 2-amino-4-methyl-3-nitrophenol

To a stirred solution of 4-methyl-2,3-dinitrophenol (1 g, 5.00 mmol, 1.0 eq) in EtOH (10 mL) at room temperature, Zn (1.62 g, 35.00 mmol, 7.0 eq) and NH₄Cl (1.38 g, 35.00 mmol, 7.0 eq) were added and the rection mixture was stirred at 80° C. for 4 hours. After completion of the reaction as indicated by TLC, the reaction mixture was filtered through Celite and washed with ethyl acetate (3×50 mL). The organic layer was concentrated under vacuum to afford the title compound as a brown solid (0.5 g, 58.92%). LCMS RT=1.70 min, m/z 169.05 (M+H)⁺; ¹H NMR (DMSO-d₆, 400 MHz): δ 10.04 (bs, 1H), 6.74 (d, J=7.6 Hz, 1H), 6.36 (d, J=8.0 Hz, 1H), 5.75 (bs, 2H), 2.24 (s, 3H).

### Step 3: Synthesis of 5-methylbenzo[d]oxazol-4-amine

To a stirred solution 2-amino-4-methyl-3-nitrophenol (3.0 g, 17.84 mmol, 1.0 eq) in triethyl orthoformate (30 mL), PTSA (1.53 g, 8.92 mmol, 0.5 eq) was added and the reaction mixture was stirred at 80° C. for 4 hours. After completion of the reaction as indicated by TLC, the reaction mixture was directly load to the column for purification (60-120 mesh silica gel, 0-30% EtOAc/hexane) to afford 5-methyl-4-nitrobenzo[d]oxazole as an off-white solid (1.5 g, 47.19%). LCMS: RT=1.744 min, m/z 179.0 (M+H)⁺.

To a stirred solution of 5-methyl-4-nitrobenzo[d]oxazole (3.0 g, 16.83 mmol, 1.0 eq) in EtOH (30 mL) at room temperature, Zn (7.70 g, 117.87 mmol, 7.0 eq) and NH₄Cl (6.30 g, 117.87 mmol, 7.0 eq) were added and the rection mixture was stirred at 80° C. for 4 hours. After completion of the reaction as indicated by TLC, the reaction mixture was filtered through Celite and wash with ethyl acetate (3×50 mL). The organic layer was concentrated under vacuum to afford the title compound as a brown solid (2.0 g, 80.16%). LCMS RT=1.573 min, m/z 149.0 (M+H)⁺; ¹H NMR: (DMSO-d₆, 400 MHz): δ 8.45 (s, 1H), 6.99 (d, J=8.0 Hz, 1H), 6.77 (d, J=8.0 Hz, 1H), 5.34 (bs, 2H), 2.16 (s, 3H).

### Step 4: Synthesis of N-(3-bromo-5-methylpyridin-2-yl)-5-methylbenzo[d]oxazol-4-amine

To a stirred solution of 2,3-dibromo-5-methylpyridine (0.50 g, 1.99 mmol, 1.0 eq), 5-methylbenzo[d]oxazol-4-amine (0.295 g, 1.99 mmol, 1.0 eq) and Cs₂CO₃ (1.29 g, 3.98 mmol, 2.0 eq) in dioxane (5 mL) were added. The reaction mixture was purged with N₂ gas for 5 min followed by the additional of Pd₂(dba)₃ (0.18 g, 0.199 mmol, 0.1 eq) and Xantphos (0.23 g, 0.39 mmol, 0.2 eq). The reaction mixture was stirred at 110° C. for 4 hours. After completion of the reaction as indicated by TLC, the reaction mixture was quenched with water (50 mL) and extracted with ethyl acetate (3×50 mL). The organic layer was dried over Na₂SO₄ and concentrated under vacuum. The crude product was purified by column chromatography (230-400 mesh silica gel, 0-20% EtOAc/hexane) to afford the title compound as an off-white solid (0.3 g, 47.32%). LCMS: RT=2.060 min, m/z 318.0 (M+H)⁺, ¹H NMR: (DMSO-d₆, 400 MHz): δ 8.56 (s, 1H), 7.77 (d, J=4.8 Hz, 2H), 7.71 (s, 1H), 7.52 (d, J=8.4 Hz, 1H), 7.31 (d, J=8.4 Hz, 1H), 2.24 (s, 3H), 2.14 (s, 3H).

### Step 5: Synthesis of 2-amino-5-methyl-1-(5-methylbenzo[d]oxazol-4-yl)-1H-pyrrolo[2,3-b]pyridine-3-carboxamide (Compound 553)

To a stirred solution of malononitrile (0.124 g, 1.88 mmol, 2.0 eq) and NaOEt (21% in EtOH) (3 mL) in DME (3 mL), N-(3-bromo-5-methylpyridin-2-yl)-5-methylbenzo[d]oxazol-4-amine (0.3 g, 0.94 mmol, 1.0) and PdCl₂(dppf)·DCM (0.153 g, 0.188 mmol, 0.1 eq) were added and the reaction mixture was stirred at 110° C. for 2 hours. After completion of the reaction as indicated by TLC, the reaction mixture was quenched with water and extracted with ethyl acetate (3×30 mL). The organic layer was dried over Na₂SO₄ and concentrated under vacuum. The crude was purified by PREP HPLC (CHIRALPAK, LIQ. CO₂/MEOH) to afford two compounds.

Compound 2-amino-5-methyl-1-(5-methylbenzo[d]oxazol-4-yl)-1H-pyrrolo[2,3-b]pyridine-3-carboxamide (Compound 553): As an off white solid (0.008 g, 2.64%). LCMS: RT=1.672 min, m/z 322.16 (M+H)⁺; ¹H NMR (DMSO-d₆, 400 MHz): δ 9.30-8.84 (bs, 2H), 7.91 (s, 1H), 7.65 (s, 1H), 7.40 (s, 1H), 7.14 (q, J=8.4 Hz, 1H), 7.02 (t, J=7.6 Hz, 2H), 6.75 (bs, 1H), 2.30 (s, 3H), 1.71 (d, J=12 Hz, 3H).

2-Amino-1-(2-amino-3-hydroxy-6-methylphenyl)-5-methyl-1H-pyrrolo[2,3-b]pyridine-3-carbonitrile: As an off white solid (0.010 g, 3.62%). LCMS: RT=1.755 min, m/z 294.2 (M+H)⁺; ¹H NMR (DMSO-d₆, 400 MHz): δ 9.30 (s, 1H), 7.63 (s, 1H), 7.39 (s, 1H), 6.83 (s, 2H), 6.72 (d, J=8.0 Hz, 1H), 6.41 (d, J=8.0 Hz, 1H), 4.15 (s, 2H), 2.33 (s, 3H), 1.62 (s, 3H).

### Step 6: Synthesis of 2-amino-1-(2-amino-3-hydroxy-6-methylphenyl)-5-methyl-1H-pyrrolo[2,3-b]pyridine-3-carboxamide (Compound 556)

To a stirred solution of 2-amino-1-(2-amino-3-hydroxy-6-methylphenyl)-5-methyl-1H-pyrrolo[2,3-b]pyridine-3-carbonitrile (0.01 g, 0.034 mmol, 1.0 eq) in DCM (0.5 mL) at room temperature, H₂SO₄ (0.1 mL) was added and the reaction mixture was stirred for 2 hours. After completion of the reaction as indicated by TLC, the reaction mixture was quenched with aq. NaHCO₃ and extracted with dichloromethane (3×5 mL). The organic layer was dried over Na₂SO₄ and concentrated under vacuum. The crude was purified by column chromatography (230-400 mesh silica gel, 0-10% MeOH/DCM) to afford the title compound as an off-white solid (0.002 g, 18.84%). LCMS: RT=1.320 min, m/z 312.2 (M+H)⁺; ¹H NMR (DMSO-d₆, 400 MHz): δ 9.31 (s, 1H), 7.86 (s, 1H), 7.59 (s, 1H), 6.80-6.67 (m, 5H), 6.44 (d, J=8.0 Hz, 1H), 4.01 (s, 2H), 2.31 (s, 3H), 1.64 (s, 3H).

### Example 12. Synthesis of Compound 552

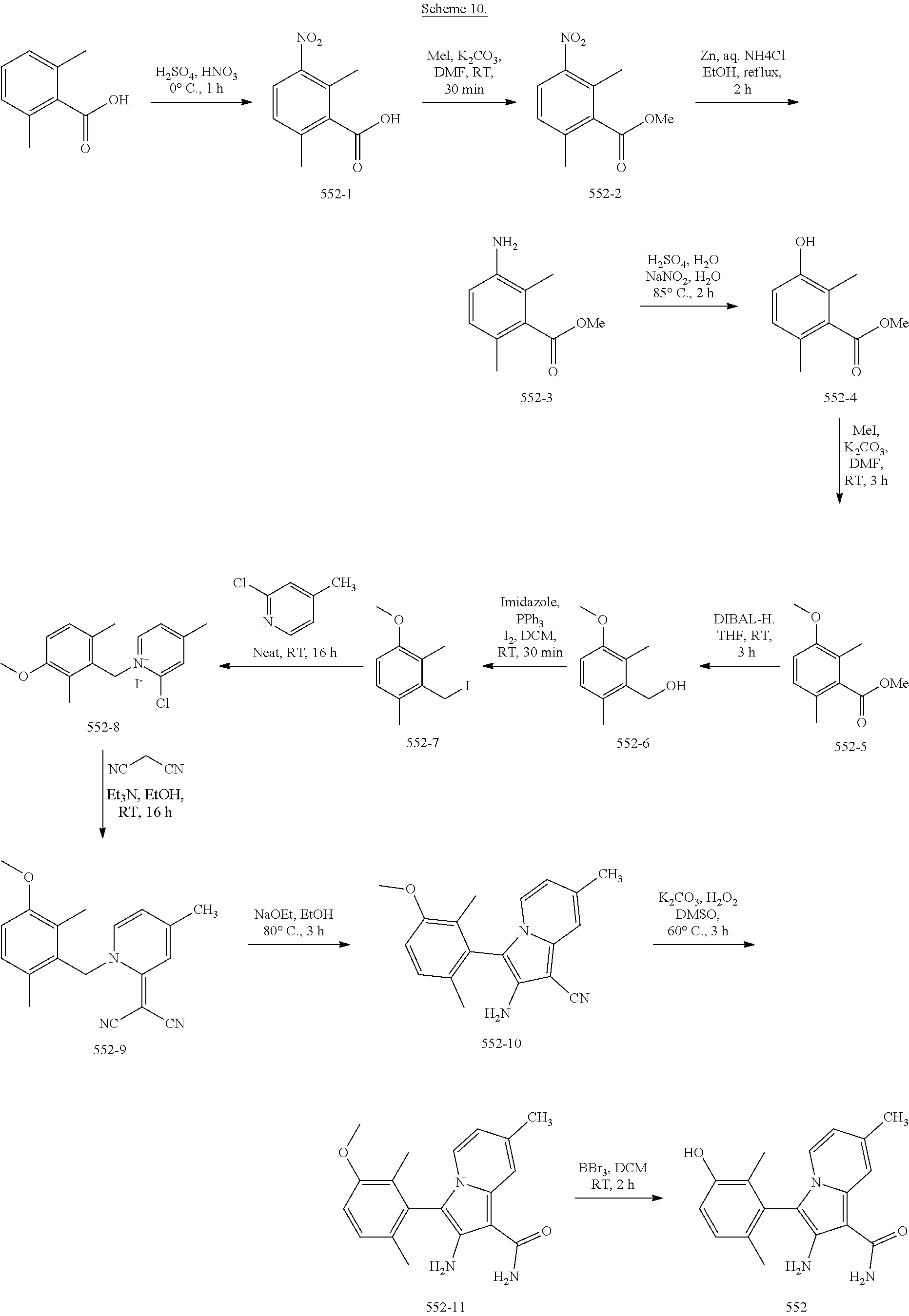

### Step 1: Synthesis of 2,6-dimethyl-3-nitrobenzoic acid

To a stirred solution of 2,6-dimethylbenzoic acid (40 g, 266.36 mmol, 1.0 eq) in H₂SO₄ (280 mL), HNO₃ (70%) (15.3 ml, 266.34 mmol, 1.0 eq) was added and the reaction was stirred at 0° C. for 1 hour. After completion of the reaction as indicated by TLC, the reaction mixture was poured into water (300 mL) and extracted with ethyl acetate (3×500 mL) The organic layer was dried over Na₂SO₄ and concentrated under vacuum to afford crude title compound as a light pink solid (62 g) (crude). LCMS: RT=1.237 min, m/z=mass not sported. ¹H NMR (DMSO-d₆, 400 MHz): δ 7.89 (d, J 8.4 Hz, 1H), 7.37 (d, J=8.4 Hz, 2.39 (s, 3H), 2.35 (s, 3H).

### Step 2: Synthesis of methyl 2,6-dimethyl-3-nitrobenzoate

To a stirred solution of 2,6-dimethyl-3-nitrobenzoic acid (54 g, 276.68 mmol, 1.0 eq) in DMF (500 mL) at 0° C., K₂CO₃ (152.94 g, 1106.72 mmol, 4.0 eq) was added. Iodomethane (117.8 g, 830.98 mmol, 3.0 eq) was added to the reaction mixture dropwise at 0° C. and the reaction was stirred at room temperature for 30 min. After completion of the reaction as indicated by TLC, the reaction mixture was quenched with water (500 mL) and extracted with diethyl ether (3×500 mL). The combined organic layer was washed with water, dried over Na₂SO₄ and concentrated under vacuum afford the title compound as a black liquid (50.0 g, 86.38%). LCMS: RT=2.210 min, mass not sported. ¹H NMR (DMSO-d₆, 400 MHz): δ 7.96 (d, J 6.8 Hz, 1H), 7.40 (d, J=8.4 Hz, 1H), 3.91 (s, 3H), 2.34 (s, 3H), 2.31 (s, 3H).

### Step 3: Synthesis of methyl 3-amino-2,6-dimethylbenzoate

To a stirred solution of 2,6-dimethyl-3-nitrobenzoic acid (49.5 g, 236.61 mmol, 1.0 eq) in ethanol:water (8:2 V), NH₄Cl (63.28 g, 1183.07 mmol, 5.0 eq) and Zn (77.34 g, 1183.07 mmol, 5.0 eq) were added and the reaction was heated to 80° C. for 2 hours. After completion of the reaction as indicated by TLC, the reaction mixture was filtered through Celite and washed with ethyl acetate (3×300 ml). The filtrate was concentrated. The crude product was diluted with water (300 mL) and extracted with ethyl acetate (3×300 mL). The organic layer was dried over Na₂SO₄ and concentrated under vacuum. The crude product was purified by column chromatography (230-400 mesh silica gel, 25% EtOAc/hexane) to afford the title compound as a brown solid (26.0 g, 61.31%). LCMS: RT=1.258 min, m/z 180.0 [M+H]⁺; ¹H NMR (DMSO-d₆, 400 MHz): δ 6.78 (d, J=8 Hz, 1H), 6.61 (d, J=8 Hz, 1H), 4.85 (bs, 2H), 3.81 (s, 3H), 2.04 (s, 3H), 1.93 (s, 3H).

### Step 4: Synthesis of methyl 3-hydroxy-2,6-dimethylbenzoate

To a stirred methyl 3-amino-2,6-dimethylbenzoate (24.0 g, 133.91 mmol, 1.0 eq) in H₂SO₄ (40 mL) at 0° C., a solution of NaNO₂ (9.23 g, 133.91 mmol, 1.0 eq) in water (20 V) was added dropwise. The reaction mixture was stirred at 0° C. for 1 hour. This reaction mixture was added dropwise to previously prepared H₂SO₄ (154 mL) and water (10 V). The reaction mixture was stirred at 85° C. for 2 hours. After completion of the reaction as indicated by TLC, the reaction mixture was quenched with water (200 mL) and extracted with ethyl acetate (3×800 mL). The combined organic layers were dried over Na₂SO₄ and concentrated under vacuum to afforded the title compound as an off-white solid (22.0 g, 91.17%). LCMS: RT=1.803 min, m/z 181.0 ¹H NMR (DMSO-d₆, 400 MHz): δ 9.43 (s, 1H), 6.87 (d, J=8.0 Hz, 1H), 6.76 (d, J=8.4 Hz, 1H), 3.82 (s, 3H), 2.08 (s, 3H), 1.99 (s, 3H).

### Step 5: Synthesis of methyl 3-methoxy-2,6-dimethylbenzoate

To a stirred solution of 3-hydroxy-2,6-dimethylbenzoate (21.5 g, 119.31 mmol, 1.0 eq) in DMF (200 mL) at 0° C., K₂CO₃ (65.95 g, 477.24 mmol, 4.0 eq) was added. Iodomethane (50.80 g, 357.93 mmol, 3.0 eq) was added to the reaction mixture dropwise at 0° C. and the reaction was stirred stir at room temperature for 3 hours. After completion of the reaction as indicated by TLC, the reaction mixture was quenched with water (500 mL) and extracted with diethyl ether (3×500 mL). The combined organic phase was dried over Na₂SO₄, filtered, and concentrated under reduced pressure. The resulting crude material was purified by flash chromatography (230-400 mesh silica gel, 15% ethyl acetate in hexane) to afford the title compound as a brown solid (8.0 g, 34.52%). LCMS: RT=2.252 min, m/z 195.1 [M+H]+; ¹H NMR (DMSO-d₆, 400 MHz): δ 7.06 (d, J=8.4 Hz, 1H), 6.93 (d, J=8.4 Hz, 1H), 3.83 (s, 3H), 3.77 (s, 3H), 2.14 (s, 3H) 2.03 (s, 3H).

### Step 6: Synthesis of (3-methoxy-2,6-dimethylphenyl)methanol

To a stirred solution of 3-methoxy-2,6-dimethylbenzoate (10.0 g, 51.48 mmol, 1.0 eq) in THF (100 ml) was added DIBAL-H (1.0 M in THF) (257.42 ml, 257.42 mmol, 5.0 eq) at −78° C. under a nitrogen atmosphere. The reaction mixture was stirred at room temperature for 3 hours. After completion of the reaction as indicated by TLC, the reaction mixture was poured into saturated NH₄Cl (300 ml) and extracted with ethyl acetate (2×500 ml). The combined organic layer was dried over Na₂SO₄ and concentrated under vacuum to afforded the title compound as an off-white solid (8.5 g, 99.32%). LCMS: RT=1.802 min, Mass Not supported; ¹H NMR (DMSO-d₆, 400 MHz): δ 6.95 (d, J=8.4 Hz, 1H), 6.76 (d, J=8.4 Hz, 1H), 4.70 (t, J=4.8 Hz, 1H), 4.46 (d, J=5.2 Hz, 2H), 3.73 (s, 3H), 2.28 (s, 3H), 2.19 (s, 3H).

### Step 7: Synthesis of 2-(iodomethyl)-4-methoxy-1,3-dimethylbenzene

To stirred a solution of triphenyl phosphine (22.45 g, 68.82 mmol, 1.3 eq), imidazole (4.68 g, 68.82 mmol, 1.3 eq) and iodine (8.73 g, 68.79 mmol, 1.3 eq) in DCM (500 ml) at room temperature for 10 min followed by (3-methoxy-2,6-dimethylphenyl)methanol (8.8 g, 52.94 mmol, 1.0 eq). The reaction mixture was stirred at room temperature for 30 min. After completion of the reaction as indicated by TLC, the reaction mixture was filtered and the filtrate was washed by sodium thiosulfate solution (500 ml×1). The combined organic layers were dried over Na₂SO₄ and concentrated under vacuum. The crude product was purified by flash chromatography (230-400 mesh silica gel, 10% ethyl acetate in hexane) to afford the title compound as a brown solid (8.0 g, 54.73%). LCMS: RT=2.769 min, Mass Not Sported; ¹H NMR (DMSO-d₆, 400 MHz): δ 6.97 (d, J=8.4 Hz, 1H), 6.80 (d, J=8.4 Hz, 1H), 4.51 (s, 2H), 3.66 (s, 3H), 2.20 (s, 3H), 2.11 (s, 3H).

### Step 8: Synthesis of 2-chloro-1-(3-methoxy-2,6-dimethylbenzyl)-4-methylpyridin-1-ium iodide

To stirred a solution of 2-(iodomethyl)-4-methoxy-1,3-dimethylbenzene (3.0 g, 10.86 mmol, 1.0 eq) was added 2-chloro-4-methylpyridine (2.07 g, 16.29 mmol, 1.5 eq) at room temperature. The reaction mixture was stirred at room temperature for 16 hours. After completion of the reaction as indicated by TLC, the reaction was trituration with diethyl ether to afford the title compound as a yellow solid (2.9 g, 96.44%). LCMS: RT=1.412 min, m/z 276 [M+H]+; ¹H NMR (DMSO-d₆, 400 MHz): δ 8.39 (s, 1H), 8.03 (d, J=6.4 Hz, 1H), 7.77 (dd, J=1.2 Hz, 6.4 Hz, 1H), 7.22 (d, J=8.4 Hz, 1H), 7.09 (d, J=8.4 Hz, 1H), 5.77 (s, 2H), 3.81 (s, 3H), 2.60 (s, 3H), 2.15 (s, 3H), 2.07 (s, 3H).

### Step 9: Synthesis of 2-(1-(3-methoxy-2,6-dimethylbenzyl)-4-methylpyridin-2(1H)-ylidene)malononitrile

To a stirred solution of 2-chloro-1-(3-methoxy-2,6-dimethylbenzyl)-4-methylpyridin-1-ium iodide (3.5 g, 12.64 mmol, 1.0 eq) in EtOH (10 V), Et3N (1.48 g, 14.66 mmol, 1.16 eq) and malononitrile (0.968 g, 14.66 mmol, 1.16 eq) were added at room temperature. The reaction mixture was stirred at room temperature for 16 hours. After completion of the reaction as indicated by TLC, the resulting reaction mixture was concentrated under reduced pressure. The crude product was diluted with water (80 ml) and the resulting precipitate was filtered and concentrated under reduced pressure. The crude product was purified by flash chromatography (230-400 mesh silica gel, 100% ethyl acetate in hexane) to afford the title compound as a yellow solid (2.4 g, 62.15%). LCMS: RT=2.351 min, m/z 306.1[M+H]⁺; ¹H NMR (DMSO-d₆, 400 MHz): δ 7.16 (t, J=8.4 Hz, 2H), 7.03 (t, J=6.8 Hz, 2H), 6.65 (d, J=7.2 Hz, 1H), 5.36 (s, 2H), 3.79 (s, 3H), 2.33 (s, 3H), 2.11 (s, 3H), 2.01 (s, 3H).

### Step 10: Synthesis of 2-amino-3-(3-methoxy-2,6-dimethylphenyl)-7-methylindolizine-1-carbonitrile

To a stirred solution of 2-(1-(3-methoxy-2,6-dimethylbenzyl)-4-methylpyridin-2(1H)-ylidene)malononitrile (0.8 g, 2.61 mmol, 1.0 eq) in ethanol (10 V), sodium ethoxide (21% solution in ethanol) (33.95 g, 104.78 mmol, 40.0 eq) was added at room temperature. The reaction mixture was stirred at 80° C. for 3 hours. After completion of the reaction as indicated by TLC, the resulting reaction mixture was concentrated under reduced pressure. The crude product was purified by column chromatography (230-400 mesh silica gel, 0-30% EtOAc/hexane) to afford the title compound as a yellow solid (0.69 g, 86.25) LCMS: RT=2.636 min, m/z 306.2[M+H]⁺; ¹H NMR (DMSO-d₆, 400 MHz): δ 7.22-7.15 (m, 3H), 7.02 (d, J=8.4 Hz, 1H), 6.55 (d, J=7.2 Hz, 1H), 4.75 (bs, 2H), 3.81 (s, 3H), 2.30 (s, 3H), 1.87 (s, 3H), 1.79 (s, 3H).

### Step 11: Synthesis of 2-amino-3-(3-methoxy-2,6-dimethylphenyl)-7-methylindolizine-1-carboxamide

To a stirred solution of 2-amino-3-(3-methoxy-2,6-dimethylphenyl)-7-methylindolizine-1-carbonitrile (0.68 g, 2.22 mmol, 1.0 eq) in DMSO (10 V), K₂CO₃ (3.07 g, 22.26 mmol, 10.0 eq) and H₂O₂ (30% w/v solution in water) (6.8 ml, 10 Vol) were added at room temperature. The reaction mixture was stirred at 60° C. for 3 hours. After completion of the reaction as indicated by TLC, the reaction mixture was diluted with ice cooled water (30 mL) and the resulting precipitate was filtered and dried under reduced pressure to afford the title compound as a yellow solid (0.25 g, 34.72%). LCMS: RT=2.142 min, m/z 324.3[M+H]⁺; ¹H NMR (DMSO-d₆, 400 MHz): δ 7.64 (s, 1H), 7.28-7.19 (m, 1H), 7.10 (d, J=6.8 Hz, 1H), 7.01 (d, J=8.4 Hz, 1H), 6.83 (bs, 2H), 6.45 (d, J=6.8 Hz, 1H), 4.88 (bs, 2H), 3.81 (s, 3H), 2.30 (s, 3H), 1.87 (s, 3H), 1.73 (s, 3H).

### Step 12: Synthesis of 2-amino-3-(3-hydroxy-2,6-dimethylphenyl)-7-methylindolizine-1-carboxamide (Compound 552)

To a stirred solution of 2-amino-3-(3-methoxy-2,6-dimethylphenyl)-7-methylindolizine-1-carboxamide (0.25 g, 0.773 mmol, 1.0 eq) in DCM (5 mL) at −78° C., BBr₃ (1M in DCM) (2.31 ml, 2.31 mmol, 3.0 eq) was added dropwise and the reaction mixture was stirred at room temperature for 2 hours. After completion of the reaction as indicated by TLC, the reaction mixture was quenched with 10% NaHCO₃ solution (30 mL) and extracted with DCM (3×30 mL). The combined organic layers were washed with water, dried over Na₂SO₄, and concentrated under vacuum. The crude product was purified by reverse phase column chromatography (18C silica gel, 0-98% ACN)/0.1% FA in water) to afford the title compound as a light gray solid (0.080 g, 33.45%). LCMS: RT=2.27 min, m/z 310.3 [M+H]⁺; ¹H NMR (DMSO-d₆, 400 MHz): δ 9.46 (bs, 1H), 7.43 (s, 1H), 7.76 (s, 1H), 7.10 (d, J=6.4 Hz, 1H), 7.01 (d, J=8.4 Hz, 1H), 6.87 (d, J=8.0 Hz, 1H), 6.81 (s, 2H), 6.45 (d, J=6.8 Hz, 1H), 4.81 (bs, 2H), 2.29 (s, 3H), 1.82 (s, 3H), 1.74 (s, 3H).

### Example 13. Synthesis of Compounds 559, 585, and 588

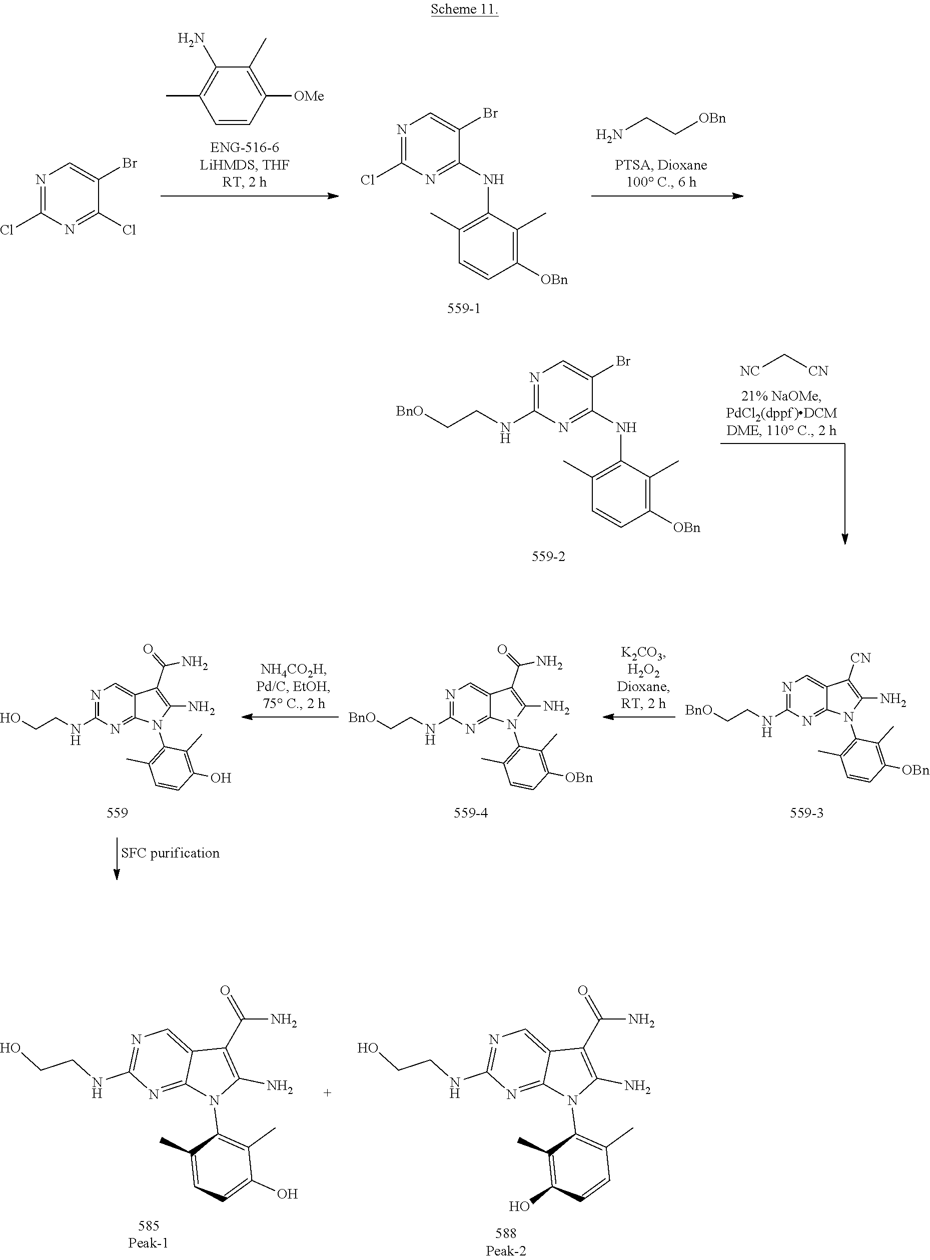

### Step 1: Synthesis of N-(3-(benzyloxy)-2, 6-dimethylphenyl)-5-bromo-2-chloropyrimidin-4-amine

To a stirred solution of 5-bromo-2, 4-dichloropyrimidine (10 g, 43.88 mmol, 1.0 eq) and 3-(benzyloxy)-2, 6-dimethylaniline (6.78 g, 29.84 mmol, 0.68 eq) in THF (100 mL) at 0° C., LiHMDS (1M in THF) (39.49 mL, 39.49 mmol, 0.9 eq) was added and the reaction was stirred at room temperature for 2 hours. After completion of the reaction as indicated by TLC, the reaction mixture was quenched with water (300 mL) and extraction with ethyl acetate (3×250 mL). The organic layer was dried over Na₂SO₄, filtered and concentrated in vacuum to afforded the crude product. The crude compound was purified by column chromatography using (230-300 mesh silica gel, 12% EtOAc/hexane) to afford the title compound as a brown solid (6.0 g, 32.65%). LCMS: RT=2.777 min, m/z=420.28 [M+H]⁺; ¹H NMR (400 MHz, DMSO-d₆): δ 9.13 (s, 1H), 8.40 (s, 1H), 7.47 (d, J=7.2 Hz, 2H), 7.40 (t, J=7.2 Hz, 2H), 7.33 (t, J=7.2 Hz, 1H), 7.10 (d, J=8.4 Hz, 1H), 6.99 (d, J=8.4 Hz, 1H), 5.13 (s, 2H), 2.03 (s, 3H), 1.99 (s, 3H).

### Step 2: Synthesis of N4-(3-(benzyloxy)-2,6-dimethylphenyl)-N2-(2-(benzyloxy)ethyl)-5-bromopyrimidine-2,4-diamine

To a stirred solution of N-(3-(benzyloxy)-2,6-dimethylphenyl)-5-bromo-2-chloropyrimidin-4-amine (1.5 g, 3.58 mmol, 1.0 eq) and 2-(benzyloxy)ethan-1-amine (0.541 g, 3.58 mmol, 1.0 eq) in dioxane (15 mL), PTSA (0.068 g, 0.358 mmol, 0.1 eq) was added and the reaction was stirred at 100° C. for 6 hours. After completion of the reaction as indicated by TLC, saturated NaHCO₃ solution (300 mL) was added and the mixture was extracted with ethyl acetate (3×250 mL). The organic layer was dried over Na₂SO₄, filtered, and concentrated in vacuum to obtain the crude compound. The crude compound was purified by column chromatography using (230-300 mesh silica gel, 31% EtOAc/hexane) to afford the title compound as a white solid (3.0 g, 39.24%). LCMS: R_{T}=2.339 min, m/z=533.2 [M+H]⁺; ¹H NMR: (400 MHz, DMSO-d₆): δ 8.10 (bs, 1H), 7.94 (s, 1H), 7.43-7.36 (m, 4H), 7.33-7.24 (m, 6H), 7.02 (d, J=8.4 Hz, 1H), 6.88 (d, J=8.4 Hz, 1H), 6.66 (bs, 1H), 5.07 (bs, 2H), 4.505-4.35 (m, 2H), 3.35 (s, 3H), 3.17 (d, J=5.2 Hz, 1H), 2.04 (s, 3H), 2.01 (s, 3H).

### Step 3: Synthesis of 6-amino-7-(3-(benzyloxy)-2, 6-dimethylphenyl)-2-((2-(benzyloxy) ethyl) amino)-7H-pyrrolo [2, 3-d]pyrimidine-5-carbonitrile

To stirred solution of malononitrile (0.148 g, 2.24 mmol, 2.0 eq) in DME (6 mL), NaOEt (21% in EtOH) (6.14 ml, 19.04 mmol, 17.0 eq) was added and the reaction mixture was stirred for 10 min. N4-(3-(benzyloxy)-2,6-dimethylphenyl)-N2-(2-(benzyloxy)ethyl)-5-bromopyrimidine-2,4-diamine (0.6 g, 1.12 mmol, 1.0 eq) and PdCl₂(dppf).DCM (0.045 g, 0.056 mmol, 0.05 eq) were added and the mixture was degassed with argon for 5 min. The reaction mixture was stirred at 110° C. for 2 hours. After completion of the reaction as indicated by TLC, the reaction mixture was quenched with water (30 mL) and extracted with ethyl acetate (3×40 mL). The organic layer dried over Na₂SO₄, filtered, and concentrated in vacuum to afforded the crude product. The crude compound was purified by column chromatography using (230-300 mesh silica gel, 31% EtOAc/hexane) to afford the title compound as an off-white solid (1.5 g, 51.43%). LCMS: R_{T}=2.152 m/z, 519.5 [M+H]+; ¹H NMR: (400 MHz, DMSO-d₆): δ 8.14 (s, 1H), 7.48 (s, J=7.2 Hz, 1H), 7.41 (t, J=7.2 Hz, 2H), 7.35 (d, J=7.2 Hz, 1H), 7.323-7.21 (m, 4H), 7.19 (s, 2H), 7.14 (d, J=8.4 Hz, 1H), 6.87 (s, 2H), 6.72 (t, J=5.6 Hz, 1H), 5.14 (s, 2H), 4.40 (s, 2H), 3.47 (t, J=6 Hz, 2H), 1.83 (s, 3H), 1.79 (s, 3H).

### Step 4: Synthesis of 6-amino-7-(3-hydroxy-2, 6-dimethylphenyl)-2-((2-hydroxyethyl)amino)-7H-pyrrolo[2,3-d]pyrimidine-5-carboxamide (Compound 559)

To a stirred solution of 6-amino-7-(3-(benzyloxy)-2,6-dimethylphenyl)-2-((2-(benzyloxy)ethyl)amino)-7H-pyrrolo[2,3-d]pyrimidine-5-carbonitrile (1.4 g, 2.69 mmol, 1.0 eq) in DMSO (15 mL), K₂CO₃ (3.73 g, 26.99 mmol, 10.0 eq) was added and the reaction mixture was stirred at 0° C. for 5 min. H₂O₂ (30%) (2.75 mL, 26.99 mmol, 10.0 eq) was added dropwise and the reaction to stirred at room temperature for 2 hours. After completion of the reaction as indicated by TLC, the reaction mixture was diluted with water (30 mL) and extracted with ethyl acetate (3×50 mL). The organic layer dried over Na₂SO₄, filtered, and concentrated under vacuum to afforded the crude product. The crude compound was purified by column chromatography using (230-300 mesh silica gel, 5% ethyl MeOH/MDC) to afford 6-amino-7-(3-(benzyloxy)-2, 6-dimethylphenyl)-2-((2-(benzyloxy) ethyl) amino)-7H-pyrrolo [2, 3-d]pyrimidine-5-carboxamide as an off-white solid (0.2 g, 13.81%). LCMS: R_{T}=2.021 m/z, 537.5 [M+H]⁺.

To a stirred solution of 6-amino-7-(3-(benzyloxy)-2, 6-dimethylphenyl)-2-((2-(benzyloxy) ethyl) amino)-7H-pyrrolo [2, 3-d]pyrimidine-5-carboxamide (0.20 g, 0.37 mmol, 1.0 eq) in EtOH (10 mL), 10% Pd/C (w/w) (0.2 g) and NH₄HCO₂ (0.822 g, 13.04 mmol, 35.0 eq) were added and the reaction mixture was stirred at 75° C. for 2 hours. The reaction mixture was filtered through Celite, washed with EtOH (100 ml), and concentrated under vacuum. The crude compound was purified by column chromatography (230-300 mesh silica gel, 7% MeOH/MDC) to afford the title compound as an off-white solid (0.050 g, 37.64%). LCMS: R_{T}=1.083 m/z 357.32 [M+H]+; 1H NMR: (400 MHz, DMSO-d₆): δ 9.50 (s, 1H), 8.53 (s, 1H), 7.04 (d, J=8 Hz, 1H), 6.89 (d, J=8.4 Hz, 1H), 6.70 (bs, 2H), 6.62 (bs, 2H), 6.40 (t, J=5.6 Hz, 1H), 4.61 (bs, 1H), 3.44 (d, J=4.8 Hz, 2H), 3.22 (q, J=6.0 Hz, 12 Hz, 2H), 1.79 (s, 3H), 1.71 (s, 3H).

### Step 5: Synthesis of 6-amino-7-(3-hydroxy-2,6-dimethylphenyl)-2-((2-hydroxyethyl)amino)-7H-pyrrolo[2,3-d]pyrimidine-5-carboxamide (Peak-1) (Compound 585) and 6-amino-7-(3-hydroxy-2,6-dimethylphenyl)-2-((2-hydroxyethyl)amino)-7H-pyrrolo[2,3-d]pyrimidine-5-carboxamide (Peak-2) (Compound 588)

Compound 559 was purified by SFC using (YMC CELLULOSE SC, 250×20 mm, 5 μm), MOBILE PHASE A: NH3 (7N in methanol) in HEPTANE MOBILE PHASE B: M·NH₃-MEOH-MTBE (50:50) to afford the title compounds as an off-white solid.

Compound 585 (Peak-1): 0.028 g, LCMS: RT=1.098 min, m/z=357.32 [M+H]⁺, HPLC: RT=3.51 min, Chiral HPLC: RT=7.724 min time, ¹H NMR (400 MHz, DMSO-d₆) δ 9.57 (s, 1H), 8.51 (s, 1H), 7.05 (d, J=8 Hz, 1H), 6.90 (d, J=8.4 Hz, 1H), 6.73 (bs, 2H), 3.44 (t, J=4.8 Hz, 2H), 3.22 (q, J=6.0 Hz, 12 Hz, 2H), 1.80 (s, 3H), 1.72 (s, 3H). (—OH and —NH proton not observed in NMR).

Compound 588 (Peak-2): 0.0121 g; LCMS: RT=1.153 min, m/z=357.37 [M+H]⁺; HPLC: RT=3.59 min; Chiral HPLC: RT=8.27 min time; ¹H NMR (400 MHz, DMSO-d₆) δ 9.55 (s, 1H), 8.53 (s, 1H), 7.04 (d, J=8.4 Hz, 1H), 6.89 (d, J=8.4 Hz, 1H), 6.71 (bs, 2H), 6.64 (bs, 2H), 6.42 (t, J=5.6 Hz, 1H), 4.62 (bs, 1H), 3.44-1.43 (m, 2H), 3.22 (q, J=6.0 Hz, 11.6 Hz, 2H), 1.80 (s, 3H), 1.71 (s, 3H).

### Example 14. Synthesis of Compound 565

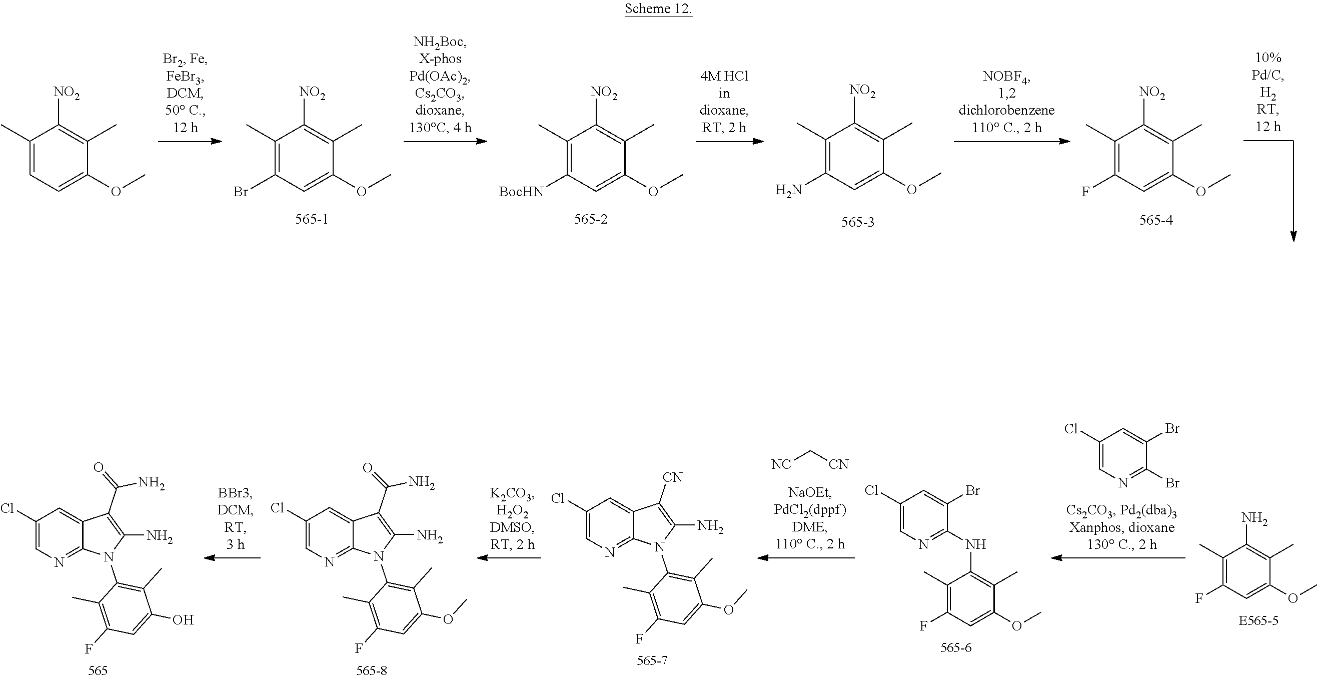

### Step 1: Synthesis of 1-bromo-5-methoxy-2,4-dimethyl-3-nitrobenzene

To a stirred solution of 1-methoxy-2,4-dimethyl-3-nitrobenzene (30.0 g, 165.57 mmol, 1.0 eq) in DCM (100 mL), Fe (2.77 g, 49.67 mmol, 0.3 eq) and FeBr₃ (0.978 g, 3.31 mmol, 0.02 eq) were added at 10° C. Next Br₂ (34.39 g, 215.24 mmol, 1.3eq) was added and the reaction mixture was stirred at 50° C. for 12 hours. After completion of the reaction as indicated by TLC, the reaction mixture was quenched with sat. Na₂S₂O₅ (200 mL) and filtered. The reaction was extracted with dichloromethane (3×200 mL). The combined organic layers were dried over Na₂SO₄ and concentrated under vacuum. The crude compound was purified by column chromatography (230-400 mesh silica gel, 0-20% EtOAc/hexane) to afford the title compound as a brown solid (10.0 g, 23.22%). LCMS: RT=2.753 min (mass not supported), ¹H NMR (DMSO-d₆, 400 MHz): δ 7.44 (s, 1H), 3.87 (s, 3H), 2.18 (s, 3H), 1.99 (s, 3H).

### Step 2: Synthesis of tert-butyl (5-methoxy-2,4-dimethyl-3-nitrophenyl)carbamate

To 1-bromo-5-methoxy-2,4-dimethyl-3-nitrobenzene (10 g, 38.44 mmol, 1.0 eq), was added tert-butyl carbamate (9.0 g, 76.89 mmol, 2.0 eq) and Cs₂CO₃ (25.05 g, 76.89 mmol, 2.0 eq) in dioxane (100 mL). The reaction mixture was purged with N₂ gas for 5 min. Pd(OAc)₂ (0.86 g, 3.84 mmol, 0.1 eq) and X-Phos (3.66 g, 7.68 mmol, 0.2 eq) were added and the reaction mixture was stirred at 130° C. for 4 hours. After completion of the reaction as indicated by TLC, the reaction mixture was quenched with water and extracted with ethyl acetate (3×200 mL). The combined organic layers were dried over Na₂SO₄ and concentrated under vacuum. The crude compound was purified by column chromatography (230-400 mesh silica gel, 0-30% EtOAc/hexane) to afford the title compound as an off-white solid (7.0 g, 61.44%). LCMS: RT=2.623 min (mass not supported), ¹H NMR (DMSO-d₆, 400 MHz): δ 8.91 (s, 1H), 7.17 (s, 1H), 3.81 (s, 3H), 1.99 (s, 3H), 1.97 (s, 3H), 1.46 (s, 9H).

### Step 3: Synthesis of 5-methoxy-2,4-dimethyl-3-nitroaniline

To a solution of tert-butyl (5-methoxy-2,4-dimethyl-3-nitrophenyl)carbamate (7.0 g, 23.62 mmol, 1.0 eq) was added 4M HCl in dioxane (70 mL) and the reaction was stirred at room temperature for 2 hours. After completion of the reaction as indicated by TLC, the reaction mixture was quenched with sat. NaHCO₃ (150 mL) and extracted by ethyl acetate (3×150 mL). The combined organic layers were dried over Na₂SO₄ and concentrated under vacuum to afford the title compound as an off white solid (4.0 g, 86.30%). LCMS: RT=2.050 min, ¹H NMR (DMSO-d₆, 400 MHz): δ 6.45 (s, 1H), 5.34 (bs, 2H), 3.73 (s, 3H), 1.85 (s, 3H), 1.83 (s, 3H).

### Step 4: Synthesis of 1-fluoro-5-methoxy-2,4-dimethyl-3-nitrobenzene

To a stirred solution of 5-methoxy-2,4-dimethyl-3-nitroaniline (4.0 g, 20.38 mmol, 1.0 eq) in 1,2 dichlorobenzene (40 mL), nitrosonium tetrafluoroborate (3.33 g, 38.54 mmol, 1.4 eq) was added and the reaction mixture was stirred at 110° C. for 2 hours. After completion of the reaction as indicated by TLC, the reaction mixture was directly load onto a column (60-120 mesh silica gel, 0-10% EtOAc/hexane) to afford the title compound as a light brown solid (3.0 g, 73.88%). ¹H NMR (DMSO-d₆, 400 MHz): δ 7.19 (d, J=11.6 Hz, 1H), 3.85 (s, 3H), 2.06 (s, 3H), 2.01 (s, 3H).

### Step 5: Synthesis of 3-bromo-5-chloro-N-(3-fluoro-5-methoxy-2,6-dimethylphenyl)pyridin-2-amine

To a stirred solution of 1-fluoro-5-methoxy-2,4-dimethyl-3-nitrobenzene (3.0 g, 15.06 mmol, 1.0 eq) in MeOH (30 mL) at room temperature, 10% Pd/C (w/w) was added and the reaction mixture was purged with H₂ gas. The reaction mixture was stirred at room temperature under H₂ atmosphere for 12 hours. After completion of the reaction as indicated by TLC, the reaction mixture was filtered through Celite and wash with MeOH (3×50 mL). The combined organic layers were dried over Na₂SO₄ and concentrated under vacuum. The crude compound was purified by column chromatography (230-400 mesh silica gel, 0-50% EtOAc/hexane) to afford 3-fluoro-5-methoxy-2,6-dimethylaniline as a brown solid (1.5 g, 58.86%). LCMS: RT=2.006 min, m/z 170.1 (M+H)⁺.

To a stirred mixture of 2,3-dibromo-5-chloropyridine (1.60 g, 5.91 mmol, 1.0 eq), 3-fluoro-5-methoxy-2,6-dimethylaniline (1.0 g, 5.91 mmol, 1.0 eq) and Cs₂CO₃ (3.85 g, 11.8 mmol, 2.0 eq) in dioxane (100 mL) was added. The reaction mixture was purged with N₂ gas for 5 min. Pd₂(dba)₃ (0.54 g, 0.59 mmol, 0.1 eq) and Xantphos (0.68 g, 1.18 mmol, 0.2 eq) were added and the reaction mixture was stirred at 130° C. for 2 hours. After completion of the reaction as indicated by TLC, the reaction mixture was quenched with water (100 mL) and extracted with ethyl acetate (3×100 mL). The combined organic layers were dried over Na₂SO₄ and concentrated under vacuum. The crude compound was purified by column chromatography (230-400 mesh silica gel, 0-50% EtOAc/hexane) to afford the title compound as an off-white solid (0.6 g, 28.23%). LCMS: RT=2.906 min, m/z 359.2 (M+H)⁺

### Step 6: Synthesis of 2-amino-5-chloro-1-(3-fluoro-5-methoxy-2,6-dimethylphenyl)-1H-pyrrolo[2,3-b]pyridine-3-carbonitrile

To a stirred solution of malononitrile (0.22 g, 3.33 mmol, 2.0 eq) in DME (6 mL) at 0° C., NaOEt (21% in EtOH) was added and the reaction was stirred for 10 min. 3-Bromo-5-chloro-N-(3-fluoro-5-methoxy-2,6-dimethylphenyl)pyridin-2-amine (0.6 g, 1.66 mmol, 1.0 eq) and PdCl₂(dppf) (0.123 g, 0.166 mmol, 0.1 eq) were added and the reaction mixture was stirred at 110° C. for 2 hours. After completion of the reaction as indicated by TLC, the reaction mixture was quenched with water (50 mL) and extracted with ethyl acetate (3×30 mL). The combined organic layers were dried over Na₂SO₄ and concentrated under vacuum. The crude compound was purified by column chromatography (230-400 mesh silica gel, 0-50% EtOAc/hexane) to afford the title compound as an off-white solid (0.3 g, 52.15%). LCMS: RT=2.555 min, m/z 345.21 (M+H)⁺; ¹H NMR (DMSO-d₆, 400 MHz): δ 7.82 (d, J=2.0 Hz, 1H), 7.70 (d, J=1.6 Hz, 1H), 7.42 (bs, 2H), 7.12 (d, J=11.6 Hz, 1H), 3.85 (s, 3H), 1.69 (s, 3H), 1.64 (s, 3H).

### Step 7: Synthesis of 2-amino-5-chloro-1-(3-fluoro-5-methoxy-2,6-dimethylphenyl)-1H-pyrrolo[2,3-b]pyridine-3-carboxamide

To a stirred solution of 2-amino-5-chloro-1-(3-fluoro-5-methoxy-2,6-dimethylphenyl)-1H-pyrrolo[2,3-b]pyridine-3-carbonitrile (0.3 g, 0.87 mmol, 1.0 eq) and K₂CO₃ (0.24 g, 1.74 mmol, 2.0 eq) in DMSO (3 mL), H₂O₂ (30%) (1 mL) was added and the reaction mixture was stirred at room temperature for 2 hours. After completion of the reaction as indicated by TLC, the reaction mixture was quenched with water (50 mL) and was extracted with ethyl acetate (3×30 mL). The combined organic layers were dried over Na₂SO₄ and concentrated under vacuum. The crude compound was purified by column chromatography (230-400 mesh silica gel, 0-5% MeOH/DCM) to afford the title compound as an off-white solid (0.15 g, 47.52%). LCMS: RT=2.184 min, m/z 363.17 (M+H)⁺; ¹H NMR (DMSO-d₆, 400 MHz): δ 8.16 (s, 1H), 7.76 (s, 1H), 7.28 (bs, 2H), 7.13 (d, J=11.6 Hz, 1H), 6.89 (bs, 2H), 3.86 (s, 3H), 1.69 (s, 3H), 1.64 (s, 3H).

### Step 8: Synthesis of 2-amino-5-chloro-1-(3-fluoro-5-hydroxy-2,6-dimethylphenyl)-1H-pyrrolo[2,3-b]pyridine-3-carboxamide (Compound 565)

To a stirred solution of 2-amino-5-chloro-1-(3-fluoro-5-methoxy-2,6-dimethylphenyl)-1H-pyrrolo[2,3-b]pyridine-3-carboxamide (0.15 g, 0.41 mmol, 1.0 eq) in DCM (1.5 mL) at 0° C., BBr₃ (1M in DCM) (1 mL) was added and the reaction mixture was stirred at room temperature for 3 hours. After completion of the reaction as indicated by TLC, the reaction mixture was quenched with aq. NaHCO₃ (30 mL) and extracted with DCM (3×20 mL). The combined organic layers were dried over Na₂SO₄ and concentrated under vacuum. The crude compound was purified by column chromatography (230-400 mesh silica gel, 0-10% MeOH/DCM) to afford the title compound as an off-white solid (0.035 g, 24.27%). LCMS: RT=1.879 min, m/z 349.2 (M−H)⁺; ¹H NMR (DMSO-d₆, 400 MHz): δ 10.05 (s, 1H), 8.15 (s, 1H), 7.76 (s, 1H), 7.26 (bs, 2H), 6.88 (bs, 2H), 6.80 (d, J=11.2 Hz, 1H), 1.65 (s, 3H), 1.61 (s, 3H).

### Example 15. Synthesis of Compound 568

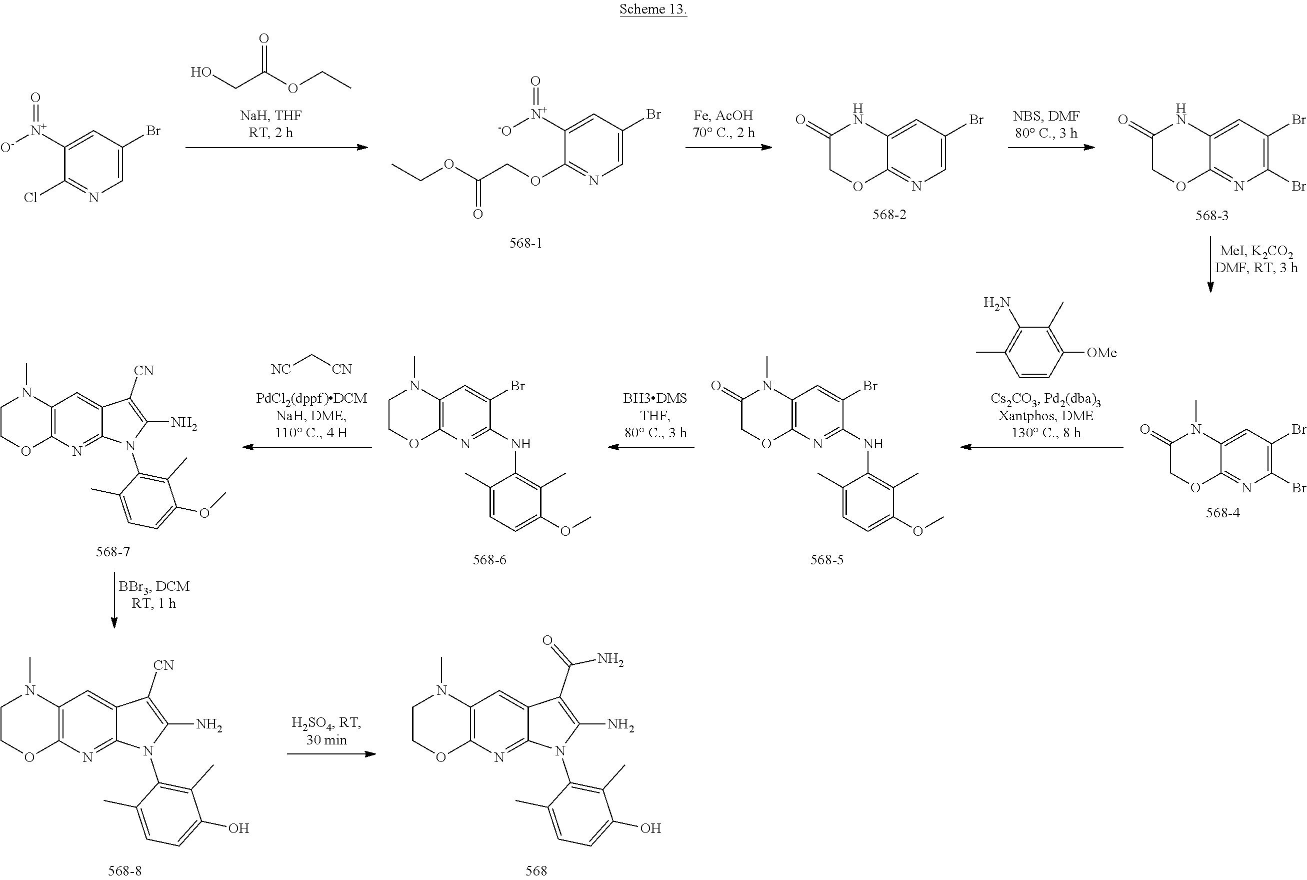

### Step 1: Synthesis of 7-bromo-1H-pyrido[2,3-b][1,4]oxazin-2(3H)-one

To a mixture of 5-bromo-2-chloro-3-nitropyridine (0.1 g, 0.42 mmol, 1.0 eq) and ethyl 2-hydroxyacetate (0.056 g, 0.54 mmol, 1.3 eq) in THF (2 mL) at 0° C., NaH (60% in mineral oil) (0.050 g, 1.26 mmol, 3.0 eq) was added portion wise and the reaction was stirred at room temperature for 2 hours. After completion of the reaction as indicated by TLC, the reaction mixture was quenched by water (20 ml) and extracted with ethyl acetate (3×20 mL). The combined organic layers were dried over Na₂SO₄ and concentrated under vacuum to afford ethyl 2-((5-bromo-3-nitropyridin-2-yl)oxy)acetate as a brown solid (0.12 g, 93.39%) (crude). LCMS: RT=2.248 min, m/z 305.51 [M+H]⁺.

To a solution of ethyl 2-((5-bromo-3-nitropyridin-2-yl)oxy)acetate (3.5 g, 11.47 mmol, 1.0 eq) in AcOH (35 mL), Fe (3.20 g, 57.36 mmol, 5.0 eq) was added and the reaction mixture was stirred at 70° C. for 2 hours. After completion of the reaction as indicated by TLC, the reaction mixture was filtered through Celite and washed with ethyl acetate (30 mL). The filtrate was concentrated under vacuum to afford the crude compound. The crude compound was purified by column chromatography (230-400 mesh silica gel, 0-70% EtOAc/hexane) to afford the title compound as a brown solid (1.23 g, 46.81%). LCMS: RT=1.293 min, m/z 229.00 [M+H]+; ¹H NMR (DMSO-d₆, 400 MHz): δ 10.95 (s, 1H), 7.88 (d, J=2 Hz, 1H), 7.33 (d, J=2 Hz, 1H), 4.81 (s, 2H).

### Step 2: Synthesis of 6,7-dibromo-1H-pyrido[2,3-b][1,4]oxazin-2(3H)-one

To a stirred solution of 7-bromo-1H-pyrido[2,3-b][1,4]oxazin-2(3H)-one (0.5 g, 2.18 mmol, 1.0 eq) in DMF (10 mL) at room temperature, N-bromosuccinimide (1.94 g, 10.91 mmol, 5.0 eq) was added and the reaction mixture was stirred at 80° C. for 3 hours. After completion of the reaction as indicated by TLC, the reaction mixture was poured in ice water (50 mL) and the resulting precipitate was filtered and dried under vacuum to afford the title compound as a light brown solid (0.37 g, 55.04%). LCMS: RT=1.689 min, m/z 306.91 [M+H]⁺; ¹H NMR (DMSO-d₆, 400 MHz): δ 11.04 (s, 1H), 7.43 (s, 1H), 4.84 (s, 2H).

### Step 3: Synthesis of 6,7-dibromo-1-methyl-1H-pyrido[2,3-b][1,4]oxazin-2(3H)-one

To a stirred solution of 6,7-dibromo-1H-pyrido[2,3-b][1,4]oxazin-2(3H)-one (0.3 g, 0.97 mmol, 1.0 eq) in DMF (3 mL) at 0° C., K₂CO₃ (0.40 g, 2.92 mmol, 3.0 eq) and Mel (0.41 g, 2.92 mmol, 3.0 eq) were added and the mixture was stirred at room temperature for 3 hours. After completion of the reaction as indicated by TLC, the reaction mixture was poured in ice water (50 mL) and the resulting precipitate was filtered and dried under vacuum to afford the title compound as a light-yellow solid (0.26 g, 82.89%). LCMS: RT=1.887 min, m/z 320.9 [M+H]⁺; ¹H NMR (DMSO-d₆, 400 MHz): δ 7.88 (s, 1H), 4.92 (s, 2H), 3.24 (s, 3H).

### Step 4: Synthesis of 7-bromo-6-((3-methoxy-2,6-dimethylphenyl)amino)-1-methyl-1H-pyrido[2,3-b][1,4]oxazin-2(3H)-one

To a stirred solution of 6,7-dibromo-1-methyl-1H-pyrido[2,3-b][1,4]oxazin-2(3H)-one (4.0 g, 12.42 mmol, 1.0 eq) in 1,2-DME (40 mL), CS₂CO₃ (12.14 g, 37.27 mmol, 3.0 eq) and 3-methoxy-2,6-dimethylaniline (1.5 g, 9.93 mmol, 0.8 eq) were added and the reaction mixture was purged with N₂ gas for 15 mins. Xantphos (1.43 g, 2.48 mmol, 0.2 eq) and Pd₂(dba)₃ (1.13 g, 1.24 mmol, 0.1 eq) were added and the reaction mixture was heated to 130° C. for 8 hours. After completion of the reaction as indicated by TLC, the resulting reaction mixture was cooled to ambient temperature, poured into water (100 mL), and extracted with ethyl acetate (3×100 mL). The combined organic phase was dried over Na₂SO₄, filtered, and concentrated under reduced pressure. The resulting crude material was purified by flash chromatography (230-400 mesh silica gel, 0-70% EtOAc/hexane) to afford the title compound as a light brown solid (1.6 g, 32.83%). LCMS: RT=2.370 min, m/z 392.32 [M+H]⁺. ¹H NMR (DMSO-d₆, 400 MHz): δ 7.70 (s, 1H), 7.60 (s, 1H), 7.03 (d, J=8.0 Hz, 1H), 6.81 (d, J=8.4 Hz, 1H), 4.64 (s, 2H), 3.77 (s, 3H), 3.19 (s, 3H), 2.02 (s, 3H), 1.93 (s, 3H).

### Step 5: Synthesis of 7-bromo-N-(3-methoxy-2,6-dimethylphenyl)-1-methyl-2,3-dihydro-1H-pyrido[2,3-b][1,4]oxazin-6-amine

To a stirred solution of 7-bromo-6-((3-methoxy-2,6-dimethylphenyl)amino)-1-methyl-1H-pyrido[2,3-b][1,4]oxazin-2(3H)-one (0.31 g, 0.79 mmol, 1.0 eq) in THF (3 mL) at 0° C., BH₃-DMS (2.0 M in THF) (0.79 mL, 1.58 mmol, 2.0 eq) was added and the reaction mixture was stirred at 80° C. for 3 hours. After completion of the reaction as indicated by TLC, the reaction mixture was quenched with methanol (10 mL) and concentrated under vacuum. The crude product was purified by column chromatography (230-400 mesh silica gel, 0-50% EtOAc/hexane) to afford the title compound as a light-yellow solid (0.23 g, 76.94%). LCMS: RT=2.723 min, m/z 378.17 [M+H]⁺; ¹H NMR (DMSO-d₆, 400 MHz): δ 7.24 (s, 1H), 7.01 (d, J=8.4 Hz, 1H), 6.92 (bs, 1H), 6.76 (d, J=8.0 Hz, 1H), 4.20 (t, J=4.8 Hz, 2H), 3.76 (s, 3H), 3.01 (t, J=4.8 Hz, 2H), 2.71 (s, 3H), 2.02 (s, 3H), 1.92 (s, 3H).

### Step 6: Synthesis of 7-amino-6-(3-methoxy-2,6-dimethylphenyl)-1-methyl-1,2,3,6-tetrahydropyrrolo [3′,2′:5,6]pyrido[2,3-b][1,4]oxazine-8-carbonitrile

To a stirred solution of malononitrile (0.080 g, 1.21 mmol, 2.0 eq) in 1,2-DME (3 mL) at 0° C., NaOEt (21% in ethanol) (2.3 mL, 10.0 V) was added and the reaction mixture was stirred at room temperature for 10 min. 7-Bromo-N-(3-methoxy-2,6-dimethylphenyl)-1-methyl-2,3-dihydro-1H-pyrido[2,3-b][1,4]oxazin-6-amine (0.23 g, 0.60 mmol, 1.0 eq) and PdCl₂(dppf) dichloromethane complex (0.048 g, 0.060 mmol, 0.1 eq) were added and the reaction mixture was purged with argon for 10 min. The reaction was stirred at 110° C. for 4 hours. After completion of the reaction as indicated by TLC, the reaction mixture was quenched with water (10 mL) and extracted with ethyl acetate (3×50 mL). The combined organic phase was dried over Na₂SO₄, filtered, and concentrated under reduced pressure. The resulting crude material was purified by flash chromatography (230-400 mesh silica gel, 0-70% EtOAc/hexane) to afford the title compound as a light-yellow solid (0.15 g, 67.88%). LCMS: RT=2.101 min m/z 364.22 [M+H]+. ¹H NMR (DMSO-d₆, 400 MHz): δ 7.20 (d, J=8.4 Hz, 1H), 7.06 (d, J=8.4 Hz, 1H), 6.97 (s, 1H), 6.52 (bs, 2H), 4.25 (bs, 2H), 3.83 (d, J=5.2 Hz, 3H), 3.15 (d, J=4.8 Hz, 2H), 2.85 (s, 3H), 1.78 (s, 3H), 1.69 (s, 3H).

### Step 7: Synthesis of 7-amino-6-(3-methoxy-2,6-dimethylphenyl)-1-methyl-1,2,3,6-tetrahydro pyrrolo[3′,2′:5,6]pyrido[2,3-b][1,4]oxazine-8-carbonitrile

A stirred solution of 7-amino-6-(3-methoxy-2,6-dimethylphenyl)-1-methyl-1,2,3,6-tetrahydropyrrolo[3′,2′:5,6]pyrido[2,3-b][1,4]oxazine-8-carbonitrile (0.25 g, 0.68 mmol, 1.0 eq) in dichloromethane (3 mL) at 0° C., BBr₃ (LOM in DCM) (2.06 mL, 2.06 mmol, 3.0 eq) was added and the reaction mixture was stirred at room temperature for 1 hour. After completion of the reaction as indicated by TLC, the reaction mixture was quenched with methanol (5 mL) and concentrated under vacuum. The resulting crude material was purified by reverse phase column chromatography (C18, 0-70% water/ACN) to afford the title compound as a light green solid (0.090 g, 37.45%). LCMS: RT=1.82 min m/z 350 [M+H]⁺. ¹H NMR (DMSO-d₆, 400 MHz): δ 9.49 (s, 1H), 7.02 (d, J=8.4 Hz, 1H), 6.96 (s, 1H), 6.88 (d, J=8.0 Hz, 1H), 6.47 (bs, 2H), 4.25 (t, J=4 Hz, 2H), 3.14 (t, J=4 Hz, 2H), 2.85 (s, 3H), 1.74 (s, 3H), 1.65 (s, 3H).

### Step 8: Synthesis of 7-amino-6-(3-hydroxy-2,6-dimethylphenyl)-1-methyl-1,2,3,6-tetrahydropyrrolo [3′,2′:5,6]pyrido[2,3-b][1,4]oxazine-8-carboxamide (Compound 568)

A solution of 7-amino-6-(3-methoxy-2,6-dimethylphenyl)-1-methyl-1,2,3,6-tetrahydro pyrrolo [3′,2′:5,6]pyrido[2,3-b][1,4]oxazine-8-carbonitrile (0.080 g, 0.22 mmol, 1.0 eq) in H₂SO₄ (1 mL) was stirred at room temperature for 30 min. After completion of the reaction as indicated by TLC, the reaction mixture was quenched with 10% NaHCO₃ (20 mL) and extracted with ethyl acetate (3×10 mL). The combined organic phase was dried over Na₂SO₄, filtered and concentrated under reduced pressure. The resulting crude material was purified by reverse phase chromatography (C18, 0-50% water/ACN) to afford the title compound as a light green solid (0.010 g, 11.89%). LCMS: RT=1.473 min, m/z 368.4 [M+H]⁺. ¹H NMR (DMSO-d₆, 400 MHz): δ 9.52 (bs, 1H), 7.39 (s, 1H), 7.02 (d, J=8.4 Hz, 1H), 6.88 (d, J=8.4 Hz, 1H), 6.67 (s, 2H), 6.57 (s, 2H), 4.24 (bs, 2H), 3.12 (bs, 2H), 2.89 (s, 3H), 1.71 (s, 3H), 1.65 (s, 3H).

### Example 16. Synthesis of Compound 566

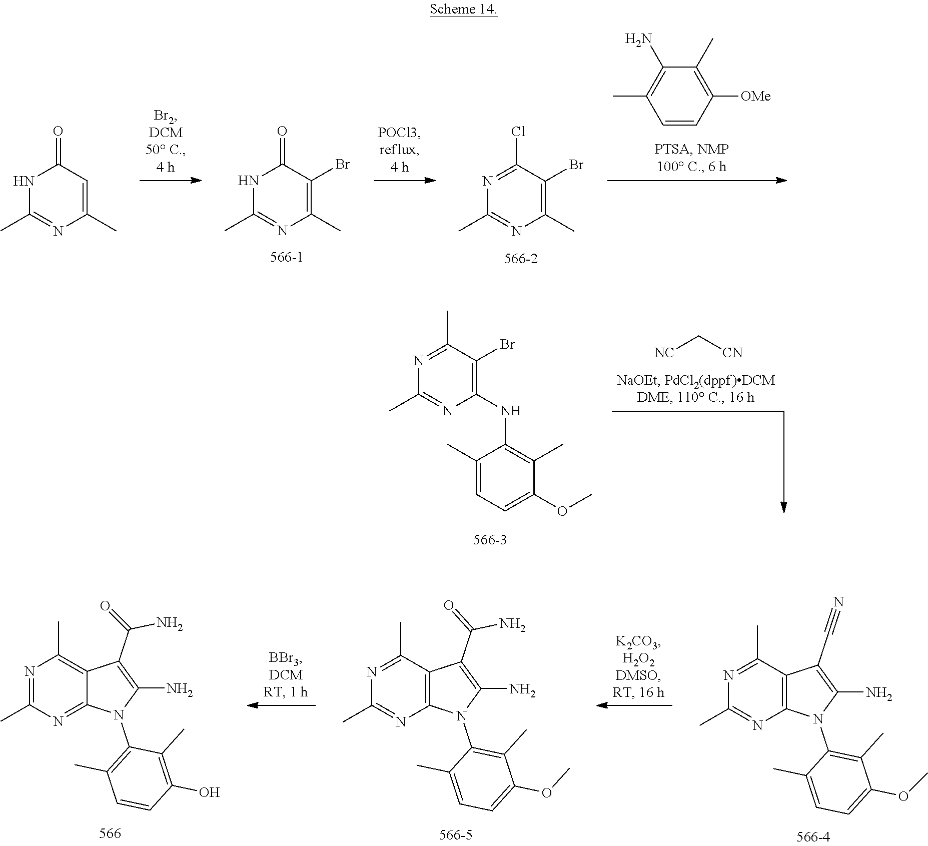

### Step 1: Synthesis of 5-bromo-2,6-dimethylpyrimidin-4(3H)-on

To a stirred solution of 2,6-dimethylpyrimidin-4(3H)-one (15 g, 120.83 mmol, 1.0 eq) in dichloromethane (150 mL) was added Br₂ (19.31 g, 120.83 mmol, 1.0 eq) dropwise at 0° C. The reaction was stirred at 50° C. for 4 hours. After completion of the reaction as indicated by TLC, the resulting mixture was quenched with 10% Na₂S₂O₃ (200 mL) and extracted with DCM (3×200 mL). The organic layer was dried over Na₂SO₄ and concentrated under vacuum. The crude compound was purified by column chromatography using (silica gel mesh 230-300, 2% MeOH/DCM) to afford the title compound as a brown solid (9.0 g, 36.68%). ¹H NMR (DMSO-d₆, 400 MHz): 12.76 (s, 1H), 1.31 (s, 3H), 1.15 (s, 3H).

### Step 2: Synthesis of 5-bromo-4-chloro-2,6-dimethylpyrimidine

A solution of 5-bromo-2,6-dimethylpyrimidin-4(3H)-one (9.0 g, 44.32 mmol, 1.0 eq) in POCl₃ (50 mL) was stirred at 110° C. for 4 hours. After completion of the reaction as indicated by TLC, the reaction mixture was concentrated under vacuum. The crude was diluted with 10% NaHCO₃ solution (100 mL) and extracted with ethyl acetate (3×100 mL). The organic layer was dried over Na₂SO₄ and concentrated under vacuum. The crude compound was purified by column chromatography (230-400 mesh silica gel, 0-5% EtOAc/hexane) to afford the title compound as a yellow solid (7.0 g, 71.30%). LCMS: RT=1.812 min, m/z 220.9 (M+H)⁺. ¹H NMR (CDCl₃, 400 MHz): δ 2.65 (s, 3H), 2.63 (s, 3H).

### Step 3: Synthesis of 5-bromo-N-(3-methoxy-2,6-dimethylphenyl)-2,6-dimethylpyrimidin-4-amine

To a stirred solution of 5-bromo-4-chloro-2,6-dimethylpyrimidine (5.0 g, 22.57 mmol, 1.0 eq) in NMP (50 mL), 3-methoxy-2,6-dimethylaniline (6.82 g, 45.15 mmol, 2.0 eq) and PTSA (0.429 g, 2.25 mmol, 0.1 eq) were added and the reaction was stirred at 100° C. for 6 hours. After completion of the reaction as indicated by TLC, the resulting mixture was quenched with water (100 mL) and extracted with ethyl acetate (3×100 mL). The organic layer was dried over Na₂SO₄ and concentrated under vacuum to afforded the crude product. The crude compound was purified by column chromatography (230-400 mesh silica gel, 0-8% EtOAc/hexane) to afford the title compound as a white solid (3.5 g, 46.11%). LCMS: RT=1.499 min, m/z 336.2 (M+H)⁺. ¹H NMR (DMSO-d₆, 400 MHz): δ 8.35 (s, 1H), 7.06 (d, J=8.4 Hz, 1H), 6.84 (d, J=8.0 Hz, 1H), 3.78 (s, 3H), 2.405 (s, 3H), 2.14 (s, 3H), 2.02 (s, 3H), 1.92 (s, 3H).

### Step 4: Synthesis of 6-amino-7-(3-methoxy-2,6-dimethylphenyl)-2,4-dimethyl-7H-pyrrolo[2,3-d]pyrimidine-5-carbonitrile

To a stirred solution of malononitrile (0.78 g, 11.89 mmol, 2.0 eq) in DME (20 mL) at 0° C., NaOEt (21% in ethanol) (20 mL, 20 V) was added and the reaction was stirred for 15 min. 5-Bromo-N-(3-methoxy-2,6-dimethylphenyl)-2,6-dimethylpyrimidin-4-amine (2.0 g, 5.94 mmol, 1.0 eq) and PdCl₂(dppf) dichloromethane complex (0.485 g, 0.59 mmol, 0.1 eq) were added and the reaction was stirred at 110° C. for 16 hours. After completion of the reaction as indicated by TLC, the reaction was quenched with water (100 mL) and extracted with ethyl acetate (3×100 mL). The organic layer was dried over Na₂SO₄ and concentrated in vacuum. The crude product was purified by column chromatography (230-400 mesh silica gel, 10% EtOAc/hexane) to afford the title compound as a light-yellow solid (0.9 g, 47.08%). LCMS: RT=1.401 min, m/z 323.21 (M+H)⁺. ¹H NMR (DMSO-d₆, 400 MHz): δ 7.24 (d, J=8.4 Hz, 1H), 7.15 (bs, 2H), 7.10 (d, J=8.4 Hz, 1H), 3.84 (s, 3H), 2.61 (s, 3H), 2.38 (s, 3H), 1.78 (s, 3H), 1.69 (s, 3H).

### Step 5: Synthesis of 6-amino-7-(3-hydroxy-2,6-dimethylphenyl)-2,4-dimethyl-7H-pyrrolo[2,3-d]pyrimidine-5-carboxamide

To a stirred solution of 6-amino-7-(3-methoxy-2,6-dimethylphenyl)-2,4-dimethyl-7H-pyrrolo[2,3-d]pyrimidine-5-carbonitrile (0.9 g, 2.65 mmol, 1 eq) in DMSO (15 mL), K₂CO₃ (7.32 g, 53.03 mmol, 20 eq) and H₂O₂ (30%) (15.03 g, 132.58 mmol, 50 eq) were added and the reaction mixture was stirred at room temperature for 16 hours. After completion of the reaction as indicated by TLC, the resulting mixture was quenched with water (50 mL) and extracted with ethyl acetate (3×30 mL). The organic layer was dried over Na₂SO₄ and concentrated under vacuum. The crude product was purified by column chromatography (230-400 mesh silica gel, 50% EtOAc/hexane) to afford the title compound as a brown solid (0.16 g, 16.83%). LCMS: RT=1.265 min, m/z 340.2 (M+H)⁺. ¹H NMR (DMSO-d₆, 400 MHz): δ 7.25 (d, J=8.8 Hz, 1H), 7.10 (d, J=8.4 Hz, 1H), 6.89 (bs, 2H), 6.54 (bs, 2H), 3.85 (s, 3H), 2.70 (s, 3H), 2.28 (s, 3H), 1.79 (s, 3H), 1.69 (s, 3H).

### Step 6: Synthesis of 6-amino-7-(3-hydroxy-2,6-dimethylphenyl)-2,4-dimethyl-7H-pyrrolo[2,3-d]pyrimidine-5-carboxamide (Compound 566)

To a stirred solution of 6-amino-7-(3-hydroxy-2,6-dimethylphenyl)-2,4-dimethyl-7H-pyrrolo[2,3-d]pyrimidine-5-carboxamide (0.16 g, 0.47 mmol, 1.0 eq) in DCM (15 mL), BBr3 (1M in DCM) (1.41 mL, 1.41 mmol, 3.0 eq) was added and the reaction mixture was stirred at room temperature for 1 hour. After completion of the reaction as indicated by TLC, the resulting mixture was quenched with MeOH (5 ml), and concentrated under vacuum. The crude was diluted with water (20 mL) and extracted with dichloromethane (3×10 mL). The organic layer was dried over Na₂SO₄ and concentrated under vacuum. The crude compound was purified by column chromatography (230-400 mesh silica gel, 5% MeOH/MDC) to afford the title compound as a white solid (0.013 g, 8.48%). LCMS: RT=4.434 min, m/z 325 (M+H)⁺. ¹H NMR (DMSO-d₆, 400 MHz): δ 9.57 (s, 1H), 7.07 (d, J=8.4 Hz, 1H), 6.92 (d, J=8.0 Hz, 1H), 6.88 (bs, 2H), 6.51 (bs, 2H), 2.71 (s, 3H), 2.39 (s, 3H), 1.75 (s, 3H), 1.65 (s, 3H).

### Example 17. Synthesis of Compound 579

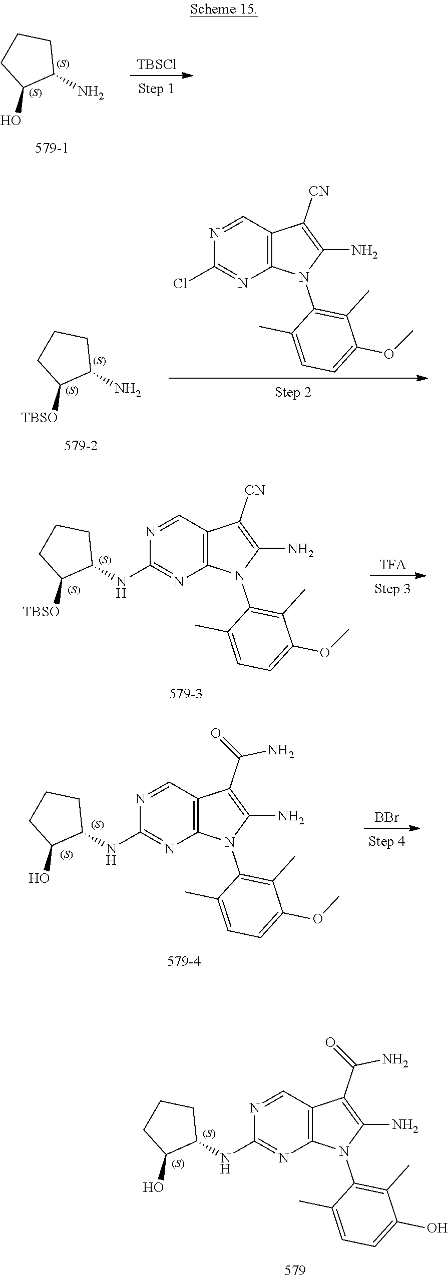

### Step 1: Synthesis of (1S,2S)-2-((tert-butyldimethylsilyl)oxy)cyclopentanamine

To a stirred solution of (1S,2S)-2-aminocyclopentanol (1.0 g, 7.27 mmol) in dichloromethane (15 mL) were added TBSCl (2.19 g, 14.53 mmol), Et₃N (2.21 g, 21.80 mmol), and DMAP (89 mg, 0.73 mmol). The reaction was stirred at 20° C. for 12 hours. The mixture was quenched with water (30 mL) and extracted with dichloromethane (20 mL×2). The combined extracts were washed with brine (30 mL), dried over anhydrous sodium sulfate, filtered, and concentrated under reduced pressure. The residue was purified by flash column chromatography (silica gel, 100-200 mesh, 0-10% methanol in dichloromethane) to afford the title compound (950 mg, 61%) as a white solid. ¹H NMR (400 MHz, CDCl₃) δ=3.98-3.93 (m, 1H), 3.19-3.13 (m, 1H), 2.10-1.93 (m, 2H), 1.75-1.71 (m, 2H), 1.55-1.50 (m, 2H), 0.89 (s, 9H), 0.11-0.07 (m, 6H).

### Step 2: Synthesis of 6-amino-2-(((1S,2S)-2-((tert-butyldimethylsilyl)oxy)cyclopentyl)amino)-7-(3-methoxy-2,6-dimethylphenyl)-7H-pyrrolo[2,3-d]pyrimidine-5-carbonitrile

To a solution of (1S,2S)-2-((tert-butyldimethylsilyl)oxy)cyclopentanamine 6-amino-2-chloro-7-(3-methoxy-2,6-dimethylphenyl)-7H-pyrrolo[2,3-d]pyrimidine-5-carbonitrile (250 mg, 0.76 mmol) in NMP (2.5 mL) was added DIEA (246 mg, 1.91 mmol). The mixture was stirred at 180° C. for 3 hours under microwave. The reaction mixture was quenched with water (30 mL) and extracted with ethyl acetate (30 mL×2). The combined extracts were washed with brine (20 mL×3), dried over anhydrous sodium sulfate, filtered, and concentrated under reduced pressure. The residue was purified by flash column chromatography (silica gel, 100-200 mesh, 0-100% ethyl acetate in petroleum ether) to afford the title compound (75 mg, 19%) as a yellow oil. LCMS R_{T}=1.520 min, m/z=507.1 [M+H]⁺.

### Step 3: Synthesis of 6-amino-2-(((1S,2S)-2-hydroxycyclopentyl)amino)-7-(3-methoxy-2,6-dimethylphenyl)-7H-pyrrolo[2,3-d]pyrimidine-5-carboxamide

A solution of 6-amino-2-(((1S,2S)-2-((tert-butyldimethylsilyl)oxy)cyclopentyl)amino)-7-(3-methoxy-2,6-dimethylphenyl)-7H-pyrrolo[2,3-d]pyrimidine-5-carbonitrile (75 mg, 0.15 mmol) in TFA (2.0 mL) was stirred at 65° C. for 3 hours. The mixture was concentrated under reduced pressure. The residue was purified by RP-HPLC (0 to 34% of acetonitrile in water and 0.1% TFA) to afford the title compound (100 mg, 100%) as a light yellow solid. LCMS R_{T}=0.967 min, m/z=411.0 [M+H]⁺.

### Step 4: Synthesis of 6-amino-7-(3-hydroxy-2,6-dimethylphenyl)-2-(((1S,2S)-2-hydroxycyclopentyl)amino)-7H-pyrrolo[2,3-d]pyrimidine-5-carboxamide (Compound 579)

To a solution of 6-amino-2-(((1S,2S)-2-hydroxycyclopentyl)amino)-7-(3-methoxy-2,6-dimethylphenyl)-7H-pyrrolo[2,3-d]pyrimidine-5-carboxamide (100 mg, 0.19 mmol) in 1,2-dichloroethane (4.0 mL) was added BBr₃ (2 M in CH₂Cl₂, 0.95 mL). The reaction was stirred at 80° C. for 4.5 hours and then quenched with MeOH (1 mL) and water (0.5 mL). The mixture was concentrated under reduced pressure. The residue was purified by RP-HPLC (0 to 40% acetonitrile in water and 10% NH₄HCO₃) to afford the crude title compound (19.1 mg, 25%) as a white solid. LCMS R_{T}=0.955 min, m/z=397.1 [M+H]⁺. ¹H NMR (400 MHz, DMSO-d₆) δ=9.51 (s, 1H), 8.53 (s, 1H), 7.04 (d, J=8.0 Hz, 1H), 6.88 (d, J=8.4 Hz, 1H), 6.71-6.62 (m, 4H), 6.55 (d, J=5.6 Hz, 1H), 4.95 (s, 1H), 3.80-3.70 (m, 2H), 1.95-1.93 (m, 1H), 1.80-1.70 (m, 7H), 1.58-1.54 (m, 2H), 1.43-1.37 (m, 2H).

### Example 18. Synthesis of Compound 580

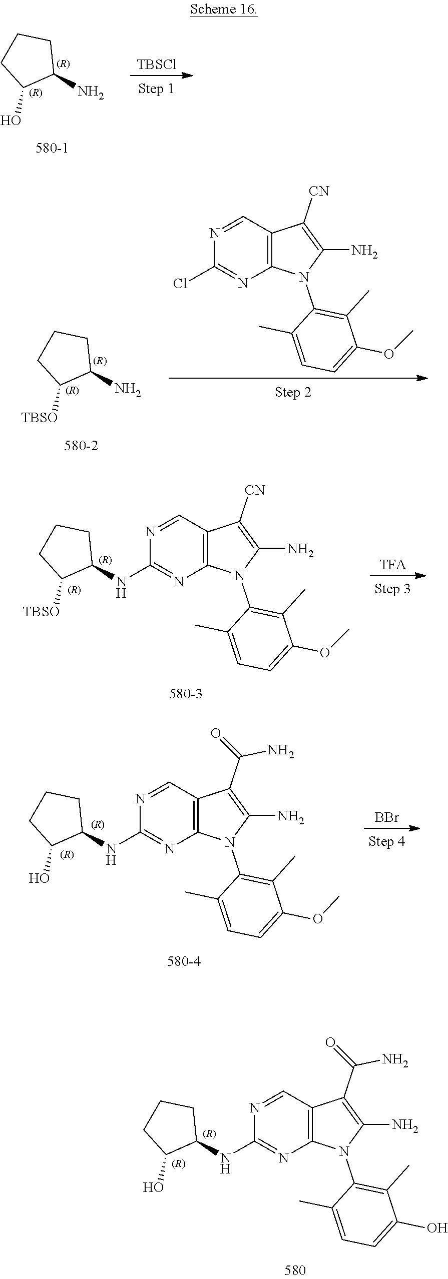

### Step 1: Synthesis of (1R,2R)-2-((tert-butyldimethylsilyl)oxy)cyclopentanamine

To a stirred solution of (1R,2R)-2-aminocyclopentanol (1.0 g, 7.27 mmol) in dichloromethane (15 mL) were added TBSCl (2.19 g, 14.53 mmol), Et₃N (2.21 g, 21.80 mmol), and DMAP (89 mg, 0.73 mmol). The reaction was stirred at 20° C. for 12 hours. The mixture was quenched with water (30 mL) and extracted with dichloromethane (20 mL×2). The combined extracts were washed with brine (30 mL), dried over anhydrous sodium sulfate, filtered, and concentrated under reduced pressure. The residue was purified by flash column chromatography (silica gel, 100-200 mesh, 0-10% methanol in dichloromethane) to afford the title compound (750 mg, 48%) as a white solid. ¹H NMR (400 MHz, CDCl₃) δ=4.25-4.23 (m, 1H), 3.35-3.27 (m, 1H), 2.22-2.02 (m, 2H), 1.84-1.56 (m, 4H), 0.89 (s, 9H), 0.16-0.10 (m, 6H).

### Step 2: Synthesis of 6-amino-2-(((1R,2R)-2-((tert-butyldimethylsilyl)oxy)cyclopentyl)amino)-7-(3-methoxy-2,6-dimethylphenyl)-7H-pyrrolo[2,3-d]pyrimidine-5-carbonitrile

To a solution of (1R,2R)-2-((tert-butyldimethylsilyl)oxy)cyclopentanamine (246 mg, 1.14 mmol) and 6-amino-2-chloro-7-(3-methoxy-2,6-dimethylphenyl)-7H-pyrrolo[2,3-d]pyrimidine-5-carbonitrile (250 mg, 0.76 mmol) and in NMP (2.5 mL) was added DIEA (246 mg, 1.91 mmol). The reaction was stirred at 180° C. for 3 hours under microwave. The reaction mixture was quenched with water (30 mL) and extracted with ethyl acetate (30 mL×2). The combined extracts were washed with brine (20 mL×3), dried over anhydrous sodium sulfate, filtered, and concentrated under reduced pressure. The residue was purified by flash column chromatography (silica gel, 100-200 mesh, 0-100% ethyl acetate in petroleum ether) to afford the title compound (85 mg, 22%) as a yellow oil. LCMS R_{T}=1.520 min, m/z=507.1 [M+H]⁺.

### Step 3: Synthesis of 6-amino-2-(((1R,2R)-2-hydroxycyclopentyl)amino)-7-(3-methoxy-2,6-dimethylphenyl)-7H-pyrrolo[2,3-d]pyrimidine-5-carboxamide

A solution of 6-amino-2-(((1R,2R)-2-((tert-butyldimethylsilyl)oxy)cyclopentyl)amino)-7-(3-methoxy-2,6-dimethylphenyl)-7H-pyrrolo[2,3-d]pyrimidine-5-carbonitrile (85 mg, 0.17 mmol) in TFA (2.0 mL) was stirred at 65° C. for 3 hours. The mixture was concentrated under reduced pressure. The residue was purified by RP-HPLC (0 to 34% acetonitrile in water and 0.1% TFA) to afford the title compound (100 mg, 100%) as a light yellow solid. LCMS R_{T}=0.973 min, m/z=411.0 [M+H]⁺.

### Step 4: Synthesis of 6-amino-7-(3-hydroxy-2,6-dimethylphenyl)-2-(((1R,2R)-2-hydroxycyclopentyl)amino)-7H-pyrrolo[2,3-d]pyrimidine-5-carboxamide (Compound 580)

To a solution of 6-amino-2-(((1R,2R)-2-hydroxycyclopentyl)amino)-7-(3-methoxy-2,6-dimethylphenyl)-7H-pyrrolo[2,3-d]pyrimidine-5-carboxamide (100 mg, 0.19 mmol) in 1,2-dichloroethane (4.0 mL) was added BBr₃ (2 M in CH₂C₂, 0.95 mL). The reaction was stirred at 60° C. for 4.5 hours. The mixture was quenched with MeOH (1 mL) and water (0.5 mL) and then concentrated under reduced pressure. The residue was purified by RP-HPLC (0 to 40% acetonitrile in water and 10% NH₄HCO₃) to afford the title compound (18.4 mg, 24%) as a white solid. LCMS R_{T}=0.955 min, m/z=397.1 [M+H]⁺. ¹H NMR (400 MHz, DMSO-d₆) δ=9.51 (s, 1H), 8.53 (s, 1H), 7.04 (d, J=7.6 Hz, 1H), 6.88 (d, J=8.4 Hz, 1H), 6.71-6.62 (m, 4H), 6.55 (d, J=5.6 Hz, 1H), 4.95 (s, 1H), 3.80-3.71 (m, 2H), 1.95-1.93 (m, 1H), 1.80-1.70 (m, 7H), 1.58-1.54 (m, 2H), 1.43-1.37 (m, 2H).

### Example 19. Synthesis of Compound 581

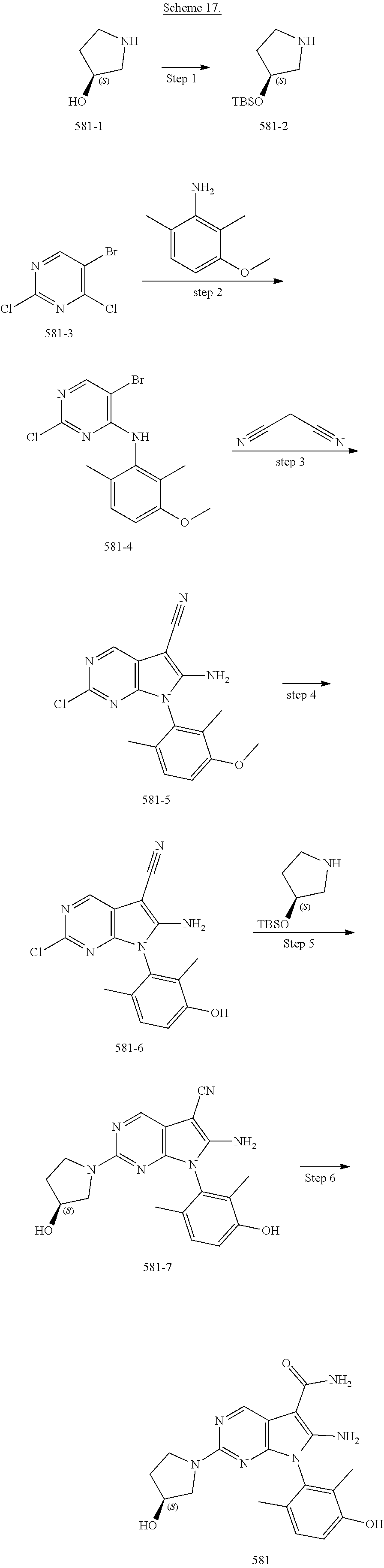

### Step 1: Synthesis of tert-butyl-dimethyl-[(3S)-pyrrolidin-3-yl]oxy-silane

To a solution of (3S)-pyrrolidin-3-ol (2 g, 22.96 mmol) in ACN (20 mL) were added Et₃N (5.81 g, 57.39 mmol) and TBSCl (5.19 g, 34.44 mmol). The reaction was stirred at 25° C. for 16 hours, and was then concentrated under vacuum. The residue was suspended in water (100 mL) and extracted with ethyl acetate (50 mL*3). The combined extracts were washed with brine (100 mL), dried over anhydrous sodium sulfate, filtered, and concentrated under reduced pressure. The residue was dissolved with methanol/dichloromethane (30 mL, v/v=1/10), filtered, and the filtrate was concentrated. The residue was washed by petroleum ether (50 mL) to afford tert-butyl-dimethyl-[(3S)-pyrrolidin-3-yl]oxy-silane (1.7 g, 37% yield) as a white solid. ¹H NMR (400 MHz, CDCl₃) δ 10.08-9.54 (m, 2H), 4.57-4.51 (m, 1H), 3.51-3.34 (m, 3H), 3.25-3.15 (m, 1H), 2.10-1.94 (m, 2H), 1.80-1.74 (m, 2H), 0.89 (s, 9H), 0.09 (d, J=4.0 Hz, 6H).

### Step 2: Synthesis of 5-bromo-2-chloro-N-(3-methoxy-2,6-dimethyl-phenyl)pyrimidin-4-amine

To a solution of 5-bromo-2,4-dichloro-pyrimidine (16.58 g, 72.75 mmol, 9.31 mL) in THF (120 mL) were added 3-methoxy-2,6-dimethyl-aniline (10 g, 66.14 mmol) and LiHMDS (1 M, 119.04 mL) at 0° C. under N₂. The reaction was stirred at 0° C. for 1 hour, quenched with water (150 mL), and extracted with ethyl acetate (200 mL*3). The organic extracts were dried over anhydrous sodium sulfate, filtered, and concentrated. The residue was purified by flash column chromatography (silica gel, 100-200 mesh, 0-16% ethyl acetate in petroleum ether) to afford the title compound (14.6 g, 64%) as a yellow oil. LCMS R_{T}=0.754 min, m/z=343.8 [M+H]⁺.

### Step 3: Synthesis of 6-amino-2-chloro-7-(3-methoxy-2,6-dimethyl-phenyl)pyrrolo[2,3-d]pyrimidine-5-carbonitrile

To a solution of propanedinitrile (2.75 g, 41.59 mmol, 2.62 mL) in DME (120 mL) was added NaH (1.77 g, 60% dispersion in mineral oil, 44.36 mmol) at 0° C. After stirring for 0.2 hour, 5-bromo-2-chloro-N-(3-methoxy-2,6-dimethyl-phenyl)pyrimidin-4-amine (9.5 g, 27.73 mmol) and Pd(dppf)Cl₂·CH₂Cl₂ (2.26 g, 2.77 mmol) were added. The reaction was stirred at 110° C. under N₂ for 4 hours, diluted with 500 mL of ethyl acetate, and then treated with Na₂CO₃ (600 mg) and N-acetyl-L-cysteine (500 mg) in 100 mL in H₂O. The combined organic layers were washed with H₂O (100 mL) and brine (100 mL), dried over Na₂SO₄, filtered, and concentrated under reduced pressure. The residue was purified by flash column chromatography (silica gel, 100-200 mesh, 0-10% ethyl acetate in petroleum ether) to afford the title compound (4 g, 44%) as a white solid. LCMS R_{T}=0.693 min, m/z=328.0 [M+H]⁺.

### Step 4: Synthesis of 6-amino-2-chloro-7-(3-hydroxy-2,6-dimethyl-phenyl)pyrrolo[2,3-d]pyrimidine-5-carbonitrile

To a solution of 6-amino-2-chloro-7-(3-methoxy-2, 6-dimethyl-phenyl)pyrrolo[2,3-d]pyrimidine-5-carbonitrile (300 mg, 0.91 mmol) in 1,2-dichloroethane (3.0 mL) was added BBr₃ (2 M in dichloromethane, 4.58 mL). The reaction was stirred at 20° C. for 12 hours, quenched with saturated NaHCO₃ (20 mL) at 0° C., and then extracted with ethyl acetate (30 mL×3). The combined organic layers were washed with brine (20 mL×2), dried over Na₂SO₄, filtered, and concentrated under reduced pressure. The residue was purified by flash column chromatography (silica gel, 100-200 mesh, 0-60% ethyl acetate in petroleum ether) to afford the title compound (0.23 g, 80%) as a yellow solid. LCMS R_{T}=0.635 min, m/z=313.9 [M+H]⁺.

### Step 5: Synthesis of 6-amino-7-(3-hydroxy-2,6-dimethyl-phenyl)-2-[(3S)-3-hydroxypyrrolidin-1-yl]pyrrolo[2,3-d]pyrimidine-5-carbonitrile

To a solution of tert-butyl-dimethyl-[(3S)-pyrrolidin-3-yl]oxy-silane (116 mg, 0.57 mmol) in NMP (3.0 mL) were added 6-amino-2-chloro-7-(3-hydroxy-2,6-dimethyl-phenyl)pyrrolo[2,3-d]pyrimidine-5-carbonitrile (150 mg, 0.48 mmol) and DIEA (185 mg, 1.43 mmol). The mixture was stirred at 180° C. for 2 hours in a microwave oven under a nitrogen atmosphere. The mixture was diluted with water (10 mL) and extracted with ethyl acetate (10 mL×3). The combined extracts were washed with brine (10 mL×2), dried over anhydrous sodium sulfate, and filtered. The filtrate was concentrated under reduced pressure and purified by flash column chromatography on silica gel (0% to 70% of ethyl acetate in petroleum ether) to afford 6-amino-7-(3-hydroxy-2,6-dimethyl-phenyl)-2-[(3S)-3-hydroxypyrrolidin-1-yl]pyrrolo[2,3-d]pyrimidine-5-carbonitrile (117 mg, 64% yield) as yellow oil. LCMS R_{T}=0.488 min, m/z=365.1 [M+H]⁺.

### Step 6: Synthesis of 6-amino-7-(3-hydroxy-2,6-dimethyl-phenyl)-2-[(3S)-3-hydroxypyrrolidin-1-yl]pyrrolo[2,3-d]pyrimidine-5-carboxamide (Compound 581)

A solution of 6-amino-7-(3-hydroxy-2,6-dimethyl-phenyl)-2-[(3S)-3-hydroxypyrrolidin-1-yl]pyrrolo[2,3-d]pyrimidine-5-carbonitrile (50 mg, 0.14 mmol) in H₂SO₄ (0.6 mL) was stirred at 25° C. for 1 hour. The reaction mixture was quenched with ice water (40 mL) and adjust to pH˜8 by NaHCO₃, and then concentrated under reduced pressure. The residue was washed with methanol/dichloromethane (60 mL, v/v=1/10), filtered, and concentrated under vacuum. The residue was treated with HCl/MeOH (5.0 mL) and was stirred at 25° C. for 1 hour and was then concentrated under vacuum. The crude product was purified by preparative HPLC [Column: Welch Xtimate C18 150*30 mm*5 um; Mobile phase: water (FA)-MeCN; B %: 0-24%; Gradient time: 30 min; Flow Rate: 30 ml/min] to afford 6-amino-7-(3-hydroxy-2,6-dimethyl-phenyl)-2-[(3S)-3-hydroxypyrrolidin-1-yl]pyrrolo[2,3-d]pyrimidine-5-carboxamide (6.2 mg, 12% yield) as a white solid. ¹H NMR (400 MHz, DMSO-d₆) δ 9.51 (s, 1H), 8.58 (s, 1H), 7.05 (d, J=8.4 Hz, 1H), 6.89 (d, J=8.4 Hz, 1H), 6.68-6.59 (m, 4H), 4.80 (s, 1H), 4.26 (s, 1H), 3.26-3.18 (m, 4H), 1.92-1.77 (m, 5H), 1.72 (s, 3H). LCMS R_{T}=0.900 min, m/z=383.4 [M+H]⁺.

### Example 20. Synthesis of Compound 582

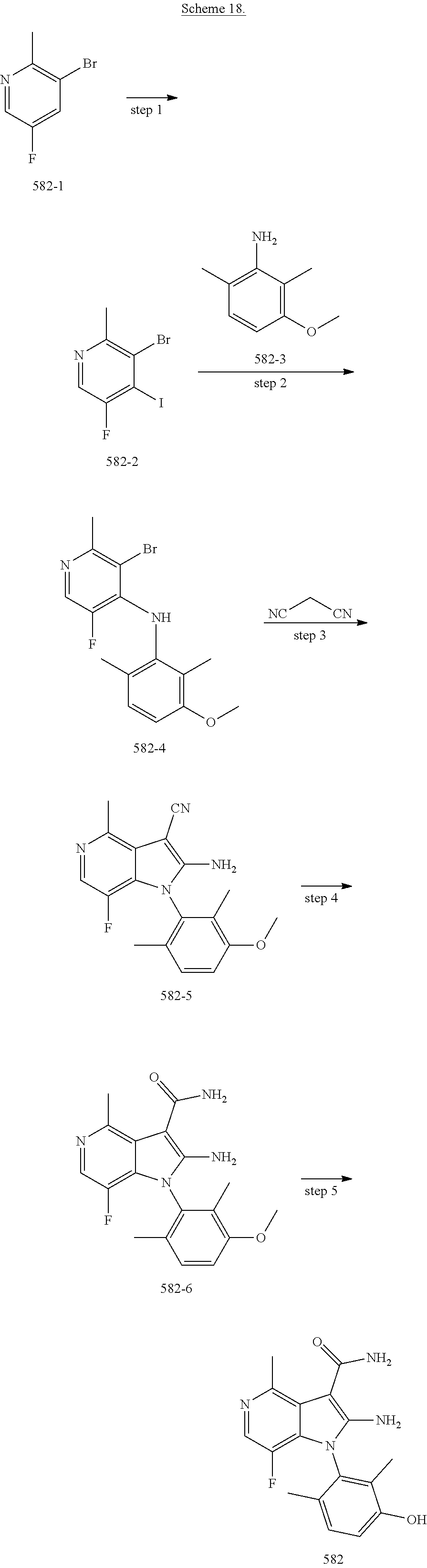

### Step 1: Synthesis of 3-bromo-5-fluoro-4-iodo-2-methyl-pyridine

To a solution of 3-bromo-5-fluoro-2-methyl-pyridine (3.0 g, 15.79 mmol) in tetrahydrofuran (10 mL) was added lithium diisopropylamide (2 M, 11.84 mL) at −78° C. and the reaction was stirred for 0.25 hours under a nitrogen atmosphere. Then iodide (4.8 g, 18.95 mmol) in tetrahydrofuran (10 mL) was added. After stirring at −78° C. for 2.25 hours, the mixture was quenched with saturated ammonium chloride (20 mL) and extracted with ethyl acetate (50 mL×3). The combined extracts were washed with brine (100 mL), dried over sodium sulfate, filtered, and concentrated to dryness under reduced pressure. The residue was purified by column chromatography (silica gel, 100-200 mesh, 0-100% ethyl acetate in petroleum ether) to afford the title compound (4.1 g, 82%) as a yellow solid. ¹H NMR (400 MHz, DMSO) δ 8.35 (s, 1H), 2.68 (s, 3H).

### Step 2: Synthesis of 3-bromo-5-fluoro-N-(3-methoxy-2,6-dimethyl-phenyl)-2-methyl-pyridin-4-amine

To a solution of 3-methoxy-2,6-dimethyl-aniline (718 mg, 4.75 mmol) in toluene (20 mL) were added 3-bromo-5-fluoro-4-iodo-2-methyl-pyridine (1.5 g, 4.75 mmol), Pd₂(dba)₃ (217 mg, 0.24 mmol), Xantphos (275 mg, 0.47 mmol) and cesium carbonate (4.6 g, 14.24 mmol). After stirring at 100° C. for 16 hours under a nitrogen atmosphere, the mixture was filtered and the filtrate was concentrated under reduced pressure. The residue was purified by column chromatography (silica gel, 100-200 mesh, 0-15% ethyl acetate in petroleum ether) to afford the title compound (1.1 g, 57%) as a yellow solid. ¹H NMR (400 MHz, CDCl₃) δ 7.46-7.36 (m, 1H), 7.03 (d, J=8.4 Hz, 1H), 6.76 (d, J=8.4 Hz, 1H), 6.08 (s, 1H), 3.84 (s, 3H), 2.64 (s, 3H), 216 (s, 3H), 2.11 (s, 3H). LCMS R_{T}=0.725 min, m/z=340.7 [M+H]⁺.

### Step 3: Synthesis of Synthesis of 2-amino-7-fluoro-1-(3-methoxy-2,6-dimethyl-phenyl)-4-methyl-pyrrolo[3,2-c]pyridine-3-carbonitrile

To a solution of propanedinitrile (78 mg, 1.18 mmol) in 1,2-dimethoxyethane (3.0 mL) was added sodium ethoxide (382 mg, 1.18 mmol) in ethyl alcohol (1.0 mL), followed by Pd(dppf)Cl₂·CH₂Cl₂ (48 mg, 0.06 mmol) and 3-bromo-5-fluoro-N-(3-methoxy-2,6-dimethyl-phenyl)-2-methyl-pyridin-4-amine (200 mg, 0.59 mmol). After stirring at 130° C. for 4 hours in a microwave under a nitrogen atmosphere, the mixture was filtered and the filtrate was concentrated under reduced pressure. The residue was purified by prep-TLC (SiO₂, dichloromethane:methanol=10:1). to afford the title compound (100 mg, 50%) as a yellow solid. LCMS R_{T}=0.498 min, m/z=325.0 [M+H]⁺.

### Step 4: Synthesis of 2-amino-7-fluoro-1-(3-methoxy-2,6-dimethyl-phenyl)-4-methyl-pyrrolo[3,2-c]pyridine-3-carboxamide

A solution of 2-amino-7-fluoro-1-(3-methoxy-2,6-dimethyl-phenyl)-4-methyl-pyrrolo[3,2-c]pyridine-3-carbonitrile (100 mg, 0.31 mmol) in concentrated sulfuric acid (1.0 mL) was stirred at 20° C. for 1 hour, then quenched with ice water (5.0 mL) slowly and adjusted with sodium hydroxide solution (1 N) to pH˜8. The mixture was extracted with dichloromethane (10 mL*3), washed with brine (10 mL*2), dried over anhydrous sodium sulfate, filtered, and concentrated under reduced pressure to afford the title compound (55 mg, 38%) as a yellow solid. LCMS R_{T}=0.457 min, m/z=343.1 [M+H]⁺.

### Step 5: Synthesis of 2-amino-7-fluoro-1-(3-hydroxy-2,6-dimethyl-phenyl)-4-methyl-pyrrolo[3,2-c]pyridine-3-carboxamide (Compound 582)

To a solution of 6-amino-2-(3-hydroxyazetidin-1-yl)-7-(3-methoxy-2,6-dimethyl-phenyl)pyrrolo[2,3-d]pyrimidine-5-carboxamide (20 mg, 0.05 mmol) in 1,2-dichloroethane (1 mL) was added boron tribrothermide (4 M, 65.37 μL) at 0° C. The mixture was stirred at 20° C. for 1 hours under a nitrogen atmosphere. The reaction mixture was poured into ice water (2 mL) and filtered. The residue was purified by RP-HPLC (0 to 30% acetonitrile in water and 0.225% formic acid) to afford the title compound (7.2 mg, 13%) as a white solid. LCMS R_{T}=0.767 min, m/z=328.9 [M+H]. ¹H NMR (400 MHz, DMSO-d₆) δ 9.60 (s, 1H), 7.82 (s, 1H), 7.11-7.00 (m, 3H), 6.91 (d, J=8.4 Hz, 1H), 6.17 (s, 2H), 2.70 (s, 3H), 1.80 (s, 3H), 1.71 (s, 1H).

### Example 21. Synthesis of Compound 583

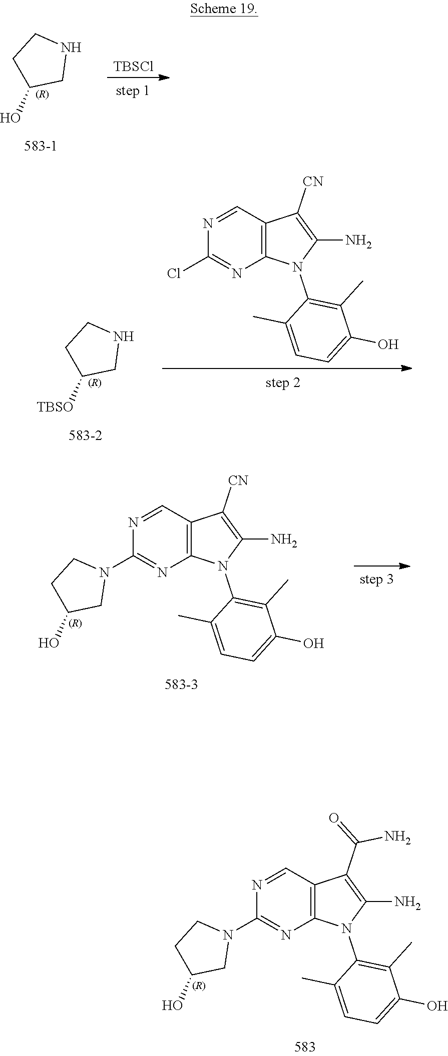

### Step 1: Synthesis of tert-butyl-dimethyl-[(3R)-pyrrolidin-3-yl]oxy-silane

To a mixture of (3R)-pyrrolidin-3-ol (2 g, 22.96 mmol, 1.90 mL) and TBSCl (5.19 g, 34.44 mmol, 4.22 mL) in MeCN (40 mL) was added Et₃N (5.81 g, 57.39 mmol) in one portion at 20° C. under N₂. The reaction mixture was stirred at 20° C. for 16 hours, and then treated with saturated NH₄Cl solution to pH=6. The mixture was diluted with brine and extracted with ethyl acetate (50 mL×2). The organic extracts were dried over anhydrous magnesium sulfate and concentrated. The crude product was triturated with petroleum ether (20 mL) at 20° C. for 5 min, and then dried under vacuum to afford tert-butyl-dimethyl-[(3R)-pyrrolidin-3-yl]oxy-silane (2.0 g, 43% yield) as a yellow solid. ¹H NMR (400 MHz, DMSO-d₆) δ: 4.54 (s, 1H), 3.26-3.10 (m, 3H), 3.02-2.88 (m, 1H), 2.03-1.72 (m, 2H), 0.86 (d, J=1.2 Hz, 9H), 0.08 (s, 6H)

### Step 2: Synthesis of 6-amino-7-(3-hydroxy-2,6-dimethyl-phenyl)-2-[(3R)-3-hydroxypyrrolidin-1-yl]pyrrolo[2,3-d]pyrimidine-5-carbonitrile

To a mixture of 6-amino-2-chloro-7-(3-hydroxy-2,6-dimethyl-phenyl)pyrrolo[2,3-d]pyrimidine-5-carbonitrile (160 mg, 0.510 mmol) and tert-butyl-dimethyl-[(3R)-pyrrolidin-3-yl]oxy-silane (154 mg, 0.765 mmol) in NMP (3.0 mL) was added DIEA (197 mg, 1.53 mmol) in one portion at 20° C. under N₂. The reaction mixture was stirred at 180° C. for 2 hours under microwave, diluted with brine (20 mL) and extracted with ethyl acetate (30 mL). The combined extracts were washed with brine (20 mL), dried over Na₂SO₄, filtered, and concentrated under reduced pressure. The crude product was purified by column chromatography on silica gel (gradient 0˜100% ethyl acetate in petroleum ether) to afford 6-amino-7-(3-hydroxy-2,6-dimethyl-phenyl)-2-[(3R)-3-hydroxypyrrolidin-1-yl]pyrrolo[2,3-d]pyrimidine-5-carbonitrile (110 mg, 55% yield) as yellow oil. LCMS R_{T}=0.397 min, m/z=365.0 [M+H]⁺.

### Step 3: Synthesis of 6-amino-7-(3-hydroxy-2,6-dimethyl-phenyl)-2-[(3R)-3-hydroxypyrrolidin-1-yl]pyrrolo[2,3-d]pyrimidine-5-carboxamide (Compound 583)

A solution of 6-amino-7-(3-hydroxy-2,6-dimethyl-phenyl)-2-[(3R)-3-hydroxypyrrolidin-1-yl]pyrrolo[2,3-d]pyrimidine-5-carbonitrile (50 mg, 0.137 mmol) in concentrated H₂SO₄ (0.50 mL) was stirred at 20° C. for 1 hour, and then quenched with saturated NaHCO₃ solution (50 mL). The mixture was concentrated under vacuum, then triturated with MeOH (20 mL) and dichloromethane (20 mL) at 20° C. for 5 min. The filtrate was concentrated under reduced pressure to afford a yellow oil (30 mg, 100% yield). LCMS R_{T}=0.374 min, m/z=463.0 [M+H]⁺.

The above obtained oil (50 mg) was suspended in 4M HCl/MeOH (1.72 mL) and stirred at 25° C. for 1 hour. The mixture was concentrated under vacuum. The residue was purified by preparative HPLC [Column: Welch Xtimate C18 150*30 mm*5 um; Mobile phase: [water (FA)-MeCN]; B %: 0˜22%; Gradient time: 30 min] to afford 6-amino-7-(3-hydroxy-2,6-dimethyl-phenyl)-2-[(3R)-3-hydroxypyrrolidin-1-yl]pyrrolo[2,3-d]pyrimidine-5-carboxamide (5.7 mg, 14% yield) as a yellow solid. LCMS R_{T}=1.101 min, m/z=383.4 [M+H]⁺. ¹H NMR (400 MHz, DMSO-d₆) δ 9.53 (s, 1H), 8.58 (s, 1H), 8.13 (s, 1H), 7.05 (d, J=8.4 Hz, 1H), 6.89 (d, J=8.0 Hz, 1H), 6.63 (d, J=17.6 Hz, 4H), 4.83 (s, 1H), 4.26 (s, 1H), 3.23 (d, J=11.2 Hz, 4H), 1.90 (td, J=4.0, 12.4 Hz, 1H), 1.83-1.69 (m, 7H).

### Example 22. Synthesis of Compound 593

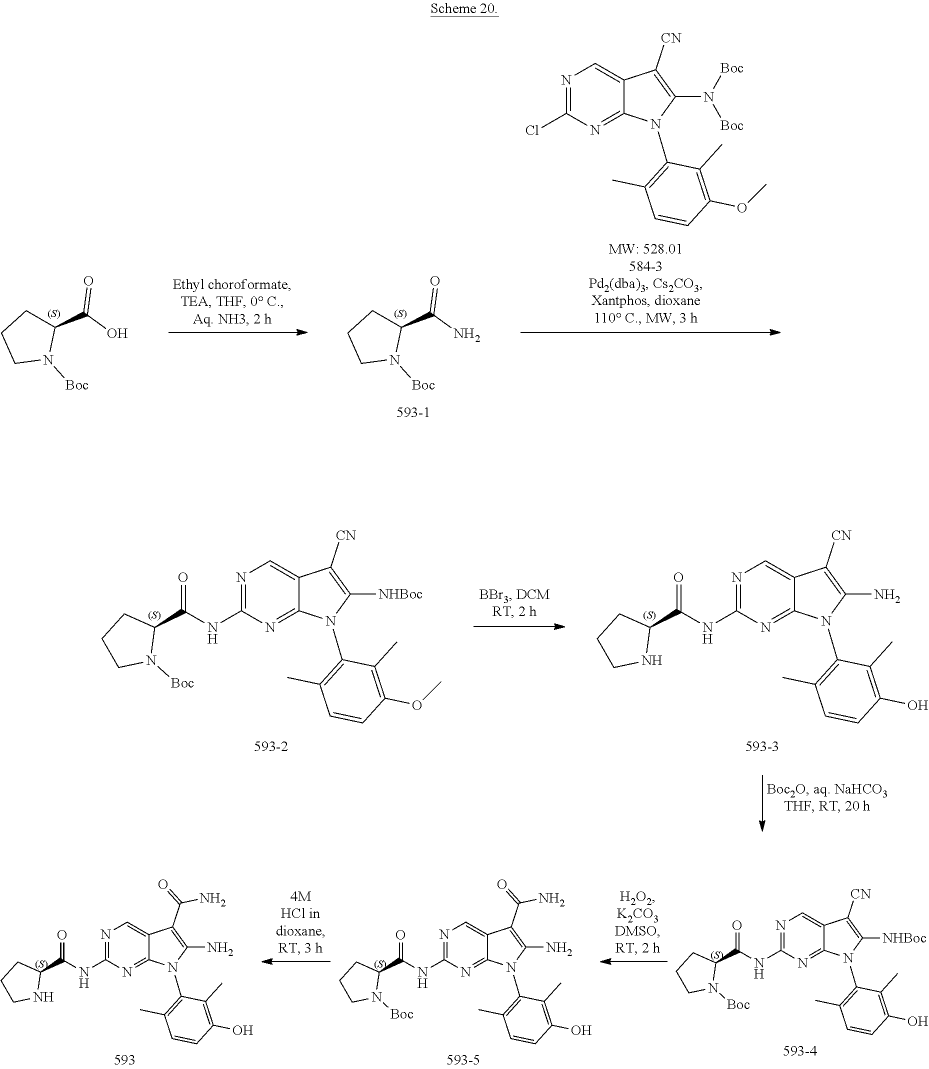

### Step 1. Synthesis of tert-butyl (S)-2-((6-((tert-butoxy carbonyl) amino)-5-cyano-7-(3-methoxy-2,6-dimethylphenyl)-7H-pyrrolo[2,3-d]pyrimidin-2-yl) carbamoyl) pyrrolidine-1-carboxylate

To a stirred solution of (tert-butoxy carbonyl)-L-proline (5.0 g, 23.25 mmol, 1.0 eq) in THF (50 mL) was added Et₃N (3.3 mL, 23.25 mmol, 1.0 eq) followed by ethyl chloroformate, (5.5 g, 51.1 mmol, 2.2 eq) at 0° C. and the mixture was stirred for 30 min. Then aq. NH₃ (7 mL) was added and the reaction mixture was stirred at room temperature for 1 6 hours. After completion of the reaction as indicated by TLC, the reaction mixture was poured in water (100 mL) and extracted with ethyl acetate (3×100 mL). The combined organic layers were dried over Na₂SO₄ and concentrated under vacuum to afford tert-butyl (S)-2-carbamoylpyrrolidine-1-carboxylate (Compound 593) as a light-yellow solid (3.5 g, 70.32%). LCMS: RT=1.355 min, m/z 215.2 (M+H)⁺.

To a stirred solution tert-butyl (2-chloro-5-cyano-7-(3-methoxy-2,6-dimethylphenyl)-7H-pyrrolo[2,3-d]pyrimidin-6-yl)dicarbamate (0.3 g, 0.568 mmol, 1.0 eq) and tert-butyl (S)-2-carbamoylpyrrolidine-1-carboxylate (Compound 593) (0.18 g, 0.852 mmol, 1.5 eq) in dioxane (10 mL), CS₂CO₃ (0.55 g, 1.70 mmol, 3.0 eq) was added and the reaction mixture was purged with argon for 15 min. Pd₂(dba)₃ (0.051 g, 0.056 mmol, 0.1 eq) and Xantphos (0.65 g, 0.113 mmol, 0.2 eq) were added and the reaction mixture was stirred at 130° C. for 3 hours in a microwave. After completion of the reaction as indicated by TLC, the resulting reaction mixture was poured into water (50 mL) and extracted with ethyl acetate (50 mL×3). The combined organic layers were dried over Na₂SO₄, filtered, and concentrated under reduced pressure. The crude compound was purified by column chromatography (230-400 mesh silica gel, 0-60% EtOAc/hexane) to afford title compound as a yellow solid (0.72 g, 41.84%). LCMS: RT=2.51 min, m/z 606.8 (M+H)⁺. ¹H NMR (DMSO-d₆, 400 MHz): δ 10.65 (s, 1H), 10.03 (t, J=3.6 Hz, 1H), 9.06 (d, J=5.6 Hz, 1H), 7.24 (d, J=8.4 Hz, 1H), 7.10 (d, J=8.8 Hz, 1H), 4.42 (s, 1H), 3.83 (s, 3H), 3.28 (m, 2H), 1.80 (m, 6H), 1.68 (m, 4H), 1.39 (s, 9H), 1.36 (s, 3H), 1.18 (s, 6H).

### Step 2. Synthesis of tert-butyl (S)-2-((6-((tert-butoxy carbonyl) amino)-5-cyano-7-(3-hydroxy-2,6-dimethylphenyl)-7H-pyrrolo[2,3-d]pyrimidin-2-yl) carbamoyl) pyrrolidine-1-carboxylate

To a stirred solution of tert-butyl (S)-2-((6-((tert-butoxy carbonyl) amino)-5-cyano-7-(3-methoxy-2,6-dimethylphenyl)-7H-pyrrolo[2,3-d]pyrimidin-2-yl)carbamoyl) pyrrolidine-1-carboxylate (0.670 g, 1.11 mmol, 1.0 eq) in dichloromethane (10 mL) was added BBr₃ (1 M in DCM) (11.0 mL, 11.06 mmol, 10.0 eq) at 0° C. The reaction was stirred under N₂ at room temperature for 2 hours. After completion of the reaction as indicated by TLC, the reaction was quenched with MeOH (10 mL) and concentrated under reduced pressure. The crude compound was purified by trituration using diethyl ether to afford (S)—N-(6-amino-5-cyano-7-(3-hydroxy-2,6-dimethylphenyl)-7H-pyrrolo[2,3-d]pyrimidin-2-yl) pyrrolidine-2-carboxamide (593-3) as yellow solid (0.630 g, quantitative). LCMS: RT=1.67 min, m/z 392.4 (M+H)⁺.

To a stirred solution of (S)—N-(6-amino-5-cyano-7-(3-hydroxy-2,6-dimethylphenyl)-7H-pyrrolo[2,3-d]pyrimidin-2-yl) pyrrolidine-2-carboxamide (0.630 g, 1.61 mmol, 1.0 eq) (593-3) in THF (10 mL), sat·NaHCO₃ solution (5 mL) and Boc anhydride (3.5 g, 16.10 mmol, 10.0 eq) were added at room temperature. The reaction mixture was stirred at room temperature for 24 hours. After completion of the reaction as indicated by TLC, the resulting reaction mixture was poured into water (30 mL) and extracted with ethyl acetate (30 mL×3). The combined organic layers were dried over Na₂SO₄, filtered, and concentrated under reduced pressure. The crude compound was purified by column chromatography (230-400 mesh silica gel, 0-50% EtOAc/hexane) to afford title compound as brown solid (0.230 g, 24.15%). LCMS: RT=2.35 min, m/z 592.7 (M+H)⁺. ¹H NMR (DMSO-d₆, 400 MHz): δ 10.37 (s, 1H), 8.49 (s, 1H), 7.41 (s, 2H), 7.33 (s, 2H), 4.40 (bs, 1H), 1.91 (s, 4H), 1.73 (t, J=4.8 Hz, 6H), 1.50 (s, 9H), 1.34 (s, 3H), 1.19 (s, 6H).

### Step 3. Synthesis of tert-butyl (S)-2-((6-amino-5-carbamoyl-7-(3-hydroxy-2,6-dimethylphenyl)-7H-pyrrolo[2,3-d]pyrimidin-2-yl) carbamoyl) pyrrolidine-1-carboxylate

To a stirred solution of tert-butyl (S)-2-((6-((tert-butoxy carbonyl) amino)-5-cyano-7-(3-hydroxy-2,6-dimethylphenyl)-7H-pyrrolo[2,3-d]pyrimidin-2-yl) carbamoyl) pyrrolidine-1-carboxylate (0.2 g, 0.338 mmol, 1.0 eq) in DMSO (5 mL), K₂CO₃ (0.467 g, 3.380 mmol, 10.0 eq) was added at 0° C. and the mixture was stirred for 5 min. H₂O₂ (30% in H₂O) (2 mL) was added dropwise at 0° C. and the mixture was stirred at room temperature for 2 hours. After completion of the reaction as indicated by TLC, the reaction mixture was purified by reverse phase chromatography (0-40% ACN/Water) to afford the title compound as a light-yellow solid (0.140 g, 81.28%). LCMS: RT=1.601 min, m/z 510.5 (M+H)⁺

¹H NMR (DMSO-d₆, 400 MHz): δ 10.21 (s, 1H), 9.60 (s, 1H), 8.84 (d, J=4.8 Hz, 1H), 7.07 (d, J=4.8 Hz, 3H), 6.94-6.89 (m, 3H), 4.35 (bs, 1H), 3.29 (t, J=7.2 Hz, 2H), 1.95 (bs, 2H), 1.78 (d, J=4.8 Hz, 5H), 1.69 (t, J=4.4 Hz, 3H), 1.37 (s, 3H), 1.20 (s, 6H).

### Step 4. Synthesis of (S)-6-amino-7-(3-hydroxy-2,6-dimethylphenyl)-2-(pyrrolidine-2-carboxamido)-7H-pyrrolo[2,3-d]pyrimidine-5-carboxamide (Compound 593)

To a solution of tert-butyl (S)-2-((6-amino-5-carbamoyl-7-(3-hydroxy-2,6-dimethylphenyl)-7H-pyrrolo[2,3-d]pyrimidin-2-yl) carbamoyl) pyrrolidine-1-carboxylate (0.130 g, 0.255 mmol, 1.0 eq) was added 4M HCl in dioxane (2 mL) and the mixture was stirred at room temperature for 3 hours. After completion of the reaction as indicated by TLC, the reaction mixture was concentrated under vacuum. The crude compound was purified by trituration using diethyl ether to afford the title compound as a yellow solid (0.043 g, 41.16%). LCMS: RT=1.77 min, m/z 410.5 (M+H)⁺. ¹H NMR (DMSO-d₆, 400 MHz): δ 11.54 (bs, 1H), 10.07 (s, 1H), 9.80 (s, 1H), 8.88 (s, 1H), 8.66 (s, 1H), 7.35 (s, 2H), 7.09 (d, J=8.4 Hz, 2H), 7.00 (d, J=8.4 Hz, 2H), 4.41 (bs, 1H), 3.232-3.177 (m, 2H), 2.24 (bs, 2H), 1.84 (d, J=9.2 Hz, 2H), 1.80 (d, J=3.6 Hz, 3H), 1.72 (d, J=4.8 Hz, 3H).

### Example 23. Synthesis of Compound 584

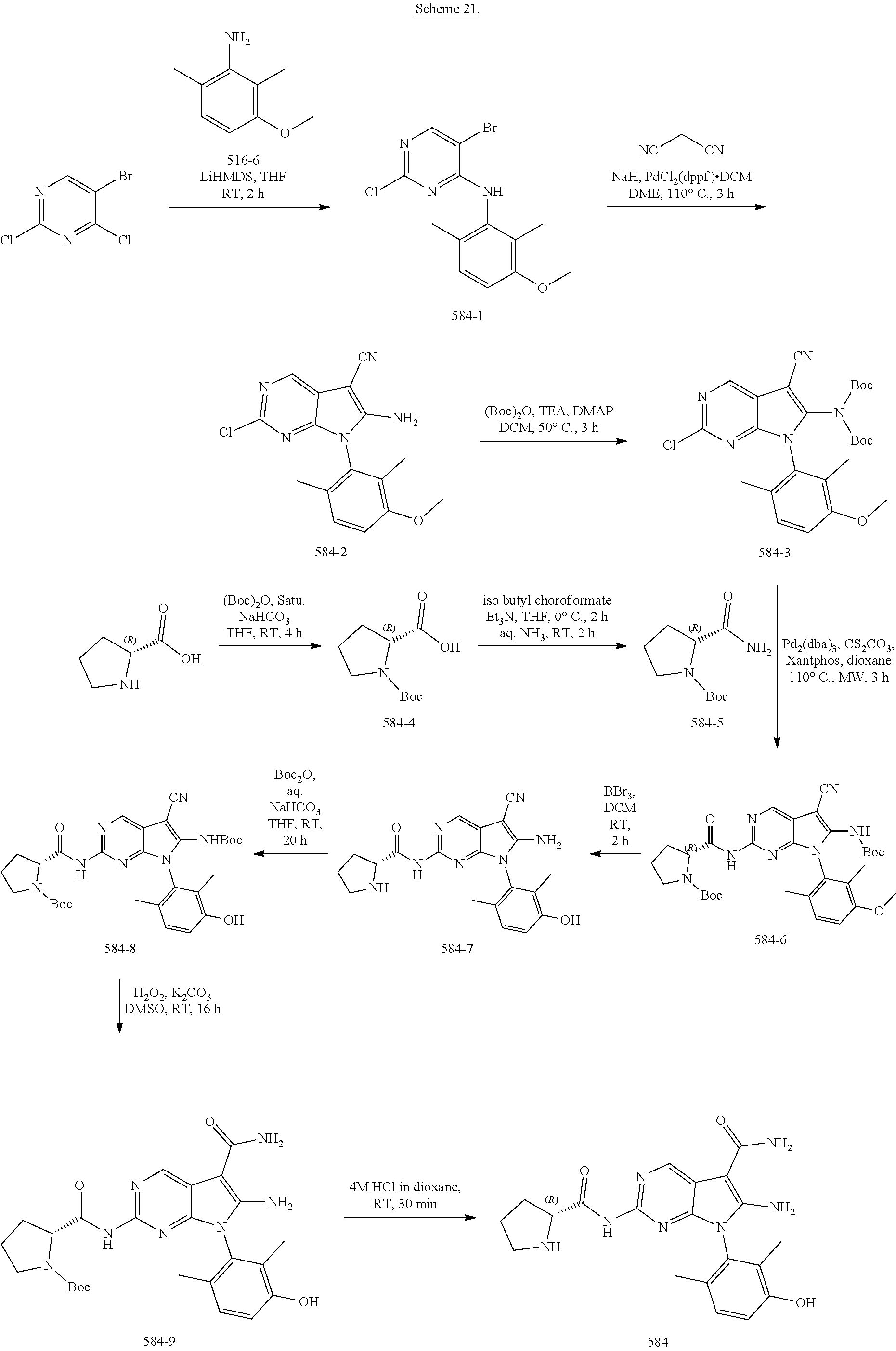

### Step 1. Synthesis of 5-bromo-2-chloro-N-(3-methoxy-2,6-dimethylphenyl) pyrimidin-4-amine

To a stirred solution of 3-methoxy-2,6-dimethylaniline (10.0 g, 66.22 mmol, 1.0 eq) and 5-bromo-2,4-dichloropyrimidine (30.14 g, 132.2 mmol, 2.0 eq) in THF (100 mL) at 0° C., LiHMDS (1M in THF) (132 mL, 132.4 mmol, 2.0 eq) was added and the reaction was stirred at room temperature for 2 hours. After completion of the reaction as indicated by TLC, the resulting mixture was quenched with water (100 mL) and extracted with ethyl (3×100 mL). The organic layer was dried over Na₂SO₄ and concentrated under vacuum. The crude product was purified by column chromatography (230-400 mesh silica gel, 0-8% EtOAc/hexane) to afford the title compound as a brown solid (7.0 g, 30.89%). LCMS: RT=2.451 min, m/z 342 (M+H)⁺. ¹H NMR (DMSO-d₆, 400 MHz): δ 9.12 (s, 1H), 8.40 (s, 1H), 7.11 (d, J=8.4 Hz, 1H), 6.90 (d, J=8.4 Hz, 1H), 3.79 (s, 3H), 2.03 (s, 3H), 1.93 (s, 3H).

### Step 2. Synthesis of 6-amino-2-chloro-7-(3-methoxy-2,6-dimethylphenyl)-7H-pyrrolo[2,3-d]pyrimidine-5-carbonitrile

To a stirred solution of malononitrile (2.6 g, 40.8 mmol, 2.0 eq) in DME (70 mL) at 0° C., NaH (55% in mineral oil) (1.63 g, 40.8 mmol, 2.0 eq) was added and the reaction was stirred for 15 min at 0° C. 5-Bromo-2-chloro-N-(3-methoxy-2,6-dimethylphenyl) pyrimidin-4-amine (7.0 g, 20.43 mmol, 1.0 eq) and PdCl₂(dppf)DCM (1.67 g, 2.04 mmol, 0.1 eq) were added and the reaction was stirred at 120° C. for 3 hours. After completion of the reaction as indicated by TLC, the reaction was diluted with water (100 mL) and extracted with ethyl acetate (3×100 mL). The organic layer was dried over Na₂SO₄ and concentrated under vacuum. The crude product was purified by column chromatography (230-400 mesh silica gel, 0-30% EtOAc/hexane) to afford the title compound as a light brown solid (2.2 g, 32.85%). LCMS: RT=2.156 min, m/z 328 (M+H)⁺. ¹H NMR (DMSO-d6, 400 MHz): δ 8.53 (s, 1H), 7.65 (s, 1H), 7.28 (d, J=8.4 Hz, 1H), 7.14 (d, J=8.4 Hz, 1H), 3.85 (s, 3H), 1.81 (s, 3H), 1.72 (s, 3H).

### Step 3. Synthesis of tert-butyl (tert-butoxycarbonyl)(2-chloro-5-cyano-7-(3-methoxy-2,6-dimethylphenyl)-7H-pyrrolo[2,3-d]pyrimidin-6-yl)carbamate

To a stirred solution of 6-amino-2-chloro-7-(3-methoxy-2,6-dimethylphenyl)-7H-pyrrolo[2,3-d]pyrimidine-5-carbonitrile (2.2 g, 6.72 mmol, 1.0 eq) in DCM (25 mL), Boc anhydride (7.33 g, 33.6 mmol, 5.0 eq), Et₃N (2.9 mL, 20.1 mmol, 3.0 eq), and DMAP (0.081 g, 0.67 mmol, 0.1 eq) were added and the reaction mixture was stirred at room temperature for 3 hours. After completion of the reaction as indicated by TLC, the resulting mixture was quenched with water (50 mL) and extracted with DCM (3×100 mL). The organic layer was dried over Na₂SO₄ and concentrated under vacuum. The crude was purified by column chromatography (230-400 mesh silica gel, 0-20% EtOAc/hexane) to afford the title compound as a brown solid (2.0 g, 56.43%). LCMS: RT=3.029 min, m/z 528 (M+H)+. ¹H NMR (DMSO-d₆, 400 MHz): δ 9.37 (s, 1H), 7.28 (d, J=8.4 Hz, 1H), 7.18 (d, J=8.4 Hz, 1H), 3.84 (s, 3H), 1.81 (s, 3H), 1.66 (s, 3H), 1.33 (s, 18H).

### Synthesis of (tert-butoxy carbonyl)-D-proline

To stirred a solution of D-proline (2.0 g, 17.37 mmol, 1.0 eq) in THF (20 mL), sat·NaHCO₃ solution (30 mL) and Boc anhydride (4.55 g, 20.86 mmol, 1.2 eq) were added and the reaction mixture was stirred at room temperature for 4 hours. After completion of the reaction as indicated by TLC, the resulting reaction mixture was poured into water (50 mL) and extracted with ethyl acetate (50 mL×3). The combined organic layers were dried over Na₂SO₄, filtered, and concentrated under reduced pressure. The crude compound was purified by column chromatography (230-400 mesh silica gel, 0-10% MeOH/DCM) to afford the title compound as an off white solid (2.1 g, 56.16%). LCMS: RT=1.57 min, m/z 216 (M+H)+

### Synthesis of tert-butyl (R)-2-carbamoylpyrrolidine-1-carboxylate

To stirred a solution of (tert-butoxy carbonyl)-D-proline (2.1 g, 9.76 mmol, 1.0 eq) in THF (20 mL), triethylamine (0.98 g, 9.76 mmol, 1.0 eq) and isobutyl chloroformate (2.92 g, 21.48 mmol, 2.2 eq) were added at 0° C. The reaction mixture was stirred at room temperature for 30 min. After 30 min, aq. ammonia (3 mL, 1.5 vol) was added and the reaction mixture was stirred at room temperature for 2 hours. After completion of the reaction as indicated by TLC, the resulting reaction mixture was poured into water (30 mL) and extracted with (50 mL×3). The combined organic layers were dried over Na₂SO₄, filtered and concentrated under reduced pressure. The crude compound was purified by column chromatography (230-400 mesh silica gel, 0-20% MeOH/DCM) to afford the title compound as an off-white solid (1.7 g, 81.32%). LCMS: RT=1.33 min, m/z 215 (M+H)⁺.

### Step 4. Synthesis of tert-butyl (R)-2-((6-((tert-butoxy carbonyl) amino)-5-cyano-7-(3-methoxy-2,6-dimethylphenyl)-7H-pyrrolo[2,3-d]pyrimidin-2-yl) carbamoyl) pyrrolidine-1-carboxylate

To a stirred solution 584-3 (0.3 g, 0.568 mmol, 1.0 eq) and tert-butyl (R)-2-carbamoylpyrrolidine-1-carboxylate (0.18 g, 0.852 mmol, 1.5 eq) in dioxane (10 mL), CS₂CO₃ (0.55 g, 1.70 mmol, 3.0 eq) was added and the reaction mixture was purged with argon for 15 min. Pd₂(dba)₃ (0.052 g, 0.056 mmol, 0.1 eq) and Xantphos (0.65 g, 0.113 mmol, 0.2 eq) were added and the reaction mixture was stirred at 130° C. for 3 hours in a microwave. After completion of the reaction as indicated by TLC, the resulting reaction mixture was poured into water (30 mL) and extracted with ethyl acetate (50 mL×3). The combined organic layers were dried over Na₂SO₄, filtered, and concentrated under reduced pressure. The crude compound was purified by column chromatography (230-400 mesh silica gel, 0-60% EtOAc/hexane) to afford the title compound as a yellow solid (0.4 g, 23.25%). LCMS: RT=2.55 min, m/z 606 (M+H)⁺. ¹H NMR (DMSO-d₆, 400 MHz): δ 10.65 (s, 1H), 10.03 (t, J=3.2 Hz, 1H), 9.05 (d, J=5.6 Hz, 1H), 7.24 (d, J=8.4 Hz, 1H), 7.10 (d, J=8.4 Hz, 1H), 4.41 (bs, 1H), 3.84 (d, J=2.8 Hz, 3H), 2.02 (s, 2H), 1.80 (q, J=11.2 Hz, 5H), 1.68 (t, J=4 Hz, 3H), 1.39 (s, 9H), 1.23 (s, 3H), 1.18 (s, 6H).

### Step 5. Synthesis of tert-butyl 2-((6-((tert-butoxy carbonyl) amino)-5-cyano-7-(3-hydroxy-2,6-dimethylphenyl)-7H-pyrrolo[2,3-d]pyrimidin-2-yl) carbamoyl) pyrrolidine-1-carboxylate

To a stirred solution of tert-butyl (R)-2-((6-((tert-butoxy carbonyl) amino)-5-cyano-7-(3-methoxy-2,6-dimethylphenyl)-7H-pyrrolo[2,3-d]pyrimidin-2-yl) carbamoyl) pyrrolidine-1-carboxylate (0.4 g, 0.661 mmol, 1.0 eq) in DCM (4 mL) was added BBr₃ (1 M in DCM) (3.96 mL, 3.96 mmol, 6.0 eq). The reaction was stirred under N₂ at room temperature for 2 hours. After completion of the reaction as indicated by TLC, the reaction was quenched with MeOH (10 mL) and concentrated under reduced pressure. The crude compound was purified by reverse phase chromatography using (C18, 0-60% ACN/Water) to afford N-(6-amino-5-cyano-7-(3-hydroxy-2,6-dimethylphenyl)-7H-pyrrolo[2,3-d]pyrimidin-2-yl) pyrrolidine-2-carboxamide (584-7) as an off-white solid (0.2 g, 77.37%). LCMS: RT=1.23 and 1.27 min, m/z 392 (M+H)⁺.

To stirred a solution of N-(6-amino-5-cyano-7-(3-hydroxy-2,6-dimethylphenyl)-7H-pyrrolo[2,3-d]pyrimidin-2-yl) pyrrolidine-2-carboxamide (0.2 g, 0.51 mmol, 1.0 eq) in THF (2 mL), sat·NaHCO₃ solution (0.8 mL) and Boc anhydride (0.446 g, 2.043 mmol, 4.0 eq) were added. The reaction mixture was stirred at room temperature for 20 hours. After completion of the reaction as indicated by TLC, the resulting reaction mixture was poured in to water (30 mL) and extracted with ethyl acetate (30 mL×3). The combined organic layers were dried over Na₂SO₄, filtered, and concentrated under reduced pressure. The crude compound was purified by column chromatography (230-400 mesh silica gel, 0-50% EtOAc/hexane) to afford the title compound as a brown solid (0.065 g, 21.50%). LCMS: RT=2.30 min, m/z 592.6 (M+H)⁺. ¹H NMR (DMSO-d₆, 400 MHz): δ 10.37 (s, 1H), 8.48 (d, J=7.6 Hz, 1H), 7.41 (s, 2H), 7.33 (s, 2H), 4.39 (bs, 1H), 3.27 (q, J=10.4 Hz, 2H), 1.90 (d, J=6.8 Hz, 4H), 1.72 (t, J=4.4 Hz, 6H), 1.50 (s, 9H), 1.36 (s, 3H), 1.18 (s, 6H).

### Step 6. Synthesis of tert-butyl 2-((6-((tert-butoxy carbonyl) amino)-5-carbamoyl-7-(3-hydroxy-2,6-dimethylphenyl)-7H-pyrrolo[2,3-d]pyrimidin-2-yl) carbamoyl) pyrrolidine-1-carboxylate

To a stirred solution of tert-butyl 2-((6-((tert-butoxy carbonyl) amino)-5-cyano-7-(3-hydroxy-2,6-dimethylphenyl)-7H-pyrrolo[2,3-d]pyrimidin-2-yl) carbamoyl) pyrrolidine-1-carboxylate (0.06 g, 0.101 mmol, 1.0 eq) in DMSO (1 mL), K₂CO₃ (0.139 g, 1.014 mmol, 10.0 eq) was added at 0° C. and the reaction mixture was stirred for 5 min. Then H₂O₂ (30%) (1 mL) was added dropwise at 0° C. and the reaction mixture was stirred at room temperature for 2 hours. After completion of the reaction as indicated by TLC, the reaction mixture was purified by reverse phase chromatography (0-40% ACN/Water) to afford the title compound as a white solid (0.045 g, 87.08%). LCMS: RT=1.591 min, m/z 510.5 (M+H)⁺. ¹H NMR (DMSO-d₆, 400 MHz, D₂O): δ 10.20 (s, 1H), 8.84 (s, 1H), 7.06 (d, J=8 Hz, 1H), 6.90 (d, J=8.4 Hz, 1H), 4.24 (bs, 1H), 3.29 (d, J=6.4 Hz, 2H), 2.50 (bs, 2H), 2.05 (bs, 2H), 1.77 (d, J=4.8 Hz, 3H), 1.69 (t, J=4.8 Hz, 3H), 1.36 (s, 3H), 1.20 (s, 6H).

### Step 7. Synthesis of 6-amino-7-(3-hydroxy-2,6-dimethylphenyl)-2-(pyrrolidine-2-carboxamido)-7H-pyrrolo[2,3-d]pyrimidine-5-carboxamide (Compound 584)

A solution of tert-butyl 2-((6-((tert-butoxy carbonyl) amino)-5-carbamoyl-7-(3-hydroxy-2,6-dimethylphenyl)-7H-pyrrolo[2,3-d]pyrimidin-2-yl) carbamoyl) pyrrolidine-1-carboxylate (0.030 g, 0.058 mmol, 1.0 eq) wand 4M HCl in dioxane (1 mL) was stirred at room temperature for 1 hour. After completion of the reaction as indicated by TLC, the reaction mixture was concentrated under vacuum. The crude compound was purified by reverse phase chromatography (C18, 0-40% ACN/Water) to afford the title compound as an off-white solid (0.015 g, 62.23%). LCMS: RT=1.77 min, m/z 410.5 (M+H)⁺. ¹H NMR (MeOD, 400 MHz): δ 8.76 (s, 1H), 7.15 (d, J=8.4 Hz, 1H), 6.97 (d, J=8.4 Hz, 1H), 4.49 (bs, 1H), 3.50-3.41 (m, 2H), 2.50 (bs, 2H), 2.11 (bs, 2H), 1.93 (s, 3H), 1.86 (s, 3H).

### Example 24. Synthesis of Compound 587

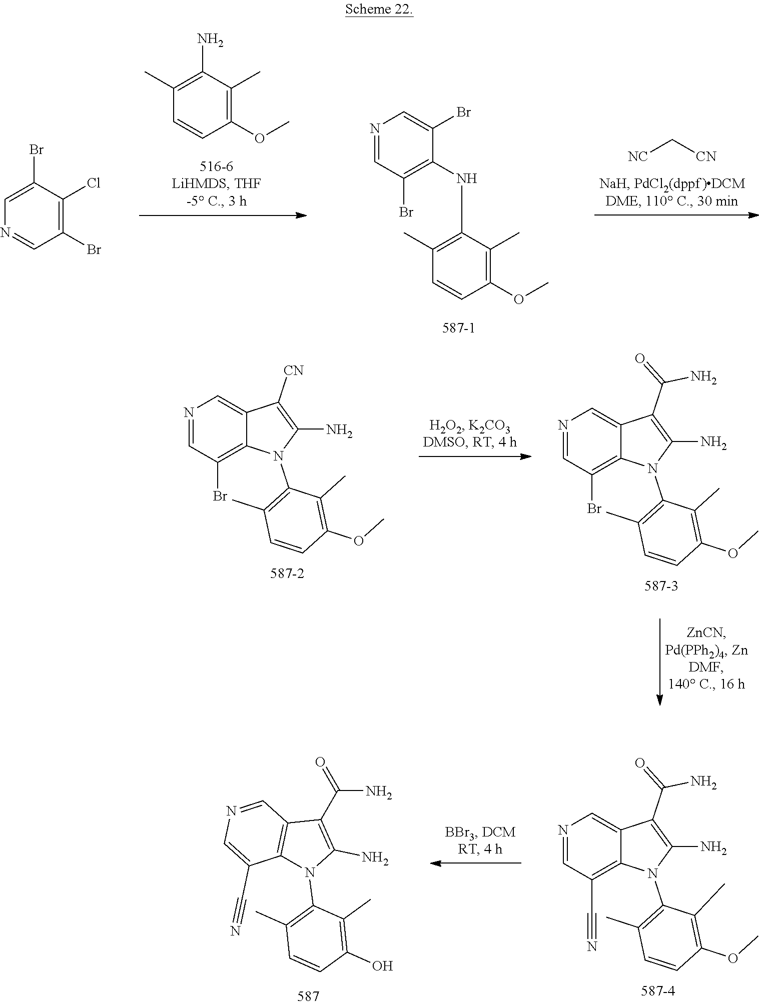

### Step 1: Synthesis of 3,5-dibromo-N-(3-methoxy-2,6-dimethylphenyl) pyridin-4-amine

To a stirred solution of 3,5-dibromo-4-chloropyridine (30 g, 111.59 mmol, 1.0 eq) and 3-methoxy-2,6-dimethylaniline (16.85 g, 111.59 mmol, 1.0 eq) in THF (300 mL) at −5° C., LiHMDS (1M in THF) (223 mL, 223.19 mmol, 2.0 eq) was added and the reaction was stirred at −5° C. for 3 hours. After completion of the reaction as indicated by TLC, the resulting mixture was quenched with water (500 mL) and extracted with ethyl acetate (3×500 mL). The organic layer was dried over Na₂SO₄ and concentrated under vacuum. The crude product was purified by Reverse phase column chromatography (C18 silica, 0-65% ACN/Water) to afford the title compound as a brown solid (18.0 g, 42.17%). LCMS: RT=2.372 min, m/z 387 (M+H)⁺.

### Step 2: Synthesis of 2-amino-7-bromo-1-(3-methoxy-2,6-dimethylphenyl)-1H-pyrrolo[3,2-c]pyridine-3-carbonitrile

To a stirred solution of malononitrile (6.16 g, 93.24 mmol, 2.0 eq) in DME (200 mL) at 0° C., sodium ethoxide (21% in ethanol) (61.6 mL, 10 V) was added and the reaction mixture was stirred for 15 min at 0° C. 3,5-Dibromo-N-(3-methoxy-2,6-dimethylphenyl) pyridin-4-amine (18.0 g, 46.62 mmol, 1.0 eq) and PdCl₂(dppf)DCM (1.9 g, 2.33 mmol, 0.05 eq) were added and the reaction was stirred at 110° C. for 30 min. After completion of the reaction as indicated by TLC, the reaction was quenched with water (200 mL) and extracted with ethyl acetate (3×300 mL). The organic layer was dried over Na₂SO₄ and concentrated under vacuum. The crude product was purified by column chromatography (230-400 mesh silica gel, 0-80% EtOAc/hexane) to afford the title compound as an off-white solid (8.0 g, 46.22%). LCMS: RT=1.369 min, m/z 371 (M+H)⁺. ¹H NMR (DMSO-d₆, 400 MHz): δ 8.43 (s, 1H), 8.11 (s, 1H), 7.21 (t, J=8.8 Hz, 3H), 7.12 (d, J=8.4 Hz, 1H), 3.83 (s, 3H), 1.78 (s, 3H), 1.70 (s, 3H).

### Step 3: Synthesis of 2-amino-7-bromo-1-(3-methoxy-2,6-dimethylphenyl)-1H-pyrrolo[3,2-c]pyridine-3-carboxamide

To a stirred solution of 2-amino-7-bromo-1-(3-methoxy-2,6-dimethylphenyl)-1H-pyrrolo[3,2-c]pyridine-3-carbonitrile (8.0 g, 21.55 mmol, 1.0 eq) in DMSO (80 mL), K₂CO₃ (29.7 g, 215.5 mmol, 10.0 eq) was added at 0° C. and the reaction was stirred for 5 min. H₂O₂ (30% in H₂O) (80 mL) was added dropwise at 0° C. and the reaction was stirred at room temperature for 3 hours. After completion of the reaction as indicated by TLC, the reaction mixture was poured into water (100 mL) and extracted with ethyl acetate (3×100 mL) to afford the crude as an off-white solid (1.1 g, 13.11%). The crude was used in the next step without purification LCMS: RT=1.480 min, m/z 389 (M+H)⁺. ¹H NMR (DMSO-d₆, 400 MHz): δ 8.90 (s, 1H), 8.07 (s, 1H), 7.24 (d, J=8.4 Hz, 1H), 7.13 (d, J=8.4 Hz, 1H), 6.98 (s, 2H), 6.92 (s, 2H), 3.84 (s, 3H), 1.79 (s, 3H), 1.71 (s, 3H).

### Step 4: Synthesis of 2-amino-7-cyano-1-(3-methoxy-2,6-dimethylphenyl)-1H-pyrrolo[3,2-c]pyridine-3-carboxamide

To a stirred solution of 2-amino-7-bromo-1-(3-methoxy-2,6-dimethylphenyl)-1H-pyrrolo[3,2-c]pyridine-3-carboxamide (0.8 g, 2.06 mmol, 1.0 eq) in DMF (10 mL) at room temperature, zinc cyanide (0.129 g, 1.1 mmol, 0.54 eq), Zn metal (0.026 g, 0.41 mmol, 0.2 eq), BINAP (0.127 g, 0.2 mmol, 0.1 eq), and Pd(OAc)₂ (0.045 g, 0.2 mmol, 0.1 eq) were added and the reaction mixture was stirred under an inert argon atmosphere at 140° C. for 16 hours. After completion of the reaction as indicated by TLC, the reaction mixture was poured into water (50 mL) and extracted with ethyl acetate (3×50 mL) to afford the crude compound. The crude compound was purified by column chromatography (230-400 mesh silica gel, 0-70% EtOAc/hexane) to afford the title compound as an off-white solid (0.06 g, 8.71%). LCMS: RT=1.434 min, m/z 336 (M+H)⁺. ¹H NMR (DMSO-d₆, 400 MHz): δ 9.14 (s, 1H), 8.37 (s, 1H), 7.29 (d, J=8.4 Hz, 1H), 7.19 (d, J=4 Hz, 3H), 7.06 (s, 2H), 3.85 (s, 3H), 1.81 (s, 3H), 1.73 (s, 3H).

### Step 5: Synthesis of 2-amino-7-cyano-1-(3-hydroxy-2,6-dimethylphenyl)-1H-pyrrolo[3,2-c]pyridine-3-carboxamide (Compound 587)

To a stirred solution of 2-amino-7-cyano-1-(3-methoxy-2,6-dimethylphenyl)-1H-pyrrolo[3,2-c]pyridine-3-carboxamide (0.06 g, 0.179 mmol, 1.0 eq) in DCM (5 mL) at 0° C. was added BBr₃ (1M in DCM) (0.5 mL, 0.53 mmol, 3.0 eq). The reaction mixture was stirred at room temperate for 4 hours. After completion of the reaction as indicated by TLC, the reaction mixture was quenched with methanol and concentrated under vacuum. The crude product was purified by reverse phase column chromatography using (C18, 0-50% water/ACN) to afford the compound as an off-white solid (0.022 g, 38.27%).

LCMS: RT=1.201 min, m/z 322 (M+H)⁺. ¹H NMR (DMSO-d₆, 400 MHz): δ 9.71 (bs, 1H), 9.13 (s, 1H), 8.37 (s, 1H), 7.16 (s, 2H), 7.09 (d, J=8 Hz, 1H), 7.05 (s, 2H), 6.98 (d, J=8 Hz, 1H), 1.77 (s, 3H), 1.68 (s, 3H).

### Example 25. Synthesis of Compound 592

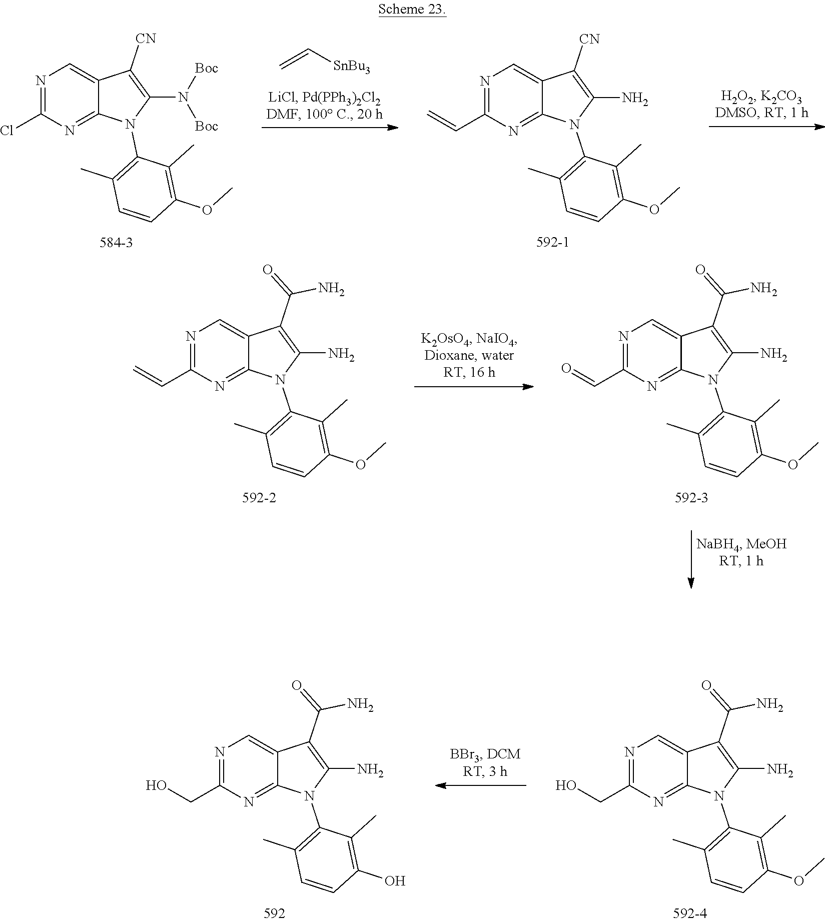

### Step 1: Synthesis of 6-amino-7-(3-methoxy-2,6-dimethylphenyl)-2-vinyl-7H-pyrrolo[2,3-d]pyrimidine-5-carbonitrile

To a stirred solution of tert-butyl (2-chloro-5-cyano-7-(3-methoxy-2,6-dimethylphenyl)-7H-pyrrolo[2,3-d]pyrimidin-6-yl) dicarbamate (3.0 g, 5.68 mmol, 1.0 eq) and tributyl(vinyl)stannane (9.0 g, 28.41 mmol, 5.0 eq) in DMF (30 mL) at room temperature, LiCl (0.235 g, 5.68 mmol, 1.0 eq) and Pd(PPh₃)₂Cl₂ (0.398 g, 0.56 mmol, 0.1 eq) were added and the reaction was stirred at 100° C. for 20 hours. After completion of the reaction as indicated by TLC, the resulting mixture was quenched with water (100 mL) and extracted with ethyl acetate (3×100 mL). The organic layer was dried over Na₂SO₄ and concentrated under vacuum. The crude product was purified by column chromatography (230-400 mesh silica gel, 20% EtOAc/hexane) to afford the title compound as a yellow sticky solid (1.3 g, 71.64%). LCMS: RT=1.93 min, m/z 320 (M+H)⁺. ¹H NMR (DMSO-d₆, 400 MHz): δ 8.60 (s, 1H), 7.46 (s, 2H), 7.27 (d, J=8.4 Hz, 1H), 7.12 (d, J=8.4 Hz, 1H), 6.61 (q, J=10.4 Hz, 1H), 6.20 (dd, J=2.4 Hz, J=17.5 Hz, 1H), 5.43 (dd, J=2 Hz, J=10.4 Hz, 1H), 3.85 (s, 3H), 1.81 (s, 3H), 1.72 (s, 3H).

### Step 2: Synthesis of 6-amino-7-(3-methoxy-2,6-dimethylphenyl)-2-vinyl-7H-pyrrolo[2,3-d]pyrimidine-5-carboxamide

To a stirred solution of 6-amino-7-(3-methoxy-2,6-dimethylphenyl)-2-vinyl-7H-pyrrolo[2,3-d]pyrimidine-5-carbonitrile (1.3 g, 4.07 mmol, 1.0 eq) in DMSO (13 mL), K₂CO₃ (5.5 g, 40 mmol, 10.0 eq) and H₂O₂ (30% in H₂O) (4.5 mL, 40 mmol, 10.0 eq) were added and the reaction was stirred at room temperature for 1 hour. After completion of the reaction as indicated by TLC, the resulting reaction mixture was poured into water (50 mL) and extracted with ethyl acetate (100 mL×3). The combined organic layers were dried over Na₂SO₄, filtered, and concentrated under reduced pressure. The crude compound was purified by column chromatography (230-400 mesh silica gel, 0-70% EtOAc/hexane) to afford the title compound as an off white solid (1.2 g, 87.38%). LCMS: RT=1.576 min, m/z 338 (M+H)⁺. ¹H NMR (DMSO-d₆, 400 MHz): δ 8.98 (s, 1H), 7.28 (d, J=8.8 Hz, 1H), 7.23 (s, 1H), 7.12 (d, J=8.4 Hz, 1H), 6.95 (s, 2H), 6.62 (q, J=10.4 Hz, 1H), 6.17 (dd, J=1.6 Hz, J=17.2 Hz, 1H), 5.40 (dd, J=1.6 Hz, J=10.4 Hz, 1H), 3.86 (s, 3H), 1.81 (s, 3H), 1.72 (s, 3H).

### Step 3: Synthesis of 6-amino-2-formyl-7-(3-methoxy-2,6-dimethylphenyl)-7H-pyrrolo[2,3-d]pyrimidine-5-carboxamide

To stirred a solution of 6-amino-7-(3-methoxy-2,6-dimethylphenyl)-2-vinyl-7H-pyrrolo[2,3-d]pyrimidine-5-carboxamide (1.2 g, 3.56 mmol, 1.0 eq) in dioxane:water (10:2 mL) were added K₂OSO₄ (0.0032 g, 0.0089 mmol, 0.0025eq) and NaIO₄ (7.45 g, 35.6 mmol, 10.0 eq) and the reaction was stirred at room temperature for 16 hours. After completion of the reaction as indicated by TLC, the resulting reaction mixture was poured into water (50 mL) and extracted with dichloromethane (50 mL×3). The combined organic layers were dried over Na₂SO₄, filtered, and concentrated under reduced pressure. The crude compound was purified by column chromatography (230-400 mesh silica gel, 0-3% MeOH/DCM) to afford the title compound as a light brown solid (0.65 g, 53.85%). LCMS: RT=1.670 min, m/z 340 (M+H)⁺. ¹H NMR (DMSO-d₆, 400 MHz): δ 9.77 (s, 1H), 9.21 (s, 1H), 7.67 (s, 2H), 7.31 (d, J=8.4 Hz, 1H), 7.17 (d, J=8.4 Hz, 3H), 3.87 (s, 3H), 1.80 (s, 3H), 1.71 (s, 3H).

### Step 4: Synthesis of 6-amino-2-(hydroxymethyl)-7-(3-methoxy-2,6-dimethylphenyl)-7H-pyrrolo [2,3-d]pyrimidine-5-carboxamide

To stirred a solution of 6-amino-2-formyl-7-(3-methoxy-2,6-dimethylphenyl)-7H-pyrrolo[2,3-d]pyrimidine-5-carboxamide (0.65 g, 1.92 mmol, 1.0 eq) in MeOH (13 mL) was added NaBH₄ (0.141 g, 3.84 mmol, 2.0 eq) and the reaction was stirred at room temperature for 1 hour. After completion of the reaction as indicated by TLC, the resulting reaction mixture was concentrated under reduced pressure. The crude compound was purified by column chromatography (230-400 mesh silica gel, 0-5% MeOH/DCM) to afford the title compound as a light brown solid (0.45 g, 68.82%). LCMS: RT=1.275 min, m/z 342 (M+H)⁺. ¹H NMR (DMSO-d₆, 400 MHz): δ 8.97 (s, 1H), 7.27 (d, J=8.4 Hz, 1H), 7.17 (s, 2H), 7.12 (d, J=8.8 Hz, 1H), 6.92 (s, 2H), 5.038 (t, J=6 Hz, 1H), 4.40 (d, J=6 Hz, 2H), 3.85 (s, 3H), 1.81 (s, 3H), 1.71 (s, 3H).

### Step 5: Synthesis of 6-amino-7-(3-hydroxy-2,6-dimethylphenyl)-2-(hydroxymethyl)-7H-pyrrolo[2,3-d]pyrimidine-5-carboxamide (Compound 592)

To stirred a solution of 6-amino-2-(hydroxymethyl)-7-(3-methoxy-2,6-dimethylphenyl)-7H-pyrrolo[2,3-d]pyrimidine-5-carboxamide (0.44 g, 1.29 mmol, 1.0 eq) in dichloromethane (10 mL) was added BBr₃ (1M in DCM) (3.8 mL, 3.87 mmol, 3.0 eq) and the reaction was stirred at room temperature for 3 hours. After completion of the reaction as indicated by TLC, the resulting reaction mixture was quenched with MeOH and the reaction mixture was concentrated under reduced pressure. The crude compound was purified by column chromatography (230-400 mesh silica gel, 0-3% MeOH/DCM) to afford the title compound as a white solid (0.39 g, 92.43%). LCMS: RT=1.053 min, m/z 328(M+H)⁺. ¹H NMR (DMSO-d₆, 400 MHz): δ 9.61 (s, 1H), 8.95 (s, 1H), 7.12 (s, 2H), 7.07 (d, J=8.4 Hz, 1H), 6.98 (d, J=8.4 Hz, 1H), 6.91 (s, 2H), 5.07 (t, J=6 Hz, 1H), 4.40 (d, J=5.6 Hz, 2H), 1.74 (s, 3H), 1.65 (s, 3H).

### Example 26. Synthesis of Compounds 571 and 572

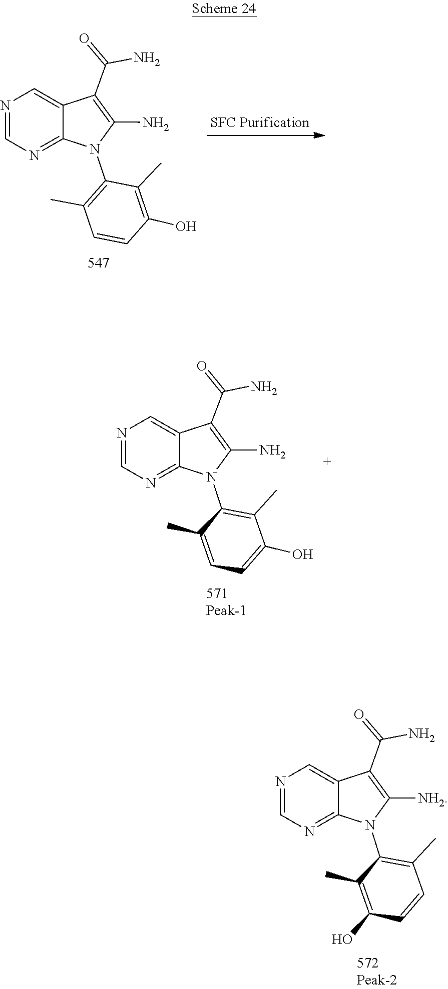

### 6-amino-7-(3-hydroxy-2,6-dimethylphenyl)-7H-pyrrolo[2,3-d]pyrimidine-5-carboxamide (Peak-1) (Compound 571) and 6-amino-7-(3-hydroxy-2,6-dimethylphenyl)-7H-pyrrolo[2,3-d]pyrimidine-5-carboxamide (Peak-2) (Compound 572)

Compound 547 was purified by SFC using (YMC CELLULOSE SC, 250×20 mm, 5 μm), MOBILE PHASE A: 1% NH₃ (7 N in methanol) in n-Heptane MOBILE PHASE B: 0.1% 7 N NH₃ (in MEOH): MTBE (50:50) to afford the title compounds as an off-white solid.

Compound 571 (Peak-1) (0.095 g, 13.57%); LCMS: RT=1.072 min, m/z=297 [M+H]⁺; HPLC: RT=3.70 min; Chiral HPLC: RT=8.26 min time; ¹H NMR (400 MHz, DMSO) δ 9.67 (bs, 1H), 8.99 (s, 1H), 8.40 (s, 1H), 7.20 (bs, 2H), 7.07 (d, J=7.6 Hz, 1H), 6.94 (m, 3H), 1.74 (s, 3H), 1.66 (s, 3H).

Compound 572 (Peak-2) (0.050 g, 7.14%); LCMS: RT=1.045 min, m/z=297 [M+H]⁺; HPLC: RT=3.99 min; Chiral HPLC: RT=8.45 min time; ¹H NMR (400 MHz, DMSO) δ 9.67 (bs, 1H), 8.99 (s, 1H), 8.40 (s, 1H), 7.20 (bs, 2H), 7.07 (d, J=7.6 Hz, 1H), 6.94 (m, 3H), 1.74 (s, 3H), 1.66 (s, 3H).

### Example 27. Synthesis of Compounds 575 and 576

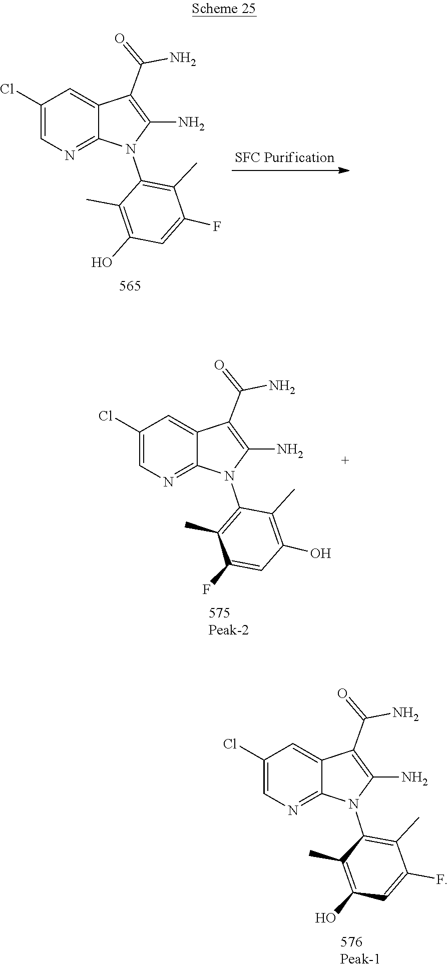

### 2-amino-5-chloro-1-(3-fluoro-5-hydroxy-2,6-dimethylphenyl)-1H-pyrrolo[2,3-b]pyridine-3-carboxamide (Peak-1) (Compound 576) and 2-amino-5-chloro-1-(3-fluoro-5-hydroxy-2,6-dimethylphenyl)-1H-pyrrolo[2,3-b]pyridine-3-carboxamide (Peak-2) (Compound 575)

Compound 565 was purified by SFC using (CHIRALPAK IBN (250×10) mm, 5 m), MOBILE PHASE A: 0.1% NH₃ (7 N in methanol) in n-Heptane MOBILE PHASE B: IPA:MTBE (05:95) to afford the title compounds as an off-white solid.

Compound 576 (Peak-1) (0.045 g); LCMS: RT=1.916 min, m/z 349.2 (M+H)⁺; HPLC: RT=8.762 min; Chiral HPLC: RT=9.11 min time; ¹H NMR (400 MHz, DMSO) δ 10.05 (s, 1H), 8.15 (s, 1H), 7.76 (s, 1H), 7.26 (bs, 2H), 6.89 (bs, 2H), 6.81 (d, J=11.2 Hz, 1H), 1.65 (s, 3H), 1.61 (s, 3H).

Compound 575 (Peak-2) (0.047 g); LCMS: RT=1.912 min, m/z 349.2 (M+H)⁺; HPLC: RT=6.758 min; Chiral HPLC: RT=11.62 min time; ¹H NMR (400 MHz, DMSO) δ 10.06 (s, 1H), 8.16 (s, 1H), 7.76 (s, 1H), 7.26 (bs, 2H), 6.89 (bs, 2H), 6.81 (d, J=11.2 Hz, 1H), 1.65 (s, 3H), 1.61 (s, 3H).

### Example 28. Synthesis of Compounds 577 and 578

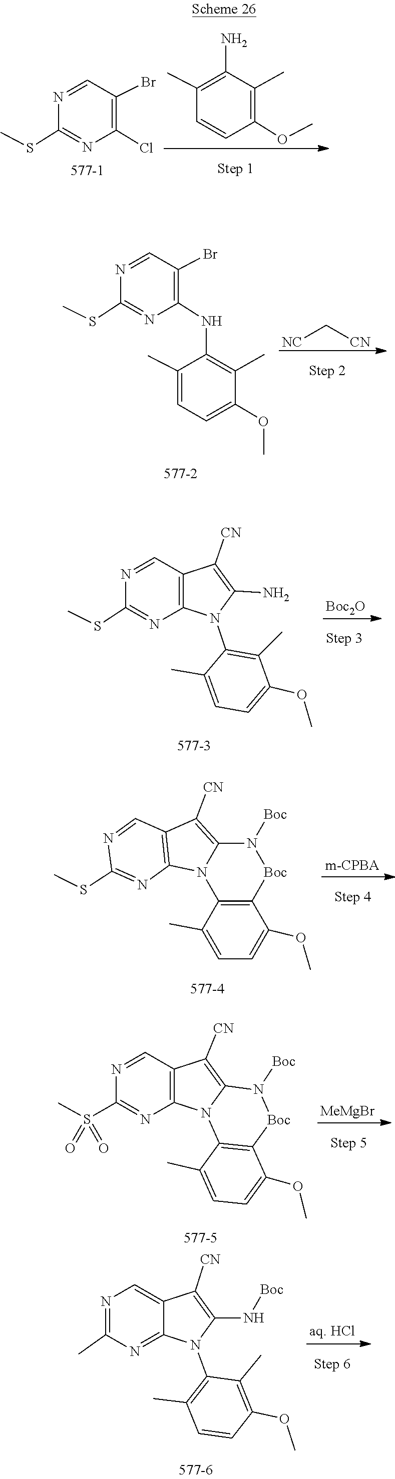

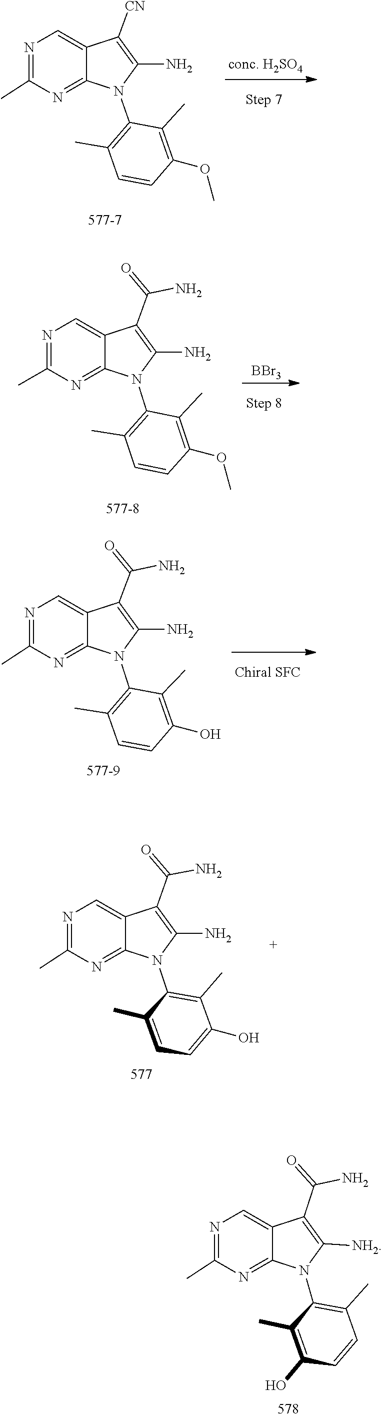

### Step 1: Synthesis of 5-bromo-N-(3-methoxy-2,6-dimethylphenyl)-2-(methylthio)pyrimidin-4-amine

To a solution of 3-methoxy-2,6-dimethyl-aniline (44.1 g, 291.62 mmol) in THF (600 mL) were added 5-bromo-4-chloro-2-methylsulfanyl-pyrimidine (63.5 g, 265.11 mmol) and LiHMDS (1 M solution in THF, 397.67 mL) at 0° C. The reaction was stirred at 0° C. for 1.5 hours, and was then quenched with water (300 mL) at 0° C. The resultant suspension was extracted with ethyl acetate (300 mL*2). The combined organic layers were washed with brine (200 mL), dried over sodium sulfate, filtered, and concentrated under reduced pressure. The residue was purified by flash column chromatography (silica gel, 100-200 mesh, gradient 0˜100% ethyl acetate in petroleum ether) to afford the title compound (70.0 g, 75%) as a yellow solid. ¹H NMR (400 MHz, DMSO-d₆) δ=8.76 (br s, 1H), 8.34 (s, 1H), 7.13 (d, J=8.0 Hz, 1H), 6.92 (d, J=8.4 Hz, 1H), 3.84 (s, 3H), 2.23 (s, 3H), 2.08 (s, 3H), 1.99 (s, 3H).

### Step 2: Synthesis of 6-amino-7-(3-methoxy-2,6-dimethylphenyl)-2-(methylthio)-7H-pyrrolo[2,3-d]pyrimidine-5-carbonitrile

To a solution of 2-isocyanoacetonitrile (14.6 g, 220.17 mmol) in 1,2-dimethoxyethane (650 mL) was added sodium hydride (9.4 g, 60% dispersion in mineral oil, 234.85 mmol) at 0° C. The mixture was stirred at 0° C. for 10 mins, and then 5-bromo-N-(3-methoxy-2,6-dimethyl-phenyl)-2-methylsulfanyl-pyrimidin-4-amine (52.0 g, 146.8 mmol) and Pd(dppf)Cl₂ dichloromethane adduct (12.0 g, 14.68 mmol) were added. The reaction was stirred at 110° C. under a nitrogen atmosphere for 4 hours. After cooling, the reaction mixture was quenched with water (300 mL) and extracted with ethyl acetate (200 mL*3). The combined organic layers were washed with brine (100 mL), dried over sodium sulfate, filtered, and concentrated under reduced pressure. The residue was purified by flash column chromatography (silica gel, 100-200 mesh, gradient 0˜100% ethyl acetate in petroleum ether) to afford the title compound (42.0 g, 84%) as a yellow solid. LCMS R_{T}=0.695 min, m/z=340.0 [M+H]⁺.

### Step 3: Synthesis of tert-butyl N-tert-butoxycarbonyl-N-[5-cyano-7-(3-methoxy-2,6-dimethyl-phenyl)-2-methylsulfanylpyrrolo[2,3-d]pyrimidin-6-yl]carbamate

To a solution of 6-amino-7-(3-methoxy-2,6-dimethyl-phenyl)-2-methylsulfanyl-pyrrolo[2,3-d]pyrimidine-5-carbonitrile (20.0 g, 58.93 mmol) in dichloromethane (50 mL) were added triethylamine (16.40 mL, 117.85 mmol), N,N-dimethylpyridin-4-amine (720 mg, 5.89 mmol) and di-tert-butyl dicarbonate (28.3 g, 129.64 mmol). The mixture was stirred at 20° C. for 1 hour, diluted with water (100 mL), and extracted with ethyl acetate (200 mL*2). The combined extracts were washed with brine (200 mL), dried over sodium sulfate, filtered, and concentrated under reduced pressure to afford the title compound (27.0 g, 85%) as a brown solid, which would be used directly in the next step without further purification. LCMS R_{T}=0.835 min, m/z=540.2 [M+H]⁺.

### Step 4: Synthesis of tert-butyl N-tert-butoxycarbonyl-N-[5-cyano-7-(3-methoxy-2,6-dimethyl-phenyl)-2-methylsulfonyl-pyrrolo[2,3-d]pyrimidin-6-yl]carbamate

To a solution of tert-butyl N-tert-butoxycarbonyl-N-[5-cyano-7-(3-methoxy-2,6-dimethyl-phenyl)-2-methylsulfanylpyrrolo[2,3-d]pyrimidin-6-yl]carbamate (30.0 g, 55.6 mmol) in dichloromethane (400 mL) was added 3-chlorobenzenecarboperoxoic acid (33.9 g, 166.78 mmol). The reaction mixture was stirred at 20° C. for 2 hours and then filtered. The filtrate was washed with saturated sodium bicarbonate solution (100 mL*2) and brine (300 mL*2), dried over sodium sulfate, filtered, and concentrated to afford the title compound (23.1 g, 73%) as a yellow solid, which was used directly in the next step without further purification. LCMS R_{T}=0.858 min, m/z=572.3 [M+H]⁺.

### Step 5: Synthesis of tert-butyl (5-cyano-7-(3-methoxy-2,6-dimethylphenyl)-2-methyl-7H-pyrrolo[2,3-d]pyrimidin-6-yl)carbamate

To a solution of tert-butyl N-tert-butoxycarbonyl-N-[5-cyano-7-(3-methoxy-2,6-dimethyl-phenyl)-2-methylsulfonylpyrrolo[2,3-d]pyrimidin-6-yl]carbamate (40.0 g, 69.97 mmol) in tetrahydrofuran (400 mL) was added MeMgBr (3 M solution in THF, 139.95 mL). The reaction mixture was stirred at 0° C. for 2 hours, quenched with water (300 mL), and extracted with ethyl acetate (300 mL). The organic extracts were washed with ammonium chloride aqueous solution (300 mL) and brine (200 mL), dried over sodium sulfate, filtered, and concentrated under reduced pressure to afford the title compound (29.0 g, 90%) as a yellow solid, which was used directly in the next step without further purification. LCMS R_{T}=0.692 min, m/z=408.1 [M+H]⁺. ¹H NMR (400 MHz, DMSO-d₆) δ=10.06 (br s, 1H), 9.07 (s, 1H), 7.25 (d, J=8.0 Hz, 1H), 7.12 (d, J=8.0 Hz, 1H), 3.85 (s, 3H), 2.57 (s, 3H), 1.76 (s, 3H), 1.65 (s, 3H), 1.39 (s, 9H).

### Step 6: Synthesis of 6-amino-7-(3-methoxy-2,6-dimethyl-phenyl)-2-methyl-pyrrolo[2,3-d]pyrimidine-5-carbonitrile

Tert-butyl N-[5-cyano-7-(3-methoxy-2,6-dimethyl-phenyl)-2-methyl-pyrrolo[2,3-d]pyrimidin-6-yl]carbamate (28.0 g, 68.72 mmol) was dissolved in hydrochloric acid (6 M aqueous, 112 mL) and ethyl alcohol (280 mL). The mixture was stirred at 80° C. for 30 minutes and was then cooled and concentrated under reduced pressure to afford the title compound (15.0 g, 65%) as a yellow solid, which would be used directly in the next step without further purification. LCMS R_{T}=0.443 min, m/z=308.1 [M+H]⁺. ¹H NMR (400 MHz, DMSO-d₆) δ=8.48 (s, 1H), 7.30 (br s, 2H), 7.25 (d, J=8.8 Hz, 1H), 7.11 (d, J=8.4 Hz, 1H), 3.85 (s, 3H), 2.44 (s, 3H), 1.79 (s, 3H), 1.70 (s, 3H).

### Step 7: Synthesis of 6-amino-7-(3-methoxy-2,6-dimethyl-phenyl)-2-methyl-pyrrolo[2,3-d]pyrimidine-5-carboxamide

A solution of 6-amino-7-(3-methoxy-2,6-dimethyl-phenyl)-2-methyl-pyrrolo[2,3-d]pyrimidine-5-carbonitrile (15.0 g, 48.80 mmol) in sulfuric acid (200 mL, 98%) was stirred at 20° C. for 1 hour, then poured onto ice-water (500 mL) and adjusted to pH=8 with NaOH solution. The precipitate was collected by filtration and dried under vacuum to afford the title compound (13.0 g, 77%) as a yellow solid, which was used directly in the next step without further purification. LCMS R_{T}=0.828 min, m/z=326.2 [M+H]⁺. ¹H NMR (400 MHz, DMSO-d₆) δ=8.89 (s, 1H), 7.26 (d, J=8.4 Hz, 1H), 7.11 (d, J=8.4 Hz, 1H), 7.09 (br s, 2H), 6.85 (br s, 2H), 3.85 (s, 3H), 2.43 (s, 3H), 1.80 (s, 3H), 1.70 (s, 3H).

### Step 8: Synthesis of 6-amino-7-(3-hydroxy-2,6-dimethyl-phenyl)-2-methyl-pyrrolo[2,3-d]pyrimidine-5-carboxamide atropisomers (Compound 577 and 578)

To a solution of 6-amino-7-(3-methoxy-2,6-dimethyl-phenyl)-2-methyl-pyrrolo[2,3-d]pyrimidine-5-carboxamide (13.0 g, 39.96 mmol) in dichloromethane (130 mL) was added tribromoborane (40.0 g, 159.82 mmol) dropwise at 0° C. The reaction was stirred at 20° C. for 1 hour under nitrogen atmosphere, and then quenched dropwise with saturated sodium bicarbonate solution (200 mL). The mixture was extracted with ethyl acetate (200 mL*3). The combined extracts were washed with brine (100 mL*2), dried over anhydrous sodium sulfate, filtered, and concentrated under reduced pressure. The residue was purified by chiral SFC [Column: Daicel Chiralcel OX (250 mm*30 mm, 10 um); Mobile phase: [EtOH (0.1% NH₃ H₂O)]; B %: 45%] to give two fractions (t₁=1.368 min and t₂=1.856 min).

Fractions 1 (t₁=1.368 min) was purified by RP-HPLC (0 to 30% acetonitrile in water and 0.225% formic acid) to afford the title compound (Compound 577, 4.1 g, 32%) as a yellow solid. LCMS R_{T}=1.302 min, m/z=312.2 [M+H]⁺. ¹H NMR (400 MHz, DMSO-d₆) δ=9.59 (br s, 1H), 8.88 (s, 1H), 8.14 (s, 1H of formic acid), 7.08 (d, J=8.4 Hz, 1H), 7.05 (br s, 2H), 6.93 (d, J=8.4 Hz, 1H), 6.84 (br s, 2H), 2.43 (s, 3H), 1.75 (s, 3H), 1.67 (s, 3H).

Fractions 2 (t₂=1.856 min) was purified by RP-HPLC (0 to 30% acetonitrile in water and 0.225% formic acid) to afford the title compound (Compound 578, 4.4 g, 34%) as a yellow solid. LCMS R_{T}=1.050 min, m/z=312.2 [M+H]⁺. ¹H NMR (400 MHz, DMSO-d₆) δ=9.59 (br s, 1H), 8.88 (s, 1H), 8.14 (s, 1H of formic acid), 7.08 (d, J=8.4 Hz, 1H), 7.05 (br s, 2H), 6.93 (d, J=8.4 Hz, 1H), 6.84 (br s, 2H), 2.43 (s, 3H), 1.75 (s, 3H), 1.67 (s, 3H).

### Example 29. Synthesis of Compound 595

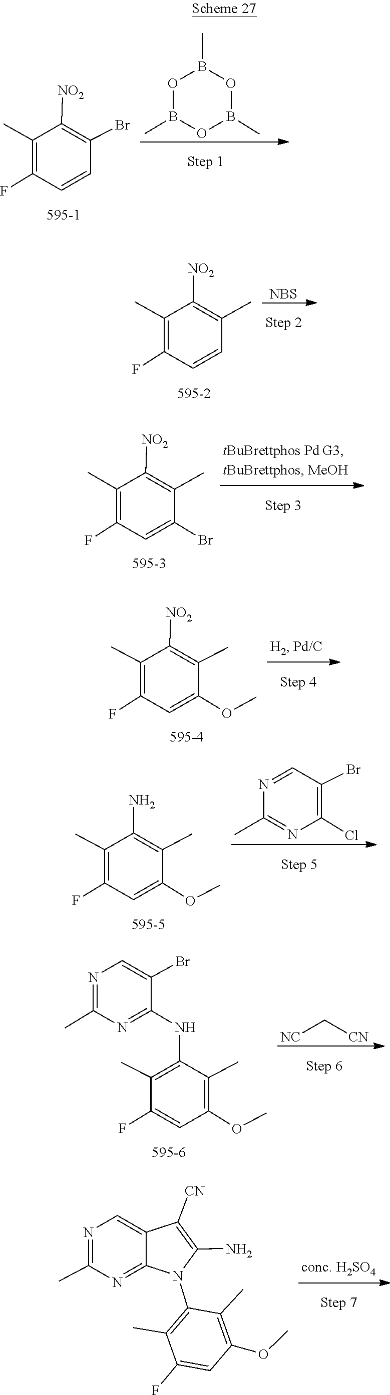

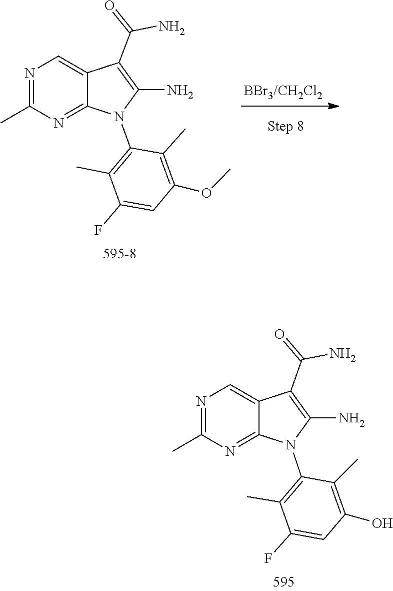

### Step 1: Synthesis of 1-fluoro-2,4-dimethyl-3-nitro-benzene

To a solution of 1-bromo-4-fluoro-3-methyl-2-nitro-benzene (5.0 g, 21.37 mmol) in dioxane (90 mL) and water (18 mL) were added 2,4,6-trimethyl-1,3,5,2,4,6-trioxatriborinane (2.7 g, 21.37 mmol), potassium carbonate (5.9 g 42.73 mmol) and Pd(dppf)Cl₂ (1.6 g, 2.14 mmol). The reaction mixture was stirred at 110° C. for 1 hour, cooled, diluted with water (100 mL), and extracted with ether ethyl acetate (100 mL*2). The combined organic layers were washed with brine (60 mL), dried over sodium sulfate, and concentrated. The residue was purified by flash column chromatography (silica gel, 100-200 mesh, gradient 0˜7% ethyl acetate in petroleum) to afford the title compound (4.0 g, 55%) as yellow oil. ¹H NMR (400 MHz, DMSO-d₆) δ 7.40-7.30 (m, 2H), 2.22 (s, 3H), 2.14 (s, 3H).

### Step 2: Synthesis of 1-bromo-5-fluoro-2,4-dimethyl-3-nitro-benzene

To a solution of 1-fluoro-2,4-dimethyl-3-nitro-benzene (4.0 g, 23.65 mmol) in sulfuric acid (38 mL) and trifluoroacetic acid (13 mL) was added N-bromosuccinimide (4.2 g, 23.65 mmol). The mixture was stirred at 20° C. for 12 hours, then quenched by ice-water (50 mL) and extracted with ethyl acetate (50 mL*2). The combined organic layers were washed with brine (50 mL), dried over anhydrous sodium sulfate and concentrated. The residue was purified by flash column chromatography (silica gel, 100-200 mesh, gradient 0˜10% ethyl acetate in petroleum) to afford the title compound (3.9 g, 22%) as a white solid, which would be used directly for next step. ¹H NMR (400 MHz, CDCl₃) δ 7.41 (d, J=8.4 Hz, 1H), 2.31 (s, 3H), 2.16 (s, 3H).

### Step 3: Synthesis of 1-fluoro-5-methoxy-2,4-dimethyl-3-nitro-benzene

To a solution of 1-bromo-5-fluoro-2,4-dimethyl-3-nitro-benzene (3.9 g, 15.64 mmol) in 1,4-dioxane (45 mL) and methanol (2.5 g, 78.21 mmol) were added cesium carbonate (7.6 g, 23.46 mmol), t-BuBrettPhos Pd G3 (401 mg, 46.9 mmol) and t-BuBrettPhos (227 mg, 46.92 mmol). The mixture was stirred at 80° C. for 16 hours, then cooled and concentrated. The residue was purified by flash column chromatography (silica gel, 100-200 mesh, gradient 0˜6% ethyl acetate in petroleum) to afford the title compound (2.6 g, 84%) as a yellow solid, which would be used directly for next step. _¹H NMR (400 MHz, DMSO-d₆) δ 7.13 (d, J=11.6 Hz 1H), 3.83 (s, 3H), 2.03 (s, 3H). 1.99 (s, 3H).

### Step 4: Synthesis of 3-fluoro-5-methoxy-2,6-dimethyl-aniline

To a solution of 1-fluoro-5-methoxy-2,4-dimethyl-3-nitro-benzene (2.6 g, 13.05 mmol) in methanol (50 mL) was added 10% Pd/C (830 mg). The reaction mixture was stirred at 25° C. for 2 hours under hydrogen (15 psi) and then filtered. The filtrate was diluted with water (50 mL) and extracted with ethyl acetate (50 mL*2). The combined extracts were washed with brine (40 mL), dried over anhydrous sodium sulfate and concentrated. The residue was purified by flash column chromatography (silica gel, 100-200 mesh, gradient 0˜5% ethyl acetate in petroleum) to afford the title compound (1.78 g, 81%) as a yellow solid. _¹H NMR (400 MHz, DMSO-d₆) δ 6.07 (d, J=12.0 Hz, 1H), 4.85 (s, 2H), 3.66 (s, 3H), 1.93 (s, 3H), 1.89 (s, 3H).

### Step 5: Synthesis of 5-bromo-N-(3-fluoro-5-methoxy-2,6-dimethyl-phenyl)-2-methyl-pyrimidin-4-amine

To a solution of 5-bromo-4-chloro-2-methyl-pyrimidine (300 mg, 1.45 mmol) and 3-fluoro-5-methoxy-2,6-dimethyl-aniline (245 mg, 1.45 mmol) in tetrahydrofuran (5.0 mL) was added NaHMDS (1 M, 2.89 mL) at 0° C. The mixture was stirred at 0° C. for 1 hour under nitrogen atmosphere, then quenched by water (10 mL) and extracted with ethyl acetate (10 mL*3). The combined extracts were washed with brine (10 mL*2), dried over anhydrous sodium sulfate, and filtered. The filtrate was concentrated under reduced pressure. The residue was purified by flash column chromatography (silica gel, 100-200 mesh, gradient 0˜20% ethyl acetate in petroleum ether) to afford the title compound (350 mg, 65%) as a yellow solid. LCMS R_{T}=0.602 min, m/z=341.9 [M+H]⁺.

### Step 6: Synthesis of 6-amino-7-(3-fluoro-5-methoxy-2,6-dimethyl-phenyl)-2-methyl-pyrrolo[2,3-d]pyrimidine-5-carbonitrile

To a solution of propanedinitrile (44 mg, 0.66 mmol) in tert-butanol (0.50 mL) and 1,2-dimethoxyethane (1.5 mL) was added sodium tert-butoxide (85 mg, 0.88 mmol). The mixture was pre-stirred at 25° C. for 30 minutes, and then 5-bromo-N-(3-fluoro-5-methoxy-2,6-dimethyl-phenyl)-2-methyl-pyrimidin-4-amine (150 mg, 0.44 mmol) and Pd(dppf)Cl₂ (54 mg, 0.07 mmol) were added. The reaction was stirred at 110° C. for 3 hours under a nitrogen atmosphere in the microwave oven, then cooled, filtered, and concentrated under reduced pressure. The residue was purified by flash column chromatography (silica gel, 100-200 mesh, gradient 0˜5% methanol in dichloromethane) to afford the title compound (90 mg, 60%) as a yellow solid. LCMS R_{T}=0.473, 0.505 min, m/z=326.0 [M+H]⁺.

### Step 7: Synthesis of 6-amino-7-(3-fluoro-5-methoxy-2,6-dimethyl-phenyl)-2-methyl-pyrrolo[2,3-d]pyrimidine-5-carboxamide

A solution of 6-amino-7-(3-fluoro-5-methoxy-2,6-dimethyl-phenyl)-2-methyl-pyrrolo[2,3-d]pyrimidine-5-carbonitrile (60 mg, 0.18 mmol) in sulfuric acid (1.0 mL, 98% purity) was stirred at 20° C. for 1 hour and then quenched with ice-water (10 mL) slowly. The mixture was adjusted to pH=8 with saturated sodium hydroxide solution, and then filtered to collect the precipitate, which was further dried under vacuum to afford the title compound (55 mg, crude) as a yellow solid.

### Step 8: Synthesis of 6-amino-7-(3-fluoro-5-hydroxy-2,6-dimethyl-phenyl)-2-methyl-pyrrolo[2,3-d]pyrimidine-5-carboxamide (Compound 595)

To a solution of 6-amino-7-(3-fluoro-5-methoxy-2,6-dimethyl-phenyl)-2-methyl-pyrrolo[2,3-d]pyrimidine-5-carboxamide (55 mg, 0.82 mmol) in dichloromethane (2.0 mL) was added boron tribromide (1 M solution, 0.15 mL) dropwise at 0° C. The reaction mixture was stirred at 0° C. for 2 hours under a nitrogen atmosphere and was then quenched with methanol (5.0 mL) and concentrated under reduced pressure. The residue was purified by reversed-phase HPLC (Welch Xtimate C18 150*30 mm*5 um; Mobile phase: [water(FA)-MeCN]; B %: 0˜22; Gradient time:25 min) to afford the title compound (23 mg, 43%) as a white solid. LCMS R_{T}=0.781 min, m/z=330.0 [M+H]⁺. ¹H NMR (400 MHz, CD₃OD) δ=8.81 (s, 1H), 6.80 (d, J=11.2 Hz, 1H), 2.59 (s, 3H), 1.79 (s, 6H).

### Example 30. Synthesis of Compounds 606 and 607

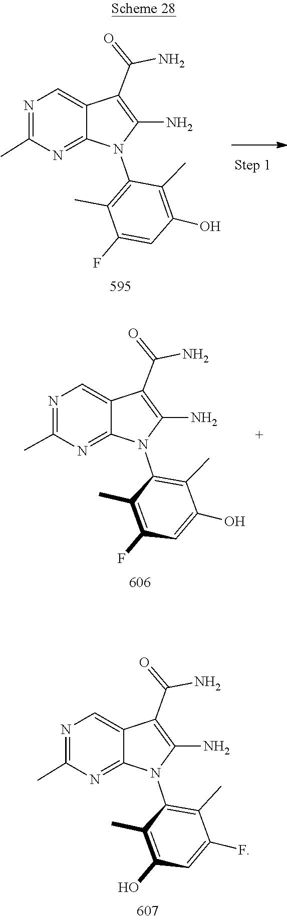

### 6-amino-7-(3-fluoro-5-hydroxy-2,6-dimethyl-phenyl)-2-methyl-pyrrolo[2,3-d]pyrimidine-5-carboxamide & 6-amino-7-(3-fluoro-5-hydroxy-2,6-dimethyl-phenyl)-2-methyl-pyrrolo[2,3-d]pyrimidine-5-carboxamide

Racemic 6-amino-7-(3-fluoro-5-hydroxy-2,6-dimethyl-phenyl)-2-methyl-pyrrolo[2,3-d]pyrimidine-5-carboxamide (200 mg crude, 0.82 mmol) was separated by chiral SFC {Column: Daicel chiralcel OX (250 mm*30 mm, 10 um); Mobile phase: [MeCN/EtOH (0.1% NH₃H₂O)]; B %: 45%) give two component peaks (Rt₁=1.139 min and Rt₂=1.569 min).

Component 1 (Rt₁=1.139 min) was purified by RP-HPLC (0 to 20% acetonitrile in water and 0.225% formic acid) to afford the title compound (34.2 mg, 43%) as a white solid. LCMS R_{T}=0.428 min, m/z=330.3 [M+H]⁺. ¹H NMR (400 MHz, DMSO-d₆) δ=10.11 (s, 1H), 8.89 (s, 1H), 7.17 (s, 2H), 6.93-6.75 (m, 3H), 2.44 (s, 3H), 1.66 (s, 3H), 1.62 (s, 3H).

Component 2 (Rt₂=1.569 min) was purified by RP-HPLC (0 to 20% acetonitrile in water and 0.225% formic acid) to afford the title compound (40.8 mg, 51%) as a white solid. LCMS R_{T}=0.350 min, m/z=330.4 [M+H]⁺. ¹H NMR (400 MHz, DMSO-d₆) δ=10.12 (s, 1H), 8.89 (s, 1H), 7.17 (s, 2H), 6.93-6.75 (m, 3H), 2.44 (s, 3H), 1.66 (s, 3H), 1.62 (s, 3H).

### Example 31. Synthesis of Compound 597

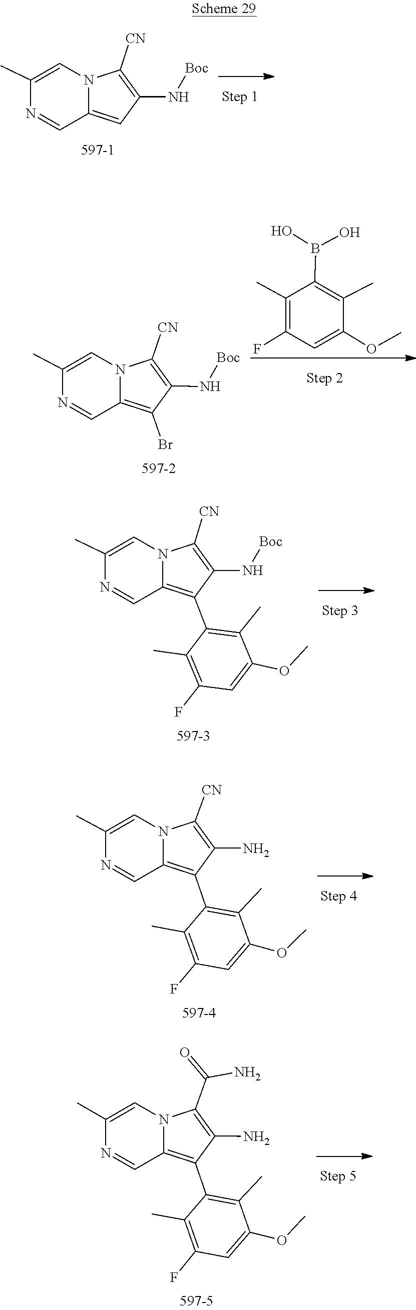

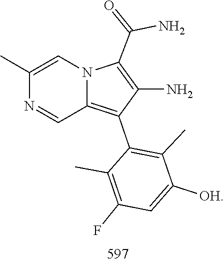 [SMILES: Cc1cn2c(C(N)=O)c(N)c(-c3c(C)c(O)cc(F)c3C)c2cn1]

### Step 1: Synthesis of tert-butyl N-(8-bromo-6-cyano-3-methyl-pyrrolo[1,2-a]pyrazin-7-yl)carbamate

To a solution of tert-butyl N-(6-cyano-3-methyl-pyrrolo[1,2-a]pyrazin-7-yl)carbamate (265 mg, 0.97 mmol) in DMF (3.0 mL) was added NBS (156 mg, 0.88 mmol) at 0° C. The mixture was stirred at 20° C. for 1 hour, quenched with H₂O (10 mL) and extracted with ethyl acetate (20 mL*3). The combined organic layers were washed with brine (50 mL), dried over anhydrous sodium sulfate, filtered, and concentrated under reduced pressure. The residue was purified by flash column chromatography on silica gel (gradient 0˜23% ethyl acetate in petroleum ether) to afford tert-butyl N-(8-bromo-6-cyano-3-methyl-pyrrolo[1,2-a]pyrazin-7-yl)carbamate (244 mg, 68% yield) as a yellow solid. LCMS R_{T}=0.724 min, m/z=352.9 [M+H]⁺.

### Step 2: Synthesis of tert-butyl N-[6-cyano-8-(3-fluoro-5-methoxy-2,6-dimethyl-phenyl)-3-methyl-pyrrolo[1,2-a]pyrazin-7-yl]carbamate

To a solution of tert-butyl N-(8-bromo-6-cyano-3-methyl-pyrrolo[1,2-a]pyrazin-7-yl)carbamate (100 mg, 0.28 mmol) and (3-fluoro-5-methoxy-2,6-dimethyl-phenyl)boronic acid (90 mg, 0.46 mmol) in dioxane (2.0 mL) and H₂O (0.20 mL) were added Pd₂(dba)₃ (26 mg, 0.028 mmol), SPhos (23 mg, 0.057 mmol) and Cs₂CO₃ (278 mg, 0.85 mmol). The reaction mixture was stirred at 130° C. for 1 hour under N₂ in a microwave oven. After cooling, the reaction mixture was extracted with ethyl acetate (50 mL*3). The combined extracts were washed with brine (100 mL), dried over anhydrous sodium sulfate, filtered, and concentrated under vacuum. The residue was purified by flash column chromatography on silica gel (gradient 0˜10% ethyl acetate in petroleum ether) to afford tert-butyl N-[6-cyano-8-(3-fluoro-5-methoxy-2,6-dimethyl-phenyl)-3-methyl-pyrrolo[1,2-a]pyrazin-7-yl]carbamate (47 mg, 31% yield) as a yellow oil. LCMS R_{T}=0.815 min, m/z=425.1 [M+H]⁺.

### Step 3: Synthesis of 7-amino-8-(3-fluoro-5-methoxy-2,6-dimethyl-phenyl)-3-methyl-pyrrolo[1,2-a]pyrazine-6-carbonitrile

A solution of tert-butyl N-[6-cyano-8-(3-fluoro-5-methoxy-2,6-dimethyl-phenyl)-3-methyl-pyrrolo[1,2-a]pyrazin-7-yl]carbamate (32 mg, 0.075 mmol) in 4M HCl/MeOH (1.0 mL) was stirred at 25° C. for 1 hour. The reaction mixture was concentrated under vacuum to give crude 7-amino-8-(3-fluoro-5-methoxy-2,6-dimethyl-phenyl)-3-methyl-pyrrolo[1,2-a]pyrazine-6-carbonitrile (24 mg) as a yellow oil.

### Step 4: Synthesis of 7-amino-8-(3-fluoro-5-methoxy-2,6-dimethyl-phenyl)-3-methyl-pyrrolo[1,2-a]pyrazine-6-carboxamide

A solution of 7-amino-8-(3-fluoro-5-methoxy-2,6-dimethyl-phenyl)-3-methyl-pyrrolo[1,2-a]pyrazine-6-carbonitrile (30 mg, 0.092 mmol) in H₂SO₄ (0.50 mL) was stirred at 25° C. for 1 hour. The reaction solution was quenched with ice-water (10 mL), adjusted to pH=8 by NaHCO₃, and extracted with ethyl acetate (10 mL*3). The organic extracts were washed with brine (30 mL), dried over anhydrous sodium sulfate, filtered, and concentrated under reduced pressure. The residue was purified by flash column chromatography on silica gel (gradient 0˜68% ethyl acetate in petroleum ether) to afford 7-amino-8-(3-fluoro-5-methoxy-2,6-dimethyl-phenyl)-3-methyl-pyrrolo[1,2-a]pyrazine-6-carboxamide (20 mg, 57% yield) as a yellow oil. LCMS R_{T}=0.546 min, m/z=343.0 [M+H]⁺.

### Step 5: Synthesis of 7-amino-8-(3-fluoro-5-hydroxy-2,6-dimethyl-phenyl)-3-methyl-pyrrolo[1,2-a]pyrazine-6-carboxamide (Compound 597)

To a solution of 7-amino-8-(3-fluoro-5-methoxy-2,6-dimethyl-phenyl)-3-methyl-pyrrolo[1,2-a]pyrazine-6-carboxamide (18 mg, 0.053 mmol) in dichloromethane (0.50 mL) was added BBr₃ (66 mg, 0.26 mmol). The reaction mixture was stirred at 25° C. for 2 hours, then quenched with MeOH (1.0 mL) and H₂O (0.20 mL). The so-formed suspension was concentrated under vacuum. The residue was purified by preparative HPLC [Column: Welch Xtimate C18 150*30 mm*5 um; mobile phase: water (FA)-MeCN; B %: 0˜26; Gradient time: 25 min; Flow Rate: 30 mL/min] to afford 7-amino-8-(3-fluoro-5-hydroxy-2,6-dimethyl-phenyl)-3-methyl-pyrrolo[1,2-a]pyrazine-6-carboxamide (8.4 mg, 48% yield) as a yellow solid. ¹H NMR (400 MHz, DMSO-d₆) δ 9.63 (br s, 1H), 8.90 (s, 1H), 8.01 (s, 1H), 7.28 (br s, 2H), 6.67 (d, J=11.2 Hz, 1H), 4.82 (br s, 2H), 2.35 (s, 3H), 1.80-1.76 (m, 6H). LCMS R_{T}=0.722 min, m/z=328.9 [M+H]⁺.

### Example 32. Synthesis of Compound 599

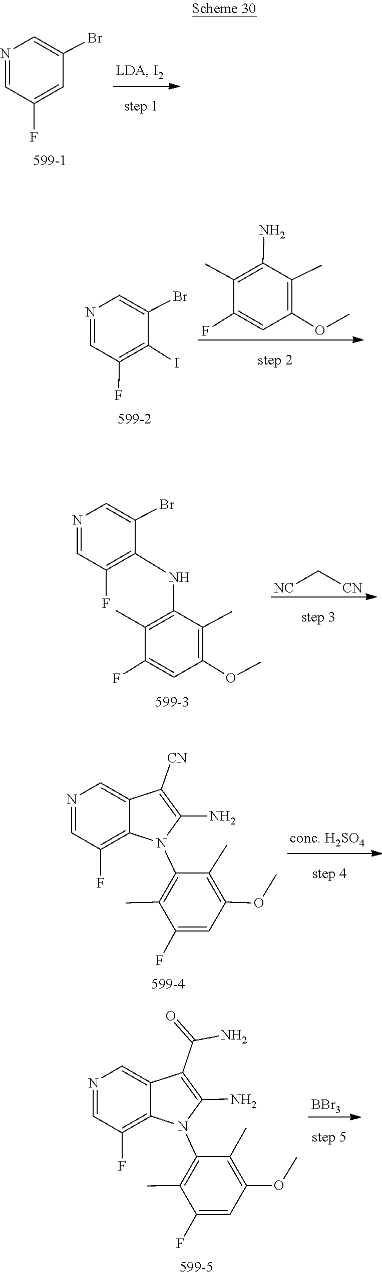

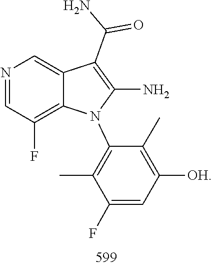 [SMILES: Cc1c(O)cc(F)c(C)c1-n1c(N)c(C(N)=O)c2cncc(F)c21]

### Step 1: Synthesis of 3-bromo-5-fluoro-4-iodopyridine

To a solution of 3-bromo-5-fluoro-pyridine (4.0 g, 22.7 mmol) in tetrahydrofuran (15 mL) was added (diisopropylamino)lithium (2.0 M, 17.05 mL) dropwise at −70° C. within 30 minutes, followed by iodine (6.92 g, 27.3 mmol) at −70° C. The mixture was stirred at −70° C. for 2 hours, and was then quenched with saturated ammonium chloride (10 mL) to adjust to pH to 6-7 at 0° C. The resulting mixture was diluted with water (30 mL) and extracted with ethyl acetate (30 mL*2). The combined extracts were washed with brine (20 mL*2), dried over anhydrous sodium sulfate, filtered, and concentrated under reduced pressure. The residue was purified by flash column chromatography (silica gel, 100-200 mesh, gradient 0˜3% ethyl acetate in petroleum ether) to afford the title compound (5.8 g, 82%) as a colorless solid. LCMS R_{T}=0.587 min, m/z=301.6 [M+H]⁺.

### Step 2: Synthesis of 3-bromo-5-fluoro-N-(3-fluoro-5-methoxy-2,6-dimethylphenyl)pyridin-4-amine

A mixture of 3-fluoro-5-methoxy-2,6-dimethyl-aniline (400 mg, 2.36 mmol), 3-bromo-5-fluoro-4-iodo-pyridine (2.0 g, 6.62 mmol), cesium carbonate (1.5 g, 4.73 mmol), (5-diphenylphosphanyl-9,9-dimethyl-xanthen-4-yl)-diphenyl-phosphane (137 mg, 0.24 mmol) and palladium acetate (60 mg, 0.26 mmol) in dioxane (25 mL) was stirred at 90° C. for 16 hours under N₂ atmosphere. After cooling, the reaction mixture was filtered and the filtrate was concentrated under reduced pressure. The residue was purified by flash column chromatography (silica gel, 100-200 mesh, gradient 0˜10% ethyl acetate in petroleum ether) to afford the title compound (610 mg, 68%) as a colorless solid. LCMS R_{T}=0.552 min, m/z=344.9 [M+H]⁺.

### Step 3: Synthesis of 2-amino-7-fluoro-1-(3-fluoro-5-methoxy-2,6-dimethylphenyl)-1H-pyrrolo[3,2-c]pyridine-3-carbonitrile

To a solution of propanedinitrile (230 mg, 3.50 mmol) in 1,2-dimethoxyethane (18 mL) was added NaH (140 mg, 60% dispersion in mineral oil, 3.50 mmol) portion wise within 30 minutes, followed by 3-bromo-5-fluoro-N-(3-fluoro-5-methoxy-2,6-dimethyl-phenyl)pyridin-4-amine (400 mg, 1.17 mmol) and Pd(dppf)Cl₂·CH₂Cl₂ (145 mg, 175 mmol). The mixture was stirred at 110° C. for 2 hours under nitrogen atmosphere. After cooling, the reaction mixture was quenched with water (15 mL) and extracted with ethyl acetate (10 mL*2). The combined organic extracts were washed with brine (10 mL), dried over anhydrous sodium sulfate, filtered and concentrated under reduced pressure. The residue was purified by flash column chromatography (silica gel, 100-200 mesh, gradient 0˜100% ethyl acetate in petroleum ether) to afford the title compound (370 mg, 77%) as a black solid. LCMS R_{T}=0.999 min, m/z=328.9 [M+H]⁺.

### Step 4: Synthesis of Synthesis of 2-amino-7-fluoro-1-(3-fluoro-5-methoxy-2,6-dimethylphenyl)-1H-pyrrolo[3,2-c]pyridine-3-carboxamide

2-Amino-7-fluoro-1-(3-fluoro-5-methoxy-2,6-dimethyl-phenyl)pyrrolo[3,2-c]pyridine-3-carbonitrile (150 mg, 0.46 mmol) was dissolved in sulfuric acid (2.0 mL) and the solution was stirred at 20° C. for 1 hour, and was then poured onto ice water (5.0 mL). The resulting mixture was adjusted to pH=7 and extracted with ethyl acetate (10 mL*2). The organic phase was washed with brine (10 mL), dried over anhydrous sodium sulfate, filtered, and concentrated under reduced pressure to afford the title compound (160 mg, 81%) as a colorless solid. LCMS R_{T}=0.406 min, m/z=346.9 [M+H]⁺.

### Step 5: Synthesis of 2-amino-7-fluoro-1-(3-fluoro-5-hydroxy-2,6-dimethylphenyl)-1H-pyrrolo[3,2-c]pyridine-3-carboxamide (Compound 599)

A solution of 2-amino-7-fluoro-1-(3-fluoro-5-methoxy-2,6-dimethyl-phenyl)pyrrolo[3,2-c]pyridine-3-carboxamide (40 mg, 0.09 mmol) in dichloromethane (3.0 mL) was treated with boron tribromide (208 mg, 0.83 mmol), and the mixture was stirred at 100° C. for 2 hours. After cooling, the reaction solution was quenched with 1M hydrochloric acid (0.5 mL), filtered, and the filtrate was concentrated under reduced pressure. The residue was purified by RP-HPLC (0 to 24% acetonitrile in water and 0.225% formic acid) to give the title compound (20 mg, 33%) as a yellow solid. ¹H NMR (400 MHz, DMSO-d₆) δ=10.16 (br s, 1H), 8.80 (d, J=1.6 Hz, 1H), 8.14 (s, 1H), 7.98 (d, J=2.8 Hz, 1H), 7.06 (br s, 2H), 6.95 (br s, 2H), 6.82 (d, J=10.8 Hz, 1H), 1.71 (s, 3H), 1.67 (s, 3H). LCMS R_{T}=0.864 min, m/z=333.3 [M+H]⁺. ¹⁹F NMR (400 MHz, DMSO-d₆) δ=−118.126, −154.838.

### Example 33. Synthesis of Compound 602

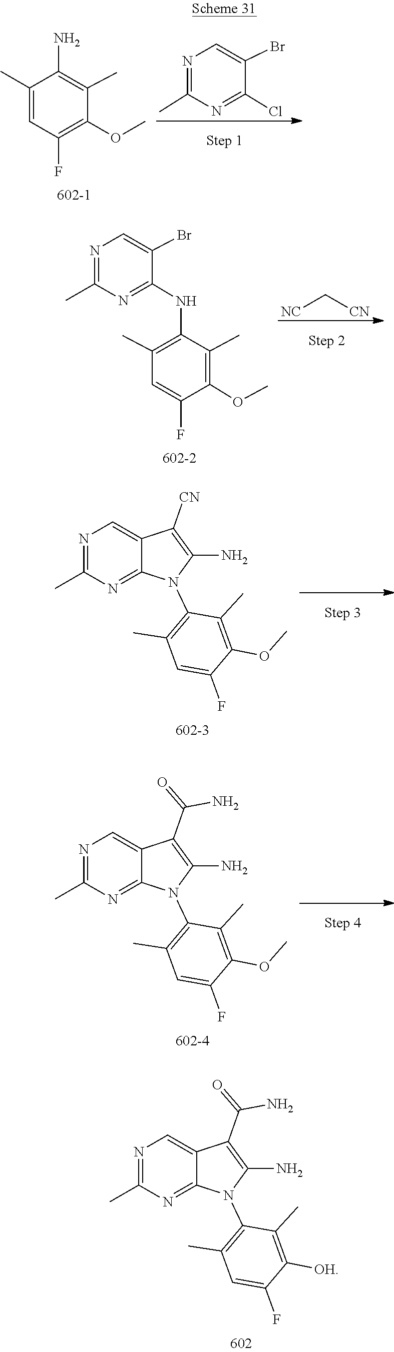

### Step 1: Synthesis of 5-bromo-N-(4-fluoro-3-methoxy-2,6-dimethyl-phenyl)-2-methyl-pyrimidin-4-amine

To a solution of 5-bromo-4-chloro-2-methyl-pyrimidine (500 mg, 2.41 mmol) and 4-fluoro-3-methoxy-2,6-dimethyl-aniline (408 mg, 2.41 mmol) in THF (15 mL) was added NaHMDS (1 M solution, 4.82 mL) dropwise at 0° C. The mixture was stirred at 0° C. for 1 hour and was then quenched with water (20 mL) and extracted with ethyl acetate (50 mL*3). The combined extracts were washed with brine (100 mL), dried over anhydrous sodium sulfate, filtered, and concentrated under vacuum. The residue was purified by flash column chromatography on silica gel (ethyl acetate in petroleum ether from 0% to 12%) to afford 5-bromo-N-(4-fluoro-3-methoxy-2,6-dimethyl-phenyl)-2-methyl-pyrimidin-4-amine (750 mg, 90% yield) as yellow oil. LCMS R_{T}=0.577 min, m/z=341.9 [M+H]⁺.

### Step 2: Synthesis of 6-amino-7-(4-fluoro-3-methoxy-2,6-dimethyl-phenyl)-2-methyl-pyrrolo[2,3-d]pyrimidine-5-carbonitrile

To a solution of propanedinitrile (117 mg, 1.76 mmol) in t-BuOH (0.8 mL) and DME (2.4 mL) was added t-BuONa (226 mg, 2.35 mmol). The mixture was stirred at 25° C. for 30 mins, then 5-bromo-N-(4-fluoro-3-methoxy-2,6-dimethyl-phenyl)-2-methyl-pyrimidin-4-amine (400 mg, 1.18 mmol) and Pd(dppf)Cl₂ (129 mg, 0.18 mmol) were added. The reaction mixture was stirred at 110° C. for 3 hours under N₂ atmosphere in a microwave oven. After cooling, the mixture was concentrated under vacuum. The residue was purified by flash column chromatography on silica gel (gradient 0˜30% ethyl acetate in petroleum ether) to afford 6-amino-7-(4-fluoro-3-methoxy-2,6-dimethyl-phenyl)-2-methyl-pyrrolo[2,3-d]pyrimidine-5-carbonitrile (259 mg, 61% yield) as light yellow solid. LCMS R_{T}=0.476 min, m/z=326.1 [M+H]⁺.

### Step 3: Synthesis of 6-amino-7-(4-fluoro-3-methoxy-2,6-dimethyl-phenyl)-2-methyl-pyrrolo[2,3-d]pyrimidine-5-carboxamide

A solution of 6-amino-7-(4-fluoro-3-methoxy-2,6-dimethyl-phenyl)-2-methyl-pyrrolo[2,3-d]pyrimidine-5-carbonitrile (50 mg, 0.15 mmol) in H₂SO₄ (0.50 mL) was stirred at 25° C. for 1 hour, and then poured onto ice water (10 mL). The mixture was adjusted to pH=8 with saturated NaHCO₃ solution and extracted with ethyl acetate (10 mL*3). The combined extracts were washed with brine (30 mL), dry over anhydrous sodium sulfate, filtered, and concentrated under reduced pressure to give 6-amino-7-(4-fluoro-3-methoxy-2,6-dimethyl-phenyl)-2-methyl-pyrrolo[2,3-d]pyrimidine-5-carboxamide (60 mg, crude) as light yellow solid. LCMS R_{T}=0.523 min, m/z=344.0 [M+H]⁺.

### Step 4: Synthesis of 6-amino-7-(4-fluoro-3-hydroxy-2,6-dimethyl-phenyl)-2-methyl-pyrrolo[2,3-d]pyrimidine-5-carboxamide (Compound 602)

To a solution of 6-amino-7-(4-fluoro-3-methoxy-2,6-dimethyl-phenyl)-2-methyl-pyrrolo[2,3-d]pyrimidine-5-carboxamide (30 mg, 0.087 mmol) in 1,2-dichloroethane (1.0 mL) was added BBr₃ (109 mg, 0.44 mmol). The reaction mixture was stirred at 25° C. for 2 hours, and was then quenched with MeOH (1.0 mL) and H₂O (0.20 mL). The mixture was concentrated under vacuum. The residue was purified by RP-HPLC [Column: Welch Xtimate C18 150*30 mm*5 um; mobile phase: water (FA)-MeCN; B %: 0-20; Gradient time: 25 min; Flow Rate: 30 mL/min] to afford 6-amino-7-(4-fluoro-3-hydroxy-2,6-dimethyl-phenyl)-2-methyl-pyrrolo[2,3-d]pyrimidine-5-carboxamide (9.0 mg, 31% yield) as white solid. ¹H NMR (400 MHz, DMSO-d₆) δ 9.66 (s, 1H), 8.88 (s, 1H), 7.13 (t, J=6.0 Hz, 3H), 6.84 (s, 2H), 2.44 (s, 3H), 1.77 (s, 3H), 1.71 (s, 3H). LCMS R_{T}=1.392 min, m/z=330.4 [M+H]⁺. ¹⁹F NMR (400 MHz, DMSO-d₆) δ=−133.712.

### Example 34. Synthesis of Compound 603

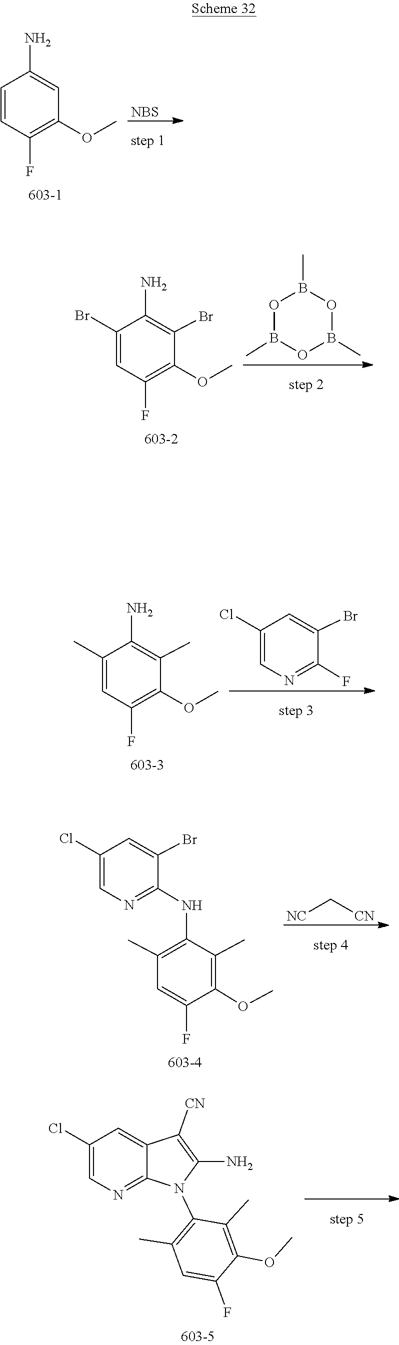

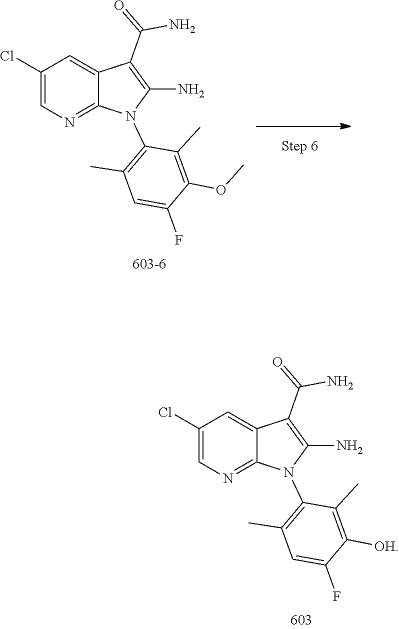 [SMILES: COc1c(F)cc(C)c(-n2c(N)c(C(N)=O)c3cc(Cl)cnc32)c1C ; Cc1cc(F)c(O)c(C)c1-n1c(N)c(C(N)=O)c2cc(Cl)cnc21]

### Step 1: Synthesis of 2,6-dibromo-4-fluoro-3-methoxyaniline

To a solution of 4-fluoro-3-methoxyaniline (10 g, 70.85 mmol) in AcOH (100 mL) was added NBS (27.74 g, 155.87 mmol). The mixture was stirred at 25° C. for 2 hours and then concentrated to remove most of the volatiles. The residue was partitioned between ethyl acetate (100 mL) and 5 M aqueous NaOH (100 mL) at 0° C. The aqueous phase was diluted with water (100 mL) and extracted with ethyl acetate (150 mL*2). The combined organic layers were washed with brine (100 mL), dried over anhydrous sodium sulfate, filtered, and concentrated. The residue was purified by flash column chromatography (silica gel, 100-200 mesh, gradient 0-25% dichloromethane in petroleum ether) to afford the title compound (6.0 g, 28%) as a light yellow solid. ¹H NMR (400 MHz, DMSO-d₆) δ=7.52 (d, J=10.4 Hz, 1H), 5.22 (br s, 2H), 3.82 (s, 3H).

### Step 2: Synthesis of 4-fluoro-3-methoxy-2,6-dimethylaniline

To a solution of 2,6-dibromo-4-fluoro-3-methoxyaniline (5.6 g, 18.73 mmol) in 1,4-dioxane (80 mL) were added Cs₂CO₃ (21.36 g, 65.57 mmol), 2,4,6-trimethyl-1,3,5,2,4,6-trioxatriborinane (14.11 g, 56.20 mmol), and Pd(dppf)Cl₂ dichloromethane adduct (1.53 g, 1.87 mmol). The mixture was stirred at 100° C. for 10 hours under a nitrogen atmosphere, then cooled and filtered. The filtrate was diluted with water (150 mL) and extracted with ethyl acetate (100 mL*2). The combined organic layers were washed with brine (80 mL*2), dried over anhydrous Na₂SO₄, filtered, and concentrated. The residue was purified by flash column chromatography (silica gel, 100-200 mesh, gradient 0-15% ethyl acetate in petroleum ether) to afford the title compound (2.6 g, 82%) as a yellow oil. ¹H NMR (400 MHz, DMSO-d₆) δ=6.70 (d, J=12.0 Hz, 1H), 4.43 (br s, 2H), 3.69 (s, 3H), 2.03 (s, 3H), 2.00 (s, 3H).

### Step 3: Synthesis of 3-bromo-5-chloro-N-(4-fluoro-3-methoxy-2,6-dimethyl-phenyl)pyridin-2-amine

To a solution of 4-fluoro-3-methoxy-2,6-dimethyl-aniline (800 mg, 4.73 mmol) and 3-bromo-5-chloro-2-fluoro-pyridine (1.09 g, 5.20 mmol) in THF (15 mL) was added dropwise LiHMDS (1 M, 8.51 mL) at 0° C. The reaction was stirred at 0° C. for 1 hour. After being quenched with H₂O (20 mL) at 0° C., the mixture was extracted with ethyl acetate (30 mL*3). The combined organic layers were washed with brine (20 mL*2), dried over anhydrous Na₂SO₄, filtered, and concentrated under reduced pressure. The residue was purified by flash column chromatography (silica gel, 100-200 mesh, gradient 0-100% ethyl acetate in petroleum ether) to afford the title compound (1.67 g, 98%) as an orange solid. LCMS R_{T}=0.888 min, m/z=360.8 [M+H]⁺.

### Step 4: Synthesis of 2-amino-5-chloro-1-(4-fluoro-3-methoxy-2,6-dimethyl-phenyl)pyrrolo[2,3-b]pyridine-3-carbonitrile

To a solution of propanedinitrile (193 mg, 2.92 mmol) in DME (12 mL) was added NaH (124 mg, 60% dispersion in mineral oil, 3.11 mmol) and the mixture was stirred at 0° C. for 10 mins. Then 3-bromo-5-chloro-N-(4-fluoro-3-methoxy-2,6-dimethyl-phenyl)pyridin-2-amine (700 mg, 1.95 mmol) and Pd(dppf)Cl₂ dichloromethane adduct (159 mg, 0.19 mmol) were added. The reaction was stirred at 110° C. under N₂ atmosphere for 4 hours. After cooling, the reaction mixture was quenched with H₂O (10 mL) at 0° C. and extracted with ethyl acetate (20 mL*3). The combined organic layers were washed with brine (20 mL*2), dried over anhydrous Na₂SO₄, filtered, and concentrated under reduced pressure. The residue was purified by flash column chromatography (silica gel, 100-200 mesh, gradient 0-100% ethyl acetate in petroleum ether) to afford the title compound (410 mg, 61%) as a white solid. LCMS R_{T}=0.801 min, m/z=345.0 [M+H]⁺.

### Step 5: Synthesis of 2-amino-5-chloro-1-(4-fluoro-3-methoxy-2,6-dimethyl-phenyl)pyrrolo[2,3-b]pyridine-3-carboxamide

A mixture of 2-amino-5-chloro-1-(4-fluoro-3-methoxy-2,6-dimethyl-phenyl)pyrrolo[2,3-b]pyridine-3-carbonitrile (300 mg, 0.87 mmol) in concentrated H₂SO₄ (1.0 mL) was stirred at 20° C. for 1 hour, then diluted with H₂O (5.0 mL), treated with 1 M aqueous NaOH till pH=7, and extracted with ethyl acetate (10 mL*3). The combined organic layers were washed with brine (10 mL), dried over anhydrous Na₂SO₄, filtered, and concentrated under reduced pressure to afford the title compound (250 mg, 79%) as a yellow solid, which was used directly in the next step without further purification. LCMS R_{T}=0.629 min, m/z=363.1 [M+H]⁺.

### Step 6: Synthesis of 2-amino-5-chloro-1-(4-fluoro-3-hydroxy-2,6-dimethyl-phenyl)pyrrolo[2,3-b]pyridine-3-carboxamide (Compound 603)

To a solution of 2-amino-5-chloro-1-(4-fluoro-3-methoxy-2,6-dimethyl-phenyl)pyrrolo[2,3-b]pyridine-3-carboxamide (150 mg, 0.41 mmol) in 1,2-dichloroethane (3.0 mL) was added BBr₃ (517 mg, 2.07 mmol). The mixture was stirred at 20° C. for 1 hour, then quenched with MeOH (1.0 mL) and H₂O (1.0 mL) at 0° C. The mixture was concentrated under reduced pressure. The residue was purified by RP-HPLC (2 to 42% acetonitrile in water and 0.05% hydrochloric acid) to afford the title compound (51.5 mg, 31%). LCMS R_{T}=0.634 min, m/z=349.0 [M+H]⁺. ¹H NMR (400 MHz, DMSO-d₆) δ=9.62 (br s, 1H), 8.14 (d, J=2.0 Hz, 1H), 7.75 (d, J=2.0 Hz, 1H), 7.21 (br s, 2H), 7.11 (d, J=11.6 Hz, 1H), 6.87 (br s, 2H), 1.75 (s, 3H), 1.70 (s, 3H).

### Example 35. Synthesis of Compound 604

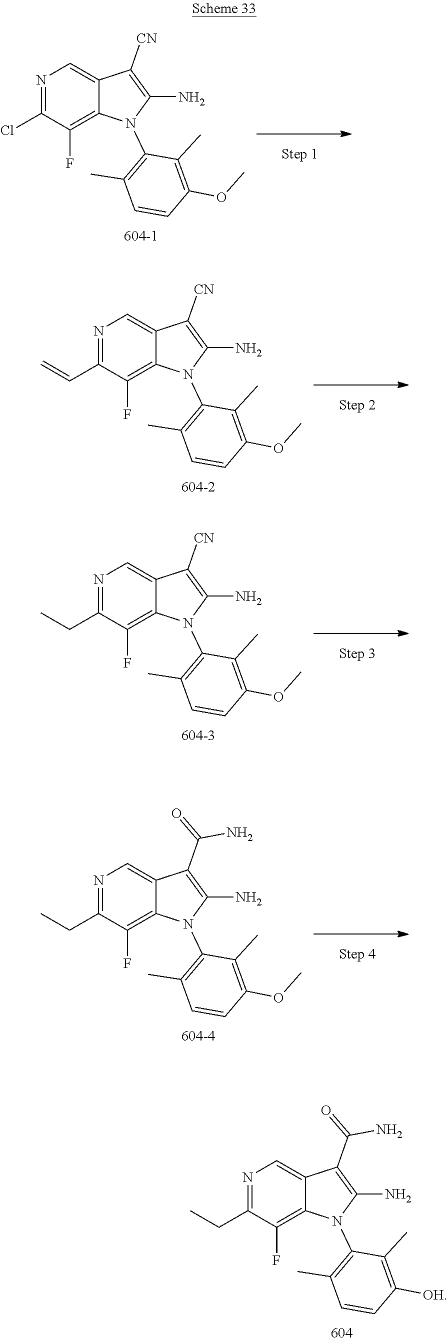

### Step 1: Synthesis of 2-amino-7-fluoro-1-(3-methoxy-2,6-dimethyl-phenyl)-6-vinyl-pyrrolo[3,2-c]pyridine-3-carbonitrile

To a solution of 2-amino-6-chloro-7-fluoro-1-(3-methoxy-2,6-dimethyl-phenyl)pyrrolo[3,2-c]pyridine-3-carbonitrile (400 mg, 1.16 mmol) in toluene (4.0 mL) and water (0.40 mL) were added vinylboronic acid (417 mg, 5.80 mmol), potassium phosphate (1.20 g, 5.80 mmol), XPhos Pd G3 (98 mg, 0.12 mmol) and XPhos (55 mg, 0.12 mmol). The reaction was stirred at 95° C. for 16 hours. After cooling, the mixture was filtered and the filtrate was concentrated under reduced pressure. The crude product was purified by column chromatography on silica gel (silica gel, 100-200 mesh, gradient 0˜24% ethyl acetate in petroleum ether) to afford the title compound (260 mg, 60% yield) as a yellow oil. LCMS R_{T}=0.591 min, m/z=337.0 [M+H]⁺.

### Step 2: Synthesis of 2-amino-6-ethyl-7-fluoro-1-(3-methoxy-2,6-dimethyl-phenyl)pyrrolo[3,2-c]pyridine-3-carbonitrile

To a solution of 2-amino-7-fluoro-1-(3-methoxy-2,6-dimethyl-phenyl)-6-vinyl-pyrrolo[3,2-c]pyridine-3-carbonitrile (260 mg, 0.77 mmol) in ethyl acetate (1.0 mL) was added 10% palladium on charcoal (82 mg). The reaction was stirred at 20° C. for 1 hour under a hydrogen atmosphere (15 psi), then the mixture was filtered and the filtrate was concentrated under reduced pressure to afford the title compound (260 mg, 99% yield) as a yellow solid, which was used directly in the next step without further purification. ¹H NMR (400 MHz, DMSO-d₆) δ=8.24 (s, 1H), 7.23 (d, J=8.4 Hz, 1H), 7.09 (d, J=8.4 Hz, 3H), 7.08 (br s, 2H), 3.84 (s, 3H), 2.74-2.60 (m, 2H), 1.85 (s, 3H), 1.76 (s, 3H), 1.16 (t, J=4.8 Hz, 3H).

### Step 3: Synthesis of 2-amino-6-ethyl-7-fluoro-1-(3-methoxy-2,6-dimethyl-phenyl)pyrrolo[3,2-c]pyridine-3-carboxamide

A solution of 2-amino-6-ethyl-7-fluoro-1-(3-methoxy-2,6-dimethyl-phenyl)pyrrolo[3,2-c]pyridine-3-carbonitrile (260 mg, 0.77 mmol) in 98% sulfuric acid (3.0 mL) was stirred at 20° C. for 30 mins, and was then quenched with NaOH solution till pH=8 and filtered to collect the formed precipitate. The filter cake was concentrated under vacuum to afford the title compound (260 mg, 75% yield) as a yellow solid. LCMS R_{T}=0.402 min, m/z=357.1 [M+H]⁺.

### Step 4: Synthesis of 2-amino-6-ethyl-7-fluoro-1-(3-hydroxy-2,6-dimethyl-phenyl)pyrrolo[3,2-c]pyridine-3-carboxamide (Compound 604)

To a solution of 2-amino-6-ethyl-7-fluoro-1-(3-methoxy-2,6-dimethyl-phenyl)pyrrolo[3,2-c]pyridine-3-carboxamide (130 mg, 0.36 mmol) in dichloromethane (1.0 mL) was added boron tribromide (457 mg, 1.82 mmol) dropwise at 0° C. The reaction was stirred at 20° C. for 1 hour under a nitrogen atmosphere, and was then quenched with methanol (5 mL). The mixture was concentrated under vacuum. The residue was purified by RP-HPLC [Column: Welch Xtimate C18 150*30 mm*5 um; Mobile phase: water (0.225% formic acid)-acetonitrile (gradient 20-50%); Flow rate: 25 mL/min] to afford the title compound (35.6 mg, 28% yield) as a yellow solid. LCMS R_{T}=1.527 min, m/z=343.1 [M+H]⁺. ¹H NMR (400 MHz, DMSO-d₆) δ=9.59 (br s, 1H), 8.67 (s, 1H), 8.13 (s, 1H of formic acid), 7.06 (d, J=8.4 Hz, 1H), 6.92 (d, J=8.4 Hz, 1H), 6.85 (br s, 4H), 2.76-2.60 (m, 2H), 1.80 (s, 3H), 1.72 (s, 3H), 1.16 (t, J=7.2 Hz, 3H).

### Example 36. Synthesis of Compound 605

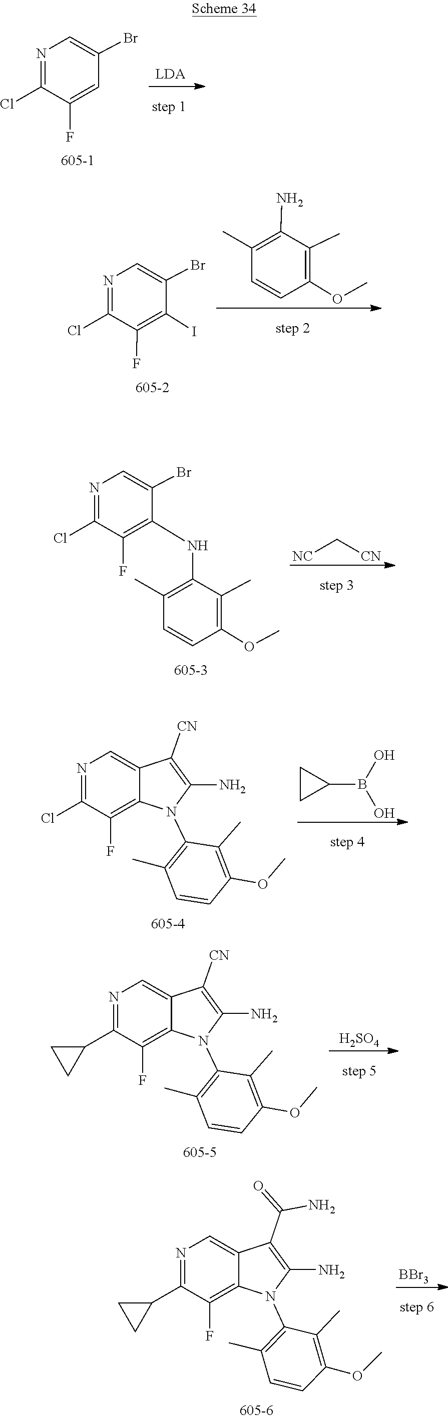

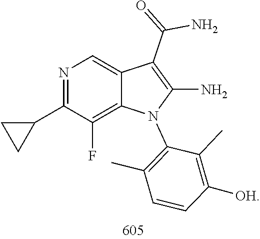 [SMILES: Cc1ccc(O)c(C)c1-n1c(N)c(C(N)=O)c2cnc(C3CC3)c(F)c21]

### Step 1: Synthesis of 5-bromo-2-chloro-3-fluoro-4-iodopyridine

To a solution of 5-bromo-2-chloro-3-fluoro-pyridine (5.0 g, 23.76 mmol) in THF (35 mL) was added (diisopropylamino)lithium (2.0 M in THF, 18 mL) at −70° C. within 15 minutes, followed by iodine (7.3 g, 28.51 mmol) in THF (20 mL) at −70° C. The reaction was stirred at −70° C. for 2 hours, and was then quenched with ammonium chloride solution (10 mL) till pH=6-7 at 0° C., diluted with water (30 mL) and extracted with ethyl acetate (30 mL*2). The combined organic layers were washed with brine (20 mL*2), dried over anhydrous sodium sulfate, filtered, and concentrated under reduced pressure. The residue was purified by flash column chromatography (silica gel, 100-200 mesh, gradient 0˜3% ethyl acetate in petroleum ether) to afford the title compound (4.0 g, 47%) as a colorless solid. LCMS R_{T}=0.650 min, m/z=337.6 [M+H]⁺.

### Step 2: Synthesis of 5-bromo-2-chloro-3-fluoro-N-(3-methoxy-2,6-dimethylphenyl)pyridin-4-amine

To a solution of 5-bromo-2-chloro-3-fluoro-4-iodo-pyridine (3.70 g, 10.91 mmol) in 1,4-dioxane (45 mL) were added 3-methoxy-2,6-dimethyl-aniline (1.5 g, 9.92 mmol), palladium acetate (225 mg, 0.992 mmol), XantPhos (574 mg, 0.992 mmol) and Cs₂CO₃ (6.50 g, 19.84 mmol). The reaction was stirred at 90° C. for 16 hours under N₂ atmosphere. After cooling, the reaction mixture was filtered and concentrated under reduced pressure. The residue was purified by flash column chromatography (silica gel, 100-200 mesh, gradient 0˜10% ethyl acetate in petroleum ether) to afford the title compound (2.3 g, 61%) as a colorless solid. LCMS R_{T}=0.689 min, m/z=360.8 [M+H]⁺.

### Step 3: Synthesis of 2-amino-6-chloro-7-fluoro-1-(3-methoxy-2,6-dimethylphenyl)-1H-pyrrolo[3,2-c]pyridine-3-carbonitrile

To a solution of propanedinitrile (275 mg, 4.17 mmol) in 1,2-dimethoxyethane (25 mL) and t-butanol (8.0 mL) was added t-BuONa (535 mg, 5.56 mmol) within 30 minutes at 0° C., followed by 5-bromo-2-chloro-3-fluoro-N-(3-methoxy-2,6-dimethyl-phenyl)pyridin-4-amine (1.0 g, 2.78 mmol) and Pd(dppf)Cl₂ dichloromethane adduct (340 mg, 0.417 mmol). The reaction was stirred at 110° C. for 3 hours under nitrogen atmosphere. After cooling, the reaction mixture was quenched with water (15 mL) slowly and extracted with ethyl acetate (10 mL*2). The combined extracts were washed with brine (10 mL), dried over anhydrous Na₂SO₄, filtered, and concentrated under reduced pressure. The residue was purified by flash column chromatography (silica gel, 100-200 mesh, gradient 0-29% ethyl acetate in petroleum ether) to afford the title compound (940 mg, 47%) as a brown solid. LCMS R_{T}=0.768 min, m/z=345.0 [M+H]⁺.

### Step 4: Synthesis of 2-amino-6-cyclopropyl-7-fluoro-1-(3-methoxy-2,6-dimethylphenyl)-1H-pyrrolo[3,2-c]pyridine-3-carbonitrile

To a solution of 2-amino-6-chloro-7-fluoro-1-(3-methoxy-2,6-dimethyl-phenyl)pyrrolo[3,2-c]pyridine-3-carbonitrile (450 mg, 1.31 mmol) in toluene (15 mL) and water (1.5 mL) were added cyclopropylboronic acid (560 g, 6.53 mmol), K₃PO₄ (1.39 g, 6.53 mmol), XPhos Pd G3 (110 mg, 0.130 mmol) and XPhos (50 mg, 0.104 mmol). The reaction was stirred at 110° C. for 2 hours under nitrogen atmosphere. After cooling, the mixture was quenched with water (15 mL) and extracted with ethyl acetate (10 mL*2). The combined organic layers were washed with brine (10 mL), dried over anhydrous sodium sulfate, filtered, and concentrated under reduced pressure. The residue was purified by flash column chromatography (silica gel, 100-200 mesh, gradient 0˜35% ethyl acetate in petroleum ether) to afford the title compound (500 mg, 90%) as a yellow solid. LCMS R_{T}=0.465 min, m/z=350.9 [M+H]⁺.

### Step 5: Synthesis of 2-amino-6-cyclopropyl-7-fluoro-1-(3-methoxy-2,6-dimethylphenyl)-1H-pyrrolo[3,2-c]pyridine-3-carboxamide

A solution of 2-amino-6-cyclopropyl-7-fluoro-1-(3-methoxy-2,6-dimethyl-phenyl)pyrrolo[3,2-c]pyridine-3-carbonitrile (250 mg, 0.71 mmol) in sulfuric acid (2.0 mL) was stirred at 20° C. for 30 minutes, then poured into ice water (5.0 mL) and adjusted to pH=7 and extracted with ethyl acetate (8 mL*2). The combined extracts were washed with brine (10 mL), dried over anhydrous sodium sulfate, filtered, and concentrated under reduced pressure to afford the title compound (250 mg, 64%) as a colorless solid. LCMS R_{T}=0.442 min, m/z=369.0 [M+H]⁺.

### Step 6: Synthesis of 2-amino-6-cyclopropyl-7-fluoro-1-(3-hydroxy-2,6-dimethylphenyl)-1H-pyrrolo[3,2-c]pyridine-3-carboxamide (Compound 605)

To a solution of 2-amino-6-cyclopropyl-7-fluoro-1-(3-methoxy-2,6-dimethyl-phenyl)pyrrolo[3,2-c]pyridine-3-carboxamide (120 mg, 0.32 mmol) in dichloromethane (2.0 mL) was added dropwise boron tribromide (650 mg, 2.61 mmol) at 0° C. The reaction was stirred at 100° C. for 2 hours, and was then cooled and quenched with 1M hydrochloric acid (0.50 mL). The mixture was filtered to remove the insoluble solid and the filtrate was concentrated under reduced pressure. The residue was purified by RP-HPLC (0 to 30% acetonitrile in water with 0.225% formic acid) to give the title compound (28.7 mg, 24.8%) as a yellow solid. ¹H NMR (400 MHz, DMSO-d₆) δ=9.59 (br s, 1H), 8.58 (s, 1H), 7.06 (d, J=8.0 Hz, 1H), 6.92 (d, J=8.0 Hz, 1H), 6.82 (br s, 4H), 2.17-2.06 (m, 1H), 1.81 (s, 3H), 1.73 (s, 3H), 0.92-0.84 (m, 4H). ¹⁹F NMR (400 MHz, DMSO-d₆) δ=−158.784. LCMS R_{T}=1.519 min, m/z=355.3 [M+H]⁺.

### Example 37. Synthesis of Compound 622

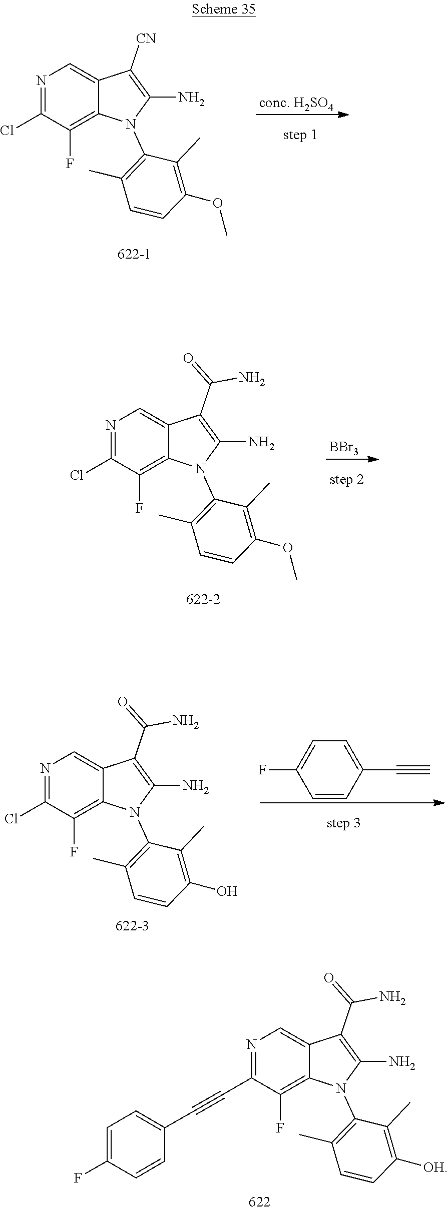

### Step 1: Synthesis of 2-amino-6-chloro-7-fluoro-1-(3-methoxy-2,6-dimethylphenyl)-1H-pyrrolo[3,2-c]pyridine-3-carboxamide

A solution of 2-amino-6-chloro-7-fluoro-1-(3-methoxy-2,6-dimethyl-phenyl)pyrrolo [3,2-c]pyridine-3-carbonitrile (1.2 g, 3.48 mmol) in 98% sulfuric acid (15 mL) was stirred at 20° C. for 1 hour, then poured into ice water (10 mL) and adjusted to pH=7 with NaOH solution. The mixture was extracted with ethyl acetate (10 mL*2). The organic extracts were washed with brine (10 mL*2), dried over anhydrous Na₂SO₄, filtered, and concentrated under reduced pressure to afford the title compound (1.1 g, 74%) as a colorless solid. LCMS R_{T}=0.625 min, m/z=363.0 [M+H]⁺.

### Step 2: Synthesis of 2-amino-6-chloro-7-fluoro-1-(3-hydroxy-2,6-dimethylphenyl)-1H-pyrrolo[3,2-c]pyridine-3-carboxamide

To a solution of 2-amino-6-chloro-7-fluoro-1-(3-methoxy-2,6-dimethyl-phenyl) pyrrolo[3,2-c]pyridine-3-carboxamide (1.5 g, 4.13 mmol) in dichloromethane (13 mL) was added tribromoborane (2 M solution in dichloromethane, 10.34 mL). The mixture was stirred at 20° C. for 1 hour, quenched with water (10 mL), and then extracted with ethyl acetate (15 mL*3). The organic extracts were dried over anhydrous Na₂SO₄, filtered, and concentrated under reduced pressure to give the title compound (1.08 g, 67%) as a yellow solid. LCMS R_{T}=0.481 min, m/z=349.0 [M+H]⁺.

### Step 3: Synthesis of 2-amino-7-fluoro-6-((4-fluorophenyl)ethynyl)-1-(3-hydroxy-2,6-dimethylphenyl)-1H-pyrrolo[3,2-c]pyridine-3-carboxamide (Compound 622)

To a solution of 2-amino-6-chloro-7-fluoro-1-(3-hydroxy-2,6-dimethyl-phenyl) pyrrolo[3,2-c]pyridine-3-carboxamide (280 mg, 0.8 mmol) in acetonitrile (20 mL) were added 1-ethynyl-4-fluoro-benzene (965 mg, 8.03 mmol), N,N-dicyclohexyl-methanamine (470 mg, 2.41 mmol) and XPhos Pd G3 (135 mg, 0.16 mmol). The reaction was stirred at 90° C. for 16 hours under nitrogen atmosphere. After cooling, the mixture was filtered and the filtrate was concentrated under reduced pressure. The residue was purified by RP-HPLC [gradient 18˜58% acetonitrile in water (0.225% formic acid as additive)] to give the title compound (160 mg, 35%) as a yellow solid. ¹H NMR (400 MHz, DMSO-d₆) δ=9.66 (br s, 1H), 8.80 (d, J=1.6 Hz, 1H), 7.59 (dd, J=8.8, 5.6 Hz, 2H), 7.26 (t, J=8.8 Hz, 2H), 7.12 (br s, 2H), 7.09 (d, J=8.4 Hz, 1H), 7.04 (br s, 2H), 6.94 (d, J=8.0 Hz, 1H), 1.82 (s, 3H), 1.74 (s, 3H). ¹⁹F NMR (400 MHz, DMSO-d₆) δ=−110.082, −146.126. LCMS R_{T}=1.943 min, m/z=433.1 [M+H]⁺.

### Example 38. Synthesis of Compound 623

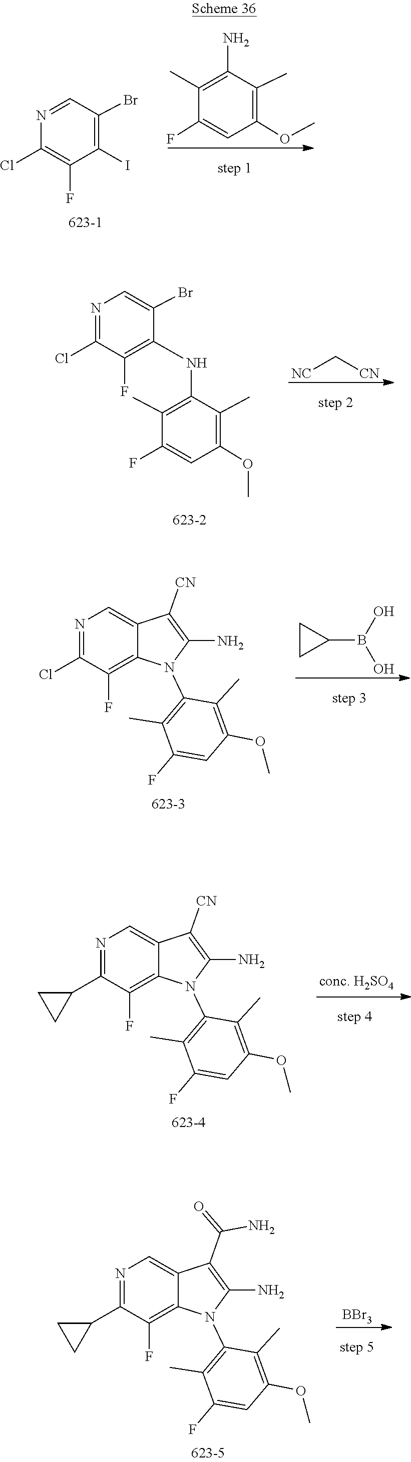

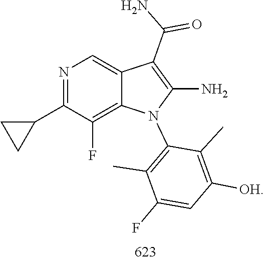 [SMILES: Cc1c(O)cc(F)c(C)c1-n1c(N)c(C(N)=O)c2cnc(C3CC3)c(F)c21]

### Step 1: Synthesis of 5-bromo-2-chloro-3-fluoro-N-(3-fluoro-5-methoxy-2,6-dimethyl-phenyl)pyridin-4-amine

To a solution of 3-fluoro-5-methoxy-2,6-dimethyl-aniline (1.0 g, 5.91 mmol) and 5-bromo-2-chloro-3-fluoro-4-iodo-pyridine (2.4 g, 7.09 mmol) in dioxane (10 mL) were added Pd(OAc)₂ (66 mg, 0.30 mmol), XantPhos (342 mg, 0.59 mmol) and cesium carbonate (3.9 g, 11.82 mmol). The mixture was stirred at 100° C. for 16 hours under nitrogen atmosphere. After cooling, the mixture was filtered and the filtrate was concentrated under reduced pressure. The residue was purified by flash column chromatography (silica gel, 100-200 mesh, gradient 0˜6% ethyl acetate in petroleum ether) to afford the title compound (1.5 g, 50%) as a yellow solid. LCMS R_{T}=0.809 min, m/z=378.9 [M+H]⁺. ¹H NMR (400 MHz, DMSO-d₆) δ=8.28 (br s, 1H), 8.17 (s, 1H), 6.88 (d, J=11.6 Hz, 1H), 3.79 (s, 3H) 1.97 (s, 3H), 1.92 (s, 3H).

### Step 2: Synthesis of 2-amino-6-chloro-7-fluoro-1-(3-fluoro-5-methoxy-2,6-dimethyl-phenyl)pyrrolo[3,2-c]pyridine-3-carbonitrile

To a solution of propanedinitrile (367 mg, 5.56 mmol) in 1,2-dimethoxyethane (10 mL) was added a solution of sodium tert-butoxide (713 mg, 7.42 mmol) in tert-butanol (2.0 mL) at 0° C. The mixture was stirred at 20° C. for 30 minutes under nitrogen atmosphere, followed by the addition of Pd(dppf)Cl₂·CH₂Cl₂ (303 mg, 0.37 mmol) and 5-bromo-2-chloro-3-fluoro-N-(3-fluoro-5-methoxy-2,6-dimethyl-phenyl)pyridin-4-amine (1.4 g, 3.71 mmol). The mixture was stirred at 110° C. for 16 hours under nitrogen atmosphere. After cooling, the reaction mixture was quenched with water (30 mL) at 0° C., and extracted with ethyl acetate (20 mL*3). The combined organic layers were washed with brine (10 mL), dried over sodium sulfate, filtered, and concentrated under reduced pressure. The residue was purified by flash column chromatography (silica gel, 100-200 mesh, gradient 0˜23% ethyl acetate in petroleum ether) to afford the title compound (500 mg, 34%) as a yellow solid. LCMS R_{T}=0.726 min, m/z=363.0 [M+H]⁺. ¹H NMR (400 MHz, DMSO-d₆) δ=8.17 (s, 1H), 7.49 (br s, 2H), 7.16 (d, J=8.0 Hz, 1H), 3.86 (s, 3H) 1.78 (s, 3H), 1.72 (s, 3H).

### Step 3: Synthesis of 2-amino-6-cyclopropyl-7-fluoro-1-(3-fluoro-5-methoxy-2,6-dimethyl-phenyl)pyrrolo[3,2-c]pyridine-3-carbonitrile

To a solution of cyclopropylboronic acid (142 mg, 1.65 mmol) and 2-amino-6-chloro-7-fluoro-1-(3-fluoro-5-methoxy-2,6-dimethyl-phenyl)pyrrolo[3,2-c]pyridine-3-carbonitrile (200 mg, 0.55 mmol) in water (0.50 mL) and toluene (5.0 mL) were added XPhos Pd G3 (47 mg, 0.06 mmol) and potassium phosphate (585 mg, 2.76 mmol). The reaction was stirred at 100° C. for 2 hours under nitrogen atmosphere. After cooling, the mixture was concentrated under reduced pressure. The residue was purified by flash column chromatography (silica gel, 100-200 mesh, gradient 0˜22% ethyl acetate in petroleum ether) to afford the title compound (150 mg, 49%) as a yellow solid. LCMS R_{T}=0.491 min, m/z=369.1 [M+H]⁺. ¹H NMR (400 MHz, DMSO-d₆) δ=8.17 (s, 1H), 7.18 (br s, 2H), 7.13 (d, J=11.6 Hz, 1H), 3.86 (s, 3H), 2.17-2.05 (m, 1H), 1.78 (s, 3H), 1.72 (s, 3H), 0.95-0.82 (m, 4H).

### Step 4: Synthesis of 2-amino-6-cyclopropyl-7-fluoro-1-(3-fluoro-5-methoxy-2,6-dimethyl-phenyl)pyrrolo[3,2-c]pyridine-3-carboxamide

A solution of 2-amino-6-cyclopropyl-7-fluoro-1-(3-fluoro-5-methoxy-2,6-dimethyl-phenyl)pyrrolo[3,2-c]pyridine-3-carbonitrile (150 mg, 0.41 mmol) in 98% sulfuric acid (1.0 mL) was stirred at 20° C. for 1 hour, then poured onto ice-water (10 mL) and adjusted to pH=8 with saturated NaOH solution. The mixture was filtered and the filter cake was concentrated under vacuum to afford the title compound (150 mg, 60%) as a yellow solid. LCMS R_{T}=0.494 min, m/z=387.0 [M+H]⁺.

### Step 5: Synthesis of 2-amino-6-cyclopropyl-7-fluoro-1-(3-fluoro-5-hydroxy-2,6-dimethyl-phenyl)pyrrolo[3,2-c]pyridine-3-carboxamide (Compound 623)

To a solution of 2-amino-6-cyclopropyl-7-fluoro-1-(3-fluoro-5-methoxy-2,6-dimethyl-phenyl)pyrrolo[3,2-c]pyridine-3-carboxamide (150 mg, 0.39 mmol) in dichloromethane (1.0 mL) was added tribromoborane (2 M, 0.97 mL) dropwise at 0° C., and the reaction mixture was stirred at 20° C. for 1 hour under nitrogen atmosphere. The mixture was quenched with methanol (5.0 ml) and concentrated under reduced pressure. The residue was purified by RP-HPLC [gradient 0˜34% acetonitrile in water, 0.225% formic acid as additive) to afford the title compound (33.9 mg, 23%) as a white solid. LCMS R_{T}=1.884 min, m/z=373.2 [M+H]⁺. ¹H NMR (400 MHz, DMSO-d₆) δ=10.10 (br s, 1H), 8.59 (s, 1H), 6.95 (br s, 2H), 6.83 (d, J=8.4 Hz, 1H), 6.80 (br s, 2H), 2.17-2.05 (m, 1H), 1.73 (s, 3H), 1.69 (s, 3H), 0.95-0.82 (m, 4H). ¹⁹F NMR (400 MHz, DMSO-d₆) δ=−118.264, −118.269, −158.904.

### Example 39. Synthesis of Compound 624

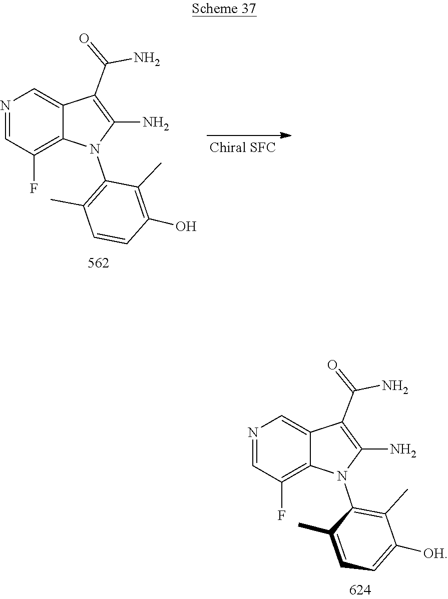

### 2-amino-7-fluoro-1-(3-hydroxy-2,6-dimethyl-phenyl)pyrrolo[3,2-c]pyridine-3-carboxamide

2-Amino-7-fluoro-1-(3-hydroxy-2,6-dimethyl-phenyl)pyrrolo[3,2-c]pyridine-3-carboxamide (Compound 562, 80 mg, 0.25 mmol) was separated by chiral SFC {Conditions: [Column: Daicel Chiralcel OX (250 mm*30 mm, 10 μm); Mobile phase: (0.1% NH₃ H₂O, EtOH); B %: 55%]} to afford two fractions.

Fraction 1 (t₁=2.659 min) was further purified by RP-HPLC [(Column: Welch Xtimate C18 150*30 mm*5 um; Mobile phase: [water (HCl)-MeCN]; B %: 0-24; Gradient time: 25 min) to afford 2-amino-7-fluoro-1-(3-hydroxy-2,6-dimethyl-phenyl)pyrrolo[3,2-c]pyridine-3-carboxamide (Compound 624), 18.9 mg, 23% yield) as a white solid. ¹H NMR (400 MHz, DMSO-d₆) δ 9.83 (br s, 1H), 9.12-9.04 (m, 1H), 8.60 (d, J=5.2 Hz, 1H), 7.52 (br s, 2H), 7.32 (br s, 2H), 7.12 (d, J=8.4 Hz, 1H), 7.06-6.97 (m, 1H), 1.82 (s, 3H), 1.74 (s, 3H). ¹⁹F NMR (400 MHz, DMSO-d₆) δ=−148.517. LCMS R_{T}=1.500 min, m/z=315.1 [M+H]⁺.

### Example 40. Synthesis of Compound 625

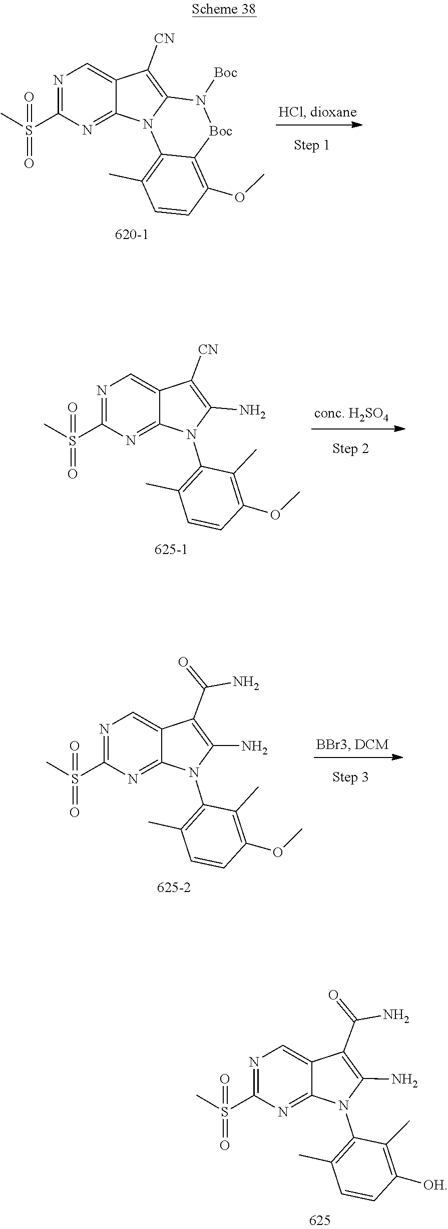

### Step 1: Synthesis of 6-amino-7-(3-methoxy-2,6-dimethylphenyl)-2-(methylsulfonyl)-7H-pyrrolo[2,3-d]pyrimidine-5-carbonitrile

To a 1-dram vial charged with a stir bar was added tert-butyl (tert-butoxycarbonyl)(5-cyano-7-(3-methoxy-2,6-dimethylphenyl)-2-(methylsulfonyl)-7H-pyrrolo[2,3-d]pyrimidin-6-yl)carbamate (150.0 mg, 1 Eq, 262.4 μmol) in 1,4-dioxane (1 mL) and 6 M HCl/Dioxane (1 mL, 6 mmol). The reaction was stirred at 80° C. for 3 hours. The reaction was poured into water (15 mL), the pH was adjusted to 8 with sat. sodium bicarbonate solution and the mixture was extracted with ethyl acetate (2×20 mL). The combined organics were washed with brine, dried over anhydrous sodium sulfate, filtered, and evaporated. The crude residue was purified on a 4 g silica gel column from 40% EtOAc/Hex to 60% EtOAc/Hex to give 6-amino-7-(3-methoxy-2,6-dimethylphenyl)-2-(methylsulfonyl)-7H-pyrrolo[2,3-d]pyrimidine-5-carbonitrile (75.7 mg, 204 μmol, 77.7%) as a yellow oil. LCMS R_{T}=0.89 min, m/z=372.2 [M+H]⁺.

### Step 2: Synthesis of 6-amino-7-(3-methoxy-2,6-dimethylphenyl)-2-(methylsulfonyl)-7H-pyrrolo[2,3-d]pyrimidine-5-carboxamide

6-Amino-7-(3-methoxy-2,6-dimethylphenyl)-2-(methylsulfonyl)-7H-pyrrolo[2,3-d]pyrimidine-5-carbonitrile (75.0 mg, 1 eq, 202 μmol) was stirred with concentrated sulfuric acid (2.0 mL) at room temperature for 1 hour To the reaction mixture was added crushed ice (approx. 50 gm), followed by dropwise addition of saturated sodium bicarbonate solution (10 mL), followed by the dropwise addition of 2M aqueous NaOH maintaining the temperature below 5° C. and with stirring to pH 8. The product was then extracted with ethyl acetate (15 mL×3). The combined organic extracts were washed with brine (15 mL), dried (sodium sulfate) and concentrated under reduced pressure to obtain 6-amino-7-(3-methoxy-2,6-dimethylphenyl)-2-(methylsulfonyl)-7H-pyrrolo[2,3-d]pyrimidine-5-carboxamide (58.2 mg, 149 μmol, 74.0%) as a white solid, which was used in the next step without further purification. LCMS R_{T}=0.792 min, m/z=390.2 [M+H]⁺.

### Step 3: Synthesis of 6-amino-7-(3-hydroxy-2,6-dimethylphenyl)-2-(methylsulfonyl)-7H-pyrrolo[2,3-d]pyrimidine-5-carboxamide (Compound 625)

To a solution of 6-amino-7-(3-methoxy-2,6-dimethylphenyl)-2-(methylsulfonyl)-7H-pyrrolo[2,3-d]pyrimidine-5-carboxamide (30.0 mg, 1 eq, 77.0 μmol) in anhydrous dichloromethane (1 mL) was added a solution of boron tribromide solution in dichloromethane (231 μL, 1 molar, 3 eq, 231 μmol) at room temperature. The solution slowly became cloudy and a yellow precipitate was observed. The reaction was continued at room temperature for 1 hour. After this, the reaction mixture was concentrated under reduced pressure. To the residue was added methanol (1 mL) and the solution was concentrated and the process was repeated twice. To the residue was added triethylamine (0.5 mL) and methanol (0.5 mL) and the mixture was concentrated under reduced pressure. The crude residue was dissolved in DMSO (2 mL) and purified using ACCQPrep HPLC system using 0 to 90% acetonitrile (0.1% v/v formic acid) in water (0.1% v/v formic acid) over 15 min. The fractions containing product were concentrated under reduced pressure and lyophilized to give 6-amino-7-(3-hydroxy-2,6-dimethylphenyl)-2-(methylsulfonyl)-7H-pyrrolo[2,3-d]pyrimidine-5-carboxamide (19.8 mg, 52.7 mol, 68.5%) as an off-white solid. LCMS R_{T}=0.596 min, m/z=376.2 [M+H]⁺; ¹H NMR (500 MHz, DMSO) δ 9.70 (s, 1H), 9.10 (s, 1H), 7.56 (s, 2H), 7.16-7.10 (m, 3H), 6.98 (d, J=8.3 Hz, 1H), 3.24 (s, 3H), 1.77 (s, 3H), 1.68 (s, 3H).

### Example 41. Synthesis of Compound 626

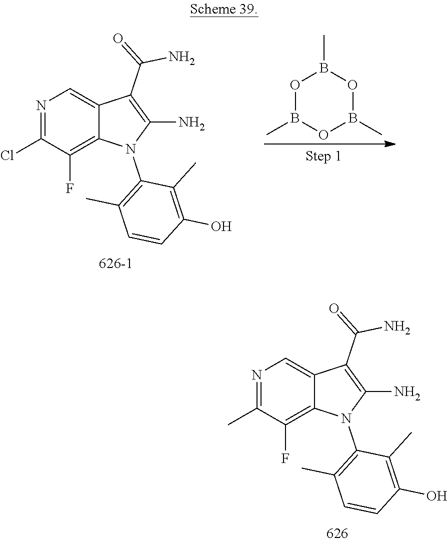

### Synthesis of 2-amino-7-fluoro-1-(3-hydroxy-2,6-dimethyl-phenyl)-6-methyl-pyrrolo[3,2-c]pyridine-3-carboxamide

To a solution of 2-amino-6-chloro-7-fluoro-1-(3-hydroxy-2,6-dimethyl-phenyl)pyrrolo [3,2-c]pyridine-3-carboxamide (50 mg, 0.14 mmol) and 2,4,6-trimethyl-1,3,5,2,4,6-trioxatriborinane (108 mg, 0.43 mmol) in 1,2-dimethoxyethane (1.0 mL) were added Pd(dppf)Cl₂ (21 mg, 0.03 mmol) and sodium tert-butoxide (41 mg, 0.43 mmol). The reaction was stirred at 120° C. for 1 hour under nitrogen atmosphere. After cooling, the mixture was filtered and the filtrate was concentrated under reduced pressure. The residue was purified by RP-HPLC [Column: Welch Xtimate C18 150*30 mm*5 um; Mobile phase: water (FA)-MeCN; B %: 0-24; Gradient time: 25 min; Flow Rate: 20 mL/min] to afford 2-amino-7-fluoro-1-(3-hydroxy-2,6-dimethyl-phenyl)-6-methyl-pyrrolo[3,2-c]pyridine-3-carboxamide (22.8 mg, 47% yield) as white solid. ¹H NMR (400 MHz, DMSO-d₆) δ 9.60 (br s, 1H), 8.63 (d, J=1.6 Hz, 1H), 7.05 (d, J=8.4 Hz, 1H), 6.91 (d, J=8.0 Hz, 1H), 6.85 (br s, 2H), 6.84 (br s, 2H), 2.33 (d, J=3.2 Hz, 3H), 1.80 (s, 3H), 1.71 (s, 3H). ¹⁹F NMR (400 MHz, DMSO-d₆) δ=−152.996. LCMS R_{T}=1.640 min, m/z=329.1 [M+H]⁺.

### Example 42. Synthesis of Compound 627

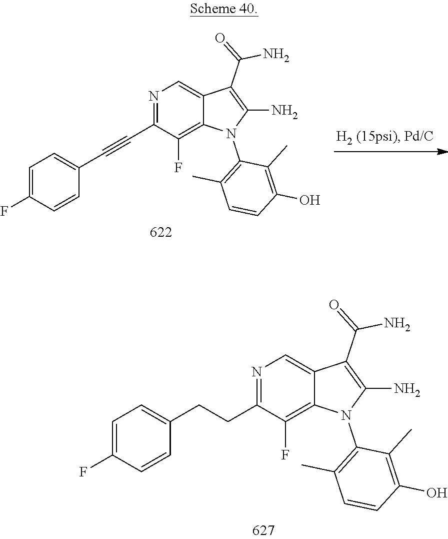 [SMILES: Cc1ccc(O)c(C)c1-n1c(N)c(C(N)=O)c2cnc(C#Cc3ccc(F)cc3)c(F)c21 ; Cc1ccc(O)c(C)c1-n1c(N)c(C(N)=O)c2cnc(CCc3ccc(F)cc3)c(F)c21]

### Synthesis of 2-amino-7-fluoro-6-(4-fluorophenethyl)-1-(3-hydroxy-2,6-dimethylphenyl)-1H-pyrrolo[3,2-c]pyridine-3-carboxamide

To a solution of 2-amino-7-fluoro-6-[2-(4-fluorophenyl)ethynyl]-1-(3-hydroxy-2,6-dimethyl-phenyl)pyrrolo[3,2-c]pyridine-3-carboxamide (100 mg, 0.231 mmol) in methanol (2.0 mL) was added 5% Pd/C (15 mg). The reaction mixture was stirred at 20° C. for 1 hour under hydrogen atmosphere, then filtered through a Celite pad and the filtrate was concentrated under reduced pressure. The residue was purified by RP-HPLC [gradient 0 to 38% acetonitrile in water (0.225% formic acid as additive)] to give the title compound (47.9 mg, 47%) was obtained as a yellow solid. ¹H NMR (400 MHz, DMSO-d₆) δ=9.58 (br s, 1H), 8.70 (s, 1H), 7.09 (dd, J=5.6, 3.2 Hz, 2H), 7.01 (dd, J=18.0, 8.8 Hz, 3H), 6.89 (d, J=8.4 Hz, 1H), 6.88 (br s, 2H), 6.84 (br s, 2H), 2.92 (s, 4H), 1.74 (s, 3H), 1.66 (s, 3H). ¹⁹F NMR (400 MHz, DMSO-d₆) δ=−117.652, −154.419. LCMS R_{T}=1.91 min, m/z=437.4 [M+H]⁺.

### Example 43. Synthesis of Compound 628

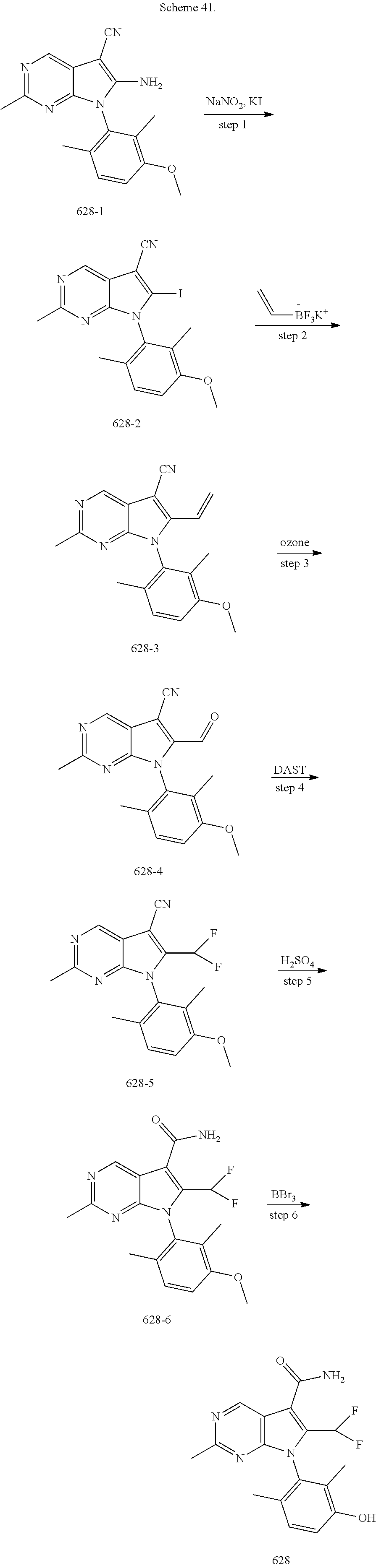

### Step 1: Synthesis of 6-iodo-7-(3-methoxy-2,6-dimethyl-phenyl)-2-methyl-pyrrolo[2,3-d]pyrimidine-5-carbonitrile

To a solution of 6-amino-7-(3-methoxy-2,6-dimethyl-phenyl)-2-methyl-pyrrolo[2,3-d]pyrimidine-5-carbonitrile (1.5 g, 4.88 mmol) and p-TsOH·H₂O (2.79 g, 14.64 mmol) in acetonitrile (25 mL) and water (10 mL) was added a solution of potassium iodide (2.03 g, 12.20 mmol) and sodium nitrite (673 mg, 9.76 mmol) in water (5.0 mL) at −10° C. dropwise. The reaction mixture was stirred at −10° C. for 3 hours, and was then extracted with dichloromethane (200 mL). The organic phase was washed with water (100 mL*2) and brine (100 mL*2), dried over sodium sulfate, filtered, and concentrated under reduced pressure. The residue was purified by Biotage® combi flash (Column: 40 g SepaFlash® Silica Flash column; Eluent: gradient 0˜23% tetrahydrofuran in petroleum ether; Gradient time: 10 min; Hold time: 5 min; Flow rate: 120 mL/min) to afford the title compound (810 mg, 30.56%) as a yellow solid. LCMS R_{T}=0.674 min, m/z=419.0 [M+H]⁺.

### Step 2: Synthesis of 7-(3-methoxy-2,6-dimethyl-phenyl)-2-methyl-6-vinyl-pyrrolo[2,3-d]pyrimidine-5-carbonitrile

To a solution of 6-iodo-7-(3-methoxy-2,6-dimethyl-phenyl)-2-methyl-pyrrolo[2,3-d]pyrimidine-5-carbonitrile (640 mg, 1.53 mmol) and vinyl potassium trifluoroborate (410 mg, 3.06 mmol) in dioxane (15 mL) and water (6.0 mL) were added potassium carbonate (846 mg, 6.12 mmol) and Pd(dppf)Cl₂ (112 mg, 0.15 mmol). The reaction was stirred at 100° C. for 15 hours under nitrogen atmosphere. After cooling, the mixture was diluted with ethyl acetate (80 mL), washed with water (30 mL*2) and brine (30 mL*2), dried over sodium sulfate, filtered, and concentrated under reduced pressure. The residue was purified by Biotage® combi flash (Column: 12 g SepaFlash® Silica Flash column; Eluent: gradient 0˜14% THF in petroleum ether; Gradient time: 10 min; Hold time: 5 min; Flow rate: 40 mL/min) to afford the title compound (410 mg, 69.01%) as a yellow solid. LCMS R_{T}=0.593 min, m/z=319.1 [M+H]⁺.

### Step 3: Synthesis of 6-formyl-7-(3-methoxy-2,6-dimethyl-phenyl)-2-methyl-pyrrolo[2,3-d]pyrimidine-5-carbonitrile

Ozone was bubbled through a solution of 7-(3-methoxy-2,6-dimethyl-phenyl)-2-methyl-6-vinyl-pyrrolo[2,3-d]pyrimidine-5-carbonitrile (320 mg, 1.01 mmol) in dichloromethane (10 mL) at −78° C. for 30 minutes, and then triphenylphosphine (527 mg, 2.01 mmol) was added to the mixture at −78° C., which was further stirred at 20° C. for 30 minutes. The reaction mixture was concentrated under reduced pressure. The residue was purified by Biotage® combi flash (Column: 12 g SepaFlash® Silica Flash column; Eluent: gradient 0˜20% THF in petroleum ether; Gradient time: 10 min; Hold time: 5 min; Flow rate: 40 mL/min) to afford the title compound (180 mg) as yellow oil. LCMS R_{T}=0.655 min, m/z=321.0 [M+H]⁺.

### Step 4: Synthesis of 6-(difluoromethyl)-7-(3-methoxy-2,6-dimethyl-phenyl)-2-methyl-pyrrolo[2,3-d]pyrimidine-5-carbonitrile

To a solution of 6-formyl-7-(3-methoxy-2,6-dimethyl-phenyl)-2-methyl-pyrrolo[2,3-d]pyrimidine-5-carbonitrile (160 mg, 0.499 mol) in dichloromethane (10 mL) was added DAST (241 mg, 1.50 mmol) dropwise at 0° C. The reaction was stirred at 20° C. for 2 hours, and was then quenched with water (1.0 mL). The mixture was concentrated under reduced pressure, and the residue was purified by preparative TLC (silica gel, petroleum ether:ethyl acetate=1/1) to afford the title compound (92 mg, 40%) as a yellow solid. LCMS R_{T}=1.729 min, m/z=342.9 [M+H]⁺.

### Step 5: Synthesis of 6-(difluoromethyl)-7-(3-methoxy-2,6-dimethyl-phenyl)-2-methyl-pyrrolo[2,3-d]pyrimidine-5-carboxamide

A solution of 6-(difluoromethyl)-7-(3-methoxy-2,6-dimethyl-phenyl)-2-methyl-pyrrolo[2,3-d]pyrimidine-5-carbonitrile (92 mg, 0.269 mmol) in 98% sulfuric acid (1.0 mL) was stirred at 20° C. for 1 hour, and then poured onto ice water (20 mL). The so-formed suspension was adjusted to pH˜9 with NaOH solution and extracted with dichloromethane (30 mL*3). The combined extracts were washed with brine (30 mL), dried over sodium sulfate, filtered, and concentrated under reduced pressure to give the crude title compound (100 mg) as a yellow solid, which would be used in the next step directly without further purification. LCMS R_{T}=0.532 min, m/z=361.0 [M+H]⁺.

### Step 6: Synthesis of 6-(difluoromethyl)-7-(3-hydroxy-2,6-dimethyl-phenyl)-2-methyl-pyrrolo[2,3-d]pyrimidine-5-carboxamide

To a solution of 6-(difluoromethyl)-7-(3-methoxy-2,6-dimethyl-phenyl)-2-methyl-pyrrolo[2,3-d]pyrimidine-5-carboxamide (100 mg, 0.269 mmol) in dichloromethane (3.0 mL) was added tribromoborane (2 M solution in dichloromethane, 0.70 mL) dropwise at 0° C. The reaction mixture was stirred at 20° C. for 2 hours, and was then quenched with water (5.0 mL) and extracted with dichloromethane (30 mL). The organic phase was dried over sodium sulfate, filtered, and concentrated under reduced pressure. The residue was purified by RP-HPLC [(0.225% FA condition); Eluent: gradient 0˜32% acetonitrile in water; Gradient time: 25 min; Hold time: 3 min; Flow rate: 25 mL/min]. Pure fraction was lyophilized to afford the title compound (32.9 mg, 23.15%) as a white solid. LCMS R_{T}=1.080 min, m/z=347.0 [M+H]⁺. ¹H NMR (400 MHz, CD₃OD) δ=9.28 (s, 1H), 7.44 (t, J=52.0 Hz, 1H), 7.05 (d, J=8.4 Hz, 1H), 6.92 (d, J=8.4 Hz, 1H), 2.68 (s, 3H), 1.77 (s, 3H), 1.70 (s, 3H). ¹⁹F NMR (400 MHz, CD₃OD) δ=−116.940, −117.056.

### Example 44. Synthesis of Compound 629

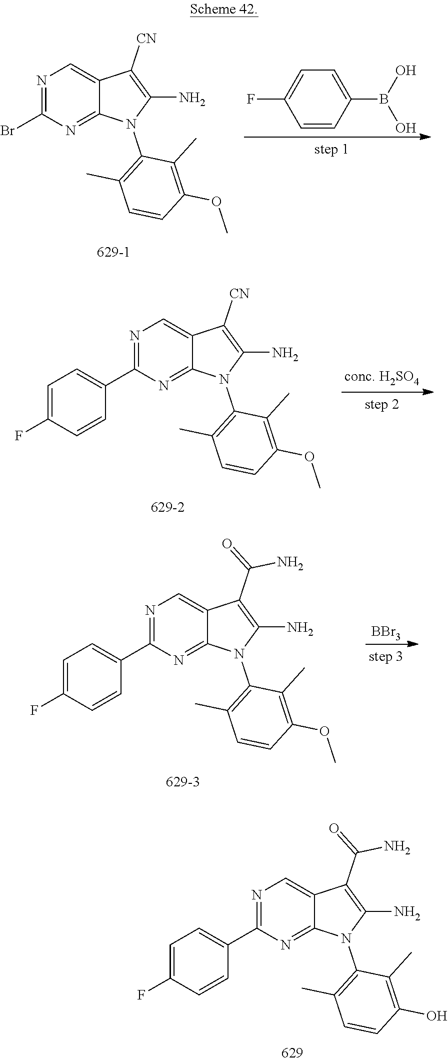 [SMILES: COc1ccc(C)c(-n2c(N)c(C#N)c3cnc(Br)nc32)c1C ; OB(O)c1ccc(F)cc1 ; COc1ccc(C)c(-n2c(N)c(C#N)c3cnc(-c4ccc(F)cc4)nc32)c1C ; COc1ccc(C)c(-n2c(N)c(C(N)=O)c3cnc(-c4ccc(F)cc4)nc32)c1C ; "BBr3" ; Cc1ccc(O)c(C)c1-n1c(N)c(C(N)=O)c2cnc(-c3ccc(F)cc3)nc21]

### Step 1: Synthesis of 6-amino-2-(4-fluorophenyl)-7-(3-methoxy-2,6-dimethyl-phenyl)pyrrolo[2,3-d]pyrimidine-5-carbonitrile

To a solution of (4-fluorophenyl)boronic acid (226 mg, 1.61 mmol) and 6-amino-2-bromo-7-(3-methoxy-2,6-dimethyl-phenyl)pyrrolo[2,3-d]pyrimidine-5-carbonitrile (200 mg, 0.54 mmol) in 1,4-dioxane (20 mL) and water (2.0 mL) were added Pd(dppf)Cl₂ dichloromethane adduct (22 mg, 0.03 mol) and Na₂CO₃ (171 mg, 1.61 mmol). The reaction was stirred at 100° C. for 16 hours under nitrogen atmosphere. After cooling, the mixture was diluted with water (10 mL) and extracted with ethyl acetate (10 mL*3). The combined extracts were washed with brine (10 mL*2), dried over anhydrous Na₂SO₄, filtered, and concentrated under reduced pressure. The residue was purified by flash column chromatography (silica gel, 100-200 mesh, gradient 0˜24% ethyl acetate in petroleum ether) to afford the title compound (192 mg, 78%) as a yellow solid. LCMS R_{T}=0.699 min, m/z=388.1 [M+H]⁺. ¹H NMR (400 MHz, DMSO-d₆) δ=8.70 (s, 1H), 8.20-8.06 (m, 2H), 7.50 (br s, 2H), 7.30 (d, J=8.4 Hz, 1H), 7.23 (t, J=8.8 Hz, 2H), 7.15 (d, J=8.4 Hz, 1H), 3.87 (s, 3H), 1.85 (s, 3H), 1.76 (s, 3H).

### Step 2: Synthesis of 6-amino-2-(4-fluorophenyl)-7-(3-methoxy-2,6-dimethyl-phenyl)pyrrolo[2,3-d]pyrimidine-5-carboxamide

A solution of 6-amino-2-(4-fluorophenyl)-7-(3-methoxy-2,6-dimethyl-phenyl)pyrrolo [2,3-d]pyrimidine-5-carbonitrile (192 mg, 0.5 mmol) in 98% H₂SO₄ (0.26 mL, 4.96 mmol) was stirring at 20° C. for 30 minutes, then poured onto ice-water (10 mL) and adjusted to pH=8 with saturated sodium bicarbonate solution. The mixture was extracted with ethyl acetate (10 mL*3). The combined extracts were washed with brine (10 mL), dried over anhydrous Na₂SO₄, filtered, and concentrated under reduced pressure to afford the title compound (90 mg, 60%) as a yellow solid, which would be used directly in the next step without further purification. LCMS R_{T}=0.587 min, m/z=406.1 [M+H]⁺.

### Step 3: Synthesis of 6-amino-2-(4-fluorophenyl)-7-(3-hydroxy-2,6-dimethyl-phenyl)pyrrolo[2,3-d]pyrimidine-5-carboxamide (Compound 629)

To a solution of 6-amino-2-(4-fluorophenyl)-7-(3-methoxy-2,6-dimethyl-phenyl) pyrrolo[2,3-d]pyrimidine-5-carboxamide (170 mg, 0.42 mmol) in dichloromethane (2.0 mL) was added tribromoborane (2 M solution in dichloromethane, 1.05 mL) at 0° C. The reaction mixture was stirred at 20° C. for 30 minutes under nitrogen atmosphere, and then quenched with methanol (5.0 mL) and concentrated under reduced pressure. The residue was purified by RP-HPLC [gradient 14 to 54% acetonitrile in water (0.225% formic acid as additive)] to afford the title compound (80.8 mg, 48%) as a white solid. ¹H NMR (400 MHz, DMSO-d₆) δ=9.62 (br s, 1H), 9.08 (s, 1H), 8.16 (dd, J=8.8, 5.6 Hz, 2H), 7.29-7.15 (m, 4H), 7.12 (d, J=8.4 Hz 1H), 6.97 (d, J=8.4 Hz, 1H), 6.96 (br s, 2H), 1.81 (s, 3H), 1.72 (s, 3H). ¹⁹F NMR (400 MHz, DMSO-d₆) δ=−113.132. LCMS R_{T}=1.111 min, m/z=392.1 [M+H]⁺.

### Example 45. Synthesis of Compound 619

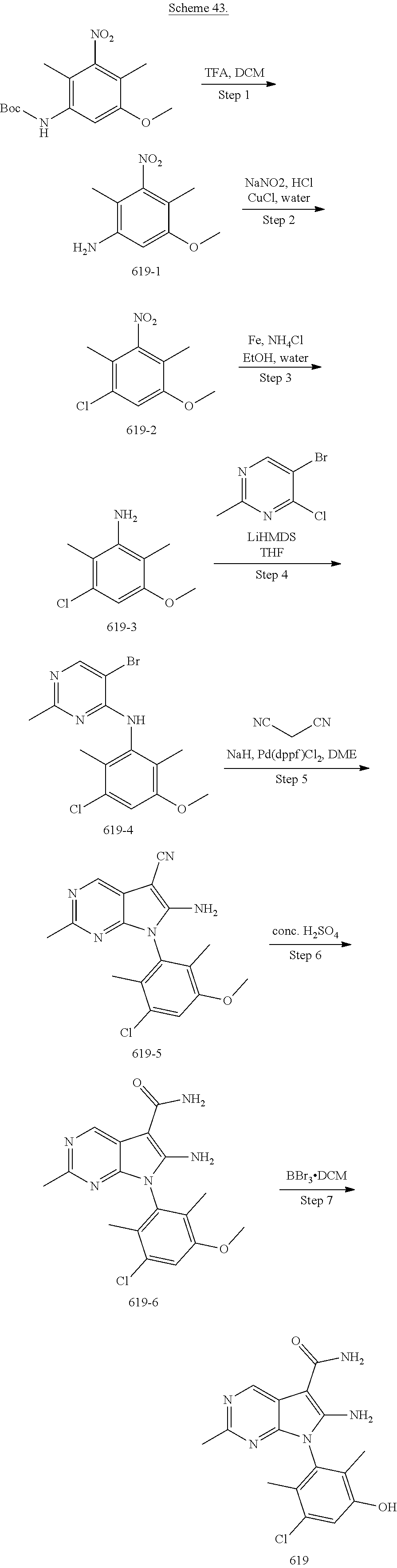

### Step 1: Synthesis of 5-methoxy-2,4-dimethyl-3-nitroaniline

To a solution of tert-butyl (5-methoxy-2,4-dimethyl-3-nitrophenyl)carbamate (10.0 g, 1 eq, 33.7 mmol) in dichloromethane (75 mL), was added TFA (25 mL, 9.6 Eq, 0.32 mol) and the reaction stirred at room temperature overnight. The reaction mixture was cooled at 0° C. and excess 2 N NaOH (170 mL) and dichloromethane (200 mL) were added and the mixture was stirred for 15 min after which the layers were separated. The organic layer was dried over anhydrous magnesium sulfate, filtered, and evaporated. The crude residue was loaded onto Celite and purified using flash chromatography on an 80 g column from 0→30%→60% EtOAc/Hex to give 5-methoxy-2,4-dimethyl-3-nitroaniline (3.818 g, 19.46 mmol, 57.7%) as a yellow-orange solid. LCMS R_{T}=0.952 min, m/z=197.1 [M+H]⁺; ¹H NMR (400 MHz, CDCl₃) δ 6.29 (s, 1H), 3.79 (d, J=1.2 Hz, 3H), 3.72 (s, 2H), 1.99 (dd, J=14.9, 1.2 Hz, 6H).

### Step 2: Synthesis of 1-chloro-5-methoxy-2,4-dimethyl-3-nitrobenzene

To a solution of 5-methoxy-2,4-dimethyl-3-nitroaniline (600.0 mg, 1 eq, 3.058 mmol) in HCl (9.0 mL, 12 molar, 35 eq, 0.11 mol) was added a solution of sodium nitrite (221.5 mg, 1.05 eq, 3.211 mmol) in water (0.6 mL) at 0° C. The reaction was stirred at 0° C. for 1 hour, and then copper(I) chloride (605.5 mg, 2 eq, 6.116 mmol) was added at 0° C. The reaction was stirred at room temperature overnight. The reaction was poured into water (50 mL), and the solution brought to pH˜7 with the addition of potassium carbonate. The mixture was extracted with ethyl acetate (3×50 mL). The combined organics were washed with brine (50 mL), dried over anhydrous sodium sulfate, filtered, and concentrated. The crude residue was loaded onto Celite and purified using flash chromatography on a 12 g silica gel column from 0 to 20% EtOAc/Hex to give 1-chloro-5-methoxy-2,4-dimethyl-3-nitrobenzene (448.6 mg, 2.080 mmol, 68.03%) as a white solid. LCMS R_{T}=1.282 min.

### Step 3: Synthesis of 3-chloro-5-methoxy-2,6-dimethylaniline

In a round bottom flask equipped with a stir bar was dissolved 1-chloro-5-methoxy-2,4-dimethyl-3-nitrobenzene (445.0 mg, 1 eq, 2.064 mmol) in ethanol (16 mL) and water (4.0 mL). Iron (1.153 g, 10 eq, 20.64 mmol) and ammonium chloride (1.104 g, 10 eq, 20.64 mmol) were added and the reaction mixture was heated at 80° C. for 1 hour. The mixture was filtered through a pad of Celite and washed with ethyl acetate. The filtrate was concentrated, and the resultant material was suspended in water, and extracted with ethyl acetate. The combined organics were washed with sat. NaHCO₃ solution, brine, dried over anhydrous sodium sulfate, filtered, and concentrated to give 3-chloro-5-methoxy-2,6-dimethylaniline (372.5 mg, 2.006 mmol, 97.23%) as a white solid that was used in the next step without further purification. LCMS R_{T}=1.09 min, m/z=186.1 [M+H]⁺; ¹H NMR (500 MHz, CDCl₃) δ 6.41 (s, 1H), 3.77 (s, 3H), 3.71 (s, 2H), 2.19 (s, 3H), 2.02 (s, 3H).

### Step 4: Synthesis of 5-bromo-N-(3-chloro-5-methoxy-2,6-dimethylphenyl)-2-methylpyrimidin-4-amine

5-Bromo-4-chloro-2-methylpyrimidine (122.9 mg, 1.1 eq, 592.5 μmol) was added to THF (5 mL) in a round bottom. flask followed by 3-chloro-5-methoxy-2,6-dimethylaniline (100.0 mg, 1 eq, 538.6 μmol) and cooled at 0° C. LiHMDS (700.2 μL, 1 molar, 1.3 eq, 700.2 μmol) was added and the reaction stirred at room temperature overnight. Saturated ammonium chloride solution (50 mL) was added to quench the reaction and the aqueous layer was extracted with ether (2×30 mL). The combined organics were washed with brine, dried over anhydrous sodium sulfate, filtered, and evaporated. The crude residue was loaded onto Celite and purified using flash chromatography on a Redi-Sep Gold column (24 g) from 0→20% EtOAc/Hex to give 5-bromo-N-(3-chloro-5-methoxy-2,6-dimethylphenyl)-2-methylpyrimidin-4-amine (42.2 mg, 118 μmol, 22.0%) as a cream solid. LCMS R_{T}=0.962 min, m/z=356.1, 358.1 [M+H]⁺.

### Step 5: Synthesis of 6-amino-7-(3-chloro-5-methoxy-2,6-dimethylphenyl)-2-methyl-7H-pyrrolo[2,3-d]pyrimidine-5-carbonitrile

To a solution of malononitrile (15.6 mg, 2.1 eq, 236 μmol) in 1,2-Dimethoxyethane (0.3 mL) in a microwave vial, was added sodium hydride (10.3 mg, 60% Wt, 2.3 eq, 258 μmol). The resulting mixture was stirred for 5 min, and then a solution of 5-bromo-N-(3-chloro-5-methoxy-2,6-dimethylphenyl)-2-methylpyrimidin-4-amine (40.0 mg, 1 eq, 112 μmol) and PdCl₂(dppf) (4.10 mg, 0.05 Eq, 5.61 μmol) in 1,2-dimethoxyethane (0.8 mL) was added. The reaction was sparged with nitrogen for 2 min and microwaved at 120° C. for 3 hours. The mixture was cooled to room temperature, diluted with water (15 mL) and extracted with ethyl acetate (2×15 mL). The combined organics were washed with brine, dried over anhydrous sodium sulfate, filtered, and concentrated. The resultant residue was loaded onto Celite and purified by flash chromatography on a 4 g gold column from 20 to 60% EtOAc/Hex to give 6-amino-7-(3-chloro-5-methoxy-2,6-dimethylphenyl)-2-methyl-7H-pyrrolo[2,3-d]pyrimidine-5-carbonitrile (26.3 mg, 76.9 μmol, 68.6%) as a brown solid. LCMS R_{T}=0.819 min, m/z=342.1 [M+H]⁺.

### Step 6: Synthesis of 6-amino-7-(3-chloro-5-methoxy-2,6-dimethylphenyl)-2-methyl-7H-pyrrolo[2,3-d]pyrimidine-5-carboxamide

6-Amino-7-(3-chloro-5-methoxy-2,6-dimethylphenyl)-2-methyl-7H-pyrrolo[2,3-d]pyrimidine-5-carbonitrile (26.0 mg, 1 eq, 76.1 μmol) was stirred with concentrated sulfuric acid (1.0 mL) at room temperature for 1 hour. To the reaction mixture was carefully added crushed ice (approx. 50 gm), followed by dropwise addition of saturated sodium bicarbonate solution (5 mL) with stirring followed by the dropwise addition of 2M aqueous NaOH maintaining the temperature below 5° C. and with stirring to pH 8. The product was then extracted with ethyl acetate (15 mL×3). The combined organic extracts were washed with brine (15 mL), dried (sodium sulfate), and concentrated under reduced pressure to obtain 6-amino-7-(3-chloro-5-methoxy-2,6-dimethylphenyl)-2-methyl-7H-pyrrolo[2,3-d]pyrimidine-5-carboxamide (27.4 mg, 76.2 μmol, 100%) as an off brown solid, which was used in the next step without further purification. LCMS R_{T}=0.626 min, m/z=360.2 [M+H]⁺.

### Step 7: Synthesis of 6-amino-7-(3-chloro-5-hydroxy-2,6-dimethylphenyl)-2-methyl-7H-pyrrolo[2,3-d]pyrimidine-5-carboxamide (Compound 619)

To a solution of 6-amino-7-(3-chloro-5-methoxy-2,6-dimethylphenyl)-2-methyl-7H-pyrrolo[2,3-d]pyrimidine-5-carboxamide (27.0 mg, 1 eq, 75.0 μmol) in anhydrous dichloromethane (1 mL) was added a solution of boron tribromide solution in dichloromethane (0.23 mL, 1 molar, 3.1 eq, 0.23 mmol) at room temperature. The solution slowly became cloudy and a yellow precipitate was observed. The reaction was continued at room temperature for 1 hour. After this, the reaction mixture was concentrated under reduced pressure. To the residue was added methanol (1 mL) and the solution was concentrated and the process was repeated twice. To the residue was added triethylamine (0.5 mL) and methanol (0.5 mL) and the mixture was concentrated under reduced pressure. The crude residue was dissolved in DMSO and purified using an ACCQPrep HPLC system using 0 to 90% acetonitrile (0.1% v/v formic acid) in water (0.1% v/v formic acid) over 15 min. The fractions containing product were concentrated under reduced pressure and lyophilized to give 6-amino-7-(3-chloro-5-hydroxy-2,6-dimethylphenyl)-2-methyl-7H-pyrrolo[2,3-d]pyrimidine-5-carboxamide (20.1 mg, 58.1 μmol, 77.5%) as an off-white solid. LCMS R_{T}=0.59 min, m/z=346.2 [M+H]⁺; ¹H NMR (500 MHz, DMSO) δ 10.11 (s, 1H), 8.89 (s, 1H), 7.18 (s, 2H), 7.08 (s, 1H), 6.86 (s, 2H), 2.44 (s, 3H), 1.77 (s, 3H), 1.64 (s, 3H).

### Example 46. Synthesis of Compound 567

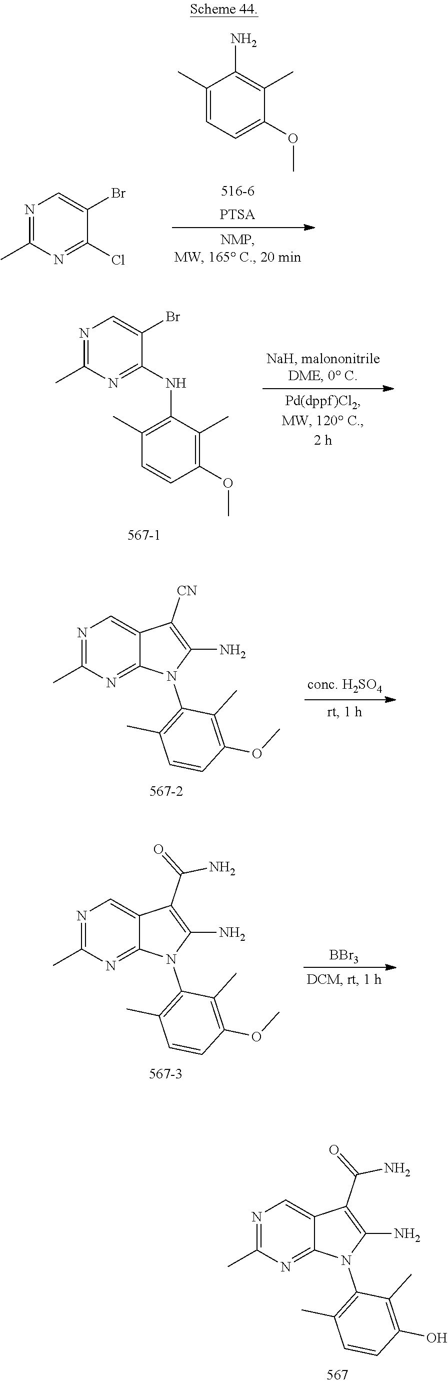

### Step 1: Synthesis of 5-bromo-N-(3-methoxy-2,6-dimethylphenyl)-2-methylpyrimidin-4-amine

To a mixture of 5-bromo-4-chloro-2-methylpyrimidine (315 mg, 1 eq, 1.52 mmol) and 3-methoxy-2,6-dimethylaniline (253 mg, 1.1 eq, 1.67 mmol) in NMP (5 mL) was added 4-methylbenzenesulfonic acid (26.1 mg, 0.1 eq, 152 μmol). The reaction flask was capped with a rubber septum and was heated at 110° C. for 12 hour. The reaction mixture was allowed to cool to room temperature and was then poured in saturated sodium bicarbonate solution (25 mL). The crude product was extracted with ethyl acetate (25 mL×3). The combined organic extracts were washed with water (25 mL), brine (25 mL), and dried (sodium sulfate). After filtering the drying agent, the filtrate was concentrated under reduced pressure and purified using ISCO RediSep Rf Silica 12 g. Eluted with 5%-25% ethyl acetate in hexanes to obtain 5-bromo-N-(3-methoxy-2,6-dimethylphenyl)-2-methylpyrimidin-4-amine (400 mg, 1.24 mmol, 81.8%) as a light yellow solid. ¹H NMR (300 MHz, CDCl₃) δ 8.29 (d, J=0.9 Hz, 1H), 7.07 (d, J=8.4 Hz, 1H), 6.78 (d, J=8.4 Hz, 1H), 6.61 (s, 1H), 3.84 (d, J=0.9 Hz, 3H), 2.37 (d, J=0.9 Hz, 3H), 2.14 (s, 3H), 2.07 (s, 3H).

### Step 2: Synthesis of 6-amino-7-(3-methoxy-2,6-dimethylphenyl)-2-methyl-7H-pyrrolo[2,3-d]pyrimidine-5-carbonitrile

To a 20 mL microwave vial with a rubber septum was taken malononitrile (82.0 mg, 2 eq, 1.24 mmol) and anhydrous degassed DME (10 mL) under nitrogen. The solution was cooled to 0° C. and sodium hydride (30 mg, 2 eq, 1.24 mmol) was added. The mixture was stirred for 15 min, then it was connected to vacuum and degassed for 2 min. Next 5-bromo-N-(3-methoxy-2,6-dimethylphenyl)-2-methylpyrimidin-4-amine (200 mg, 1 eq, 621 μmol) and [1,1′-bis(diphenylphosphino)ferrocene]dichloropalladium (II), complex with dichloromethane and methylene chloride (50.7 mg, 0.1 eq, 62.1 μmol) were added under nitrogen. The rubber septum was replaced with a microwave (Teflon) cap under positive pressure of nitrogen and the vial was sealed. The reaction mixture was subjected to microwave heating at 120° C. for 2 hours. The reaction mixture was filtered through a pad of Celite, and was washed with ethyl acetate (30 mL). The filtrate was concentrated and Celite (1 g) was added. The slurry was concentrated to obtain a plug. Purified using ISCO RediSep Rf Gold Silica 12 g column, eluting with 0%-5% methanol in dichloromethane to obtain 6-amino-7-(3-methoxy-2,6-dimethylphenyl)-2-methyl-7H-pyrrolo[2,3-d]pyrimidine-5-carbonitrile (150 mg, 488 μmol, 78.6%) as an off-white solid. ¹H NMR (300 MHz, DMSO) δ 8.52 (s, 1H), 7.38 (s, 2H), 7.24 (d, J=8.6 Hz, 1H), 7.10 (d, J=8.7 Hz, 1H), 3.83 (s, 3H), 2.43 (s, 3H), 1.77 (s, 3H), 1.68 (s, 3H).

### Step 3: Synthesis of 6-amino-7-(3-hydroxy-2,6-dimethylphenyl)-2-methyl-7H-pyrrolo[2,3-d]pyrimidine-5-carboxamide

6-Amino-7-(3-methoxy-2,6-dimethylphenyl)-2-methyl-7H-pyrrolo[2,3-d]pyrimidine-5-carbonitrile (116 mg, 1 eq, 377 μmol) was stirred with concentrated sulfuric acid (2 mL) at room temperature for 1 hr. The reaction was quenched by adding crushed ice (50 g). The mixture was stirred in an ice-bath and was slowly made alkaline (pH 8-9) by the addition of saturated sodium bicarbonate solution. The precipitated product was filtered and air-dried. The crude product was dissolved in a mixture of dichloromethane and methanol (2:1; 25 mL). To this was added Celite (1 g) and the mixture was concentrated under reduced pressure to obtain a solid plug. Purified using ISCO RediSep Rf Silica column 12 g eluting with 1%-10% methanol in dichloromethane to obtain 6-amino-7-(3-methoxy-2,6-dimethylphenyl)-2-methyl-7H-pyrrolo[2,3-d]pyrimidine-5-carboxamide (98 mg, 0.30 mmol, 80%) as a white solid. ¹H NMR (300 MHz, DMSO) δ 8.87 (s, 1H), 7.24 (d, J=8.1 Hz, 1H), 7.09 (d, J=8.1 Hz, 3H), 6.85 (s, 2H), 3.83 (s, 3H), 2.41 (s, 3H), 1.78 (s, 3H), 1.68 (s, 3H).

### Step 4: Synthesis of 6-amino-7-(3-hydroxy-2,6-dimethylphenyl)-2-methyl-7H-pyrrolo[2,3-d]pyrimidine-5-carboxamide (Compound 567)

To a suspension of 6-amino-7-(3-methoxy-2,6-dimethylphenyl)-2-methyl-7H-pyrrolo[2,3-d]pyrimidine-5-carboxamide (90 mg, 1 eq, 0.28 mmol) in dichloromethane (5 mL) at room temperature was added tribromoborane solution in dichloromethane (0.28 g, 1.1 mL, 1 molar, 4 eq, 1.1 mmol) dropwise under nitrogen. The reaction mixture was stirred at room temperature for 1 hour. The reaction mixture was cooled (ice bath) and was quenched by dropwise addition of methanol (1 mL). The mixture was then concentrated under reduced pressure to obtain a residue. The residue was dissolved in a mixture of methanol-dichloromethane (1:1; 10 mL) and Celite (1 g) was added. The mixture was concentrated under reduced pressure to obtain a solid plug. Purification using 1%-15% methanol (containing 0.1% ammonium hydroxide) in dichloromethane to obtain 6-amino-7-(3-hydroxy-2,6-dimethylphenyl)-2-methyl-7H-pyrrolo[2,3-d]pyrimidine-5-carboxamide (63 mg, 0.20 mmol, 72%, 99% Purity) as a white solid. LCMS: RT=0.49 min, m/z 312.2 (M+H)⁺. ¹H NMR (300 MHz, DMSO) δ 9.58 (s, 1H), 8.86 (s, 1H), 7.06 (d, J=8.7 Hz, 3H), 6.92 (d, J=8.2 Hz, 1H), 6.83 (s, 2H), 2.42 (s, 3H), 1.73 (s, 3H), 1.65 (s, 3H).

### Example 47. Synthesis of Compound 600

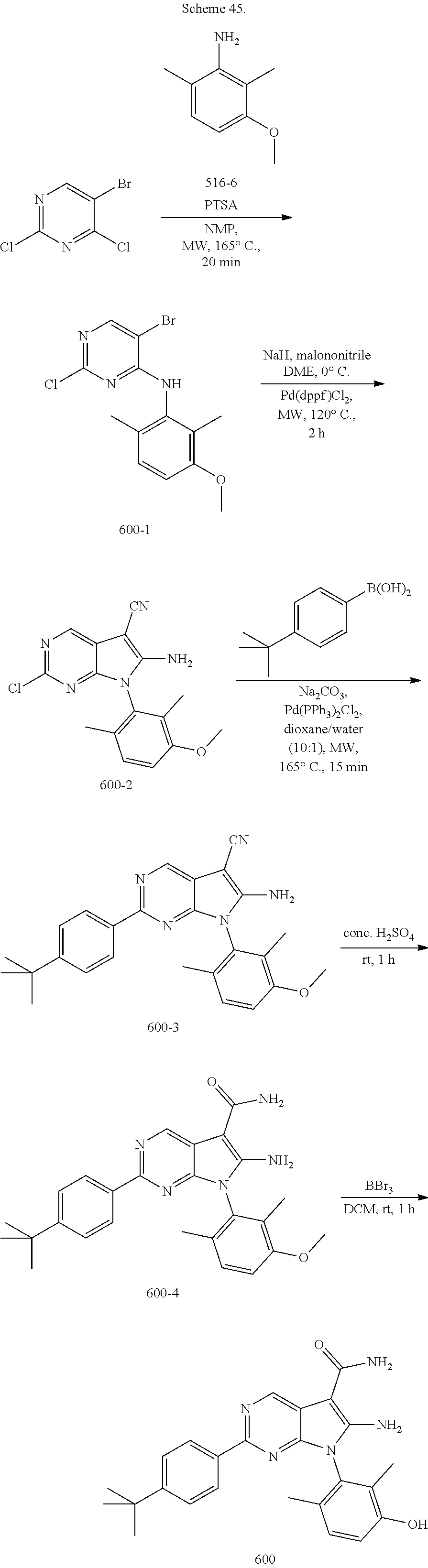

### Step 1: Synthesis of 5-bromo-2-(trifluoromethyl)pyrimidin-4(3H)-one

To a round bottom flask was taken 5-bromo-2,4-dichloropyrimidine (2.57 g, 1 eq, 11.3 mmol), 3-methoxy-2,6-dimethylaniline (1.71 g, 1.00 eq, 11.3 mmol) followed by THF (15 mL). The reaction mixture was cooled to 0° C. To the cold solution was slowly added lithium bis(trimethylsilyl)amide solution in THF (2.26 g, 13.5 mL, 1 molar, 1.2 eq, 13.5 mmol). The reaction mixture was stirred at 0° C. for 1.5 hours. It was then quenched with saturated ammonium chloride solution (1 mL), followed by the addition of saturated sodium bicarbonate solution (50 mL). The product was extracted with ethyl acetate (50 mL×3). The combined organic extracts were washed with water (50 mL), brine (25 mL), and dried (sodium sulfate). After filtering the drying agent, the filtrate was concentrated under reduced pressure. Purification using ISCO RediSep Rf Silica 40 g column. Eluted with 5%-25% ethyl acetate in hexanes to obtain 5-bromo-2-chloro-N-(3-methoxy-2,6-dimethylphenyl)pyrimidin-4-amine (2.4 g, 7.0 mmol, 62%) as a light brown solid. LCMS: RT=1.179 min, m/z 342.1; 344.0 (M+H)⁺.

### Step 2: Synthesis of 6-amino-2-chloro-7-(3-methoxy-2,6-dimethylphenyl)-7H-pyrrolo[2,3-d]pyrimidine-5-carbonitrile

To a 20 mL microwave vial with a rubber septum was taken malononitrile (0.66 g, 2 eq, 9.9 mmol) and anhydrous, degassed 1,2-dichloroethane (18 mL) under nitrogen. The solution was cooled to 0° C. and sodium hydride (0.40 g, 60% wt, 2 eq, 9.9 mmol) was added. The mixture was stirred for 15 min, then it was connected to vacuum and degassed for 2 min. After this, 5-bromo-2-chloro-N-(3-methoxy-2,6-dimethylphenyl)pyrimidin-4-amine (1.7 g, 1 eq, 5.0 mmol) and [1,1′-Bis(diphenylphosphino)ferrocene]dichloropalladium(II), complex with dichloromethane (0.20 g, 0.05 eq, 0.25 mmol) were added under nitrogen. The rubber septum was replaced with a microwave (Teflon) cap under positive pressure of nitrogen and the vial was sealed. The reaction mixture was subjected to microwave heating at 110° C. for 4 hours. The reaction mixture was filtered through a pad of Celite and washed with ethyl acetate (30 mL). The filtrate was concentrated and Celite (10 g) was added. The slurry was concentrated to obtain a plug. Purification using ISCO RediSep Rf Gold Silica 24 g column, eluting with 0%-5% methanol in dichloromethane to obtain 6-amino-2-chloro-7-(3-methoxy-2,6-dimethylphenyl)-7H-pyrrolo[2,3-d]pyrimidine-5-carbonitrile (1.1 g, 3.4 mmol, 68%) as a light yellow solid. LCMS: RT=1.039 min, m/z 328.2 (M+H)⁺.

### Step 3: Synthesis of 6-amino-2-(4-(tert-butyl)phenyl)-7-(3-methoxy-2,6-dimethylphenyl)-7H-pyrrolo[2,3-d]pyrimidine-5-carbonitrile

To a 5 mL microwave vial with a stir bar was taken 6-amino-2-chloro-7-(3-methoxy-2,6-dimethylphenyl)-7H-pyrrolo[2,3-d]pyrimidine-5-carbonitrile (100 mg, 1 eq, 305 μmol), (4-(tert-butyl)phenyl)boronic acid (81.5 mg, 1.5 eq, 458 μmol) and sodium carbonate (64.7 mg, 2 eq, 610 μmol) under nitrogen. The vial was capped with a rubber septum and evacuated under vacuum. To the vial was added 1,4-dioxane (3 mL) and water (0.3 mL) and the mixture was degassed for 2 min. To the vial under positive pressure of nitrogen was added dichlorobis(triphenylphosphine)palladium (21.4 mg, 0.1 eq, 30.5 mol) and the vial was sealed with a microwave crimp cap. The reaction mixture was heated at 165° C. for 15 min under microwave irradiation using Biotage Initiator⁺ reactor. The reaction material was filtered through a pad of Celite and washed with ethyl acetate (10 mL). The filtrated was concentrated to obtain a residue. Purification using ISCO, Redicep 12 g (gold) column; eluting with 0%-5% methanol in dichloromethane to obtain 6-amino-2-(4-(tert-butyl)phenyl)-7-(3-methoxy-2,6-dimethylphenyl)-7H-pyrrolo[2,3-d]pyrimidine-5-carbonitrile (120 mg, 282 μmol, 92.4%) as a light yellow solid. LC-MS: RT=1.394 min, m/z 426.3 (M+H)⁺. ¹H NMR (400 MHz, CDCl₃) δ 8.77 (d, J=0.9 Hz, 1H), 8.17-8.11 (m, 2H), 7.35 (d, J=8.4 Hz, 2H), 7.18 (d, J=8.4 Hz, 1H), 6.94 (d, J=8.4 Hz, 1H), 4.80 (s, 2H), 3.85 (s, 3H), 1.88 (s, 3H), 1.81 (s, 3H), 1.25 (d, J=0.9 Hz, 10H), 1.21 (s, 1H), 0.91 (d, J=6.6 Hz, 1H), 0.85-0.75 (m, 3H).

### Step 4: Synthesis of 6-amino-2-(4-(tert-butyl)phenyl)-7-(3-methoxy-2,6-dimethylphenyl)-7H-pyrrolo[2,3-d]pyrimidine-5-carboxamide

To a 10 mL round bottom flask was taken 6-amino-2-(4-(tert-butyl)phenyl)-7-(3-methoxy-2,6-dimethylphenyl)-7H-pyrrolo[2,3-d]pyrimidine-5-carbonitrile (110 mg, 1 eq, 258 μmol) and concentrated sulfuric acid (2 mL), and the reaction mixture was stirred at room temperature for 1 hr. After this, crushed ice (approx. 50 gm) was added to the reaction mixture, followed by dropwise addition of saturated sodium bicarbonate solution (10 mL) followed by the dropwise addition of 2M aqueous NaOH maintaining the temperature below 5° C. and with stirring to pH 8. The product was then extracted with ethyl acetate (15 mL×3). The combined organic extracts were washed with brine (15 mL), dried (sodium sulfate), and concentrated under reduced pressure to obtain 6-amino-2-(4-(tert-butyl)phenyl)-7-(3-methoxy-2,6-dimethylphenyl)-7H-pyrrolo[2,3-d]pyrimidine-5-carboxamide (100 mg, 225 μmol, 87.2%) as an off white solid. LC-MS: RT=1.173 min, m/z 444.3 (M+H)⁺. ¹H NMR (400 MHz, CDCl₃) δ 8.68 (s, 1H), 8.14 (d, J=8.2 Hz, 2H), 7.35 (d, J=8.2 Hz, 2H), 7.17 (d, J=8.4 Hz, 1H), 6.93 (d, J=8.4 Hz, 1H), 6.07 (s, 1H), 5.39 (s, 2H), 3.85 (s, 3H), 1.89 (s, 3H), 1.82 (s, 3H), 1.26 (d, J=0.9 Hz, 9H).

### Step 5: Synthesis of 6-amino-2-(4-(tert-butyl)phenyl)-7-(3-hydroxy-2,6-dimethylphenyl)-7H-pyrrolo[2,3-d]pyrimidine-5-carboxamide (Compound 600)

To a solution of 6-amino-2-(4-(tert-butyl)phenyl)-7-(3-methoxy-2,6-dimethylphenyl)-7H-pyrrolo[2,3-d]pyrimidine-5-carboxamide (100 mg, 1 eq, 225 μmol) in anhydrous dichloromethane (5 mL) was added a solution of tribromoborane (169 mg, 676 μL, 1M, 3 eq, 676 μmol) at room temperature. The reaction was continued to stir at room temperature for 1 hour. After this, the reaction mixture was concentrated under reduced pressure. To the residue was added methanol (2 mL) and the solution was concentrated (2×). To the residue was added triethylamine (1 mL) and methanol (1 mL) and the mixture was concentrated under reduced pressure. The crude residue was dissolved in DMSO (2 mL) and purified using ACCQPrpe. 0%-90% acetonitrile (0.1% formic acid (v/v)) in water (0.1% formic acid (v/v)) over 15 min. Pure fractions were concentrated under reduced pressure and then lyophilized to obtain 6-amino-2-(4-(tert-butyl)phenyl)-7-(3-hydroxy-2,6-dimethylphenyl)-7H-pyrrolo[2,3-d]pyrimidine-5-carboxamide (85 mg, 0.20 mmol, 88%) as a white solid. LC-MS: R_{T}=1.016 min, m/z 430.3 (M+H)⁺. ¹H NMR (400 MHz, DMSO) δ 9.54 (s, 1H), 8.98 (s, 1H), 7.97 (d, J=8.3 Hz, 2H), 7.35 (d, J=8.1 Hz, 2H), 7.13 (s, 2H), 7.05 (d, J=8.3 Hz, 1H), 6.90 (d, J=8.3 Hz, 1H), 6.86 (s, 2H), 1.73 (s, 3H), 1.65 (s, 3H), 1.20 (s, 9H).

### Example 48. Synthesis of Compound 608

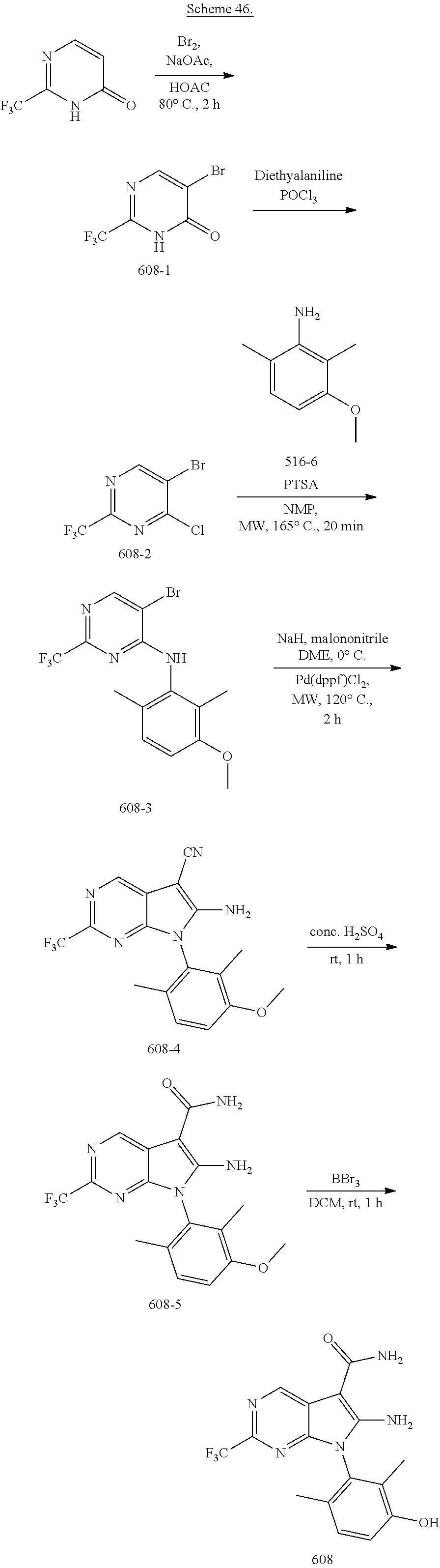

### Step 1: Synthesis of 5-bromo-2-(trifluoromethyl)pyrimidin-4(3H)-one

Bromine (1.60 g, 1 eq, 9.99 mmol) was added to a solution of 2-(trifluoromethyl)pyrimidin-4(3H)-one (1.64 g, 1 eq, 9.99 mmol) and potassium acetate (2.94 g, 3 Eq, 30.0 mmol) in acetic acid (15 mL). The mixture was heated to 80° C. for 2 hour and was then cooled and concentrated. Water was added to the concentrate and the reaction was extracted with ethyl acetate (25 mL×3). The combined organic layers were dried over anhydrous sodium sulfate, filtered, and concentrated to give 5-bromo-2-(trifluoromethyl)pyrimidin-4(3H)-one (2.1 g) as a white solid. LCMS: RT=0.687 min, m/z 243.0, 245.0 (M+H).

### Step 2: Synthesis of 5-bromo-4-chloro-2-(trifluoromethyl)pyrimidine

To a suspension of 5-bromo-2-(trifluoromethyl)pyrimidin-4(3H)-one (1.1 g, 1 eq, 4.5 mmol) in phosphoryl trichloride (17 g, 11 mL, 25 eq, 0.11 mol) was added N,N-diethylaniline (68 mg, 72 μL, 0.1 eq, 0.45 mmol). The mixture was heated to 120° C. for 2 hours. After cooling the reaction mixture to room temperature, it was carefully concentrated (approximately 5% volume) under reduced pressure. The residue was poured onto crushed ice. The mixture was then slowly neutralized with cold saturated sodium bicarbonate solution. The product was extracted with ethyl acetate (25 mL×3). The combined organic extracts were dried (sodium sulfate) and concentrated under reduced pressure to obtain 5-bromo-4-chloro-2-(trifluoromethyl)pyrimidine (0.5 g, 2 mmol, 40%) as a light brown colored residue which was immediately used in the next step without further purification.

### Step 3: Synthesis of 5-bromo-N-(3-methoxy-2,6-dimethylphenyl)-2-(trifluoromethyl)pyrimidin-4-amine

To a 20 mL microwave vial was taken 5-bromo-4-chloro-2-(trifluoromethyl)pyrimidine (450 mg, 1 eq, 1.72 mmol) and 3-methoxy-2,6-dimethylaniline (260 mg, 1 eq, 1.72 mmol) and p-toluenesulfonicacidmonohydrate (32.7 mg, 26.4 μL, 0.1 eq, 172 μmol), followed by NMP (15 mL). The vial was capped and was heated at 165° C. for 20 min under microwave irradiation (Biotage Initiator+). The reaction mixture was poured in saturated sodium bicarbonate solution (25 mL). The product was extracted with ethyl acetate (25 mL×3). The combined organic extracts were washed with water (25 mL), brine (25 mL), and dried (sodium sulfate). After filtering the drying agent, the filtrate was concentrated under reduced pressure. Purification using ISCO RediSep Rf Silica 12 g. Eluted with 5%-25% ethyl acetate in hexanes to obtain 5-bromo-N-(3-methoxy-2,6-dimethylphenyl)-2-(trifluoromethyl)pyrimidin-4-amine (360 mg, 957 μmol, 55.6%) as a light brown solid. LCMS: RT=1.256 min, m/z 376.3, 378.1 (M+H).⁺

### Step 4: Synthesis of 6-amino-7-(3-methoxy-2,6-dimethylphenyl)-2-(trifluoromethyl)-7H-pyrrolo[2,3-d]pyrimidine-5-carbonitrile

To a 20 mL microwave vial with a rubber septum was taken malononitrile (61.5 mg, 2 eq, 930 mol) and anhydrous, degassed 1,2-dimethoxyethane (15 mL) under nitrogen. The solution was cooled to 0° C. and sodium hydride (37.2 mg, 60% Wt, 2 eq, 930 μmol) was added. The mixture was stirred for 15 min, then it was connected to vacuum and degassed for 2 min. After this, 5-bromo-N-(3-methoxy-2,6-dimethylphenyl)-2-(trifluoromethyl)pyrimidin-4-amine (175 mg, 1 eq, 465 μmol) and [1,1′-Bis(diphenylphosphino)ferrocene]dichloropalladium(II) complex in dichloromethane (38.0 mg, 0.1 eq, 46.5 μmol) were added under nitrogen. The rubber septum was replaced with a microwave (Teflon) cap under positive pressure of nitrogen and the vial was sealed. The reaction mixture was subjected to microwave heating at 120° C. for 2 hours. The reaction mixture was filtered through a pad of Celite and washed with ethyl acetate (30 mL). The filtrate was concentrated and Celite (1 g) was added. The slurry was concentrated to obtain a plug. Purification using ISCO RediSep Rf Gold Silica 12 g column, eluting with 0%-5% methanol in dichloromethane to obtain 6-amino-7-(3-methoxy-2,6-dimethylphenyl)-2-(trifluoromethyl)-7H-pyrrolo[2,3-d]pyrimidine-5-carbonitrile (80 mg, 0.22 mmol, 48%) as an off-white solid. LCMS: RT=1.116 min, m/z 362.2 (M+H)⁺; ¹H NMR (500 MHz, CDCl₃) δ 8.87 (s, 1H), 7.27 (d, J=8.4 Hz, 1H), 7.04 (d, J=8.5 Hz, 1H), 3.92 (s, 3H), 1.93 (d, J=0.7 Hz, 3H), 1.86 (s, 3H).

### Step 5: Synthesis of 6-amino-7-(3-methoxy-2,6-dimethylphenyl)-2-(trifluoromethyl)-7H-pyrrolo[2,3-d]pyrimidine-5-carboxamide

To a 10 mL round bottom flask was taken 6-amino-7-(3-methoxy-2,6-dimethylphenyl)-2-(trifluoromethyl)-7H-pyrrolo[2,3-d]pyrimidine-5-carbonitrile (77 mg, 1 eq, 0.21 mmol) and concentrated sulfuric acid (2 mL), and the reaction mixture was stirred at room temperature for 1 hour. After this, crushed ice (approx. 50 gm) was added, followed by dropwise addition of saturated sodium bicarbonate solution (10 mL) followed by the dropwise addition of 2M aqueous NaOH while maintaining the temperature below 5° C. and with stirring to pH 8. The product was extracted with ethyl acetate (15 mL×3). The combined organic extracts were washed with brine (15 mL), dried (sodium sulfate), and concentrated under reduced pressure to obtain 6-amino-7-(3-methoxy-2,6-dimethylphenyl)-2-(trifluoromethyl)-7H-pyrrolo[2,3-d]pyrimidine-5-carboxamide (80.5 mg, 212 μmol, 100%) as an off white solid. LC-MS: RT=1.019 min, m/z 380.2 (M+H)⁺; ¹H NMR (500 MHz, CDCl₃) δ 8.67 (s, 1H), 7.16 (d, J=8.5 Hz, 1H), 6.93 (d, J=8.4 Hz, 1H), 3.83 (s, 3H), 1.84 (d, J=0.7 Hz, 3H), 1.77 (s, 3H). Used in the next step without further purification.

### Step 6: Synthesis of 6-amino-7-(3-hydroxy-2,6-dimethylphenyl)-2-(trifluoromethyl)-7H-pyrrolo[2,3-d]pyrimidine-5-carboxamide (Compound-608)

To a solution of 6-amino-7-(3-methoxy-2,6-dimethylphenyl)-2-(trifluoromethyl)-7H-pyrrolo[2,3-d]pyrimidine-5-carboxamide (77 mg, 1 eq, 0.20 mmol) in anhydrous dichloromethane (3 mL) was added a solution of boron tribromide solution in dichloromethane (0.15 g, 0.61 mL, 1M, 3 eq, 0.61 mmol) at room temperature for 1 hour. After this, the reaction mixture was concentrated under reduced pressure. To the residue was added methanol (2 mL) and the solution was concentrated (2×). To the residue was added triethylamine (1 mL) and methanol (1 mL) and the mixture was concentrated under reduced pressure. The crude residue was dissolved in DMSO (2 mL) and purified using ACCQPrep. 0%-90% acetonitrile (0.1% formic acid (v/v)) in water (0.1% formic acid (v/v) over 15 min. Pure fractions were concentrated under reduced pressure and then lyophilized to obtain 6-amino-7-(3-hydroxy-2,6-dimethylphenyl)-2-(trifluoromethyl)-7H-pyrrolo[2,3-d]pyrimidine-5-carboxamide as an off-white solid. LC-MS: RT=0.871 min, m/z 366.2 (M+H)⁺; ¹H NMR (500 MHz, DMSO) δ 9.69 (s, 1H), 9.13 (s, 1H), 7.50 (s, 2H), 7.13 (d, J=8.5 Hz, 3H), 6.98 (d, J=8.4 Hz, 1H), 2.55 (s, 4H), 1.76 (s, 3H), 1.68 (s, 3H).

### Example 49. Synthesis of Compound 609

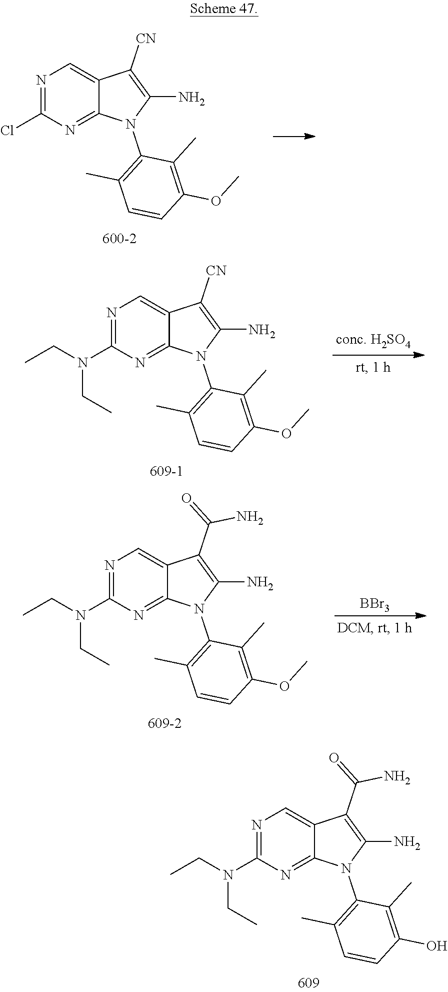

### Step 1: Synthesis of 6-amino-2-(diethylamino)-7-(3-methoxy-2,6-dimethylphenyl)-7H-pyrrolo[2,3-d]pyrimidine-5-carbonitrile

To a 5 mL microwave vial with stir bar was taken 6-amino-2-chloro-7-(3-methoxy-2,6-dimethylphenyl)-7H-pyrrolo[2,3-d]pyrimidine-5-carbonitrile (90 mg, 1 eq, 0.27 mmol) followed by the addition of DMF (4.5 mL) and diethylamine (0.20 g, 0.28 mL, 10 eq, 2.7 mmol). The vial was sealed and was heated at 180° C. for 30 min under microwave irradiation. After this, the mixture was poured in water (50 mL). The product was extracted with ethyl acetate (50 mL×2). The combined organic extracts were washed with water (50 mL×3), brine (25 mL), dried sodium sulfate, and concentrated under reduced pressure. Purification using ISCO RediSep Rf Gold Silica 12 g eluting with 0-30% ethyl acetate in hexanes to obtain 6-amino-2-(diethylamino)-7-(3-methoxy-2,6-dimethylphenyl)-7H-pyrrolo[2,3-d]pyrimidine-5-carbonitrile (63 mg, 0.17 mmol, 63%) as a light brown solid. LC-MS: RT=0.855 min, m/z 365.3 (M+H)+; ¹H NMR (500 MHz, CDCl₃) δ 8.40 (s, 1H), 7.20 (d, J=8.4 Hz, 1H), 6.96 (d, J=8.4 Hz, 1H), 4.45 (s, 2H), 3.91 (s, 3H), 3.59-3.41 (m, J=6.9 Hz, 4H), 1.97 (d, J=0.7 Hz, 3H), 1.90 (s, 3H), 1.09 (t, J=7.0 Hz, 6H).

### Step 2: Synthesis of 6-amino-2-(diethylamino)-7-(3-methoxy-2,6-dimethylphenyl)-7H-pyrrolo[2,3-d]pyrimidine-5-carboxamide

6-Amino-2-(diethylamino)-7-(3-methoxy-2,6-dimethylphenyl)-7H-pyrrolo[2,3-d]pyrimidine-5-carbonitrile (60 mg, 1 eq, 0.16 mmol) was stirred with concentrated sulfuric acid (2 mL) at room temperature for 1 hour. After this, to the reaction mixture was carefully added crushed ice (approx. 50 gm), followed by dropwise addition of saturated sodium bicarbonate solution (10 mL) with stirring followed by the dropwise addition of 2M aqueous NaOH maintaining the temperature below 5° C. and with stirring to pH 8. The product was then extracted with ethyl acetate (15 mL×3). The combined organic extracts were washed with brine (15 mL), dried (sodium sulfate), and concentrated under reduced pressure to obtain 6-amino-2-(diethylamino)-7-(3-methoxy-2,6-dimethylphenyl)-7H-pyrrolo[2,3-d]pyrimidine-5-carboxamide (62.5 mg, 163 μmol, 99%) as an off white solid. LC-MS: RT −0.782 min, m/z 383.3 (M+H)⁺; ¹H NMR (500 MHz, CDCl₃) δ 8.25 (s, 1H), 7.10 (d, J=8.4 Hz, 1H), 6.86 (d, J=8.5 Hz, 1H), 5.76 (s, 2H), 5.21 (s, 2H), 3.81 (s, 3H), 3.43 (p, J=7.0 Hz, 4H), 1.88 (s, 3H), 1.82 (s, 3H), 1.01 (t, J=7.0 Hz, 6H). Used in the next step without further purification.

### Step 3: Synthesis of 6-amino-2-(diethylamino)-7-(3-hydroxy-2,6-dimethylphenyl)-7H-pyrrolo[2,3-d]pyrimidine-5-carboxamide (Compound 609)

To a solution of 6-amino-2-(diethylamino)-7-(3-methoxy-2,6-dimethylphenyl)-7H-pyrrolo[2,3-d]pyrimidine-5-carboxamide (60 mg, 1 eq, 0.16 mmol) in anhydrous dichloromethane (3 mL) was added a solution of boron tribromide solution in dichloromethane (0.12 g, 0.47 mL, 1M, 3 eq, 0.47 mmol) at room temperature for 1 hour. After this, the reaction mixture was concentrated under reduced pressure. To the residue was added methanol (2 mL) and the solution was concentrated (2×). To the residue was added triethylamine (1 mL) and methanol (1 mL) and the mixture was concentrated under reduced pressure. The residue was dissolved in DMSO (2 mL) and purified using ACCQPrep. 0%-70% acetonitrile (0.1% formic acid (v/v)) in water (0.1% formic acid (v/v) over 15 min. LC-MS: RT=0.671 min, m/z 369.3 (M+H)⁺; ¹H NMR (500 MHz, DMSO) δ 9.48 (s, 1H), 8.58 (s, 1H), 7.06 (d, J=8.2 Hz, 1H), 6.89 (d, J=8.3 Hz, 1H), 6.64 (d, J=31.5 Hz, 4H), 3.41 (qt, J=9.3, 4.7 Hz, 4H), 1.81 (s, 3H), 1.73 (s, 3H), 1.00 (t, J=7.0 Hz, 6H).

### Example 50. Synthesis of Compound 618

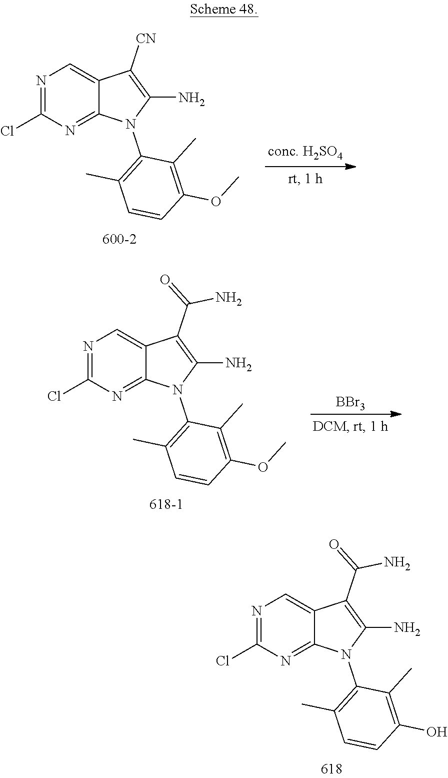 [SMILES: COc1ccc(C)c(-n2c(N)c(C#N)c3cnc(Cl)nc32)c1C ; COc1ccc(C)c(-n2c(N)c(C(N)=O)c3cnc(Cl)nc32)c1C ; "BBr3" ; Cc1ccc(O)c(C)c1-n1c(N)c(C(N)=O)c2cnc(Cl)nc21]

### Step 1: Synthesis of 6-amino-2-chloro-7-(3-methoxy-2,6-dimethylphenyl)-7H-pyrrolo[2,3-d]pyrimidine-5-carboxamide

A solution of 6-amino-2-chloro-7-(3-methoxy-2,6-dimethylphenyl)-7H-pyrrolo[2,3-d]pyrimidine-5-carbonitrile (75 mg, 1 eq, 0.23 mmol) in concentrated sulfuric acid (2 mL) was stirred at room temperature for 1 hour. To the mixture was added crushed ice (approx. 25 g). The mixture was carefully neutralized using saturated sodium bicarbonate solution. The product was extracted with ethyl acetate (25 mL×3). The combined organic extracts were dried (sodium sulfate) and concentrated under reduced pressure to obtain 6-amino-2-chloro-7-(3-methoxy-2,6-dimethylphenyl)-7H-pyrrolo[2,3-d]pyrimidine-5-carboxamide (76 mg, 0.22 mmol, 96%) as an off-white solid, which was used in the next step without further purification. LC-MS: RT=0.925 min, m/z 346.1 (M+H)⁺; ¹H NMR (500 MHz, CDCl₃) δ 8.55 (s, 1H), 7.24 (d, J=8.4 Hz, 1H), 6.99 (d, J=8.5 Hz, 1H), 6.19 (s, 2H), 5.48 (s, 2H), 3.91 (s, 3H), 1.95 (s, 3H), 1.87 (s, 3H).

### Step 2: Synthesis of 6-amino-2-chloro-7-(3-hydroxy-2,6-dimethylphenyl)-7H-pyrrolo[2,3-d]pyrimidine-5-carboxamide (Compound 618)

To a solution of 6-amino-2-chloro-7-(3-methoxy-2,6-dimethylphenyl)-7H-pyrrolo[2,3-d]pyrimidine-5-carboxamide (73 mg, 1 eq, 0.21 mmol) in dichloromethane (4 mL) was added tribromoborane solution in DCM (0.16 g, 0.63 mL, 1 molar, 3 eq, 0.63 mmol) at room temperature. A precipitate formed. This was stirred at room temperature for 1 hour. The mixture was then concentrated under reduced pressure. To the residue was added a mixture of dichloromethane and methanol (1:1; 10 mL). The mixture was concentrated under reduced pressure, followed by the addition of triethylamine and methanol. The reaction was concentrated under reduced pressure to obtain a residue. The crude residue was dissolved in DMSO (2 mL) and purified using ACCQPrep. 0%-70% acetonitrile (0.1% formic acid (v/v)) in water (0.1% formic acid (v/v) over 14 min. Pure fractions were concentrated under reduced pressure and then lyophilized to obtain 6-amino-2-chloro-7-(3-hydroxy-2,6-dimethylphenyl)-7H-pyrrolo[2,3-d]pyrimidine-5-carboxamide (58.5 mg, 176 μmol, 84%) as a white solid. LC-MS: RT=0.766 min, m/z 332.2 (M+H)⁺. ¹H NMR (500 MHz, DMSO) δ 9.67 (s, 1H), 8.90 (s, 1H), 7.32 (s, 2H), 7.11 (d, J=8.3 Hz, 1H), 7.04-6.94 (m, 2H), 1.77 (s, 3H), 1.69 (s, 2H).

### Example 51. Synthesis of Compound 663

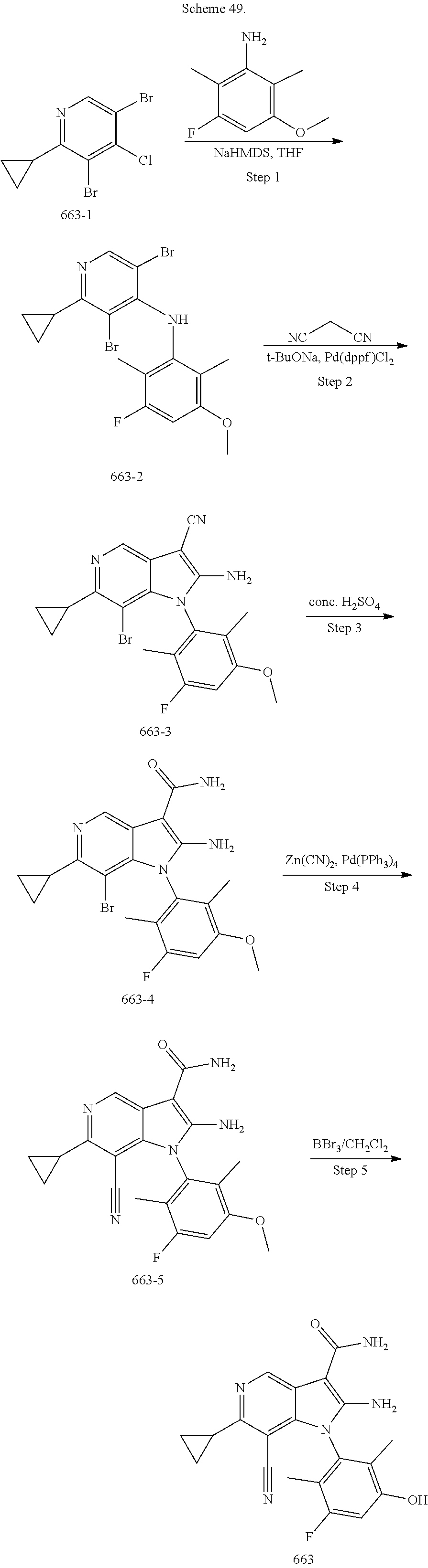

### Step 1: Synthesis of 3,5-dibromo-2-cyclopropyl-N-(3-fluoro-5-methoxy-2,6-dimethyl-phenyl)pyridin-4-amine

To a solution of 3,5-dibromo-4-chloro-2-cyclopropyl-pyridine (1.5 g, 4.82 mmol,) and 3-fluoro-5-methoxy-2,6-dimethyl-aniline (815 mg, 4.82 mmol) in tetrahydrofuran (15 mL) was added NaHMDS (1M solution in THF, 9.6 mL) dropwise at 0° C. under a nitrogen atmosphere. The reaction mixture was stirring at 20° C. for 2 hours, then quenched with saturated ammonium chloride solution and extracted with ethyl acetate (20 mL*3). The combined extracts were washed with brine (20 mL), dried over sodium sulfate, filtered, and concentrated under reduced pressure. The residue was purified by Biotage® combi flash (Column: 20 g Biotage® Silica Flash column; Eluent: gradient 0˜3% ethyl acetate in petroleum ether; Gradient time: 16 min; Hold time: 20 min; Flow rate: 45 mL/min). Pure fraction was concentrated under reduced pressure to afford the title compound (1.93 g, 72.17% yield) as a yellow oil. LCMS R_{T}=1.693 min, m/z=444.8 [M+H]⁺. ¹H NMR (400 MHz, DMSO-d₆) δ=8.14 (s, 1H), 7.70 (d, J=4.0 Hz, 1H), 6.82 (d, J=11.6 Hz, 1H), 3.77 (s, 3H), 2.50-2.35 (m, 1H), 1.90 (s, 3H), 1.84 (s, 3H), 1.04-0.86 (m, 4H).

### Step 2: Synthesis of 2-amino-7-bromo-6-cyclopropyl-1-(3-fluoro-5-methoxy-2,6-dimethyl-phenyl)pyrrolo[3,2-c]pyridine-3-carbonitrile

To a solution of propanedinitrile (574 mg, 8.69 mmol) in 1,2-dimethoxyethane (30 mL) was added sodium tert-butoxide (1.04 g, 10.86 mmol) at 0° C. and the mixture was stirring at 20° C. for 30 mins under a nitrogen atmosphere, followed by addition of 3,5-dibromo-2-cyclopropyl-N-(3-fluoro-5-methoxy-2,6-dimethyl-phenyl)pyridin-4-amine (1.93 g, 4.35 mmol) and Pd(dppf)Cl₂ (317 mg, 0.43 mmol). The reaction mixture was stirred at 100° C. under a nitrogen atmosphere for 16 hours. After cooling, the mixture was concentrated under reduced pressure. The residue was purified by Biotage @combi flash (Column: 20 g Biotage® Silica Flash column; Eluent: gradient 0˜23% ethyl acetate in petroleum ether; Gradient time: 30 min; Hold time: 15 min; Flow rate: 45 mL/min). Pure fractions were concentrated under reduced pressure to afford the title compound (940 mg, 47.87% yield) as a yellow solid. LCMS R_{T}=1.265 min, m/z=430.5 [M+H]⁺. ¹H NMR (400 MHz, DMSO-d₆) δ=8.29 (s, 1H), 7.15 (d, J=11.6 Hz, 1H), 7.08 (br s, 2H), 3.85 (s, 3H), 1.71 (s, 3H), 1.66 (s, 3H), 1.19-1.15 (m, 1H), 0.90-0.88 (m, 4H).

### Step 3: Synthesis of 2-amino-7-bromo-6-cyclopropyl-1-(3-fluoro-5-methoxy-2,6-dimethyl-phenyl)pyrrolo[3,2-c]pyridine-3-carboxamide

A solution of 2-amino-7-bromo-6-cyclopropyl-1-(3-fluoro-5-methoxy-2,6-dimethyl-phenyl)pyrrolo[3,2-c]pyridine-3-carbonitrile (920 mg, 2.14 mmol) in sulfuric acid (8.0 mL) was stirred at 20° C. for 30 mins, then adjusted to pH=7 with sodium carbonate and extracted with dichloromethane (30 mL*3). The combined extracts were washed with brine (50 mL), dried over sodium sulfate, filtered, and concentrated under reduced pressure. The residue was purified by Biotage® combi flash (Column: 20 g Biotage® Silica Flash column; Eluent: gradient 0˜5% methanol in dichloromethane; Gradient time: 16 min; Hold time: 15 min; Flow rate: 45 mL/min). Pure fractions were combined and concentrated under reduced pressure to afford the title compound (459 mg, 39.26% yield) as a white solid. LCMS R_{T}=1.085 min, m/z=448.9 [M+H]⁺. ¹H NMR (400 MHz, DMSO-d₆) δ=8.73 (s, 1H), 7.15 (d, J=11.6 Hz, 1H), 6.97-6.77 (m, 4H), 3.86 (s, 3H), 2.48-2.40 (m, 1H), 1.71 (s, 3H), 1.66 (s, 3H), 0.90-0.87 (m, 4H).

### Step 4: Synthesis of 2-amino-7-cyano-6-cyclopropyl-1-(3-fluoro-5-methoxy-2,6-dimethyl-phenyl)pyrrolo[3,2-c]pyridine-3-carboxamide

To a solution of 2-amino-7-bromo-6-cyclopropyl-1-(3-fluoro-5-methoxy-2,6-dimethyl-phenyl)pyrrolo[3,2-c]pyridine-3-carboxamide (259 mg, 0.58 mmol) in N-methyl-2-pyrrolidone (8.0 mL) were added Zn(CN)₂ (81 mg, 0.70 mmol) and Pd(PPh₃)₄ (67 mg, 0.058 mmol) under a nitrogen atmosphere. The reaction mixture was placed in a vessel, purged with nitrogen, sealed and heated to 160° C. under microwave, and then stirred for 30 minutes. After cooling to room temperature, the reaction mixture was diluted with water (50 mL) and filtered. The filter cake was washed with water (20 mL) and re-dissolved in dichloromethane (30 mL). The organic phase was dried over anhydrous sodium sulfate, filtered, and concentrated under reduced pressure. The residue was purified by Biotage® combi flash (Column: 10 g Biotage® Silica Flash column; Eluent: gradient 10˜69% ethyl acetate in petroleum ether; Gradient time: 50 min; Hold time: 20 min; Flow rate: 45 mL/min). Pure fractions were concentrated under reduced pressure to afford the title compound (161 mg, 54.42% yield) as a yellow solid. LCMS R_{T}=1.161 min, m/z=393.7 [M+H]⁺. ¹H NMR (400 MHz, DMSO-d₆) δ=8.96 (s, 1H), 7.20 (d, J=11.6 Hz, 1H), 7.10 (d, J=8.8 Hz, 2H), 6.93 (s, 2H), 3.87 (s, 3H), 2.29-2.23 (m, 1H), 1.74 (s, 3H), 1.69 (s, 3H), 1.00 (d, J=6.0 Hz, 4H).

### Step 5: Synthesis of 2-amino-7-cyano-6-cyclopropyl-1-(3-fluoro-5-hydroxy-2,6-dimethyl-phenyl)pyrrolo[3,2-c]pyridine-3-carboxamide (Compound 663)

2-Amino-7-cyano-6-cyclopropyl-1-(3-fluoro-5-methoxy-2,6-dimethyl-phenyl)pyrrolo [3,2-c]pyridine-3-carboxamide (161 mg, 0.41 mmol) was dissolved in dichloromethane (3.0 mL) and treated with tribromoborane (1.03 g, 4.09 mmol) at 0° C. The reaction mixture was stirred at 20° C. for 1.5 hours, and was then quenched with MeOH under a nitrogen atmosphere and concentrated under reduced pressure. The residue was purified by RP-HPLC [Column: Welch Xtimate C18 150*40 mm*10 μm; Mobile phase: [water (formic acid)-MeCN]; Gradient: 6-46% of B over 25 min], and then further purified by chiral SFC [Column: Daicel Chiralcel OJ (250 mm*30 mm, 10 μm); Mobile phase: [CO₂-EtOH (0.1% NH₃H₂O)]; B %: 40%, isocratic elution mode] to afford the title compound (Compound 663, 41.7 mg) as a white solid. LCMS R_{T}=2.965 min, m/z=380.1 [M+H]⁺. ¹H NMR (400 MHz, DMSO-d₆) δ=10.15 (br s, 1H), 8.94 (s, 1H), 7.09 (br s, 2H), 6.95 (br s, 2H), 6.86 (d, J=11.2 Hz, 1H), 2.29-2.23 (m, 1H), 1.70 (s, 3H), 1.65 (s, 3H), 1.00 (d, J=6.4 Hz, 4H). ¹⁹F NMR (400 MHz, DMSO-d₆) δ=−118.002.

### Example 52. Synthesis of Compound 667

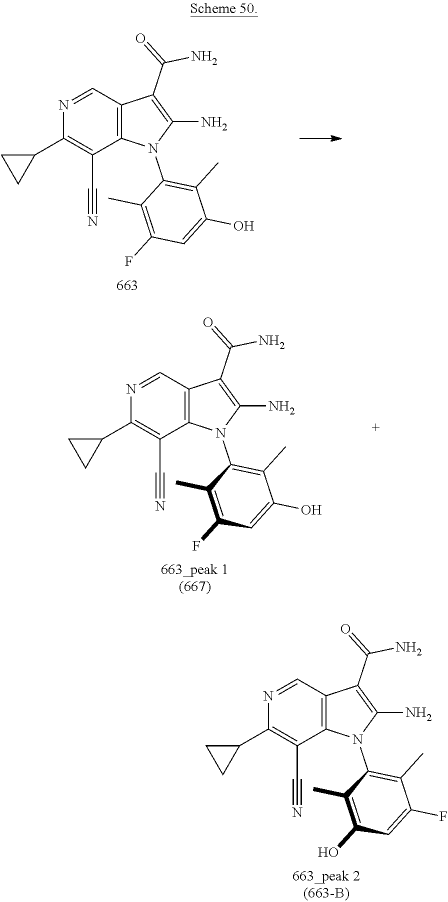

### 2-amino-7-cyano-6-cyclopropyl-1-(3-fluoro-5-hydroxy-2,6-dimethyl-phenyl)pyrrolo[3,2-c]pyridine-3-carboxamide Resolved Isomers

Racemic 2-amino-7-cyano-6-cyclopropyl-1-(3-fluoro-5-hydroxy-2,6-dimethyl-phenyl)pyrrolo[3,2-c]pyridine-3-carboxamide (170 mg, 0.448 mmol) was separated by chiral SFC [Column: Daicel Chiralcel OX (250 mm*30 mm, 10 μm); Mobile phase: [CO₂-EtOH (0.1% NH₃H₂O)]; B %: 55%, isocratic elution mode].

Fraction 1 (t₁=1.107 min) was obtained as a white solid (663_peak 1, also known as Compound 667, 63.5 mg). LCMS R_{T}=0.935 min, m/z=380.0 [M+H]⁺. ¹H NMR (400 MHz, DMSO-d₆) δ=10.11 (br s, 1H), 8.95 (s, 1H), 7.09 (br s, 2H), 6.95 (br s, 2H), 6.85 (d, J=10.8 Hz, 1H), 2.29-2.23 (m, 1H), 1.70 (s, 3H), 1.65 (s, 3H), 1.00 (d, J=6.4 Hz, 4H). ¹⁹F NMR (400 MHz, DMSO-d₆) δ=−118.023.

Fraction 2 (t₂=2.177 min) was obtained as a white solid (663_peak 2, 60.9 mg). LCMS R_{T}=0.916 min, m/z=380.0 [M+H]⁺. ¹H NMR (400 MHz, DMSO-d₆) δ=10.13 (br s, 1H), 8.95 (s, 1H), 7.09 (br s, 2H), 6.95 (br s, 2H), 6.85 (d, J=11.2 Hz, 1H), 2.29-2.23 (m, 1H), 1.70 (s, 3H), 1.65 (s, 3H), 1.00 (d, J=6.4 Hz, 4H). ¹⁹F NMR (400 MHz, DMSO-d₆) δ=−118.018.

### Example 53. Synthesis of Compound 673

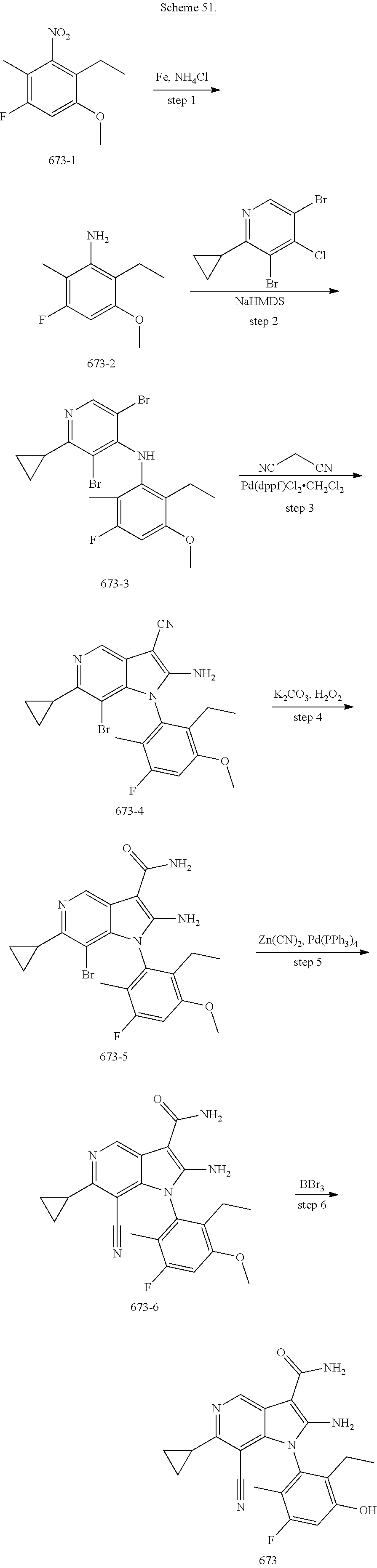

### Step 1: Synthesis of 2-ethyl-5-fluoro-3-methoxy-6-methyl-aniline

To a solution of 4-ethyl-1-fluoro-5-methoxy-2-methyl-3-nitro-benzene (4.0 g, 18.53 mmol) in methanol (40 mL) and water (10 mL) was added ammonium chloride (5.0 g, 92.63 mmol) and iron powder (5.2 g, 92.63 mmol). The reaction mixture was stirred at 80° C. for 12 hours, then cooled and filtered. The filter cake was washed with methanol (100 mL) and the filtrate was concentrated under reduced pressure to remove volatiles. The residue was purified by column chromatography (silica gel, 100-200 mesh, gradient 0-7% ethyl acetate in petroleum ether) to afford the title compound (2.9 g, 85% yield) as a yellow oil. LCMS R_{T}=0.576 min, m/z=184.1 [M+H]⁺.

### Step 2: Synthesis of 3,5-dibromo-2-cyclopropyl-N-(2-ethyl-5-fluoro-3-methoxy-6-methyl-phenyl)pyridin-4-amine

To a solution of 2-ethyl-5-fluoro-3-methoxy-6-methyl-aniline (500 mg, 2.73 mmol) and 3,5-dibromo-4-chloro-2-cyclopropyl-pyridine (850 mg, 2.73 mmol) in THF (5.0 mL) was added dropwise NaHMDS (1M solution in THF, 5.5 mL) at 0° C. The reaction mixture was stirred at 25° C. for 12 hours, then quenched with saturated ammonium chloride solution (20 mL) and extracted with ethyl acetate (15 mL*3). The combined extracts were washed with brine (25 mL), dried over sodium sulfate, filtered, and concentrated. The residue was purified by column chromatography (silica gel, 100-200 mesh, gradient 0-4% ethyl acetate in petroleum ether) to afford the title compound (450 mg, 36% yield) as a yellow oil. LCMS R_{T}=0.798 min, m/z=458.9 [M+H]⁺.

### Step 3: Synthesis of 2-amino-7-bromo-6-cyclopropyl-1-(2-ethyl-5-fluoro-3-methoxy-6-methyl-phenyl)pyrrolo[3,2-c]pyridine-3-carbonitrile

A solution of propanedinitrile (361 mg, 5.47 mmol) in 1,2-dimethoxyethane (18 mL) and tert-butanol (6.0 mL) was treated with sodium tert-butanol (700 mg, 7.29 mmol) and stirred at 0° C. for 30 mins, then 3,5-dibromo-2-cyclopropyl-N-(2-ethyl-5-fluoro-3-methoxy-6-methyl-phenyl)pyridin-4-amine (1.67 g, 3.64 mmol, 1.0 eq) and Pd(dppf)Cl₂·CH₂Cl₂ (298 mg, 0.365 mmol) were added. The reaction was heated to 110° C. under a nitrogen atmosphere and stirred for 12 hours. After cooling, the mixture was diluted with water (20 mL) and extracted with ethyl acetate (20 mL*3). The combined extracts were washed with brine (40 mL), dried over sodium sulfate, filtered, and concentrated. The residue was purified by column chromatography (silica gel, 100-200 mesh, gradient 0-45% ethyl acetate in petroleum ether) to afford the title compound (628 mg, 33% yield) as a yellow solid. LCMS R_{T}=0.551 min, m/z=444.9 [M+H]⁺. ¹H NMR (400 MHz, DMSO-d₆) δ=8.32 (s, 1H), 7.19 (br s, 2H), 7.16 (d, J=11.6 Hz, 1H), 3.78 (s, 3H), 2.48-2.39 (m, 1H), 2.17-2.08 (m, 2H), 1.71 (s, 3H), 0.98-0.93 (m, 4H), 0.92-0.86 (m, 3H).

### Step 4: Synthesis of 2-amino-7-bromo-6-cyclopropyl-1-(2-ethyl-5-fluoro-3-methoxy-6-methyl-phenyl)pyrrolo[3,2-c]pyridine-3-carboxamide

To a solution of 2-amino-7-bromo-6-cyclopropyl-1-(2-ethyl-5-fluoro-3-methoxy-6-methyl-phenyl)pyrrolo[3,2-c]pyridine-3-carbonitrile (628 mg, 1.42 mmol) in dimethyl sulfoxide (5.0 mL) were added potassium carbonate (1.96 g, 14.17 mmol) and hydrogen peroxide (3.21 g, 30% aqueous, 28.33 mmol). The reaction mixture was stirred at 25° C. for 12 hours, then quenched with saturated sodium sulfite solution (20 mL) and extracted with dichloromethane (20 mL*3). The combined extracts were washed with water (30 mL) and brine (30 mL), dried over anhydrous sodium sulfate, filtered, and concentrated under reduced pressure. The residue was purified by column chromatography (silica gel, 100-200 mesh, gradient 0-57% ethyl acetate in petroleum ether) to afford the title compound (232 mg, 29.1% yield) as a yellow solid. LCMS R_{T}=0.559 min, m/z=463.2 [M+H]⁺.

### Step 5: Synthesis of 2-amino-7-cyano-6-cyclopropyl-1-(2-ethyl-5-fluoro-3-methoxy-6-methyl-phenyl)pyrrolo[3,2-c]pyridine-3-carboxamide

To a solution of 2-amino-7-bromo-6-cyclopropyl-1-(2-ethyl-5-fluoro-3-methoxy-6-methyl-phenyl)pyrrolo[3,2-c]pyridine-3-carboxamide (50 mg, 0.108 mmol) in N-methylpyrrolidone (2.5 mL) were added zinc cyanide (15.3 mg, 0.130 mmol) and Pd(PPh₃)₄ (12.5 mg, 10.84 μmol). The mixture was placed in a vessel and purged with nitrogen. The vessel was sealed, heated to 160° C. on a microwave oven and stirred for 20 minutes. After cooling to room temperature, the reaction mixture was poured into water (50 mL) and filtered. The filter cake was washed water (30 mL) and then re-dissolved in dichloromethane (60 mL). The organic phase was dried over anhydrous sodium sulfate, filtered, and concentrated under reduced pressure. The residue was purified by column chromatography (silica gel, 100-200 mesh, gradient 0-55% ethyl acetate in petroleum ether) to afford the title compound (152 mg, 68.8% yield) as a white solid. LCMS R_{T}=0.583 min, m/z=408.2.[M+H]⁺.

### Step 6: Synthesis of 2-amino-7-cyano-6-cyclopropyl-1-(2-ethyl-5-fluoro-3-hydroxy-6-methyl-phenyl)pyrrolo[3,2-c]pyridine-3-carboxamide (Compound 673)

A solution of 2-amino-7-cyano-6-cyclopropyl-1-(2-ethyl-5-fluoro-3-methoxy-6-methyl-phenyl)pyrrolo[3,2-c]pyridine-3-carboxamide (152 mg, 0.373 mmol) in dichloromethane (2.5 mL) was treated with BBr₃ (2M solution in dichloromethane, 0.935 mL) at 0° C. and then stirred at 20° C. for 4 hours. After being quenched with water (50 mL) and filtered, the filter cake was washed with dichloromethane (30 mL). The organic filtrate was dried over anhydrous sodium sulfate, filtered, and concentrated under reduced pressure. The residue was concentrated and purified by RP-HPLC [Gradient 8-48% of MeCN in water (0.225% formic acid)] to afford the title compound (47.7 mg, 32% yield) as a white solid. LCMS R_{T}=0.972 min, m/z=394.2 [M+H]⁺. ¹H NMR (400 MHz, DMSO-d₆) δ=10.21 (br s, 1H), 8.94 (s, 1H), 7.10 (br s, 2H), 6.95 (br s, 2H), 6.88 (d, J=11.2 Hz, 1H), 2.28-2.22 (m, 1H), 2.16-2.07 (m, 2H), 1.67 (s, 3H), 1.00 (d, J=6.4 Hz, 4H), 0.85 (t, J=7.2 Hz, 3H). ¹⁹F NMR (400 MHz, DMSO-d₆) δ=−117.631.

### Example 54. Synthesis of Compound 685

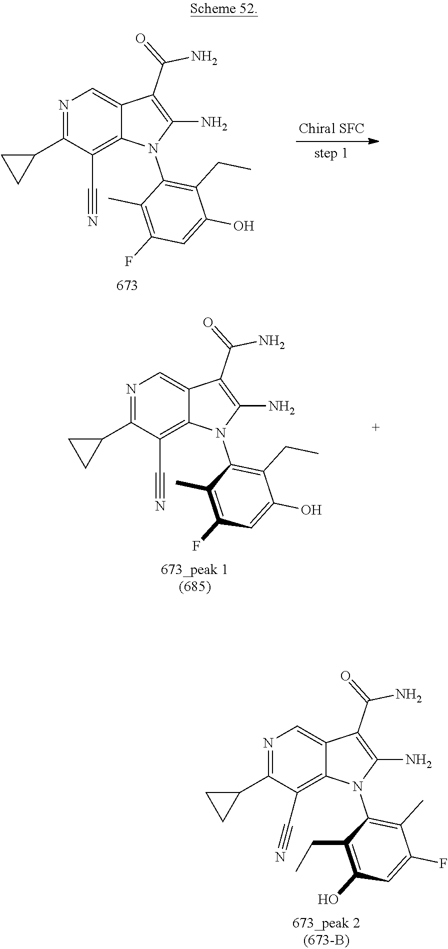

### 2-amino-7-cyano-6-cyclopropyl-1-(2-ethyl-5-fluoro-3-hydroxy-6-methyl-phenyl)pyrrolo[3,2-c]pyridine-3-carboxamide Resolved Isomers

Racemic 2-amino-7-cyano-6-cyclopropyl-1-(2-ethyl-5-fluoro-3-hydroxy-6-methyl-phenyl)pyrrolo[3,2-c]pyridine-3-carboxamide (45 mg, 0.114 mmol) was separated by chiral SFC [Column: Daicel Chiralcel OX (250 mm*30 mm, 10 μm); Mobile phase: [CO₂-EtOH (0.1% NH₃H₂O)]; B %: 55%, isocratic elution mode].

Fraction 1 (t₁=0.988 min) was obtained as a white solid (673_peak 1, also known as Compound 685, 6.3 mg). LCMS R_{T}=0.960 min, m/z=394.0 [M+H]⁺. ¹H NMR (400 MHz, CD₃OD) δ=8.81 (s, 1H), 6.82 (d, J=11.2 Hz, 1H), 2.40-2.32 (m, 1H), 2.30-2.21 (m, 2H), 1.67 (s, 3H), 1.13-1.07 (m, 2H), 1.06-0.99 (m, 2H), 0.97 (t, J=7.6 Hz, 3H). ¹⁹F NMR (400 MHz, DMSO-d₆) δ=−117.581.

Fraction 2 (t₂=1.804 min) was obtained as a white solid (673_peak 2, 11.6 mg). LCMS R_{T}=0.953 min, m/z=394.0 [M+H]⁺. ¹H NMR (400 MHz, CD₃OD) δ=8.81 (s, 1H), 6.82 (d, J=10.8 Hz, 1H), 2.42-2.33 (m, 1H), 2.29-2.19 (m, 2H), 1.67 (s, 3H), 1.12-1.06 (m, 2H), 1.06-1.00 (m, 2H), 0.97 (t, J=7.6 Hz, 3H). ¹⁹F NMR (400 MHz, DMSO-d₆) δ=−117.581.

Compounds of the disclosure can be made by the representative methods described herein in Examples 1-54.

### Biological Examples

### Example B-1: In Vitro Assay

Compounds that bind the kinase active site and directly (sterically) or indirectly (allosterically) prevent kinase binding to an immobilized ligand and reduce the amount of kinase captured on the solid support. Screening “hits” were identified by measuring the amount of kinase captured in test versus control samples by using a quantitative, precise and ultra-sensitive qPCR method that detect the associated DNA label. In a similar manner, dissociation constants (Kd's) for test compound-kinase interactions were calculated by measuring the amount of kinase captured on the solid support as a function of the test compound concentration.

Representative biochemical data is presented in Table 2, where A is <50 nM, B≥50≤100, C>100<1000, and D>1000.

TABLE 2
Representative in vitro data

| Compound ID | PKMYT1 (IC₅₀ = nM) | LCK (IC₅₀ = nM) |
|---|---|---|
| 540 | D | — |
| 542 | D | C |
| 543 | A | C |
| 544 | D | C |
| 546 | D | — |
| 547 | A | B |
| 548 | C | — |
| 550 | C | — |
| 552 | C | — |
| 553 | C | — |
| 555 | A | — |
| 556 | A | A |
| 559 | A | A |
| 560 | A | C |
| 561 | B | C |
| 565 | A | A |
| 566 | A | C |
| 567 | A | — |
| 568 | A | — |
| 571 | C | — |
| 572 | A | — |
| 573 | A | — |
| 574 | C | — |
| 575 | A | A |
| 576 | C | C |
| 577 | A | A |
| 578 | D | D |
| 579 | A | — |
| 580 | A | — |
| 581 | A | — |
| 582 | A | — |
| 583 | A | — |
| 584 | C | — |
| 585 | D | — |
| 586 | C | — |
| 587 | A | — |
| 588 | A | — |
| 591 | B | — |
| 592 | B | C |
| 593 | A | C |
| 594 | A | — |
| 595 | A | — |
| 596 | A | — |
| 599 | A | A |
| 600 | C | C |
| 602 | A | C |
| 603 | A | A |
| 604 | A | A |
| 605 | A | A |
| 606 | A | A |
| 607 | D | D |
| 608 | A | — |
| 609 | A | — |
| 616 | A | — |
| 617 | A | — |
| 618 | A | — |
| 619 | A | — |
| 620 | A | — |
| 621 | A | — |
| 622 | B | — |
| 623 | A | — |
| 624 | A | — |
| 625 | A | — |
| 630 | A | A |
| 631 | A | A |
| 632 | B | A |
| 633 | A | B |
| 634 | A | B |
| 635 | A | A |
| 638 | A | A |
| 639 | A | A |
| 640 | A | B |
| 641 | A | B |
| 642 | A | A |
| 647 | A | A |
| 648 | B | A |
| 649 | A | A |
| 650 | B | B |
| 651 | A | A |
| 652 | A | A |
| 653 | A | A |
| 655 | A | A |
| 656 | A | A |
| 659 | A | A |
| 660 | A | A |
| 661 | A | A |
| 662 | A | A |
| 663 | A | A |
| 665 | D | D |
| 666 | D | D |
| 667 | A | A |
| 668 | D | A |
| 669 | D | C |
| 670 | C | C |
| 672 | D | A |
| 673 | A | A |
| 675 | D | C |
| 676 | B | B |
| 678 | D | D |
| 680 | D | D |
| 681 | C | A |
| 682 | D | — |
| 683 | C | — |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (V), or a pharmaceutically acceptable salt thereof: 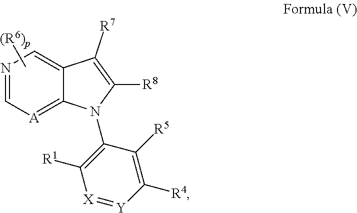
wherein;
A is N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; or
two R⁶ on adjacent atoms join together to form a 6-membered carbocyclic or heterocyclic ring;
R⁷ and R⁸ are each independently C₁-C₃ haloalkyl, —N(R^{b})₂, —C(═O)R^{a}, —C(═O)OR^{b}, or —C(═O)N(R^{b})₂;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1, 2, or 3.

2. The compound of claim 1, or a pharmaceutically acceptable salt thereof, wherein R⁷ is —C(═O)NH₂; and R⁸ is —NH₂.

3. The compound of claim 1, wherein the compound is of Formula (VI), or a pharmaceutically acceptable salt thereof: 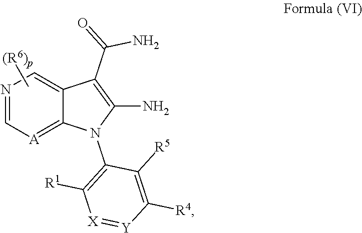
wherein;
A is N or CR⁶;
X is N or CR²;
Y is N or CR³;
R¹ and R⁵ are each independently halogen, C₁-C₆ alkyl, or C₁-C₆ haloalkyl;
R², R³, and R⁴ are each independently hydrogen, halogen, —OH, or —OR^{a}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

4. The compound of any one of claims 1-3, or a pharmaceutically acceptable salt thereof, wherein R¹ and R⁴ are each independently C₁-C₆ alkyl.

5. The compound of claim 4, or a pharmaceutically acceptable salt thereof, wherein R¹ is methyl and R⁴ is ethyl; or R⁴ is ethyl and R¹ is methyl.

6. The compound of claim 4, or a pharmaceutically acceptable salt thereof, wherein R¹ is methyl and R⁵ is methyl.

7. The compound of claim 3, wherein the compound of Formula (VI) has the structure of Formula (VIa), or a pharmaceutically acceptable salt thereof:

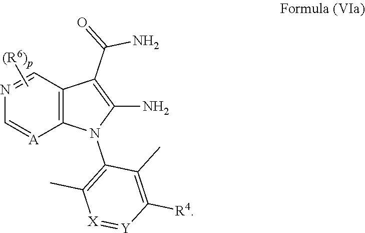

8. The compound of any one of claims 1-7, or a pharmaceutically acceptable salt thereof, wherein Y is CR³.

9. The compound of any one of claims 1-8, or a pharmaceutically acceptable salt thereof, wherein X is CR².

10. The compound of any one of claims 1-8, or a pharmaceutically acceptable salt thereof, wherein X is N.

11. The compound of any one of claims 1-9, or a pharmaceutically acceptable salt thereof, wherein one of R⁴ or R² is —OH.

12. The compound of any one of claims 1-9, or a pharmaceutically acceptable salt thereof, wherein R⁴ is OH.

13. The compound of claim 12, or a pharmaceutically acceptable salt thereof, wherein:
R² is halogen; and
R³ is hydrogen.

14. The compound of any one of claims 1-9, or a pharmaceutically acceptable salt thereof, wherein R² is —OH.

15. The compound of claim 14, or a pharmaceutically acceptable salt thereof, wherein:
R³ is hydrogen; and
R⁴ is halogen.

16. The compound of any one of claims 1-9, or a pharmaceutically acceptable salt thereof, wherein R⁴ and R⁵ together with the atoms to which they are attached form a 5 to 6 membered heteroaryl or 5 to 6 membered heterocycloalkyl.

17. The compound of any one of claims 1-9, or a pharmaceutically acceptable salt thereof, wherein R³ and R⁴ together with the atoms to which they are attached form a 5 to 6 membered heteroaryl or 5 to 6 membered heterocycloalkyl.

18. The compound of claim 1, wherein the compound is of Formula (VIIa), or a pharmaceutically acceptable salt thereof: 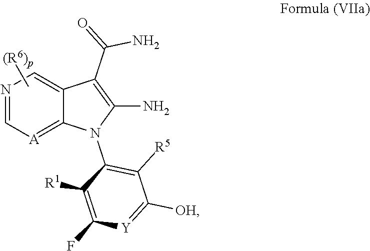
wherein,
A is N or CR⁶;
Y is N or CH;
R¹ and R⁵ are each independently halogen, C₁-C₆ alkyl, or C₁-C₆ haloalkyl;
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

19. The compound of claim 1, wherein the compound is of Formula (VIIb), or a pharmaceutically acceptable salt thereof: 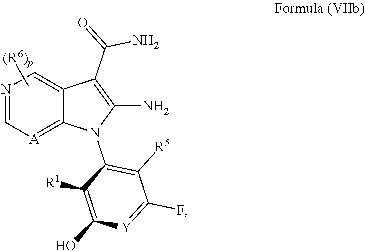
wherein;
A is N or CR⁶;
Y is N or CH;
R¹ and R⁵ are each independently halogen, C₁-C₆ alkyl, or C₁-C₆ haloalkyl;
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is 1 or 2.

20. The compound of claim 16 or 17, or a pharmaceutically acceptable salt thereof, wherein Y is CH.

21. The compound of any one of claims 1-20, or a pharmaceutically acceptable salt thereof, wherein A is N.

22. The compound of any one of claims 1-20, or a pharmaceutically acceptable salt thereof, wherein A is CR⁶.

23. The compound of any one of claims 1-22, or a pharmaceutically acceptable salt thereof, wherein each R⁶ is independently halogen, —CN, —OH, —OR^{a}, —N(R^{b})₂, —S(═O)₂R^{a}, —S(═O)₂N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}.

24. The compound of claim 23, or a pharmaceutically acceptable salt thereof, wherein each R⁶ is independently halogen, —CN, —OR^{a}, —N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl.

25. The compound of any one of claims 1-22, or a pharmaceutically acceptable salt thereof, wherein each R⁶ is independently —CH₃, —CH₂CH₃, —CN, —F, —Cl, —Br, —CH₂OH, —CH₂CH₂OH, —NH(CH₂)₂OH, cyclopropyl,

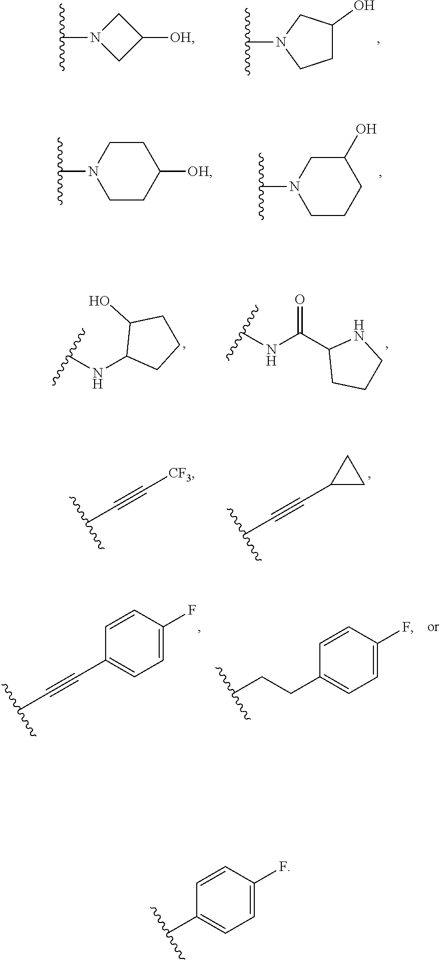

26. The compound of any one of claims 1-25, or a pharmaceutically acceptable salt thereof, wherein p is 1 or 2.

27. A compound of Formula (I), or a pharmaceutically acceptable salt thereof: 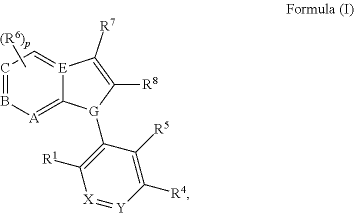
wherein;
A, B, and C are each independently N or CR⁶;
E and F are each independently N or C, wherein at least one of A, B, C, or E is N;
G is N or C;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; or
two R⁶ on adjacent atoms join together to form a 6-membered carbocyclic or heterocyclic ring;
R⁷ and Rare each independently —C₁-C₃ haloalkyl, —N(R^{b})₂, —C(═O)R^{a}, —C(═O)OR^{b}, or —C(═O)N(R^{b})₂;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-4.

28. The compound of claim 27, or a pharmaceutically acceptable salt thereof, wherein R⁷ is —C(═O)NH₂; and R⁸ is —NH₂.

29. The compound of claim 27 or 28, or a pharmaceutically acceptable salt thereof, wherein Y is N.

30. The compound of claim 27 or 28, or a pharmaceutically acceptable salt thereof, wherein Y is CR³.

31. The compound of claim 27, wherein the compound of Formula (I) has the structure of Formula (Ia), or a pharmaceutically acceptable salt thereof:

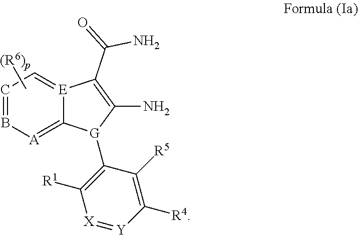

32. The compound of claim 31, or a pharmaceutically acceptable salt thereof, wherein E is C; and G is N.

33. The compound of claim 31 or 32, or a pharmaceutically acceptable salt thereof, wherein B is CR⁶.

34. The compound of claim 31 or 32, or a pharmaceutically acceptable salt thereof, wherein B is N.

35. The compound of claim 27, wherein the compound has the structure of Formula (II), or a pharmaceutically acceptable salt thereof: 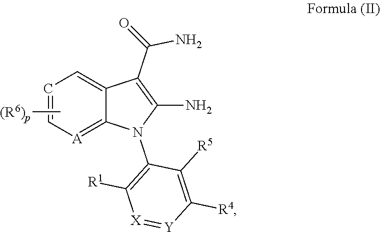
wherein;
A and C are each independently N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; or
two R⁶ on adjacent atoms join together to form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-4.

36. The compound of claim 35, or a pharmaceutically acceptable salt thereof, wherein if X is CH and R⁴ is OH; then R⁵ is not halogen or C₁-C₆ alkyl.

37. The compound of claim 35, or a pharmaceutically acceptable salt thereof, wherein if X is CH and R⁴ is OH; then R⁵ is —NH₂.

38. The compound of claim 35, or a pharmaceutically acceptable salt thereof, wherein R⁵ is hydrogen, —CN, —OH, —OR^{a}, —NO₂, —N(R^{b})₂, C₁-C₆haloalkyl, C₁-C₆hydroxyalkyl, C₁-C₆ heteroalkyl, C₃-C₆ cycloalkyl, or 4 to 6 membered heterocycloalkyl.

39. The compound of claim 38, or a pharmaceutically acceptable salt thereof, wherein R⁵ is —N(R^{b})₂.

40. The compound of claim 35, or a pharmaceutically acceptable salt thereof, wherein R⁴ is hydrogen, halogen, —CN, —OH, —OR^{a}, —NO₂, —N(R^{b})₂, C₁-C₆ alkyl, C₁-C₆ haloalkyl, C₁-C₆ hydroxyalkyl, or C₁-C₆heteroalkyl.

41. The compound of claim 40, or a pharmaceutically acceptable salt thereof, wherein R⁴ is —OH.

42. The compound of claim 35, or a pharmaceutically acceptable salt thereof, wherein R⁴ and R⁵ together with the atoms to which they are attached form a 5 to 6 membered heteroaryl or 5 to 6 membered heterocycloalkyl.

43. The compound of claim 35, or a pharmaceutically acceptable salt thereof, wherein R³ and R⁴ together with the atoms to which they are attached form a 5 to 6 membered heteroaryl or 5 to 6 membered heterocycloalkyl.

44. The compound of any one of claims 27-43, or a pharmaceutically acceptable salt thereof, wherein X is N.

45. The compound of any one of claims 37-43, or a pharmaceutically acceptable salt thereof, wherein X is CR².

46. The compound of claim 44 or 45, or a pharmaceutically acceptable salt thereof, wherein:
R¹ is C₁-C₆ alkyl, optionally substituted with one, two, three, or four substituents independently selected from R^{c};
R³ is hydrogen;
R⁴ is OH; and
R⁵ is —NH₂.

47. The compound of any one of claims 27-46, or a pharmaceutically acceptable salt thereof, wherein A is N.

48. The compound of any one of claims 27-46, or a pharmaceutically acceptable salt thereof, wherein A is CR⁶.

49. The compound of claim 27, wherein the compound of Formula (II) has the structure of Formula (IIa), or a pharmaceutically acceptable salt thereof:

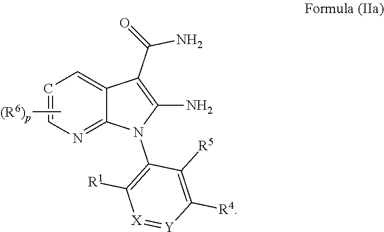

50. The compound of claim 27, or a pharmaceutically acceptable salt thereof, wherein E is N; and G is C.

51. The compound of claim 27, wherein the compound has the structure of Formula (III), or a pharmaceutically acceptable salt thereof: 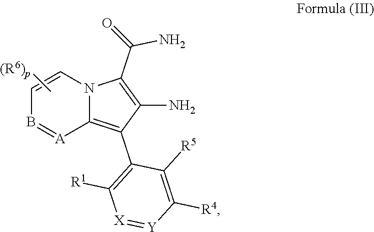
wherein;
A and B are each independently N or CR⁶;
X is N or CR²;
Y is N or CR³;
R¹, R², R³, R⁴, and R⁵ are each independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, C₁-C₆ alkyl, C₁-C₆ haloalkyl, C₁-C₆ hydroxyalkyl, C₁-C₆ heteroalkyl, C₃-C₆ cycloalkyl, or 4 to 6 membered heterocycloalkyl, each of which is optionally substituted with one, two, three or four substituents independently selected from R^{c}; or
R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c};
each R⁶ is independently hydrogen, halogen, —CN, —OH, —OR^{a}, —SH, —SR^{a}, —S(═O)R^{a}, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}; or
two R⁶ on adjacent atoms join together to form a 6-membered carbocyclic or heterocyclic ring;
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom to which they are attached to form a heterocycloalkyl, wherein the heterocycloalkyl is optionally substituted with one, two, three, or four substituents independently selected from R^{c};
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; and
p is an integer from 1-4.

52. The compound of claim 51, or a pharmaceutically acceptable salt thereof, wherein R⁴ is hydrogen, halogen, —CN, —OH, —OR^{a}, —NO₂, —N(R^{b})₂, C₁-C₆ alkyl, C₁-C₆ haloalkyl, C₁-C₆ hydroxyalkyl, or C₁-C₆ heteroalkyl.

53. The compound of claim 52, or a pharmaceutically acceptable salt thereof, wherein R⁴ is —OH.

54. The compound of any one of claims 51-53, or a pharmaceutically acceptable salt thereof, wherein R¹ and R⁵ are each independently halogen, —CN, —OH, —OR^{a}, —NO₂, —N(R^{b})₂, C₁-C₆ alkyl, C₁-C₆ haloalkyl, C₁-C₆ hydroxyalkyl, and C₁-C₆ heteroalkyl, each of which is optionally substituted with one, two, three, or four substituents independently selected from R^{c}.

55. The compound of any one of claims 51-54, or a pharmaceutically acceptable salt thereof, wherein:
R¹ and R⁵ are each independently halogen, —N(R^{b})₂, C₁-C₆ alkyl, C₁-C₆ haloalkyl, C₁-C₆ hydroxyalkyl, or C₁-C₆ heteroalkyl, each of which is optionally substituted with one, two, three or four substituents independently selected from R^{c}; and
R³ is hydrogen.

56. The compound of claim 51, or a pharmaceutically acceptable salt thereof, wherein R³ and R⁴ or R⁴ and R⁵ together with the atoms to which they are attached form a 5 to 6 membered heteroaryl or 5 to 6 membered heterocycloalkyl each of which is optionally substituted with one or two substituents independently selected from R^{c}.

57. The compound of any one of claims 51-56, or a pharmaceutically acceptable salt thereof, wherein X is N.

58. The compound of any one of claims 51-56, or a pharmaceutically acceptable salt thereof, wherein X is CR².

59. The compound of any one of claims 51-58, or a pharmaceutically acceptable salt thereof, wherein A is N.

60. The compound of claim 51, wherein the compound of Formula (III) has the structure of Formula (IIIa), or a pharmaceutically acceptable salt thereof:

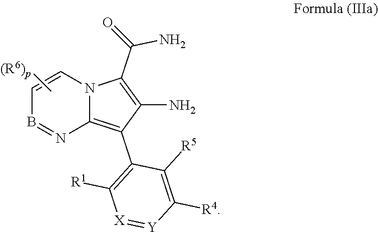

61. The compound of any one of claims 27-60, or a pharmaceutically acceptable salt thereof, wherein each R⁶ is independently halogen, —CN, —OH, —OR^{a}, —N(R^{b})₂, —S(═O)₂R^{a}, —S(═O)₂N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c}.

62. The compound of claim 61, or a pharmaceutically acceptable salt thereof, wherein each R⁶ is independently halogen, —CN, —OR^{a}, —N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl.

63. The compound of any one of claims 27-60, or a pharmaceutically acceptable salt thereof, wherein each R⁶ is independently —CH₃, —CH₂CH₃, —CN, —F, —Cl, —Br, —CH₂OH, —CH₂CH₂OH, —NH(CH₂)₂OH, cyclopropyl,

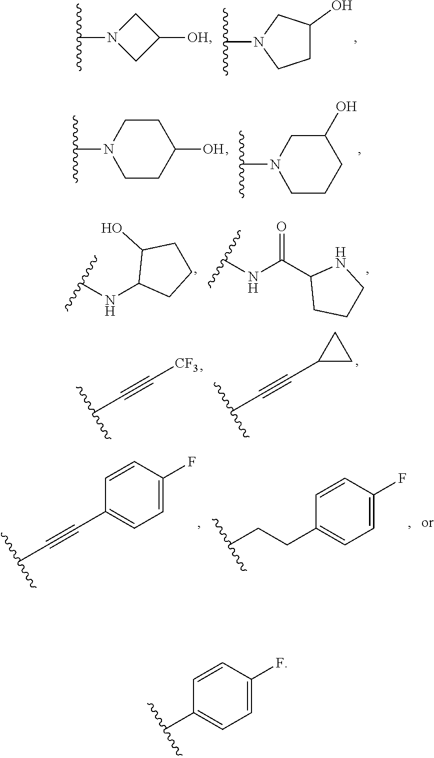

64. The compound of any one of claims 27-63, or a pharmaceutically acceptable salt thereof, wherein p is 1.

65. The compound of any one of claims 27-63, or a pharmaceutically acceptable salt thereof, wherein p is 2.

66. A compound of Formula (VIII), or a pharmaceutically acceptable salt thereof: 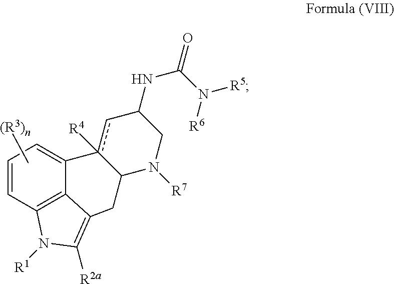
wherein;
A is N or CR^{6a};
X is N or CR²;
Y is N or CH;
R¹ and R⁵ are each independently halogen, C₁-C₆ alkyl, or C₁-C₆ haloalkyl;
R², R³, and R⁴ are each independently hydrogen, halogen, —OH, or —OR;
R^{6a} and R^{6b} are each independently hydrogen, halogen, —CN, —OH, —OR^{a}, —SH, —SR^{a}, —S(═O)W, —NO₂, —N(R^{b})₂, —S(═O)₂R^{a}, —S(═O)₂N(R^{b})₂, —C(═O)R^{a}, —C(═O)OR^{b}, —C(═O)N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₁-C₆ heteroalkyl, C₃-C₈ cycloalkyl, 4 to 8 membered heterocycloalkyl, or phenyl, each of which is optionally substituted with one, two, three, or four substituents independently selected from R^{c};
each R^{a} is independently C₁-C₆ alkyl, C₂-C₆ alkenyl, C₂-C₆ alkynyl, C₁-C₆ heteroalkyl, cycloalkyl, heterocycloalkyl, aryl, heteroaryl, —C₁-C₆ alkyl(aryl), —C₁-C₆ alkyl(heteroaryl), —C₁-C₆ alkyl(cycloalkyl), or —C₁-C₆ alkyl(heterocycloalkyl); wherein each alkyl, alkenyl, alkynyl, heteroalkyl, cycloalkyl, heterocycloalkyl, aryl, and heteroaryl is optionally substituted with one, two, three, or four substituents independently selected from R^{c};
each R^{b} is independently hydrogen, C₁-C₆ alkyl, C₂-C₆ alkenyl, C₂-C₆ alkynyl, C₁-C₆ heteroalkyl, cycloalkyl, heterocycloalkyl, aryl, or heteroaryl; wherein the alkyl, alkenyl, alkynyl, heteroalkyl, cycloalkyl, heterocycloalkyl, aryl, and heteroaryl is optionally substituted with one, two, three, or four substituents independently selected from R^{c};
or two R^{b} are taken together with the atom(s) to which they are attached to form a heterocycloalkyl, wherein the heterocycloalkyl is optionally substituted with one, two, three, or four substituents independently selected from R^{c}; and
each R^{c} is independently halogen, oxo, —CN, —OH, —NH₂, —NHCH₃, —N(CH₃)₂, —S(═O)CH₃, —S(═O)₂CH₃, —S(═O)₂NH₂, —S(═O)₂NHCH₃, —S(═O)₂N(CH₃)₂, —C(═O)CH₃, —C(═O)OH, —C(═O)OCH₃, —Si(CH₃)₃, C₁-C₆alkyl, C₁-C₆alkoxy, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl.

67. The compound of claim 66, or a pharmaceutically acceptable salt thereof, wherein R¹ and R⁵ are each independently C₁-C₆ alkyl.

68. The compound of claim 67, or a pharmaceutically acceptable salt thereof, wherein R¹ is methyl; and R⁵ is methyl or ethyl.

69. The compound of claim 67, or a pharmaceutically acceptable salt thereof, wherein R¹ is methyl or ethyl and R⁵ is methyl.

70. The compound of any one of claims 66-69, or a pharmaceutically acceptable salt thereof, wherein Y is CH.

71. The compound of any one of claims 66-70, or a pharmaceutically acceptable salt thereof, wherein X is CR².

72. The compound of any one of claims 66-71, or a pharmaceutically acceptable salt thereof, wherein R⁴ is —OH.

73. The compound of claim 72, or a pharmaceutically acceptable salt thereof, wherein R² is halogen.

74. The compound of any one of claims 66-71, or a pharmaceutically acceptable salt thereof, wherein R² is —OH.

75. The compound of claim 74, or a pharmaceutically acceptable salt thereof, wherein R² is halogen.

76. The compound of any one of claims 66-75, or a pharmaceutically acceptable salt thereof, wherein A is CR^{6a}.

77. The compound of any one of claims 66-75, or a pharmaceutically acceptable salt thereof, wherein A is N.

78. The compound of any one of claims 66-76, or a pharmaceutically acceptable salt thereof,
wherein
R^{6a} is hydrogen, halogen, —CN, C₁-C₆ alkyl, C₂-C₆ alkenyl, or C₂-C₆ alkynyl; and
R^{6b} is halogen, —CN, —OR^{a}, —N(R^{b})₂, —NHC(═O)R^{a}, C₁-C₆ alkyl, C₂-C₆ alkenyl, C₂-C₆ alkynyl, C₁-C₆ haloalkyl, C₁-C₆ hydroxyalkyl, C₃-C₆ cycloalkyl, 4 to 6 membered heterocycloalkyl, or phenyl, each of which is optionally substituted with one, two, three, or four substituents independently selected from R^{c}.

79. The compound of any one of claims 66-77, or a pharmaceutically acceptable salt thereof, wherein R^{6b} is —CH₃, —CH₂CH₃, —CN, —F, —Cl, —Br, —CH₂OH, —CH₂CH₂OH, —NH(CH₂)₂OH, cyclopropyl,

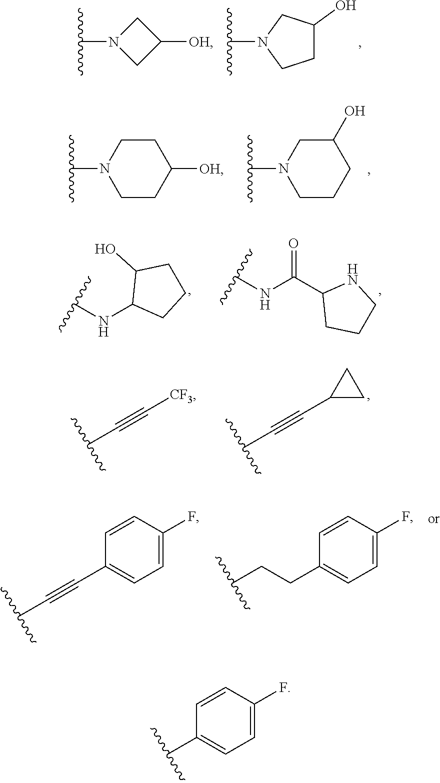

80. A compound selected from Table 1 or a pharmaceutically acceptable salt thereof.

81. A pharmaceutical composition comprising a compound of any one of claims 1-80, or a pharmaceutically acceptable salt thereof, and a pharmaceutically acceptable excipient.

82. A method of treating a disease or condition associated with inhibiting protein kinase, membrane associated tyrosine/threonine 1 (PKMYT1) in a patient in need thereof, comprising administering to the patient a compound of any one of claims 1-80, or a pharmaceutically acceptable salt thereof, or a pharmaceutical composition of claim 81.

83. The method of claim 82, wherein the condition associated with inhibition of PKMYT1 is a cancer.

84. A method of treating cancer in a patient in need thereof, comprising administering to the patient a compound of any one of claims 1-80, or a pharmaceutically acceptable salt thereof, or the pharmaceutical composition of claim 81.

85. The method of claim 84, wherein the cancer is leukemia, acute myeloid leukemia (AML), chronic myeloid leukemia, acute lymphoblastic leukemia (ALL), non-Hodgkin lymphoma (NHL), Hodgkin lymphoma (HL), or multiple myeloma (MM).

86. The method of claim 84, wherein the cancer is a solid cancer.

87. The method of claim 84, the cancer is a skin cancer, ocular cancer, gastrointestinal cancer, thyroid cancer, breast cancer, ovarian cancer, central nervous system cancer, laryngeal cancer, cervical cancer, lymphatic system cancer, genitourinary tract cancer, bone cancer, biliary tract cancer, endometrial cancer, liver cancer, lung cancer, prostate cancer, or colon cancer.
